(19) United States
(12) Patent Application Publication
(10) Pub. No.: US 2025/0177509 A1
(43) Pub. Date: Jun. 5, 2025

(54) RECOMBINANT ADENOVIRUS VACCINE FOR AFRICAN SWINE FEVER AND METHOD FOR CONSTRUCTING SAME

(71) Applicant: JIAXING ANYU BIOTECHNOLOGY CO., LTD, Jiaxing, Zhejiang (CN)

(72) Inventors: Ping CHEN, Jiaxing, Zhejiang (CN); Tingting ZHANG, Jiaxing, Zhejiang (CN); Na LI, Jiaxing, Zhejiang (CN); Xintao ZHONG, Jiaxing, Zhejiang (CN); Zhigang ZHU, Jiaxing, Zhejiang (CN); Nan LI, Jiaxing, Zhejiang (CN)

(21) Appl. No.: 18/014,874

(22) PCT Filed: Jul. 6, 2021
(86) PCT No.: PCT/CN2021/104793

§ 371 Date: Jun. 30, 2023

(30) Foreign Application Priority Data
Jul. 6, 2020 (CN) 202010642744.9
Jul. 6, 2020 (CN) 202010642745.3
Jul. 6, 2020 (CN) 202010642753.8
Jul. 6, 2020 (CN) 202010642754.2
Jul. 6, 2020 (CN) 202010642755.7

(51) Int. Cl. A61K 39/12; A61P 31/20; C12N 7/00; C12N 9/22; C12N 15/11; C12N 15/86; C12N 15/90

(52) U.S. Cl. CPC A61K 39/12; A61P 31/20; C12N 7/00; C12N 9/22; C12N 15/111; C12N 15/86; C12N 15/902; C12N 2310/20; C12N 2710/10022; C12N 2710/10034; C12N 2710/10043; C12N 2710/10052; C12N 2710/12034; C12N 2830/50

(57) ABSTRACT

An african swine fever virus vaccine includes five groups of antigens in total, and each group is respectively obtained by constructing recombinant adenovirus vectors co-expressing four antigen genes of african swine fever virus, and packaged by 293TD37 cells. The four antigenic genes of African swine fever virus in each group are 1, P72, B602L, P30 and P54; 2, CP129Rubiqutin, MGF5L6L, CP312R, and MGF110-4L; 3, L8Lubiqutin, I215L, I73RHBsAgHBsAg and E146L; 4, EP402R, EP153R, I177L, and K205Rubiqutin; 5, F317L, A151R, P34, and pp62. The construction of the recombinant adenovirus vector for co-expression of four antigen genes of the african swine fever virus mainly includes: knocking out E1, E3, E2a and E4 genes of the adenovirus vector by CRISPR/cas9 technology, constructing an ORF6/7 expression frame of E4 in an E2a region, and constructing shuttle plasmids in E1 and E4 regions for appropriately expressing four antigen genes, thereby obtaining a completely new adenovirus vector.

This application claims that benefit of priority from China's prior application no. 2020106427453, filed on Jul. 6, 2020; Priority to that prior application file in China, application no. 2020106427538, filed on Jul. 6, 2020; Priority to that prior application file in China, application no. 2021106145678, on Jul. 6, 2020; Priority is claim to that prior application of China, application no. 2020106427449, on Jul. 6, 2020; This application claim that benefit of priority from China's prior application no. 2020106427542, on Jul. 6, 2020; All of which form a part of the present invention.

### REFERENCE TO AN ELECTRONIC SEQUENCE LISTING

This application includes an electronically submitted sequence listing in .txt format. The .txt file contains a sequence listing entitled “0256_0195PUS1_ST25.txt” created on Jan. 3, 2025 and is 361,575 bytes in size.

The sequence listing contained in this .txt file is part of the specification and is hereby incorporated by reference herein in its entirety.

### TECHNICAL FIELD

The invention relates to the technical field of gene engineering and the field of immunology, in particular to a recombinant adenovirus vaccine of African swine fever virus and a construction method thereof.

### TECHNICAL BACKGROUND

African swine fever (ASF) is a highly infectious porcine virus disease. It can lead to a high mortality rate of close to 100% in domestic pigs. ASF is caused by African swine fever virus (ASFV). ASFV is a large double-stranded DNA virus that mainly replicates in the cytoplasm of macrophages. It has a 20-sided structure, with a diameter of 175-215 nm, and a genome length of 170-190 kb. It contains 151 open reading frames, encodes 150-200 proteins, and is a double-stranded linear DNA virus with a capsular sac. The structural proteins that constitute the ASFV virus particles include P30, P72, P49, P54, P220, P62, pB602L, CD2v protein, etc. Up to now, vaccines based on one or two subunits have failed to induce immunity strong enough to have a significant protective effect on the vaccinator.

ASF outbreak was discovered in China in 2018, which caused huge direct and indirect economic losses. Therefore, there is an urgent need to develop vaccines against ASFV. It has been reported that previous studies on ASFV vaccines mainly focused on inactivated vaccines and attenuated vaccines. However, inactivated vaccines do not induce an effective protective response; The biosafety of attenuated vaccine is the main limiting factor for its use, and attenuated strains are not allowed to be studied in China. However, in the absence of viable viral experiments at this stage, it is necessary to provide a vaccine to elicit an immune response against as many antigens as possible.

Therefore, new ASFV vaccines need to be developed. A potential candidate vaccine is a live vector vaccine. Compared with other vaccines, the live vector vaccine has the following advantages: (1) It can actively infect target tissues or cells, improving the efficiency of exogenous genes into the cells; (2) The vector itself has adjuvant effect, and can induce the production of cytokines and chemokines; (3) Most of them can induce long-term immune response. Advantageously, it is desirable to deliver as much pathogen protein as possible with as few live vectors as possible.

A live vector vaccine refers to the cloning of a protein-encoding gene of a pathogen into a live viral vector, which is then used to immunize an animal and express the protein in the animal to induce an immune response against the protein. As a vector for expressing african swine fever antigen protein, adenovirus type 5 has a plurality of advantages as follows: \textcircled{1} the adenovirus expression vector is replication defective and can only be produced and prepared in a unique complementary cell line, and the adenovirus does not integrate into a host cell genome, a target gene is expressed in a free state outside the host cell genome, the possibility of integrated mutation carcinogenesis is small, the gene toxicity is low, and the prepared vaccine is good in safety; \textcircled{2} the recombinant adenovirus vector can obtain higher titer, is beneficial to large-scale production, has high industrialized efficiency and low production cost; \textcircled{3} At present, the structure, characteristics and function of adenovirus type 5 have been studied in depth. The adenovirus vector is easy to copy and easy to operate, which is conducive to research; \textcircled{4} The common first-generation adenovirus vector genome knocks out 6K genes, can insert foreign genes 7.5 k, and has relatively large capacity; \textcircled{5} Adenovirus is relatively stable and can be prepared by purification, concentration and preservation.

Previous studies report some live vector vaccines. For example, ASFV P30, P54, P72 and pp62 genes are respectively recombined into a human adenovirus Ad5 vector for ‘cocktail’ immunity, and good antigen-specific CTL reaction is obtained; Then they recombined ASFV A151R, B119L, B602L, EP402R, EP153R, B438L and K205R-A104R into a replication-defective adenovirus vector, and after mixed immunity in a “cocktail” mode, they were able to induce strong humoral and cellular immune responses. However, for “cocktail” immunization, each ASFV antigen gene must be recombined into a replication-defective adenovirus vector, so a very large number of vectors are needed and there is a risk of immune response against adenovirus vector during the immune process. CN108504686A and CN108504687A provide recombinant adenovirus vectors expressing the EP153R and EP402R genes of ASFV, respectively. CN109652449A discloses a recombinant adenovirus vector for co-expression of two antigenic genes of EP153R and EP402R, and CN109735567A discloses a recombinant adenovirus vector for co-expression of two antigenic genes of EP153R and P54.

However, in order to further enhance the specific immune response to ASF, the antigenic gene capacity of the adenovirus vector needs to be further increased, and as much pathogen protein as possible needs to be delivered with as few live vectors as possible to elicit an immune response against as many antigens as possible.

CN110269932A discloses that 5-7 antigen genes of ASFV such as A104R, A151R, B119L, B602L, CD2v, K205R, P49 and the like are fused together based on an adenovirus vector for preparing a live vector vaccine. However, the fusion of multiple antigen genes has the risk of reducing immunogenicity and possibly leading to immune failure. Therefore, to improve the vaccine activity, it is necessary to express completely independent antigen genes in each adenovirus vector.

P30 protein of ASFV is an important structural protein encoded by CP204L gene. Studies have found that P30 can induce host cells to produce neutralizing antibodies that inhibit intracellular internalization, thus delaying the onset of disease or even protecting cells against viral infection. Therefore, P30 plays an important role in blocking virus-cell interaction. P30, as an early protein of virus, is mainly distributed in the cytoplasm after infected cells, and can be detected in the cytoplasm four hours after infection. P30 is also one of the most antigenic ASFV proteins with strong immunogenicity, which can induce the body to produce virus neutralizing antibodies in infected animals, so it is usually used as a diagnostic antigen. The P54 protein of ASFV is encoded by E183L gene, and its antibody has certain virus neutralization ability. In addition, P30 protein and P54 protein can interact with two different receptors or binding sites on susceptible cells to alleviate the disease course. P72 protein is one of the main detected antigens of ASFV, with a size of about 75 kd. Good stability and small variation. A series of detection products have been developed with P72 protein as antigen. PB602L protein encoded by B602L gene can stimulate the matrix to produce high-level antibody. However, in all of the previous studies, there is no recombinant adenovirus vector for co-expression of four antigen genes, and there is no recombinant adenovirus vector for co-expression of four antigen genes P72, B602L, P30 and P54 of ASFV to be applied to the development of live vector vaccines. And there is no combination of two molecular adjuvants simultaneously cloned into the vector and coexpressed with a group of ASFV antigens.

### SUMMARY OF THE INVENTION

In order to solve the problems, the invention provides an african swine fever virus vaccine, which is obtained by constructing a recombinant adenovirus vector co-expressing four antigen genes of the african swine fever virus and packaging the recombinant adenovirus vector by 293TD37 cells. There are five groups of four antigen genes of the african swine fever virus are designed in total, and the four antigen genes of any group can be used for preparing a recombinant adenovirus vector co-expressing the four antigen genes of the african swine fever virus, namely the african swine fever virus vaccine. The invention can greatly improve the capacity of the adenovirus vector vaccine and enhance the specific immune response to the african swine fever virus by simultaneously expressing four group independent antigens of the african swine fever inonly one adenovirus vector.

There are more than 160 antigen genes of African swine fever virus in total, and the inventor selects 20 antigen genes with stronger immune effect from the 160 antigen genes through a large number of screening experiments, namely as: P72, B602L, P30, P54, CP129R, MGF5L6L, CP312R, MGF110-4L, L8L, I215L, I73R, E146L, EP402R, EP153R, I177L, K205R, F317L, A15MR, P34 and pp62. The 20 antigen genes are divided into five groups according to that size of the gene fragment and the protein structure, and four antigen genes in each group can be co-expressed in the recombinant adenovirus vector pAd5LCL3 provided by the invention, that is, four antigen genes can be completely and independently expressed in the same vector. The five groups of antigen gene vaccines (including five recombinant adenovirus vectors pAd5LCL3) form a complete African swine fever virus vaccine and obtain very good immune effect. In the five groups of antigen gene vaccines, four antigen genes of each group can be well matched and assembled in the same recombinant adenovirus vector, so that the four antigen genes can be completely and independently expressed.

In one aspect, the invention provides a recombinant adenovirus vector pAd5LCL3 capable of simultaneously expressing a plurality of antigen genes, wherein the recombinant adenovirus vector pAd5LCL3 is missed E1, E3, E4 and E2a genes and has E1 regions and E4 regions capable of respectively simultaneously expressing one or more exogenous antigen genes in the E1 and E4 regions. The antigen gene may be an antigen gene of an appropriate size from any source.

Further, the E1 region and E4 region of the recombinant adenovirus vector pAd5LCL3 can express four antigen genes of different or the same origin.

Further, the sequences of ORF1 to ORF7 of the E4 region of the recombinant adenovirus vector pAd5LCL3 is missed.

Further, the E2a region (also known as DNA Binding Protein (DBP)) of the recombinant adenovirus vector pAd5LCL3 is delelted.

Further, the E4 promoter, ORF6, ORF7, and polyA sequences of the E4 region of the recombinant adenovirus vector pAd5LCL3 are placed at the E2a position.

Furthermore, the E1 region of the recombinant adenovirus vector pAd5LCL3 is placed with a SwaI restriction site.

Further, an I-sceI restriction site is preset in the E4 region of the recombinant adenovirus vector pAd5LCL3.

The study found that the genes related to adenovirus replication were E1, E2, E3, and E4. The deletion of these genes did not affect the expression of adenovirus structural proteins, but it prevented adenovirus from being replicated and packaged. Therefore, the construction of these replication-related cell lines enables replication-defective adenovirus vectors with knockout replication genes to be replicated and packaged in cell lines specific to them. At the same time, the study found that as long as the ORF6 or ORF3 in the E4 gene expressing adenovirus can replace the entire E4 gene, the adenovirus with the knockout E4 can be replicated and packaged. Through further research and analysis on the sequences of E4 and E2a genes, E4 gene can be expressed at E2a. Therefore, the invention carries out sequence analysis on the E4 gene, finds out the basic elements of promoter, ORF6/7 and polyA of E4, integrates the basic elements into a complete expression frame, constructs the complete expression frame at the sequence position where the E2a gene is knocked out, enables the ORF6 and ORF7 genes to be normally expressed, and finally obtains a replication defective adenovirus type 5 vector pAd5LCL3 which knocks out E1, E3, E4 and E2a and places the E4 expression frame at the E2a position, and can be subjected to replication and packaging in 293TD37 cells containing DBP sequences.

The study found that the E4 gene contained seven expression frames of ORF1, 2, 3, 4, 5, 6 and 7, of which, ORF6-7 could not be deleted, and once deleted (ORF6-7), it would significantly affect the adenovirus packaging and antigen gene expression. So ORF6-7 needed to be supplemented back and can not be moved out. At the same time, in order to obtain a larger vector space, ORF6-7 needed to be expressed at E2a, so as to prepare an adenovirus vector with larger capacity and better expression effect.

Further, the recombinant adenovirus vector pAd5LCL3 capable of simultaneously expressing four antigen genes can only be packaged by 293TD37 cells constructed by pcDNA3.1+(hyg)-ORF6-IRES-DBP, and the cell strain storage number of the 293TD37 cells is CCTCC NO:C201996, which is deposited in the China Type Culture Collection.

Ordinary 293 cells contain E1 gene of adenovirus type 5. Adenoviruses with knockout of E1 and E3 can be replicated in this cell line, but adenoviruses with knockout of E4 and E2a genes cannot be replicated in 293 cells.

The 293TD37 cell strain is invented by some inventgors of the present invention, and has filed the invention patent with the filing no. CN201911033247.2, which is deposited with the China Type Culture Collection on May 8, 2019 with the deposition number CCTCC NO:C201996, and is classified as human embryonic kidney transformed cell AY293-TD-37. The cell strain comprises an adenovirus E2a-DBP gene and an E4-ORF6/7 gene. It can be used for packaging the E2a-DBP gene and E4 gene-deficient second-generation adenovirus to form complete infectious second-generation adenovirus particles, the probability of RCA of the second-generation adenovirus is greatly reduced compared with that of the first-generation adenovirus, and a foundation is laid for preparing a live vector vaccine.

The invention provides a construction method of a recombinant adenovirus vector pAd5LCL3, which mainly utilizes CRISPR/cas9 technology to knock out E1, E3, E4 and E2a genes of an adenovirus vector plasmid, and puts an ORF6f7 expression frame of an E4 region at a sequence position of an E2a region that is knocked out.

The construction method of the recombinant adenovirus vector pAd5LCL3 capable of simultaneously expressing four antigen genes comprises the following steps:

Step: 1) knocking out the E1 gene of the adenovirus carrier plasmid by CRISPR/cas9 technology, introducing a SwaI restriction enzyme cutting site, seamlessly cloning the fused fragment and the carrier; knocking out the E3 gene by CRISPR/cas9 technology, and then connecting in a seamless cloning mode to obtain the adenovirus carrier plasmid pAd5 without E1 and E3 genes;
and, step 2) knocking out the E4 gene of the adenovirus vector plasmid pAd5 by CRISPR/cas9 technology, amplifying by PCR and introducing an I-sceI restriction enzyme cutting site, and obtaining the adenovirus vector plasmid pAd5ΔE4 without E1, E3 and e4 genes by a seamless cloning method;

On the basis that E1 and E3 genes are knocked out, the E4 gene is knocked out further, so that the capacity of the adenovirus vector is increased, and the immunogenicity is reduced. Meanwhile, an exogenous gene can be inserted into the E4 region, and the exogenous gene can be abundantly expressed at the E4 position but without affecting the packaging of the adenosis vector. The expression of the exogenous gene at the regions of E1 and E4 genes can avoid mutual interference of the expression of a plurality of exogenous genes in the same region, is more favorable for expression, and also simultaneously reduces unnecessary E4 related genes. That reduces the immunogenicity of the adenovirus, enables the adenovirus to exist in a host cell for a longer time, and enables the exogenous gene to be expressed for a longer time.

E4 region gene plays a key role in immunogenicity. The expression of a large number of E4 region genes will lead to a relatively strong immune response of the host and induce the production of antibodies. E4 is not conducive to the long-term expression of the target protein in the host by the adenovirus vector. Therefore, knocking out the unnecessary genes in the E4 region can reduce the immunogenicity of the adenovirus vector, so that the vector can be expressed for a longer time.

In order to completely knock out the gens in the E4 region and facilitate the connection of large vector plasmids, the CRISPR/cas9 method is used to knock out the upstream Fibro gene in the E4 region and the gene in E4, a PCR method is used to amplify part of the Fibro and introduce an I-sceI single restriction site, and then Gibson's seamless cloning method is used to connect the extra excised fragment to the vector to obtain the vector plasmid in which the I-sceI single restriction site is introduced in E4 knockdown. The vector plasmid was linearized using I-sceI, and the shuttle plasmid in the E4 region was constructed so that the foreign gene was recombined into the E4 region and abundantly expressed in the E4 region.

In one some embodiment, it further comprise step 3): knocking out the E2a gene of the adenovirus vector plasmid pAd5ΔE4 by CRISPR/cas9 technology, placing an ORF6f1 expression frame of an E4 region at the position where the E2a region is knocked out, and then using a seamless cloning method to obtain the adenovirus vector plasmid pAd5LCL3 without E1, E3, E4 and E2a genes.

The sequences from ORF1˜ORF5 in the E4 region are knocked out, and the E4 promoter, ORF6, ORF7, and polyA sequences are retained but that are inserted into the E2a position, so that the E4 position can be used for expressing the foreign gene. The DBP sequence of the E2a region is also knocked out. Adenovirus E2a gene is a DNA binding protein, which is related to the replication of adenovirus. Knocking out this gene does not affect the structural protein of adenovirus or its packaging. DBP deletions can prevent or greatly reduce reverse mutations. The knockout of the E2a and E4 partial sequences increases the carrier capacity by about 3 kb.

The shuttle plasmid is commonly used in the construction of existing adenovirus vectors, and a single restriction site needs to be found. In the invention, CRISPR/cas9 is creatively adopted to construct the recombinant adenovirus vector, appropriate E1, E3, E4 and E2a knockout sites are selected through comparison, the CRISPR site is selected according to the positions of E1, E3, E4 and E2a sequences and the number of knockout gene bases, and the optimal gRNA is designed, so that the construction of the recombinant adenovirus vector is completed.

In yet another aspect, the present invention provides a recombinant adenovirus vaccine comprising a target gene of interest that is inserted into the E1 and E4 regions of pAd5LCL3.

Studies have shown that the expression level of exogenous protein in E3 region is not high, while the expression of antigen gene in E1 and E4 regions is higher, so the four antigens can be expressed in E1 and E4 regions separately.

Because E3 genes is associated with replication, that is need to knock out and let its replication defects; the role of E3 is related to the immune escape of adenovirus; knocking out the E3 region can increase the capacity of the adenovirus vector; and enable that adenovirus vector to be normally packaged.

Further, the target gene of interest is a gene or gene fragment of a virus, bacterium, or tumor.

Further, the target gene is an african swine fever virus gene.

In another aspect, the invention provides an african swine fever virus vaccine, which is characterized in that the vaccine is obtained by constructing a recombinant adenovirus vector co-expressing four antigen genes of the african swine fever virus and packaging the recombinant adenovirus vector by 293TD37 cells.

Further, the recombinant adenovirus vector co-expressing the four antigen genes of the African swine fever virus needs to be packaged by a recombinant adenovirus of 293TD37 cells constructed by pcDNA3.1+(hyg)-ORF6-IRES-DBP, and the cell strain storage number of the 293TD37 cells is CCTCC NO:C201996, which is deposited in the China Type Culture Collection.

Further, the four antigen genes are any one of the following five groups of antigen genes, respectively: a group 1: P72, B602L, P30, and P54: Group 2: CP129Rubiqutin, MGF5L6L, CP312R, and MGF110-4L: Group 3: L8Lubiqutin, I215L, I73RHBsAg, and E146L: Group 4: EP402R, EP153R, I177L, and K205Rubiqutin: Group 5: F317L, A151R, P34, and pp62.

In some embodiments, the first group, P72 and B602L are expressed in the E1 region and P30 and P54 are expressed in the E4 region, constituting a recombinant adenovirus vector pAd5LCL3-P72-B602L-P30-P54 in which four antigen genes are co-expressed.

In second group, the CP129Rubiqutin is obtain by adding the molecular adjuvant ubiquitin on the CP129R, the CP129Rubiqutin and the MGF5L6L are express in an E1 region, the CP312R and the MGF110-4L are expressed in an E4 region, and a recombinant adenovirus vector pAd5LCL3-CP129Rubiqutin-MGF5L6L-CP312R-MGF110-4L for co-expression of four antigen genes is formed.

In that third group, L8Lubiqutin is obtain by adding the molecular adjuvant ubiquitin to L8L, I73RHBsAg is obtain by adding the molecular adjuvant HBsAg to 173R, L8Lubiqutin and I215L are expressed in an E1 region, I73RHBsAg and E146L are expressed in an E4 region, and a recombinant adenovirus vector pAd5LCL3-L8 Lubiqutin-I215L-I73R HBsAg-E146L for co-expression of four antigen gene is formed.

In that fourth group, the K205Rubiqutin is obtain by adding the molecular adjuvant ubiqutin on the K205R, the EP402R and the EP153R are express in the E1 region, the I177L and the K205Rubiqutin are expressed in the E4 region, and a recombinant adenovirus vector pAd5LCL3-EP402R-EP153R-I177L-K205rubiqutin for co-expression of four antigen genes.

In the fifth group, F317L and A151R are expressed in the E1 region, and P34 and pp62 are expressed in the E4 region, forming a recombinant adenovirus vector pAd5LCL3-F317L-A151R-P34-PP62 in which four antigen genes are co-expressed.

Through a large number of screening experiments, the inventors of the present invention selects 20 antigen genes with stronger immune effect from more than 160 african swine fever virus antigen genes, the 20 antigen genes are divided into five groups according to gene fragment size and protein structure, and the four antigen genes of each group can be co-expressed in the recombinant adenovirus vector pAd5LCL3 provided by the invention, namely, the four antigen genes can be completely and independently expressed in the same vector.

The four antigenic genes of African swine fever virus in each group were Group1, P72, B602L, P30 and P54: respectively. Group2, CP129Rubiqutin, MGF5L6L, CP312R, and MGF110-4L: Group3, L8Lubiqutin, I215L, I73RHBsAg and E146L: Group4, EP402R, EP153R, I177L, and K205Rubiqutin: Group5, F317L, A151R, P34, and pp62.

Further, the P72, B602L, P30, P54 and pAd5LCL3-P72-B602L-P30-P54 have nucleotide sequences shown in Seq ID NO.1, Seq ID NO.2, Seq ID NO.3, Seq ID NO.4 and Seq ID NO.6, respectively, in a sequence table. The CP129R, ubiqutin, MGF5L6L, CP312R, MGF110-4L, pAd5LCL3-CP129R ubiqutin-MGF5L6L-CP312R-MGF110-4L respectively have nucleotide sequences shown in Seq ID NO.14, Seq ID NO.15, Seq ID NO.16, Seq ID NO.17, Seq ID NO.18 and Seq ID NO.19 in a sequence table. The LSL, the ubiqutin, the I215L, the I73R, the HBsAg, the E146L and the pAd5LCL3-L8L ubiqutin-I215L-I73R HBsAg-E146L respectively have nucleotide sequences shown in Seq ID NO.20, Seq ID NO.21, Seq ID NO.22, Seq ID NO.23, Seq ID NO.24, Seq ID NO.25 and Seq ID NO.26 in a sequence table. The EP402R, the EP153R, the I177L, the K205R, the ubiqutin, and the pAd5LCL3-EP402R-EP153R-I177L-K205R ubiqutin respectively have nucleotide sequences shown in Seq ID NO.27, Seq ID NO.28, Seq ID NO.29, Seq ID NO.30, Seq ID NO.31 and Seq ID NO.32 in a sequence table. The F317L, AM51R, P34, pp62, and pAd5LCL3-F317L-A151R-P34-pp62 have nucleotide sequences shown in Seq ID NO.33, Seq ID NO.34, Seq ID NO.36, Seq ID NO.36 and Seq ID NO.37, respectively, in a sequence table.

In yet another aspect, the present invention provides a construction method of an african swine fever virus vaccine as described above, mainly comprising the steps of:
1) knocking out an E1 gene of an adenovirus carrier plasmid by CRISPR/cas9 technology, introducing a SwaI restriction enzyme cutting site, seamlessly cloning a fused fragment with the carrier, knocking out an E3 gene by CRISPR/cas9 technology, and connecting in a seamless cloning mode to obtain an adenovirus carrier plasmid pAd5 without E1 and E3 genes;
2) knocking out the E4 gene of the adenovirus vector plasmid pAd5 in step 1 by CRISPR/cas9 technology, amplifying by PCR and introducing an I-sceI restriction enzyme cutting site, and then obtaining the adenovirus vector plasmid pad5ΔE4 without E1, E3 and E4 genes by a seamless cloning method;
3) knocking out the E2a gene of the adenovirus vector plasmid pAd5ΔE4 in step 2 by CRISPR/cas9 technology, placing an ORF6/7 expression frame of an E4 region at a sequence position where the E2a region is knocked out, and then using a seamless cloning method to obtain the adenovirus vector plasmid pAd5LCL3: without E1, E3, E4 and E2a genes;
4) construct an adenovirus E1 region shuttle plasmid, pS5E1 was connected to P72, IRES, B602L of the first group, CP129Rubiqutin, IRES, MGF5L6L of the second group, or L8Lubiqutin, IRES, I215L of the third group, or EP402R, IRES, EP153R of the fourth group, or F317L, IRES, A151R gene fragments of the fifth group by DNA ligases to construct an African swine fever adenovirus type 5 vector E1 region shuttle plasmid, respectively, Group 1: pS5E1-P72-IRE2; Group 2: pS5E1-CP129Rubiqutin-IRES-MGF5L6L; Group 3:pS5E1-L8Lubiqutin-IRES-I215L; Group 4: pS5E1-EP402R-IRES-EP153R; Group 5: pS5E1-F317L-IRES-A151R.
5) constructing an adenovirus E4 region shuttle plasmid which is respectively connected with P30, 2A and P54 of the first group; CP312R, 2A, MGF5L6L of the second group: I73RHBsAg, 2A, E146L of the group 3 HBsAg I177L, 2A, K205Rubiqutin of the fourth group; or the P34, 2A and pp62 genes of the fifth group; are respectively name as P30-2A-P54; CP312R-2A-MGF5L6L; I73RHBsAg-2A-E146L; I177L-2A-K205Rubiqutin and P34-2A-pp62 through fusion PCR technology, and the EGFP gene is knocked out through enzyme digestion on a shuttle plasmid pS5E4-EGFP, and connecte with that gene fragment by a DNA ligase to construct an E4 region shuttle plasmid of an african swine fever adenovirus type 5 vector, namely pS5E4-P30-2A-P54; The second group: pS5E4-CP312R-2A-MGF 5L6L; The third group: pS5E4-I73RHBsAg-2A-E146L; The fourth group: pS5E4-I177L-2A-K205Rubiqutin; The fifth group: pS5E4-P34-2A-pp62.
6) the E1 region shuttle plasmid pS5E1-P72-IRES-B602L, or pS5E1-CP129Rubiqutin-IRES-MGF5L6L, or pS5E1-L8Lubiqutin-IRES-I215L, or pS5E1-EP402R-IRES-EP153R, Or pS5E1-F317L-IRES-A151R is homologously recombined with an adenovirus vector plasmid pAd5LCL3 to obtain a first group of adenovirus vector plasmids: pAd5LCL3-P72-IRES-B602L; The second group pAd5LCL3-CP129Rubiqutin-IRES-MGF5L6L; The third group: pAd5LCL3-L8Lubiqutin-IRES-I215L; The fourth group: pAd5LCL3-I177L-2A-K205Rubiqutin; The fifth group: pAd5LCL3-F317L-IRES-A151R.
7) shuttle that E4 region plasmid first group: pS5E4-P30-2A-P54; The second group: pS5E4-CP312R-2A-MGF5L6L; The third group: pS5E4-I73RHBsAg-2A-E146L; The fourth group: pS5E4-I177L-2A-K205Rubiqutin; The fifth group: pS5E4-P34-2A-pp62 and adenovirus vector plasmid The first group: pAd5LCL3-P72-IRES-B602L; The second group pAd5LCL3-CP129Rubiqutin-IRES-MGF5L6L; The third group: pAd5LCL3-L8Lubiqutin-IRES-I215L; The fourth group: pAd5LCL3-I177L-2A-K205Rubiqutin; and fifth group, performing homologous recombination of pAd5LCL3-F317L-IRES-A151R to obtain a recombinant adenovirus vaccine co-expressing four antigen genes, wherein the first group comprises pAd5LCL3-P72-B602L-P30-P54; The second group: pAd5LCL3-CP129Rubiqutin-MGF5L6L-CP312R-MGF110-4L; The third group: pAd5LCL3-L8Lubiqutin-I215L-I73RHBsAg-E146L; The fourth group: pAd5LCL3-EP402R-EP153R-I177L-K205Rubiqutin; The fifth group: pAd5LCL3-F317L-A151R-P34-PP62.

Further, the adenovirus vector plasmid described in step 1) is derived from wild-type human adenovirus type 5 virus amplified in A549 cells, virus liquid is collected and concentrated, an adenovirus type 5 genome is extracted by HirtViral DNA Extract method, and a linear adenovirus type 5 genome is constructed into an adenovirus vector plasmid by a cosmid method.

Further, the ORF6/7 expression frame gene in step 3) has a nucleotide sequence shown in Seq ID NO.7 in a sequence table; Step 4) the IRES has a nucleotide sequence shown in Seq ID NO.8 in a sequence table; and 2A in step 5) has the nucleotide sequence shown in Seq ID NO.9 in a sequence table.

Further, the shuttle plasmid pS5E1 skeleton described in step 4) adopts puc origin, amp basic elements, Ad5 left arm ITR partial sequence, right arm PIX, PIVa2 partial sequence, and CMV-MCS SV40 early polyA: The skeleton of the E4 region shuttle plasmid pS5E4-EGFP in step 5) adopts puc origin, amp basic elements, a left arm ITR sequence in the Ad5E4 region, a right arm partial fiber gene sequence and an EF1α-EGFP-HBV polyA gene; Wherein that basic element of puc origin and amp have the nucleotide sequence shown in Seq ID NO.10 in the sequence table, and the EF1α-EGFP-HBV polyA gene has the nucleotide sequence shown in Seq ID NO.11 in the sequence table.

The skeleton of shuttle plasmid pS5E1 was synthesized by Beijing BoMed Gene Technology Co., Ltd. using puc origin, amp and other basic elements (2796 bp), partial sequence of ITR in Ad5 left arm (400 bp), partial sequence of PIX and PIVa2 in her right arm (2100 bp), and CMV-MCS (944 bp) SV40 Early Polya (160 bp) in synthesis. After PCR amplification and gene fragment purification, seamless clonal connection was performed, and the connection product was converted to competent cells, coated with ampicillin resistance plate, and positive clones were selected for restriction enzyme digestion verification after culture to obtain adenovirus E1 region shuttle plasmid pS5E1.

The skeleton of the shuttle plasmid pS5E4 was composed of basic elements such as puc origin and amp, the ITR sequence of the left arm (370 bp), the partial fiber gene sequence of the right arm (1746 bp) in the Ad5E4 region, and the EF1α-EGFP-HBV polyA gene. After PCR amplification and gene fragment purification, seamless clone connection was performed, the connection product was converted to competent cells, an ampicillin resistance plate was coated, and positive clones were selected for restriction enzyme digestion verification after culture to obtain adenovirus E4 region shuttle plasmid pS5E4-EGFP.

Further, in step 6), the E1 region shuttle plasmid is homologously recombined with the adenovirus vector plasmid pAd5LCL3, and the shuttle plasmid and the adenovirus vector plasmid pAd5LCL3 are subjected to enzyme digestion by PacI and SwaI, dephosphorylation of an enzyme digestion product, gel recovery of a carrier and fragments by OMEGA Ultra-Sep Gel Extraction Kit, coating of a plate with a conversion product, and picking of colonies for XhoI enzyme digestion verification.

Further, in step 7), the E4 region shuttle plasmid is homologously recombined with the adenovirus vector plasmid, and the E4 region shuttle plasmid and the adenovirus vector plasmid are subjected to enzyme digestion by PacI and I-sceI, the enzyme digestion product is dephosphorylated, the omega ultra-sepge1 extract kit carries out gel recovery carrier and fragments, the conversion product is coated on a plate, colonies are picked, and the XhoI enzyme digestion verification is carried out.

In another aspect, the invention provides a packaging method of a recombinant adenovirus vector, which mainly comprises the following steps of: respectively packaging a first group of the recombinant adenovirus vaccines, namely, pAd5LCL3-P72-B602L-P30-P54; The second group: pAd5LCL3-CP129Rubiqutin-MGF5L6L-CP312R-MGF110-4L; The third group: pAd5LCL3-L8LUBIQUTIN-I215L-I73RHBsAg-E146L; The fourth group: pAd5LCL3-EP402R-EP153R-I177L-K205Rubiqutin; The fifth group: pAd5LCL3-F317L-A151R-P34-PP62; using PacI enzyme digestion, the linearized plasmid is used for transfection; 293TD37 cells constructed from pcDNA3.1+(hyg)-ORF6-IRES-DBP were transfected and cell suspension was collected.

The 293TD37 cell strain is deposited in the China Type Culture Collection on May 8, 2019 with the deposition number of CCTCC NO:C201996 and is classified as human embryonic kidney transformed cell AY293-TD37. The cell strain comprises adenovirus E2a and E4-ORF6/7 genes and is obtained by genetically engineering HEK293 cells and can be used for packaging second-generation recombinant adenovirus lacking E2a and E4 genes to form infectious second-generation adenovirus particles.

Further, the packaging method of the recombinant adenovirus vector is mainly prepared from the following steps:
1) respectively mixing the first group of the recombinant adenovirus vaccine: pAd5LCL3-P72-B602L-P30-P54; The second group: pAd5LCL3-CP129Rubiqutin-MGF5L6L-CP312R-MGF110-4L; The third group: pAd5LCL3-L8Lubiqutin-I215L-I73RHBsAg-E146L; The fourth group: pAd5LCL3-EP402R-EP153R-I177L-K205Rubiqutin; The fifth group: pAd5LCL3-F317L-A151R-P34-PP62; using PacI enzyme digestion, the linearized plasmid is used for transfection; 293TD37 cells were transfected with PEI transfection reagents;
2) culturing the transfected 293TD37 cells in a 5% CO₂ incubator at 37° C. for 72 to 96 hours, and collecting cell suspension, namely the TP0 generation adenovirus;
3) 293TD37 cells infected with adenovirus of 3) TP0 generation were cultured at 37° C. for 72 hours in a 5% CO₂ incubator, and cell suspension, namely adenovirus of TP1 generation, was collected;
4) repeating the step 3), and collecting the cell suspension, namely, the TP2 generation adenovirus;
5) Continuous inoculation until the cells become diseased.

In yet another aspect, that present invention provide the use of 293TD37 cells for package recombinant adenovirus vectors co-expressing four antigenic genes of african swine fever virus, the four antigenic genes bee respectively a first group: P72, B602L, P30 and P54: Group 2: CP129Rubiqutin, MGF5L6L, CP312R, and MGF110-4L: Group 3: L8Lubiqutin, I215L, I73RHBsAg, and E146L: Group 4: EP402R, EP153R, I177L, and K205Rubiqutin: Group 5: F317L, A15MR, P34, and pp62: In the first group, P72 and B602L are expressed in the E1 region, and P30 and P54 are expressed in the E4 region, forming a recombinant adenovirus vector pAd5LCL3-P72-B602L-P30-P54 for co-expression of four antigen genes; In that second group, the CP129Rubiqutin is obtain by adding the molecular adjuvant ubiquitin on the CP129R, the CP129Rubiqutin and the MGF5L6L are express in an E1 region, the CP312R and the MGF110-4L are expressed in an E4 region, and a recombinant adenovirus vector pAd5LCL3-CP129R ubiqutin-MGF5L6L-CP312R-MGF110-4L for co-expression of four antigen genes is formed; In that third group, L8Lubiqutin is obtain by adding the molecular adjuvant ubiquitin to L8L, I73RHBsAg is obtain by adding the molecular adjuvant HBsAg to 173R, L8Lubiqutin and I215L are expressed in an E1 region, I73RHBsAg and E146L are expressed in an E4 region, and a recombinant adenovirus vector pAD5 LCL3-L8 Lubiqutin-I215L-I73R HBsAg-E146L for co-expression of four antigen gene is formed. In that fourth group, the K205Rubiqutin is obtain by adding the molecular adjuvant ubiqutin on the K205R, the EP402R and the EP153R are express in the E1 region, the I177L and the K205Rubiqutin are expressed in the E4 region, and a recombinant adenovirus vector pAd5LCL3-EP402R-EP153R-I177L-K205rubiqutin for co-expression of four antigen genes is for; In the fifth group, F317L and A151R are expressed in the E1 region, and P34 and pp62 are expressed in the E4 region, forming a recombinant adenovirus vector pAd5LCL3-F317L-A151R-P34-PP62 in which four antigen genes are co-expressed;

Wherein, the 293TD37 cell is constructed by pcDNA3.1+(hyg)-ORF6-IRES-DBP, and the cell strain preservation number is CCTCC NO:C201996, which is deposited in the China Type Culture Collection.

The invention provides an african swine fever virus vaccine, which is obtained by constructing a recombinant adenovirus vector co-expressing four antigen genes of the african swine fever virus and packaging by 293TD37 cells. Wherein five groups of four antigen genes of the african swine fever virus are designed in total, and the four antigen genes of any group can be used for preparing a recombinant adenovirus vector co-expressing the four antigen genes of the african swine fever virus, namely the african swine fever virus vaccine. The construction of the recombinant adenovirus vector for co-expression of four antigen genes of the african swine fever virus mainly comprises the following steps of: knocking out E1, E3, E2a and E4 genes of the adenovirus vector by CRISPR/cas9 technology, and constructing shuttle plasmids in E1 and E4 regions for respectively expressing four antigen genes, thereby obtaining a completely new adenovirus vector. The invention has the beneficial effects that:

(1) a novel construction method of adenovirus type 5 vector is provided, the optimal knockout site and gRNA are independently designed, and the problem that a single restriction site needs to be found due to the knockout by shuttle plasmid in the past vector construction is avoided.

(2) as the E4 region gene plays a key role in immunogenicity, the expression of a large number of E4 region genes can lead a host to generate a relatively strong immune response and induce the generation of antibodies, which is not favorable for the adenovirus vector to express a target protein in the host for a long time.

(3) in the invention, the sequences of orf1 to orf5 in the E4 region are knocked out, and the E4 promoter, ORF6, ORF7 and polyA sequences are retained and inserted into the E2a position, so that the E4 position can be used for expressing foreign genes.

(4) The invention further knocks out the DBP(E2a) sequence, and the deletion of DBP can prevent or greatly reduce the reversion mutation. The partial sequence knockout of E2a and E4 increases the vector capacity by about 3 kb relative to the first-generation vector.

(5) E2a and E4 of the adenovirus vector are knocked out, and E4promoter-ORF6f7-polyA is placed in the E2a region, so that a cell line complementary to E2a(DBP sequence) can be used for rescue; meanwhile, foreign genes can be simultaneously expressed in the E1 and E4 regions without mutual interference; and at present, the adenovirus vaccine is rescued in a complementary cell line-293TD37 cell line constructed by our company, and the cell line can permanently express DBP protein.

(6) the invention constructs shuttle plasmids of E1 and E4 regions for expressing exogenous genes of E1 and E4 regions.

(7) the titer of the recombinant adenovirus virus prepared by packaging the 293TD37 cell line is higher.

Based on the above principles, the invention can greatly improve the capacity of the vaccine of the adenovirus vector, enhances the specific immune response to the african swine fever virus by simultaneously expressing four independent antigens of the african swine fever on one adenovirus vector, and can lead the domestic pigs to obtain better immune protection.

### BRIEF DESCRIPTION OF THE DRAWINGS

FIG. 1 The schematic diagram of the Ad5-E4-up-gRNA cleavage site and PAM site of example 2.

FIG. 2 (SEQ ID NOS: 164 and 165) The schematic diagram of the Ad5-E4-down-gRNA cleavage site and PAM site of example 2.

FIG. 3 The electrophoresis results of the Ad5-E4-up-gRNA, Ad5-E4-down-gRNA, and cas9 “double restriction enzyme digestion” vector plasmids of example 2, where lane 1 was Ad5-E4-up-gRNA, Ad5-E4-down-gRNA, and cas9 “double restriction enzyme digestion”, and M was DL 2,000 DNA Marker.

FIG. 4 The results of amplification and electrophoresis detection of the fiber and ITR fragments containing partial knockout in example 2, wherein lane 1 was the result of amplification of the fiber partial fragment, lane 2 was the result of amplification of the ITR partial fragment, and M was DL 2,000 DNA Marker.

FIG. 5 The result of the Fiber-ITR fusion fragment of example 2, in which lane 1 was the Fiber-ITR fusion fragment and M was DL 15,000 DNA Marker.

FIG. 6 The colony PCR electrophoresis results of example 2, in which lanes 1-24 were colonies and M was DL 2,000 DNA Marker.

FIG. 7 The electrophoresis detection results of BamHI, XhoI enzyme digestion verification of the positive clone colony plasmid of FIG. 6 in example 2, where lanes 1-5 were BamHI Enzyme digestion, lanes 6-10 for XhoI Enzyme digestion, lanes 1 and lanes 10 for pAd5 control (for indeed E4 gene), M was DL 15,000 DNA Marker.

FIG. 8 The 100 k-gRNA cleavage site and the PAM site of example 3.

FIG. 9 The protease-gRNA cleavage site and PAM site of example 3.

FIG. 10 The results of the 100 k-gRNA, protease-gRNA and cas9 “double-restriction” vector plasmids of example 3, lane 1 was the results of the 100 k-gRNA, protease-gRNA and cas9 “double-restriction” vector plasmids, and M was DL 15,000 DNA Marker.

FIG. 11 The results of the 100 k, E4 ORF6/7 expression frame and protease PCR amplification electrophoresis of example 3, where lane 1 was the E4 ORF6/7 expression frame, lane 2 was 100 k, and M was DL 2,000 DNA Marker.

FIG. 12 The fusion PCR electrophoresis of the 100 k, E4 ORF6/7 expression frame and Protease fragment in example 3, wherein lane 1 was the fragment 100 k, E4 ORF6/7 expression frame and protease fusion PCR product, and M was DL 15,000 DNA Marker.

FIG. 13 The colony PCR electrophoresis results of example 3, in which lanes 1-24 were colonies and M was DL 15,000 DNA Marker.

FIG. 14 The electrophoresis detection results of the XhoI digestion verification of colonies No. 9, 18, 21, and 24 of the colonies of FIG. 13 picked out in example 3, wherein lane 1 was the XhoI digestion of the positive clone No. 9, lane 2 was the XhoI digestion of the positive clone No. 18, lane 3 was the XhoI digestion of the positive clone No. 21, lane 4 was the XhoI digestion of the positive clone No. 24, lane 5 was the XhoI digestion of the control plasmid pAd5LCL3, and M was DL 15,000 DNA Marker.

FIG. 15 The results of the amplification electrophoresis of the CMV-MCS and SV40 earlypolyA fragments of example 4, where lane 1 was the CMV-MCS fragment, lane 2 was the SV40 earlypolyA fragment, and M was DL 2,000 DNA Marker.

FIG. 16 The amplified electrophoresis results of CMV-MCS-SV40 earlypolyA, PUC, HAd5 right arm and HAd5 left arm of example 4, in which lane 1 was the CMV-MCS-SV40 earlypolyA fusion fragment, lane 2 was PUC, lane 3 was HAd5 right arm, lane 4 was HAd5 left arm, and M was DL 2,000 DNA Marker.

FIG. 17 The results of the PCR-validated electrophoresis of colonies of competent cells ligated by the four fragments of the pS5E1 skeleton, the HAd5 left arm, the HAd5 right arm, and CMV-MCS-SV40 earlypolyA of example 4, where lanes 1-6 were colonies and M was DL 2,000 DNA Marker.

FIG. 18 The result of the digestion verification of selected colonies No. 1-6 in FIG. 17 of example 4, wherein left No. 1-6 were plasmids pS5E1 NcoI single-digestion, right No. 1-6 were plasmids pS5E1 PacI single-digestion, and M was DL 15,000 DNA Marker.

FIG. 19 The IRES fragment PCR amplification electrophoresis of example 4, in which lanes 1 and 2 were the IRES fragment PCR amplification products and M was DL 2,000 DNA Marker.

FIG. 20 The vector digestion electrophoresis of fragments IRES and pS5E1 in example 4, in which lane 1 was the digestion of fragments IRES EcoRV and NotI, lane 2 was the digestion of pS5E1 EcoRV and NotI, and M was DL 15,000 DNA Marker.

FIG. 21 The PCR validation electrophoresis result of the colonies of competent cells transformed with the pS5E1 vector and the IRES fragment of example 4, where No. 1-9 were colonies and M was DL 2,000 DNA Marker.

FIG. 22 The electrophoresis detection and verification of the pS5E1-IRES plasmids NotI and EcoRV digestion in example 4. plasmids 2 and 6 of FIG. 21 were selected for plasmid extraction and digestion verification, where lane 1 was for plasmid NotI and EcoRV digestion identification, and lane 2 was for plasmid NotI No. 6 and EcoRV digestion identification, and M was DL 15,000 DNA Marker.

FIG. 23 The vector digestion electrophoresis of P72 and pS5E1-IRES of example 4, where lane 1 was the fragment pS5E1-IRES, NotI digestion, lane 2 was the P72, NotI digestion, and M was DL 15,000 DNA Marker.

FIG. 24 The PCR validation electrophoresis result of the colonies of competent cells transformed with the P72 and pS5E1-IRES ligation products of example 4, where No. 1-10 were colonies and M was DL 2,000 DNA Marker.

FIG. 25 The plasmid digestion electrophoresis detection verification of pS5E1-P72-IRES of example 4. colonies No. 2 and 5 in FIG. 24 were selected for plasmid extraction and digestion verification, where lane 2 is the plasmid digestion verification No. 2, lane 5 was the plasmid digestion verification No. 5, and M was a Marker.

FIG. 26 The electrophoresis detection results of the vector-digested products of fragment B602L and pS5E1-P72-IRES of example 4, wherein lane 1 was pS5E1-P72-IRES, notch I and XhoI were digested, lane 2 was the fragment B602L, notch I and XhoI were digested, and M was DL 2,000 DNA Marker.

FIG. 27 The PCR-validated electrophoresis result of colonies of competent cells for the ligation product transformation of B602L and pS5E1-P72-IRES of example 4, where No. 1-7 were colonies and M was DL 5,000 DNA Marker.

FIG. 28 The validation of plasmid digestion electrophoresis detection of pS5E1-P72-IRES-B602L of example 4, wherein lanes 1, 2, 4, and 6 were the digestion identification of colony plasmids No. 1, 2, 4, and 6 NotI and XhoI selected from FIG. 27, and M was DL 15,000 DNA Marker.

FIG. 29 The results of the amplification and electrophoresis detection of the pS5E4-EGFP shuttle plasmid left arm, the pS5E4-EGFP shuttle plasmid right arm, the EF1a-EGFP-HBV, and the pS5E4-EGFP shuttle plasmid skeleton of example 5, wherein lane 1 was the pS5E4-EGFP shuttle plasmid left arm, lane 2 was the pS5E4-EGFP shuttle plasmid right arm, lane 3 was EF1α-EGFP-HBV, lane 4 was the pS5E4-EGFP shuttle plasmid skeleton, and M was DL 2,000 DNA Marker.

FIG. 30 The PCR validation electrophoresis result of colonies of competent cells for the transformation of four fragments of the shuttle plasmid of pSSE4-EGFP left arm, pSSE4-EGFP right arm, EF1α-EGFP-HBV, and pS5E4-EGFP shuttle plasmid skeleton of example 5, where lanes 1-20 were colonies and M was DL 2,000 DNA Marker.

FIG. 31 The result of the enzyme digestion verification of colonies No. 3, 4, 5, and 6 in FIG. 30 selected from example 5, wherein No. 1-4 were the three, four, five, and six positive clones PacI enzyme digestion, No. 5-8 were the three, four, five, and six positive clones HindIII enzyme digestion, M₁ and M₃ were DL 15,000 DNA Marker, and M₂ was DL 2,000 DNA Marker.

FIG. 32 The PCR amplification electrophoresis results of fragments P30, P54, and 2A of example 5, wherein lane 1 was the P30 amplified fragment, lane 2 was the P54 amplified fragment, lane 3 was the 2A amplified fragment, M₁ and M₂ were DL 2,000 DNA Marker.

FIG. 33 The result of the fusion PCR amplification electrophoresis of the P30-2A-P54 fragment of example 5, where lane 1 was the P30-2A-P54 fragment and M was DL 2,000 DNA Marker.

FIG. 34 The results of the vector enzyme digestion electrophoresis of fragments P30-2A-P54 and pS5E4-EGFP of example 5, where lanes 1 and 2 were pS5E4-EGFP, BamHI and XhoI were recovered by double enzyme digestion, lanes 3 and 4 were the recovery of fragments P30-2A-P54 BamHI and XhoI by double enzyme digestion, M₁ was DL 15,000 DNA Marker, and M₂ was DL 2,000 DNA Marker.

FIG. 35 The PCR validation electrophoresis result of a competent cell colony transformed by the ligation product of pSSE4 and P30-2A-P54 fragment of example 5, where No. 1-20 was a colony and M was DL 2,000 DNA Marker.

FIG. 36 The electrophoresis detection result of the extraction plasmids No. 2 and No. 19 of FIG. 35 picked out from example 5 for BamHI and XhoI double restriction enzyme digestion verification, wherein lane 2 was the BamHI and XhoI double restriction enzyme digestion verification of the positive clones No. 2, lane 19 is the BamHI and XhoI double restriction enzyme digestion verification of the positive clones No. 19, and M was DL 15,000 DNA Marker.

FIG. 37 The agarose gel validation electrophoresis results of pAd5LCL3 and pSSE1-P72-IRES-B602L of example 6, lane 1 was pAd5LCL3, lane 2 was pSSE1-P72-IRES-B602L, and M was DL 15,000 DNA Marker.

FIG. 38 The electrophoresis result of the plasmid pAd5LCL3-P72-IRES-B602L obtained by homologous recombination of the shuttle plasmid pSSE1-P72-IRES-B602L and the adenovirus vector plasmid pAd5LCL3 of example 6, wherein lanes 1-7 were clones of pAd5LCL3-P72-IRES-B602L and M was DL 15,000 DNA Marker.

FIG. 39 The result of the example 6 in which the No. 1 positive plasmid of FIG. 38 was selected and transformed into competent cells, and the plasmid extracted for restriction enzyme digestion. lane 1 was subjected to restriction enzyme digestion of plasmid XhoI of pAd5LCL3-P72-IRES-B602L, lane 2 was subjected to restriction enzyme digestion of plasmid PacI of pAd5LCL3-P72-IRES-B602L, and M was DL 15,000 DNA Marker.

FIG. 40 The agarose gel validation electrophoresis result of that shuttle plasmid pS5E4-P30-2A-P54 and the adenovirus vector plasmid pAd5LCL3-P72-IRES-B602L of example 6, where lane 1 was pS5E4-P30-2A-P54, lane 2 was pAd5LCL3-IRESP72-B602L, and M was DL 15,000 DNA Marker.

FIG. 41 The electrophoresis detection result of the plasmid pAd5LCL3-P72-IRES-B602L-P30-2A-P54 obtained by homologous recombination of the shuttle plasmid pS5E4-P30-2A-P54 and the adenovirus vector plasmid pAd5LCL3-P72-IRES-B602L of example 6, wherein lanes 1 to 8 were colonies and M was DL 15,000 DNA Marker.

FIG. 42 The result of the example 6 in which the No. 4 positive plasmid of FIG. 41 was selected and transformed into competent cells, and the plasmid was extracted for restriction enzyme digestion. lane 1 was subjected to restriction enzyme digestion of plasmid pAd5LCL3-P72-B602L-P30-P54 XhoI, lane 2 was subjected to restriction enzyme digestion of plasmid pAd5LCL3-P72-B602L-P30-P54 PacI, and M was DL 15,000 DNA Marker.

FIG. 43 The result of 293TD37 cells transfected with plasmid pAd5LCL3-P72-B602L-P30-P54 for 72 hours in example 7 (TP0).

FIG. 44 293TD37 cells at TP1 of example 7.

FIG. 45 293TD37 cells at TP2 of example 7.

FIG. 46 293TD37 cells at TP3 of example 7.

FIG. 47 CPE effect was observed in 293TD37 cells at TP4 of example 7.

FIG. 48 The results of Western Blot detection of P30 protein in the African swine fever multiantigen recombinant adenovirus vaccine pAd5LCL3-P72-B602L-P30-P54 in example 11.

FIG. 49 The vaccine-induced cytotoxic t cell (CTL) killing experiment in example 12 compared with the African swine fever multiantigen recombinant adenovirus pAd5LCL3-P72-B602L-P30-P54, the unrelated antigen pAd5-FMDO adenovirus group, and the normal saline group.

FIG. 50 The vector map of pAd5LCL3.

FIG. 51 The vector map of pS5E1.

FIG. 52 The vector map of pS5E1-P72-IRES-B602L.

FIG. 53 The vector map of pS5E4-EGFP.

FIG. 54 The vector map of pS5E4-P30-2A-P54.

FIG. 55 The vector map of pAd5LCL3-P72-B602L-P30-P54.

Fig. Western Blot analysis of the expression of P54 and P72 in the recombinant adenovirus vaccine pAd5LCL3-P72-B602L-P30-P54 of African swine fever in example 11, wherein M was Marker; Lane 1, P54 antibody serum; Lane 2, P72 antibody serum; Lane 3: 293TD37 cell control.

FIG. 57 The results of detecting the IgG antibody titers against the African swine fever target proteins P72 and P30 in serum by indirect ELISA in example 12 (ns, P≥0.05; *, P<0.05; **, P<0.01; ***, P<0.001; ****, P<0.0001), where the left figure was the IgG antibody titer of protein P72, and the right figure is the IgG antibody titer of P30.

FIG. 58 The schematic diagram of CD8+T cell response induced by the African swine fever recombinant adenovirus vaccine Ad5LCL3-P72-B602L-P30-P54 of example 12.

FIG. 59 The schematic diagram of CD4+T cell response induced by the African swine fever recombinant adenovirus vaccine Ad5LCL3-P72-B602L-P30-P54 of example 12.

FIG. 60 The representative diagram of the cellular immune response induced by the African swine fever recombinant adenovirus vaccine Ad5LCL3-P72-B602L-P30-P54 of example 12.

FIG. 61 The representative diagram of a blank control immune response in example 12.

FIG. 62 The results of the IRES fragment PCR amplification electrophoresis of example 14, in which lanes 1 and 2 were the IRES fragment PCR amplification products and M was DL 2,000 DNA Marker.

FIG. 63 The vector digestion electrophoresis of fragments IRES and pS5E1 of example 14, in which lane 1 was the digestion of fragments IRES EcoRV and NotI, lane 2 was the digestion of pS5E1 EcoRV and NotI, and M was DL 15,000 DNA Marker.

FIG. 64 The PCR validation electrophoresis result of colonies of competent cells transformed with the pS5E1 vector and the IRES fragment of example 14, where No. 1-9 were colonies and M was DL 2,000 DNA Marker.

FIG. 65 The illustration of the electrophoresis detection and verification of the pS5E1-IRES plasmids NotI and EcoRV digestion in example 14. plasmids 2 and 6 of FIG. 64 were selected for plasmid extraction and digestion verification, where lane 1 was for plasmid NotI No. 2 and EcoRV digestion identification, and lane 2 was for plasmid NotI No. 6 and EcoRV digestion identification.

FIG. 66 The results of the electrophoresis detection of MGF5L6L and pS5E1-IRES vector digestion of example 14, in which lane 1 was pS5E1-IRES, NotI and XhoI double-digestion, lane 2 was fragment MGF5L6L, NotI and XhoI double-digestion, and M was DL 15,000 DNA Marker.

FIG. 67 The PCR-validated electrophoresis result of colonies of competent cells transformed with the ligation product of MGF5L6L and pS5E1-IRES of example 14, where No. 1-12 were colonies and M was DL 2,000 DNA Marker.

FIG. 68 The plasmid digestion electrophoresis detection verification of pS5E1-IRES-MGF5L6L of example 14. colonies No. 2, 9 and 11 in FIG. 67 were selected for plasmid extraction and digestion verification, where lane 2 was designated as plasmid digestion verification No. 2, lane 9 as plasmid digestion verification No. 9, lane 11 as plasmid digestion verification No. 11, and M was DL 15,000 DNA Marker.

FIG. 69 The results of the fragment CP129Rubiqutin and the pS5E1-IRES-MGF5L6L vector restriction enzyme digestion product of example 14, in which lane 1 was the pS5E1-IRES-MGF5L6L plasmid, EcoRV and BamHI restriction enzyme digestion, lane 2 was the C129Rubiqutin fragment, EcoRV and BamHI restriction enzyme digestion, M was DL 15,000 DNA Marker or DL 2,000 DNA Marker.

FIG. 70 The results of the PCR-validated electrophoresis of colonies of competent cells transformed with the ligation product of pS5E1-IRES-MGF5L6L and CP129Rubiqutin of example 14, where No. 1-5 were colonies and M was DL 2,000 DNA Marker.

FIG. 71 The diagram illustrating the validation of the digestion electrophoresis detection of pS5E1-CP 129 rubiqutin-IRES-MGF5L6L plasmid of example 14, wherein lanes 1 and 2 were the digestion identification of the No. 1 and No. 2 colony plasmids BamHI and EcoRV selected from FIG. 70, and M was DL 2,000 DNA Marker.

FIG. 72 The results of the amplification electrophoresis detection of the pS5E4-EGFP shuttle plasmid left arm, the pS5E4-EGFP shuttle plasmid right arm, the EF1α-EGFP-HBV, and the pS5E4-EGFP shuttle plasmid skeleton of example 15, in which lane 1 was the pS5E4-EGFP shuttle plasmid left arm, lane 2 was the pS5E4-EGFP shuttle plasmid right arm, lane 3 was EF1α-EGFP-HBV, lane 4 was the pS5E4-EGFP shuttle plasmid skeleton, and M was DL 2,000 DNA Marker.

FIG. 73 The PCR validation electrophoresis result of a colony of competent cells for the transformation of four fragments of the pS5E4-EGFP shuttle plasmid left arm, pS5E4-EGFP shuttle plasmid right arm, EF1α-EGFP-HBV, and pS5E4-EGFP shuttle plasmid skeleton of example 15, wherein lanes 1 to 20 were colonies, and M was DL 2,000 DNA Marker.

FIG. 74 The electrophoresis detection result of the enzyme digestion verification of colonies No. 3, 4, 5, and 6 in FIG. 30 selected from example 15, wherein No. 1-4 were the three, four, five, and six positive clones PacI enzyme digestion, No. 5-8 were the three, four, five, and six positive clones HindIII enzyme digestion, M₁ and M₃ were DL 15,000 DNA Marker, and M₂ was DL 2,000 DNA Marker.

FIG. 75 The PCR amplification electrophoresis results of fragments CP312R, MGF110-4L, and 2A of example 15, wherein lane 1 was the CP312R amplified fragment, lane 2 was the 2A amplified fragment, lane 3 was the MGF110-4L amplified fragment, and M was DL 2,000 DNA Marker.

FIG. 76 The result of the fusion PCR amplification electrophoresis of the fragment CP312R-2A-MGF110-4L of example 15, where lane 1 was the fragment CP312R-2A-MGF110-4L and M was DL 2,000 DNA Marker.

FIG. 77 The results of restriction endonuclease digestion electrophoresis of fragment CP312R-2A-MGF110-4L and pS5E4-EGFP of example 15, in which lane 1 shows the recovery of fragment CP312R-2A-MGF110-4L gel, lane 2 shows the recovery of fragment pS5E4-EGFP, BamHI and XhoI double restriction endonuclease digestion, and M was DL 15,000 DNA Marker.

FIG. 78 The results of the PCR-validated electrophoresis of colonies of competent cells ligated with the fragment CP312R-2A-MGF110-4L of pS5E4 of example 15, where No. 1-12 were colonies and M was DL 15,000 DNA Marker.

FIG. 79 The electrophoresis detection result of the extraction plasmids of positive clones No. 1, 2, 3, and 4 of FIG. 78 picked out from example 15 for BamHI and XhoI double restriction enzyme digestion verification, wherein lanes 1, 2, 3, and 4 were BamHI and XhoI double restriction enzyme digestion verification of positive clones No. 1, 2, 3, and 4, respectively, and M was DL 15,000 DNA Marker.

FIG. 80 The agarose gel validation electrophoresis results of pAd5LCL3 and pS5E1-CP129R ubiqutin-IRES-MGF5L6L of example 16, where lane 1 was pS5E1-CP129R ubiqutin-IRES-MGF5L6L1 and lane 2 was pAd5LCL3, M was DL 15,000 DNA Marker.

FIG. 81 The electrophoresis detection result of the plasmid pAd5LCL3-CP129R ubiqutin-IRES-MGF5L6L obtained by homologous recombination of the shuttle plasmid pS5E1-CP129R ubiqutin-IRES-MGF5L6Land the adenovirus vector plasmid pAd5LCL3 of example 16, where lanes 1-5 were clones pAd5LCL3-CP129R ubiqutin-IRES-MGF5L6Land M was DL 15,000 DNA Marker.

FIG. 82 The result of the plasmid No. 4 selected from FIG. 81 and transformed into competent cells in example 16 and extracted for restriction endonuclease analysis. lanes 1 and 2 refer to the restriction endonuclease analysis of plasmid XhoI of pAd5LCL3-CP129R ubiqutin-IRES-MGF5L6Land M was DL 15,000 DNA Marker.

FIG. 83 The agarose gel validation electrophoresis results of the shuttle plasmid pS5E4-CP312R-2A-MGF110-4L and the adenovirus vector plasmid pAd5LCL3-CP129Rubiqutin-IRES-MGF5L6Lin example 16, where lane 1 was pS5E4-CP312R-2A-MGF110-4L, lane 2 was pAd5LCL3-CP129R ubiqutin-IRES-MGF5L6L, and M was DL 15,000 DNA Marker.

FIG. 84 The electrophoresis detection result of a plasmid pAd5LCL3-CP129R ubiqutin-IRES-MGF5L6L obtained by homologous recombination of the shuttle plasmid pS5E4-CP312R-2A-MGF110-4L and the adenovirus vector pAd5LCL3-CP129R ubiqutin-MGF5L6L-CP312R-MGF110-4L of example 16, in which lanes 1-6 were plasmids and M was DL 15,000 DNA Marker.

FIG. 85 The result of the example 16 in which the no.3 positive plasmid of FIG. 84 was selected and converted to competent cells, and the plasmid was extracted for restriction enzyme digestion. lane 1 was the pAd5LCL3-CP129R ubiqutin-IRES-MGF5L6L plasmid XhoI restriction enzyme digestion, lane 2 was the pAd5LCL3-CP129R ubiqutin-IRES-MGF5L6L plasmid PacI restriction enzyme digestion, and M was DL 15,000 DNA Marker.

FIG. 86 293TD37 cells at TP0 of example 17.

FIG. 87 293TD37 cells at TP1 of example 17.

FIG. 88 293TD37 cells at TP2 of example 17.

FIG. 89 293TD37 cells at TP3 of example 17.

FIG. 90 CPE effect was observed in 293TD37 cells at TP4 of example 17.

FIG. 91 Western Blot analysis of CP312R protein in African swine fever recombinant adenovirus vaccine pAd5LC13-CP129Rubiquitin-MGF5L6L-CP312R-MGF110-4L in example 21.

FIG. 92 The vector map for pS5E1-C129Rubiqutin-IRES-MGF5L6L.

FIG. 93 The vector map of pS5E4-EGFP.

FIG. 94 The vector map for pS5E4-CP312R-2A-MGF110-4L.

FIG. 95 The vector map for pAd5LCL3-C129Rubiquitin-MGF5L6L-CP312R-MGF110-4L

FIG. 96 The results of the CD8+T cell response induced by pAd5LC13-CP129Rubiquitin-MGF5L6L-CP312R-MGF110-4L of example 22.

FIG. 97 The results of the CD4+T cell response induced by pAd5LC13-CP129Rubiquitin-MGF5L6L-CP312R-MGF110-4L of example 22.

FIG. 98 The cellular immune response after intramuscular injection of pAd5LC13-CP129Rubiquitin-MGF5L6L-CP312R-MGF110-4L in example 22

FIG. 99 The blank control immune response of example 22.

FIG. 100 The electrophoresis detection of the cleavage of I215L with pS5E1-IRES vector in example 23, in which the lane vector was pS5E1-IRES, NotI and XhoI double-cleaved, lane I215L was fragment I215L, NotI and XhoI double-cleaved, and M was DL 15,000 DNA Marker, or DL 2,000 DNA Marker.

FIG. 101 The of the PCR-validated electrophoresis of colonies of competent cells transformed with the I215L and pS5E1-IRES ligation products of example 23, where No. 1-li were colonies and M was DL 2,000 DNA Marker.

FIG. 102 The diagram illustrating the plasmid digestion electrophoresis detection verification of pS5E1-IRES-I215L of example 23. colonies No. 5, 6, 7 and 8 in FIG. 101 were selected for plasmid extraction and digestion verification, where lane 2 was the plasmid digestion verification No. 2, lane 9 was the plasmid digestion verification No. 9, lane 11 was the plasmid digestion verification No. 11, and M was DL 15,000 DNA Marker.

FIG. 103 The result of the L8Lubiqutin fragment obtained by fusing L8L and ubiquitin of example 23 and purified by a gel recovery and purification kit, wherein lane 1 was the L8Lubiqutin fragment and M was DL 2,000 DNA Marker.

FIG. 104 The result of electrophoresis detection of the enzyme-cleaved product of fragment L8Lubiqutin and pS5E1-IRES-I215L vector of example 23, wherein lane 1 was the pS5E1-IRES-I215L plasmid; Lane 2 was the pS7E1-IRES-I215L plasmid EcoRV and BamHI restriction enzyme digestion; Lane 3 was the EcoRV and BamHI restriction endonucleases of the L8Lubiqutin fragment, M was DL 15,000 DNA Marker.

FIG. 105 The results of PCR-validated electrophoresis of colonies of competent cells transformed with the ligation product of pS5E1-IRES-I215L vector and L8Lubiqutin of example 23, where No. 1-24 were colonies and M was DL 2,000 DNA Marker.

FIG. 106 The validation of plasmid digestion electrophoresis detection of pS5E1-L8Lubiqutin-IRES-I215L in example 23, where lanes 4, 6, 9, 14, 17 and 18 were the digestion identification of colony plasmids BamHI and EcoRV No. 4, 6, 9, 14, 17 and 18 in FIG. 105, and M was DL 15,000 DNA Marker.

FIG. 107 The electrophoresis results of the fusion PCR amplified I73RHBsAg and 2A-E146L fragments of example 24, where lane 1 was the I73RHBsAg fragment, lane 2 was the 2A-E146L fragment, and M was DL 2,000 DNA Marker.

FIG. 108 The results of fragment pS5E4-EGFP vector digestion electrophoresis of example 24, in which lane 1 was fragment pS5E4-EGFP, BamHI and XhoI were recovered by double digestion, and M was DL 15,000 DNA Marker.

FIG. 109 The PCR validation electrophoresis result of the pS5E4-EGFP gel recovery vector of example 24 seamlessly clonally connected to the I73RHBsAg fragment, 2A-E146L, and transforming competent cell colonies, where No. 1-12 were colonies, and M was DL 15,000 DNA Marker.

FIG. 110 The electrophoresis detection result of the extraction plasmids No. 1, No. 2, and No. 3 positive clones of FIG. 109 picked out in example 24 for BamHI and XhoI double enzyme digestion verification, wherein lanes 1, 2, and 3 were the BamHI and XhoI positive clones No. 1, 2, and 3, respectively, and M was DL 15,000 DNA Marker.

FIG. 111 The agarose gel validation electrophoresis results of pAd5LCL3 and pS5E1-L8Lubiqutin-IRES-I215L of example 25, with lane 1 being pAd5LCL3 and lane 2 being pS5E1-L8Lubiqutin-IRES-I215L,M was DL 15,000 DNA Marker.

FIG. 112 The electrophoresis detection result of the plasmid pAd5LCL3-L8Lubiqutin-IRES-I215L obtained by homologous recombination of the shuttle plasmid pS5E1-L8Lubiqutin-IRES-I215L and the adenovirus vector plasmid pAd5LCL3 in example 25, where lanes 1-7 were clones pAd5LCL3-L8Lubiqutin-IRES-I215L and M was DL 15,000 DNA Marker.

FIG. 113 The plasmid pAd5LCL3-P72-IRES-B602L was verified by XhoI digestion, lane 1 was the positive plasmid no.6 of FIG. 112, M was DL 15,000 DNA Marker.

FIG. 114 The agarose gel validation electrophoresis results of the shuttle plasmid pS5E4-I73RHBsAg-2A-E146L and the adenovirus vector plasmid pAd5LCL3-L8Lubiqutin-IRES-I215Lin example 25, where lane 1 was pS5E4-I73RHBsAg-2A-E146L, lane 2 was pAd5LCL3-L8Lubiqutin-IRES-I215L, and M was DL 15,000 DNA Marker.

FIG. 115 The electrophoresis detection result of pAd5LCL3-L8Lubiqutin-I215L-I73RHBsAg-E146L obtained by homologous recombination of the shuttle plasmid pS5E4-I73RHBsAg-2A-E146L and the adenovirus vector plasmid pAd5LCL3-L8Lubiqutin-IRES-I215L in example 25, where lanes 1 to 8 were plasmids and M was DL 15,000 DNA Marker.

FIG. 116 The plasmid pAd5LCL3-L8Lubiqutin-I215L-I73HBsAg-E146L was verified by XhoI digestion, lane 2 was the positive plasmid no.6 of FIG. 112 was the No. 2 positive plasmid of FIG. 115, and M was DL 15,000 DNA Marker.

FIG. 117 293TD37 cells at TP0 of example 26.

FIG. 118 293TD37 cells at TP1 of example 26.

FIG. 119 293TD37 cells at TP2 of example 26.

FIG. 120 293TD37 cells at TP3 of example 26.

FIG. 121 CPE effect was observed in 293TD37 cells at TP4 of example 26.

FIG. 122 Western Blot analysis of L8Lubiqutin protein and I215L protein in African swine fever recombinant adenovirus vaccine pAd5LCL3-L8Lubiqutin-I215L-I73HBsAg-E146L in example 21, lane 1 was the 293 blank cells, and lanes 2 and 3 were the samples of the cells infected with pAd5LCL3-L8Lubiqutin-I215L-I73HBsAg-E146L, M was marker.

FIG. 123 The vector map of pS5E1-L8Lubiqutin-IRES-I215L.

FIG. 124 The vector map of pS5E4-I73RHBsAg-2A-E146L.

FIG. 125 The vector map of pAd5LCL3-CP129Rubiqutin-MGF5L6L-CP312R-MGF4L.

FIG. 126 The results of the CD8+T cell response induced by pAd5LCL3-L8Lubiqutin-I215L-I73RHBsAg-E146Lin Example 30.

FIG. 127 The results of the CD4+T cell response induced by pAd5LCL3-L8 Lubiqutin-I215L-173R HBsAg-E146L in Example 30.

FIG. 128 The cellular immune response after intramuscular injection of pAd5LCL3-L8 Lubiqutin-I215L-I73R HBsAg-E146L in example 31.

FIG. 129 The blank control immune response of example 31.

FIG. 130 The PCR-validated electrophoresis result of colonies of competent cells transformed with the ligation product of EP402R and pS5E1-IRES of example 32, where No. 1-6 were colonies and M was DL 2,000 DNA Marker.

FIG. 131 The diagram illustrating the plasmid restriction enzyme digestion electrophoresis detection verification of pS5E1-EP402R-IRES of example 32. colonies No. 1, 2, and 4 in FIG. 130 were selected for plasmid extraction and restriction enzyme digestion verification, where lane 1 was the plasmid restriction enzyme digestion verification No. 1, lane 2 was the plasmid restriction enzyme digestion verification No. 2, lane 4 was the plasmid restriction enzyme digestion verification No. 4, and M was DL 15,000 DNA Marker.

FIG. 132 The electrophoresis detection results of the enzymatic cleavage products of pSSE1-EP402R-IRES vector and EP153R vector of example 32, in which lane 1 was pS5E1-EP402R-IRES with NotI and XhoI enzymatic cleavage, lane 2 was EP153R fragment with NotI and XhoI enzymatic cleavage, and M was DL 2,000 DNA Marker.

FIG. 133 The PCR-validated electrophoresis result of colonies of competent cells transformed with the ligation product of pS5E1-EP402R-IRES vector and EP153R of example 32, where No. 1-14 were colonies and M was DL 5,000 DNA Marker.

FIG. 134 The validation of plasmid digestion electrophoresis detection of pS5E1-F317L-IRES-A151R of example 32, where lanes 1, 2, 3, 5, 11 and 13 were the digestion identification of colony plasmids BamHI and EcoRV No. 1, 2, 3, 5, 11 and 13 in FIG. 27, and M was DL 15,000 DNA Marker.

FIG. 135 The electrophoresis detection results of the fusion PCR amplified I177L-2A and K205Rubiqutin fragments of example 33, lane 1 was fragment of K205R; Lane 2 was fragment of ubiqutin; Lane 3 was fragment of K205Rubiqutin; Lane 4 was fragment 2a; Lane 5 was fragment of I177L; Lane 6 was fragment of I177L-2A and M was DL 2,000 DNA Marker.

FIG. 136 The results of fragment pS5E4-EGFP vector digestion electrophoresis of example 33, in which lane 1 was fragment of pS5E4-EGFP, digested by BamHI and XhoI, recovered by gel extraction, M was DL 15,000 DNA Marker.

FIG. 137 The colony PCR of pS5E4-I177L-2A-K205Rubiqutin of example 33, where No. 1-3 were colonies, and M was DL 15,000 DNA Marker.

FIG. 138 The electrophoresis detection result of the extraction plasmids No. 1, No. 2, and No. 3 positive clones of FIG. 137, BamHI and XhoI double enzyme digestion verification, wherein lanes 1, 2, and 3 were the BamHI and XhoI positive clones No. 1, 2, and 3, respectively, and M was DL 15,000 DNA Marker.

FIG. 139 The agarose gel validation electrophoresis results of pAd5LCL3 and pSSE1-EP402R-IRES-EP153R of Example 34, lane 1 was pAd5LCL3 and lane 2 was pSSE1-EP402R-IRES-EP153R, M was DL 15,000 DNA Marker.

FIG. 140 The electrophoresis result of the plasmid pAd5LCL3-EP402R-IRES-EP153R obtained by homologous recombination of the shuttle plasmid pAS5E1-EP402R-IRES-EP153R and the adenovirus vector plasmid pAD5LCL3 of example 34, where lanes 1-8 were clones of pAd5LCL3-EP402R-IRES-EP153R and M was DL 15,000 DNA Marker.

FIG. 141 The result of the transformation to competent cells of the No. 2 positive plasmid of FIG. 140 picked out from example 34, and the plasmid was extracted for restriction enzyme digestion. lane 1 was the plasmid XhoI restriction enzyme digestion of pAd5LCL3-EP402R-IRES-EP153R, lane 2 was the plasmid PacI restriction enzyme digestion of pAd5LCL3-EP402R-IRES-EP153R, lane 3 was the plasmid BamHI restriction enzyme digestion of pAd5LCL3-EP402R-IRES-EP153R, and M was DL 15,000 DNA Marker.

FIG. 142 The electrophoresis detection result of a recombinant adenovirus vector pAd5LCL3-EP402R-EP153R-I177L-K205Rubiqutin obtained by homologous recombination of the shuttle plasmid pSSE4-I177L-2A-K205Rubiqutin and the adenovirus vector plasmid pAd5LCL3-EP402R-IRES-EP153R of example 34, where lanes 1 to 7 were plasmids and M was DL 15,000 DNA Marker.

FIG. 143 The result of the transformation of the No. 1 positive plasmid of Pick-up to competent cells in Example 34, and the extraction of the plasmid for restriction enzyme digestion verification, where lanes 1 and 2 were the results of restriction enzyme digestion of plasmid XhoI of PD5 LCL3-EP402R-EP 153R-I177L-K205 rubiqutin. Lane 3 was BamHI restriction endonuclease of the plasmid pAd5LCL3-EP402R-EP153R-I177L-K205Rubiqutin, lane 4 was pAd5LCL3-EP402R-EP153R-I177L-K205Rubiqutin, PacI restriction endonuclease, and M was DL 15,000 DNA Marker.

FIG. 144 293TD37 cells at TP0 of example 35.

FIG. 145 293TD37 cells at TP1 of example 35.

FIG. 146 293TD37 cells at TP2 of example 35.

FIG. 147 293TD37 cells at TP3 of example 35.

FIG. 148 CPE effect was observed in 293TD37 cells at TP4 of example 35.

FIG. 149 Western Blot analysis of EP153R protein in African swine fever recombinant adenovirus vaccine pAd5LCL3-EP402R-EP153R-K205R-I177Lubiqutin in example 39, M was marker.

FIG. 150 The vector map of pSSE1-EP402R-IRES-EP153R.

FIG. 151 The vector map of pSSE4-I177L-2A-K205Rubiqutin.

FIG. 152 The vector map of pAd5LCL3-EP402R-EP153R-I177L-K205Rubiqutin.

FIG. 153 The ELISA of the IgG antibody titer against the African swine fever target protein EP402R of example 40.

FIG. 154 The of the CD8+T cell response induced by pAd5LCL3-EP402R-EP153R-K205R-I177Lubiqutin of example 40.

FIG. 155 The results of the CD4+T cell response induced by pAd5LCL3-EP402R-EP153R-K205R-I177Lubiqutin of example 40.

FIG. 156 The cellular immune response after intramuscular injection of pAd5LCL3-EP402R-EP153R-K205R-I177Lubiqutin of example 40.

FIG. 157 The blank control immune response of example 40.

FIG. 158 The results of the electrophoresis detection of F317L and pS5E1-IRES vector digestion in example 41, in which lane 1 was pS5E1-IRES, BamHI and EcoRV double digestion, lane 2 was fragment F317L, BamHI and EcoRV double digestion, and M was DL 15,000 DNA Marker.

FIG. 159 The results of the PCR-validated electrophoresis of colonies of competent cells transformed with the F317L and pS5E1-IRES ligation products of example 41, where No. 1-14 were colonies and M was DL 2,000 DNA Marker.

FIG. 160 The diagram illustrating the plasmid restriction enzyme digestion electrophoresis detection verification of pS5E1-F317L-IRES of example 41,colonies No. 9 and 10 in FIG. 159 were selected for plasmid extraction and restriction enzyme digestion verification, where lane 1 was the plasmid restriction enzyme digestion verification No. 1, lane 2 was the plasmid restriction enzyme digestion verification No. 9, and M was DL 15,000 DNA Marker.

FIG. 161 The result of electrophoresis detection of the vector-digested product of fragment A151R and pS5E1-F317L-IRES of example 41, in which lane 1 was pS5E1-F317L-IRES, digested by NotI and XhoI; Lanes 2 and 3 were fragments A151R, digested by NotI and XhoI, and M was DL 15,000 DNA Marker.

FIG. 162 The PCR validation electrophoresis result of a colony of competent cells transformed with the pS5E1-F317L-IRES vector and A151R of example 41, where No. 1-24 were colonies and M was DL 2,000 DNA Marker.

FIG. 163 The validation of plasmid digestion electrophoresis detection of pS5E1-F317L-IRES-A151R of example 41, where lanes 1, 2, 3 and 4 were the digestion identification of colony plasmids BamHI and EcoRV No. 4, 15, 23 and 24 selected from FIG. 162, and M was DL 15,000 DNA Marker.

FIG. 164 The PCR product electrophoresis result of the target fragments of P34, 2A, and pp62 of example 42, wherein lane 1 was fragment of p34; 2 was fragment of 2A; 3 was fragment of pp62, M was DL 2,000 DNA Marker.

FIG. 165 The electrophoretic detection result of the fusion PCR amplified fragment of P34-2A of example 42, in which lane 1 was fragment of P34-2A and M was DL 2,000 DNA Marker.

FIG. 166 The result of fragment pS5E4-EGFP vector digestion electrophoresis of example 42, in which lane 1 was fragment of pS5E4-EGFP, digested by BamHI and XhoI, and M was DL 15,000 DNA Marker.

FIG. 167 The PCR validation electrophoresis result of the pS5E4-EGFP gel recovery vector of example 42 which was seamlessly clonally connected to P34-2A fragment and pp62 and transformed into competent cell colonies, where No. 1-12 were colonies and M was DL 15,000 DNA Marker.

FIG. 168 The electrophoresis detection result of the extraction plasmids of the positive clones No. 1, 2, 9, and 11 of FIG. 167 picked out from example 42 for BamHI and XhoI double restriction enzyme digestion verification, wherein lanes 1, 2, 3, and 4 were the positive clones No. 1, 2, 9, and 11, respectively, BamHI and XhoI double restriction enzyme digestion verification, and M was a maker.

FIG. 169 The agarose gel validation electrophoresis results of pAd5LCL3 and pS5E1-F317L-IRES-A151R of example 43, where lane 1 was pAd5LCL3, lane 2 was pS5E1-F317L-IRES-A151R, and M was a maker.

FIG. 170 The electrophoresis detection result of the plasmid pAd5LCL3-F317L-IRES-A151R obtained by homologous recombination of the shuttle plasmid pS5E1-F317L-IRES-A151R and the adenovirus vector plasmid pAd5LCL3 of example 43, where lanes 1-7 were pAd5LCL3-F317L-IRES-A151R and M was a maker.

FIG. 171 The results of the plasmid XhoI digestion test performed on the No. 3 positive plasmid of FIG. 170 selected from example 43 and transformed to competent cells, and the extracted plasmid, lane 1 and lane 2, were pAd5LCL3-P72-IRES-B602L plasmid xhoi digestion, and M was a maker.

FIG. 172 The agarose gel validation electrophoresis result of shuttle plasmid pS5E4-P34-2A-pp62 and adenovirus vector plasmid pAd5LCL3-F317L-IRES-A151R of example 43, where lane 1 was pS5E4-P34-2A-pp62, lane 2 was pAd5LCL3-F317L-IRES-A151R, and M was a maker.

FIG. 173 The electrophoresis detection result of a plasmid of the recombinant adenovirus vector pAd5LCL3-F317L-A151R-P34-2A-pp62 obtained by homologous recombination of the shuttle plasmid pS5E4-P34-2A-pp62 and the adenovirus vector plasmid pAd5LCL3-F317L-IRES-A151R of example 43, where lane 1-4 was a plasmid and M was a maker.

FIG. 174 The result of the plasmid XhoI digestion test performed on the No. 2 positive plasmid of FIG. 173 selected from example 43 and transformed into competent cells, and the extracted plasmid was subjected to enzyme digestion verification, wherein lane 2 was the plasmid xhoi digestion of pAd5LCL3-F317L-A151R-P34-pp62, and M was a maker.

FIG. 175 293TD37 cells at TP0 of example 44.

FIG. 176 293TD37 cells at TP1 of example 44.

FIG. 177 293TD37 cells at TP2 of example 44.

FIG. 178 293TD37 cells at TP3 of example 44.

FIG. 179 CPE effect was observed in 293TD37 cells at TP4 of example 44.

FIG. 180 The schematic diagram showing the results of detecting the pp62 protein in the African swine fever multiantigen recombinant adenovirus vaccine pAd5LCL3-F317L-A151R-P34-pp62 by Western Blot in example 48.

FIG. 181 The vector map of pSSE1-F317L-IRES-A151R.

FIG. 182 The vector map of pS5E4-P34-2A-pp62.

FIG. 183 The vector map of pAd5LCL3-F317L-A151R-P34-PP62.

FIG. 184 The results of detecting the IgG antibody titer against the African swine fever target protein pp62 in serum by the ELISA method of example 49.

FIG. 185 The results of the CD8+T cell response induced by pAd5LCL3-F317L-A151R-P34-pp62 of example 49.

FIG. 186 The results of the CD4+T cell response induced by pAd5LCL3-F317L-A151R-P34-pp62 of example 49.

FIG. 187 The cellular immune response after intramuscular injection of pAd5LCL3-F317L-A151R-P34-pp62 in example 49.

FIG. 188 The blank control immune response of example 49.

### DETAILED DESCRIPTION OF THE INVENTION

Preferred embodiments of the present invention are described in further detail below with reference to the accompanying drawings, and it should be noted that the embodiments described below are intended to facilitate an understanding of the present invention and are not intended to limit the same in any way.

### Example 1: Construction of Adenovirus Vector Plasmid pAd5 with E1 and E3 Genes Deletion

The wild-type human adenovirus type 5 (ATCC® VR-1516, gene sequence AC_000008.1) virus was amplified in A549 cells (ATCC® CCL-185), and the virus liquid was collected and concentrated. The adenovirus genome was extracted by HirtVirual DNA extraction. The linear hAd5 genome was constructed into a circular supercos-Ad5 vector plasmid by a cosmid method. The E1 region of hAd5 adenovirus was excised(deleted) by CRISPR/cas9technology. The designed gRNA was as follows:

HAd5-E1 upstream gRNA:
(SEQ ID NO: 38)
GGCGGGAAAACUGAAUAAGGUUUUAGAGCUAGAAAUAGCAAGUUAAAAU
AAGGCUAGUCCGUUAUCAACUUGAAAAAGUGGCACCGAGUCGGUGCUUU
U
HAd5-E1 downstream gRNA:
(SEQ ID NO: 39)
GAGAUGAUCCAGUCGUAGCGUUUUAGAGCUAGAAAUAGCAAGUUAAAAU
AAGGCUAGUCCGUUAUCAACUUGAAAAAGUGGCACCGAGUCGGUGCUUU
U.

Designing a gRNA site at the upstream and downstream of the hAd5 E1 region, recovering a large fragment vector after cutting, designing a primer, respectively inserting the ITR and PIX sequences into the upstream and the downstream by fusion PCR and introducing a SwaI restriction enzyme cutting site, and then performing seamless cloning on the fused fragment and the vector to obtain an supercos-ad5ΔE1 adenovirus vector wherein E1 is knockout.

And then performing E3 region excision on the supercos-ad5ΔE1 plasmid, and designing the gRNA as follows:

HAd5-E3 Upstream gRNA:
(SEQ ID NO: 40)
GCGGGACAUUUCAGAUCGGGUUUUAGAGCUAGAAAUAGCAAGUUAAAAU
AAGGCUAGUCCGUUAUCAACUUGAAAAAGUGGCACCGAGUCGGUGCUUU
U
HAd5-E3 downstream gRNA:
(SEQ ID NO: 41)
GUAAGGGUACUGCUAUCGGGUUUUAGAGCUAGAAAUAGCAAGUUAAAAU
AAGGCUAGUCCGUUAUCAACUUGAAAAAGUGGCACCGAGUCGGUGCUUU
U

A gRNA site was designed upstream and downstream of the hAd5 E3 region, and the large fragment vector was recovered after cutting. Primers were then designed to perform fusion PCR on excessive excised Fiber and pVIII sequences upstream and downstream of E3, and seamless cloning was used for ligation to obtain the adenovirus vector plasmid pAd5 with E1 and E3 genes deletion and introducing the SwaI restriction site.

### Example 2: Construction of Adenovirus Vector Plasmid Pad5.ΔE4 without E1, E3 and E4 Genes

By adopting the vector plasmid pAd5 obtained in example 1 with the knocked out E1 and E3 genes, and further knocking out the E4 gene. The capacity of the adenovirus vector can be increased, and the immunogenicity can be reduced; and a part of fiber was amplified by a PCR method and a restriction site of NdeI was introduced, and then an extra excised fragment was connected to the vector by a seamless cloning method of Gibson to obtain the vector plasmid pAd5ΔE4 with the E1, E3 and E4 genes deletion, and the restriction sites of SwaI and I-sceI were introduced.

The detail steps are:

### 1. Selection of CRISPR Target Sequence of the Target Gene E4.

### 1) Selection of CRISPR Target Sequence of Fiber Gene Upstream of E4 Gene

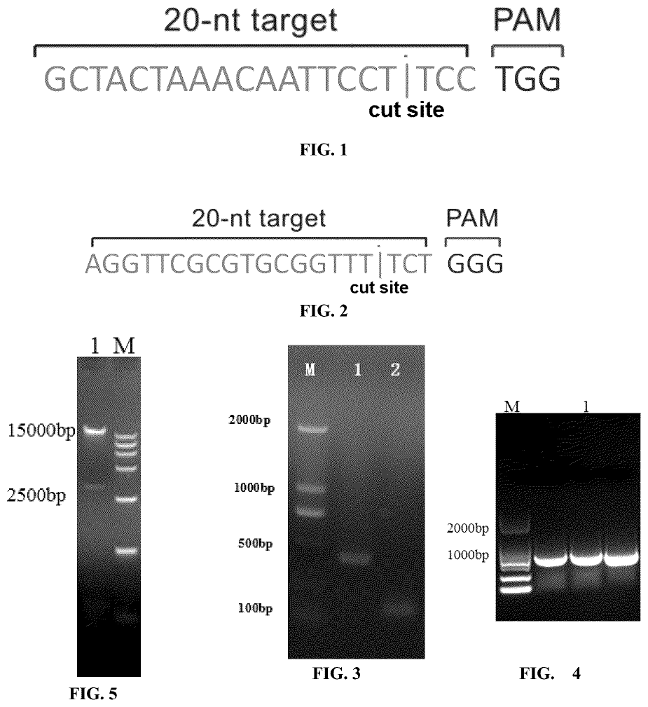

The first 400 bp of fiber gene were input using GeneArt™ CRISPR Search and Design tool(thermofisher.com/crispresign) software from Thermo Fisher Scientific. The software automatically analyzed the sequence of the 400 bp and provided six potential CRISPR target sequences. Considering the length of the knockout sequence of E4 gene and the requirement of constructing a live vector, GCTACTAAACAATTCCITCC (SEQ ID NO: 164) was selected as the targeting sequence, and the finally obtained gRNA was named Ad5-E4-up-gRNA, with the cleavage site and PAM site shown in FIG. 1.

### 2) Selection of CRISPR Target Sequence of Downstream Non-Coding Sequence of E4

Using the software of GeneArt™ CRISPR Search and Design Tool (thermofisher.com/crisprdesign) in Thermo Fisher Scientific, 300 bp downstream of E4 were input, which was automatically analyzed by the software to provide six potential CRISPR target sequences. AGGTTCGCGTGCGGTTTCT (SEQ ID NO: 165) was selected as the target sequence, and the finally obtained gRNA was named Ad5-E4-down-gRNA, with the cleavage site and PAM site shown in FIG. 2.

### 2. DNA Amplification of Ad5-E4-Up-gRNA and Ad5-E4-Down-gRNA

1) DNA template design for Ad5-E4-up-gRNA
(SEQ ID NO: 42)
5′-TAATACGACTCACTATAGTACTAAACAATTCCTTCCGTTTTAGAGC
TAGAAATAGCAAGTTAAAATAAGGCTAGTCCGTTATCAACTTGAAAAAG
TGGCACCGAGTCGGTGCTTTT-3′
2) DNA template design for Ad5-E4-down-gRNA
(SEQ ID NO: 43)
5′-TAATACGACTCACTATAGGTTCGCGTGCGGTTTTCTGTTTTAGAGC
TAGAAATAGCAAGTTAAAATAAGGCTAGTCCGTTATCAACTTGAAAAAG
TGGCACCGAGTCGGTGCTTTT-3′.

### 3. Design of Upstream and Downstream Primers for Amplifying the DNA Templates of Ad5-E4-Up-gRNA and Ad5-E4-Down-gRNA.

The upstream and downstream primers were designed for PCR amplification of the DNA template of Ad5-E4-up-gRNA and the DNA template of Ad5-E4-down-gRNA, respectively, and amplified using the Gene Art™ Precision gRNA Synthesis Kit.

### Primer Design:

Ad5-E4-up-gRNA-Forward:
(SEQ ID NO: 44)
TAATACGACTCACTATAGTACTAAACAATTCCT
Ad5-E4-up-gRNA-Reverse:
(SEQ ID NO: 45)
TTCTAGCTCTAAAACGGAAGGAATTGTTTAGTA
Ad5-E4-down-gRNA-Forward:
(SEQ ID NO: 46)
TAATACGACTCACTATAGGTTCGCGTGCGGTTT
Ad5-E4-down-gRNA-Reverse:
(SEQ ID NO: 47)
TTCTAGCTCTAAAACAGAAAACCGCACGCGAAC

### 4. Amplification of the DNA Templates of Ad5-E4-Up-gRNA and Ad5-E4-Down-gRNA

### 1) Prepare a 0.3 μM Mixture of Ad5-E4-Up-gRNA-Forward/Reverse Primers

10 μM Ad5-E4-up-gRNA-Forward primer 3 ul, 10 μM Ad5-E4-up-gRNA-Reverse primer 3 ul, and fill with water up to 100 ul.

### 2) Prepare 0.3 μM Working Mixture of AD5-E4-DOWN-GRNA-Forward/REVERSE Primers

10 μM Ad5-E4-down-gRNA-Forward primer 3 ul, 10M Ad5-E4-down-gRNA-Reverse primer 3 ul, fill with water to 100 ul.

### 3) PCR Reaction System:

The PCR reaction system for DNA template amplification of Ad5-E4-up-gRNA was: Phusion™ High-Fidelity PCR Master Mix (2×) 12.5 uL, Tracr Fragment+T7 Primer Mix 1 ul, and 0.3 μM AD5-E4-UP-gRNA-Forward/Reverse primer mixture 1 ul, filled with water to 25 ul.

The PCR reaction system for DNA template amplification of Ad5-E4-down-gRNA was: Phusion™ High-Fidelity PCR Master Mix (2λ) 12.5 uL, Tracr Fragment+T7 Primer Mix 1 ul, and 0.3 μM AD5-E4-DOWN-gRNA-Forward/Reverse primer mixture working solution 1 ul, filled with water to 25 ul.

### 4) PCR Program:

Initial denaturation 98° C., 10 sec, 1 cycle; Denaturation 98° C., 5 sec; Annealing at 55° C. for 15 sec and 32 cycles; Extension at 72° C. for 1 min and 1 cycle; Maintain 4° C.

### 5: In Vitro Transcription to Obtain Ad5-E4-Up-gRNA and Ad5-E4-Down-gRNA

In vitro transcription of the template DNA was performed using TranscriptAid™ EnzymeMix to obtain Ad5-E4-up-gRNA and Ad5-E4-down-gRNA.

The reaction system for obtaining Ad5-E4-up-gRNA through in vitro transcription was as follows: NTP mix 8 ul, E1A-gRNA DNA template 6 ul, 5×TranscriptAid™ Reaction Buffer 4 uL, TranscriptAid™ Enzyme Mix 2 uL. After incubation at 37° C. for 4 hours, add 1 ul of DNase I and incubate at 37° C. for 15 minutes.

The reaction system for obtaining Ad5-E4-down-gRNA through in vitro transcription is as follows: NTP mix 8 ul, E1B-gRNA DNA template 6 ul, 5×TranscriptAid™ Reaction Buffer 4 uL, and TranscriptAid™ Enzyme Mix 2 uL. After incubation at 37° C. for 4 hours, add 1 ul of DNase I and incubate at 37° C. for 15 minutes.

Ad5-E4-up-gRNA, Ad5-E4-down-gRNA were obtained by in vitro transcription

### 6: Purification of In Vitro Transcription Product

1) fill the transcribed reaction system to 200 ul; with nuclease-free water;
2) Add 100 ul of Binding buffer, and mix well;
3) Add 300 ul of ethanol (>96%) and mix well;
4) The mixed solution was transferred to the Gene Jet™ RNA Purification Micro Column, centrifuged at 14000×g for 30-60 seconds, and the solution was discarded.
5) Add 700 ul of Wash Buffer1 (add 13 mL of ethanol), centrifuge at 14000×g for 30-60 seconds, and discard the liquid;
6) Add 700 ul of Wash Buffer2 (add 30 mL of ethanol), centrifuge at 14000×g for 30-60 seconds, discard the liquid, and repeat the above steps once;
7) 14000×g air separation for 60 seconds, all eluents were completely removed, and the empty tube was placed in a 1.5 mL collection tube;
8) Add 10 ul of nuclease-free water to the center of the column and centrifuge at 14000×g for 60 seconds to collect gRNA.

Among them, Wash Buffer1 and Wash Buffer2 were both reagents from TranscriptAid™ EnzymeMix kit. The RNA sequences of Ad5-E4-up-gRNA and Ad5-E4-down-gRNA obtained by transcription were as follows:

Ad5-E4-up-
(SEQ ID NO: 48)
gRNA:GUACUAAACAAUUCCUUCCGUUUUAGAGCUAGAAAUAGCAAGUU
AAAAUAAGGCUAGUCCGUUAUCAACUUGAAAAAGUGGCACCGAGUCGGU
GCUUUU
Ad5-E4-down-
(SEQ ID NO: 49)
gRNA:GGUUCGCGUGCGGUUUUCUGUUUUAGAGCUAGAAAUAGCAAGUU
AAAAUAAGGCUAGUCCGUUAUCAACUUGAAAAAGUGGCACCGAGUCGGU
GCUUUU

### 7: Linearization by CRISPR/Cas9 Technology

The vector plasmid obtained in Example 1 was double-digested with Ad5-E4-up-gRNA, Ad5-E4-down-gRNA, and cas9 in a reaction system of 3 pg Cas9 protein, Ad5-E4-up-gRNA 6 pg, Ad5-E4-down-gRNA 6 pg, pAd5-REBP vector plasmid 3 pg, NEB buffer 3.1 5 ul, and supplemented with water to 50 ul.

The enzyme digestion reaction was incubated overnight at 37° C. 3 uL samples were taken for agarose gel verification, and the electrophoresis diagram of the experimental results was shown in FIG. 3. Lane 1 shows the results of “double-cleavage” of the pAd5 vector plasmid by Ad5-E4-up-gRNA, Ad5-E4-down-gRNA, and cas9, and fragments with a target size of 2500 bp to 5000 bp appeared, indicating that the cleavage results were correct. The vector was purified by Axygen™ gel extraction kit.

8: Obtaining a Fiber Fragment Containing a Partial Knockout and an ITR Fragment and Introducing an I-SceI Restriction Site, Using a Primer Containing the Knockout Partial Fiber for Knockout, Amplifying the Fiber Fragment and Introducing an I-SceI Restriction Site.

### 1) Amplification of Fragment Fiber

### Amplification Primers:

Fiber-RH-F:
(SEQ ID NO: 50)
GAGTGCTACTAAACAATTCCTTCCTGGACCCAGAATATTGG
Fiber-ISceI-ITR-R:
(SEQ ID NO: 51)
TGGTGTTATTACCCTGTTATCCCTAGCAATTGAAAAATAAACACGTTG

The amplification sequence was:

(SEQ ID NO: 52)
TGGTGTTATTACCCTGTTATCCCTAGCAATTGAAAAATAAACACGTTGA
AACATAACACAAACGATTCTTTATTCTTGGGCAATGTATGAAAAAGTGT
AAGAGGATGTGGCAAATATTTCATTAATGTAGTTGTGGCCAGACCAGTC
CCATGAAAATGACATAGAGTATGCACTTGGAGTTGTGTCTCCTGTTTCC
TGTGTACCGTTTAGTGTAATGGTTAGTGTTACAGGTTTAGTTTTGTCTC
CGTTTAAGTAAACTTGACTGACAATGTTACTTTTGGCAGTTTTACCGTG
AGATTTTGGATAAGCTGATAGGTTAGGCATAAATCCAACAGCGTTTGTA
TAGGCTGTGCCTTCAGTAAGATCTCCATTTCTAAAGTTCCAATATTCTG
GGTCCAGGAAGGAATTGTTTAGTAGCACTC.

The amplification system was as follows: 10 μM Fiber-RH-F primer 1 ul; 10 μM Fiber-ISceI-ITR-R primer 1 ul; Template pAd5 (100 ng/uL) 0.5 uL; Q5 high-fidelity enzyme 25 ul; fill with water to 50 ul.

The PCR procedure was as follows: initial denaturation at 98° C., 10 sec, 1 cycle; Denaturation 98° C., 5 sec; Annealing at 60° C. for 30 sec; Extension at 72° C., 10 sec, 35 cycle; Extension at 72° C. for 5 min and 1 cycle; Maintain 4° C. The electrophoresis diagram of amplification results was shown in FIG. 4. The results of partial fiber fragment amplification in lane 1 and DL 2,000 DNA Marker in M indicated that the amplification results were correct. The fragments were purified by Axygen™ gel extraction kit.

### 2) Amplification of ITR Fragments

### Amplification Primers:

ISceI-ITR-F:
(SEQ ID NO: 53)
TAGGGATAACAGGGTAATAACACCACTCGACACGGCAC
ITR-RH-R:
(SEQ ID NO: 54)
GGCGTAGGTTCGCGTGCGGTTTTCTGGGTGTTTTTTGTGGACTT

The amplification sequence was:

(SEQ ID NO: 55)
GGCGTAGGTTCGCGTGCGGTTTTCTGGGTGTTTTTTGTGGACTTTAA
CCGTTACGTCATTTTTTAGTCCTATATATACTCGCTCTGCACTTGGC
CCTTTTTTACACTGTGACTGATTGAGCTGGTGCCGTGTCGAGTGGTG
TTATTACCCTGTTATCCCTA.

The amplification system was as follows: 10 μM ISceI-ITR-F primer 1 ul; 10 μM ITR-RH-R primer 1 ul; Template pAd5 (100 ng/uL) 0.5 uL; Q5 high-fidelity enzyme 25 ul; fill with water to 50 ul.

The PCR procedure was as follows: initial denaturation at 98° C., 10 sec, 1 cycle; Denaturation 98° C., 5 sec; Annealing at 60° C. for 30 sec; Extension at 72° C., 10 sec, 35 cycle; Extension at 72° C. for 5 min and 1 cycle; Maintain 4° C. The amplification results were shown in FIG. 4. The amplification results of partial ITR fragments in lane 2 and DL 2,000 DNA Marker in M indicated that the amplification results were correct. The fragments were purified by an Axygen™ gel extraction kit.

### 3) Obtaining a Fusion Fragment of Fiber-ITR by Fusion PCR

The amplification systems were as follows: 1 ul of 10 μM Fiber-RH-F primer, 1 ul of 10μ 10 μM Fiber-ISceI-ITR-R ITR primer, 0.5 uL of template pAd5 (100 ng/uL), and 25 ul of Q5 high-fidelity enzyme, and fill with water to 50 ul.

The PCR procedure was as follows: initial denaturation at 98° C., 10 sec, 1 cycle; Denaturation 98° C., 5 sec; Annealing at 60° C. for 30 sec; Extension at 72° C., 20 sec, 35 cycle; Extension at 72° C. for 5 min and 1 cycle; Maintain 4° C. The amplification results were shown in FIG. 5. The fusion fragment of Fiber-ITR in lane 1 and 2,000 DNA marker in m indicated that the fusion result was correct. The fragments were purified by an Axygen™ gel extraction kit.

### 9. Vector Ligation

The Fiber-ITR fragment was connected to the vector plasmid by Gibson Assembly® Cloning Kit after the E4 is knocked out, and the connection system was as follows: 100 ng of the gel recovery product vector plasmid fragment, 50 ng of the gel recovery product fiber-ITR fragment, and 10 ul of Gibson premix solution, supplemented with water to 20 ul. Incubate at 50° C. for 40 minutes.

### 10. Transformation

Thawa tube of the prepared NEB 10β competent cells on ice for 10 minutes, add 10 ul of connecting product, carefully flick the tube 4-5 times to mix cells and DNA and put on ice for 30 minutes; The tube was placed in a 42° C. water bath and heat shock for 90 seconds. Spread 50-100 μl of each dilution onto Kanamycin selection plate and incubate 8-12 hours to overnight at 37° C.

### 11. Colony PCR Transformant Screening

The transformants were subjected to colony PCR.

Design downstream primers for colony PCR

E4-cexu-F:
(SEQ ID NO: 56)
AGTGACGATTTGAGGAAGTTG
E4-cexu-R:
(SEQ ID NO: 57)
TCAATTGCAGAAAATTTCAAGTC

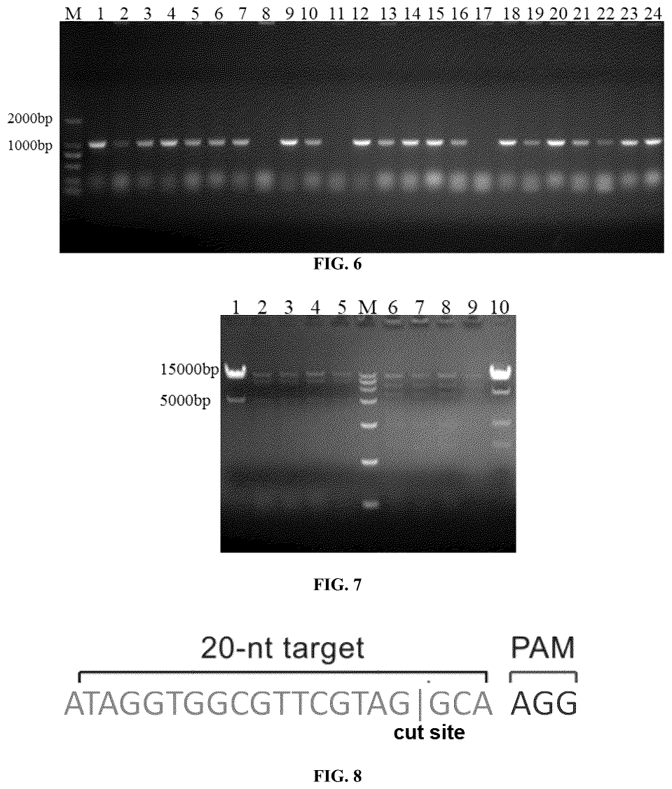

The reaction system was as follows: 1 ul of 10 μM E4-cexu-F primer, 1 ul of 10 μM E4-cexu-R primer, 10 ul of Q5 high-fidelity enzyme, and supplementing water to 20 ul. A single colony was selected into the reaction system. The PCR procedure was as follows: initial denaturation at 98° C., 10 sec, 1 cycle; Denaturation 98° C., 5 sec; Annealing at 60° C. for 30 sec, Extension at 72° C., 20 sec, 35 cycle; Extension at 72° C. for 5 min and 1 cycle; Maintain 4° C. Agarose gel electrophoresis was performed as shown in FIG. 6, and most of the colonies showed positive bands except for No. 2, 8, 11, and 17.

### 12. Plasmid Enzyme Digestion Verification

Four positive clonal colonies were selected to culture, the plasmids were extracted, and the digestion tests of BamHI and XhoI were performed. The digestion results were shown in FIG. 7. From FIG. 7, the digestion results of plasmids No. 2-5 BamHI and XhoI were correct, and the sequencing result was correct at the same time. That was to say, the adenovirus vector plasmid pAd5ΔE4 with E1, E3 and E4 genes deletion was obtained.

### Example 3: Construction of Adenovirus Vector Plasmid pAd5LCL3 without E1, E3, E4 and E2a Genes

### 1. Selection of CRISPR Target Sequence of E2a

1) Selection of CRISPR Target Sequence of 100 k Gene at upstream of E2a Gene

The first 400 bp of 100 k gene were input by GeneArt™ CRISPR Search and Design tool (thermofisher.com/crispresign) software from Thermo Fisher Scientific. The software automatically analyzed the sequence of the 400 bp and provided six potential CRISPR target sequences. Considering the length of the knockout sequence of E2a gene and the requirement of constructing a live vector, ATAGGTGGCGTTCGTAGGCA (SEQ ID NO: 166) was selected as the targeting sequence, and the finally obtained gRNA was named as 100 k-gRNA, with the cleavage site and PAM site shown in FIG. 8.

### 2) Selection of CRISPR Target Sequence of Downstream Non-Coding Sequence of E2a

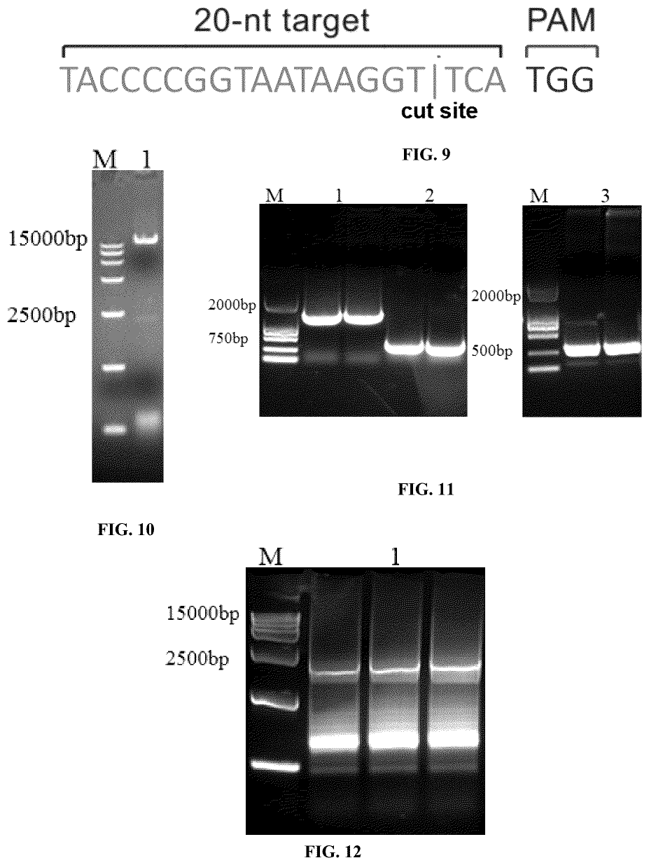

The 300 bp downstream of E4 were input by GeneArt™ CRISPR Search and Design tool(thermofisher.com/crisprdesign) software from Thermo Fisher Scientific, and the software was automatically analyzed to provide six potential CRISPR target sequences. TACCCCGGTAATAAGGTTCA (SEQ ID NO: 167) was selected as the target sequence, and the finally obtained gRNA was named as protease-gRNA, with the cleavage site and PAM site shown in FIG. 9.

2. DNA Amplification of 100 k-gRNA and Protease-gRNA

1) DNA template design of 100k-gRNA
(SEQ ID NO: 58)
5′-TAATACGACTCACTATAGAGGTGGCGTTCGTAGGCAGTTTTAGA
GCTAGAAATAGCAAGTTAAAATAAGGCTAGTCCGTTATCAACTTGAA
AAAGTGGCACCGAGTCGGTGCTTTT-3′
2) DNA template design of protease-gRNA
(SEQ ID NO: 59)
5′-TAATACGACTCACTATAGCCCCGGTAATAAGGTTCAGTTTTAGA
GCTAGAAATAGCAAGTTAAAATAAGGCTAGTCCGTTATCAACTTGAA
AAAGTGGCACCGAGTCGGTGCTTTT-3′

3. Design of Upstream and Downstream Primers for Amplifying the DNA Templates of 100 k-gRNA and Protease-gRNA

Upstream and downstream primers were designed for PCR amplification of the 100 k-gRNA DNA template and protease-gRNA DNA template, respectively, and GeneArt™ Precision gRNA Synthesis Kit was used for amplification.

### 1) Primer Design:

100k-gRNA-Foward:
(SEQ ID NO: 60)
TAATACGACTCACTATAG AGGTGGCGTTCGTAG
100k-gRNA-Reverse:
(SEQ ID NO: 61)
TTCTAGCTCTAAAAC TGCCTACGAACGCCACCT
protease-gRNA-Foward:
(SEQ ID NO: 62)
TAATACGACTCACTATAG CCCCGGTAATAAGGT
protease-gRNA-Reverse:
(SEQ ID NO: 63)
TTCTAGCTCTAAAAC TGAACCTTATTACCGGGG

2) Amplification of the DNA Templates of 100 k-gRNA and Protease-gRNA

\textcircled{1} Prepare 0.3 μM mixed working solution of 100 k-gRNA-Forward/Reverse primer, including 10 μM 100K-GrNA-forward primer 3 ul, 10 μM 100 k-gRNA-Reverse primer 3 ul, and supplement water to 100 ul.

\textcircled{2} Prepare 0.3 μM mixed working solution of protease-gRNA-Forward/reverse primer, including 10 μM Protease-grna-forward primer 3 ul, 10 μM protease-gRNA-Reverse primer 3 ul, and supplement water to 100 ul.

\textcircled{3} PCR reaction system

The PCR reaction system for DNA template amplification of 100 k-gRNA was as follows: Phusion™ High-Fidelity PCR Master Mix (2×) 12.5 uL, Tracr Fragment+T7 Primer Mix 1 ul, and 0.3 μM 100 k-gRNA-Forward/Reverse primer mixture working solution 1 ul, and water supplementing to 25 ul.

The PCR reaction system for DNA template amplification of protease-gRNA was as follows: Phusion™ High-Fidelity PCR Master Mix (2×) 12.5 uL, Tracr Fragment+T7 Primer Mix 1 ul, and 0.3 μM Protease-GrNA-Forward/Reverse primer mixture working solution 1 ul, and the water supplementing amount was up to 25 ul.

\textcircled{4} PCR Program

Initial denaturation 98° C., 10 sec, 1 cycle; Denaturation 98° C., 5 sec; Annealing at 55° C. for 15 sec and 32 cycles; Extension at 72° C. for 1 min and 1 cycle; Maintain 4° C.

4. In Vitro Transcription to Obtain 100 k-gRNA and Protease-gRNA

In vitro transcription of template DNA was performed by TranscriptAid™ EnzymeMix to obtain 100 k-gRNA and protease-gRNA.

1) In Vitro Transcription to Obtain 100 k-gRNA, Protease-gRNA

The reaction systems for obtaining 100 k-gRNA by in vitro transcription were as follows: NTP mix 8 ul, 100 k-gRNA DNA template 6 ul, 5× TranscriptAid™ Reaction Buffer 4 uL, and TranscriptAid™ Enzyme Mix 2 ul. After incubation at 37° C. for 4 hours add 1 ul of DNase I and incubate at 37° C. for 15 minutes.

The reaction systems for obtaining protease-gRNA by in vitro transcription were as follows: NTP mix 8 ul, protease-gRNA DNA template 6 ul, 5× TranscriptAid™ Reaction Buffer 4 uL, and TranscriptAid™ Enzyme Mix 2 ul. After incubation at 37° C. for 4 hours add 1 ul of DNase I and incubate at 37° C. for 15 minutes.

### 2) Purification of In Vitro Transcription Products

\textcircled{1} Fill the transcribed reaction system to 200 ul with nuclease-free water;
\textcircled{2} Add 100 ul of Binding buffer, and mix well;
\textcircled{3} Add 300 ul of ethanol (>96%) and mix well;
\textcircled{4} The mixed solution was transferred to the Gene Jet™ RNA Purification Micro Column, centrifuged at 14000×g for 30-60 seconds, and the solution was discarded.
\textcircled{5} Add 700 ul of Wash Buffer1 (add 13 mL of ethanol), centrifuge at 14,000×g for 30-60 seconds, and discard the liquid;
\textcircled{6} Add 700 ul of Wash Buffer2 (add 30 mL of ethanol), centrifuge at 14,000×g for 30-60 seconds, discard the liquid, and repeat the above steps once;

\textcircled{7} 14,000×g air separation for 60 seconds, all eluents were completely removed, and the empty tube was placed in a 1.5 mL collection tube;

\textcircled{8} Add 10 ul of nuclease-free water to the center of the column and centrifuge at 14000×g for 60 seconds to collect gRNA.

Among them, Wash Buffer1 and Wash Buffer2 were both reagents from TranscriptAid™ EnzymeMix kit. The RNA sequences of 100 k-gRNA and protease-gRNA obtained by transcription were as follows:

100k-gRNA:
(SEQ ID NO: 64)
GAGGUGGCGUUCGUAGGCAGUUUUAGAGCUAGAAAUAGCAAGUUAAA
AUAAGGCUAGUCCGUUAUCAACUUGAAAAAGUGGCACCGAGUCGGUG
CUUUU
protease-gRNA:
(SEQ ID NO: 65)
GCCCCGGUAAUAAGGUUCAGUUUUAGAGCUAGAAAUAGCAAGUUAAA
AUAAGGCUAGUCCGUUAUCAACUUGAAAAAGUGGCACCGAGUCGGUG
CUUUU

### 5. Linearization by CRISPR/Cas9 Technology

The adenovirus vector plasmid with E1, E3, and E4 genes deletion obtained in Example 2 was double-digested with 100 k-gRNA, protease-gRNA, and cas9 in a reaction system of 3 μg Cas9 protein; 100 k-gRNA 6 μg; protease-gRNA 6 μg; 3 μg; of the vector plasmid obtained in example 2; NEB buffer 3.1 5 ul; Replenish water to 50 ul.

The above enzymatic reactions were incubated overnight at 37° C. 3 ul samples were taken for agarose gel verification, and the experimental results were shown in FIG. 10. Lane 1 shows the results of “double enzyme digestion” of the vector plasmids 100 k-gRNA, protease-gRNA and cas9, and fragments with the target size of 1000-2500 bp appeared, indicating that the enzyme digestion results were correct. The vector was purified by an Axygen™ gel extraction kit.

### 6. Obtaining a Proteinase Fragment Containing a Partially-Knocked-Out 100 k, E4 ORF6/7 Expression Frame.

### 1) Partial Knockout of the 100 k, E4 ORF6/7 Expression Box and Amplification of Protease Fragment

\textcircled{1} Partial knockout 100 k amplification primers:

100k-F:
(SEQ ID NO: 66)
TGAGAATAGGTGGCGTTCGTAGGCAAGGCTGACATCCGCTATGG
100k-ORF6/7-R:
(SEQ ID NO: 67)
TACAATTCCCAACACATACAAGTTTCCTTCTCCTATAGGCAGAA

The amplification system was as follows: 10 μM 100 k-F primer 1 ul; 10 μM 100k-ORF6/7-R primer 1 ul; Template pAd5ΔE4 (100 ng/uL) 0.5 uL; Q5 high-fidelity enzyme 25 ul; Replenish water to 50 ul.

The PCR procedure was as follows: initial denaturation at 98° C., 10 sec, 1 cycle; Denaturation 98° C., 5 sec; Annealing at 60° C. for 30 sec; Extension at 72° C., 20 sec, 35 cycle; Extension at 72° C. for 5 min and 1 cycle; Maintain 4° C.

\textcircled{2} E4 ORF6/7 expression frame amplification primers:

ORF6/7-F:
(SEQ ID NO: 68)
ACTTGTATGTGTTGGGAATTGTA
ORF6/7-R:
(SEQ ID NO: 69)
ATCGTTTGTGTTATGTTTCAACG

The amplification system was as follows: ORF6/7-F primer 1 ul; 10 μM ORF6/7-R primer 1 uL; Template ORF6/7 expression cassette gene (100 ng/uL) 0.5 uL; Q5 high-fidelity enzyme 25 ul; Replenish water to 50 ul.

The PCR procedure was as follows: initial denaturation at 98° C., 10 sec, 1 cycle; Denaturation 98° C., 5 sec; Annealing at 60° C. for 30 sec; Extension at 72° C., 10 sec, 35 cycle; Extension at 72° C. for 5 min and 1 cycle; Maintain 4° C.

\textcircled{3} Amplification of partially knockout protease fragments

ORF6/7-Protease-F:
(SEQ ID NO: 70)
CCCACCCTTGCCGTCTGCGCCGTATCGTTTGTGTTATGTTTCAACG
Protease-R:
(SEQ ID NO: 71)
ATGGATCACAACCCCACCATGAACCTTATTACCGGGGTACCCA

The amplification system was as follows: 10 M ORF6/7-Protease-F primer 1 μL; 10 μM Protease-R primer 1 ul; Template PD5ΔE4 (100 ng/uL) 0.5 uL; Q5 high-fidelity enzyme 25 ul; Replenish water to 50 ul.

The PCR procedure was as follows: initial denaturation at 98° C., 10 sec, 1 cycle; Denaturation 98° C., 5 sec; Annealing at 60° C. for 30 sec; Extension at 72° C., 10 sec, 35 cycle; Extension at 72° C. for 5 min and 1 cycle; Maintain 4° C.

\textcircled{4} the results of 100 k, E4 ORF6/7 expression frame and protease PCR amplification were shown in FIG. 11, where lane 1 was E4 ORF6/7 expression frame, lane 2 was 100 k, and M was DL 15,000 DNA Marker.

It could be seen that the amplification results were correct. The fragments were separately purified by gel recovery by an Axygen™ gel extraction kit.

### 7. Fusion PCR was Performed to Obtain the Fusion Fragments of the 100 k, E4 ORF6/7 Expression Frames and the Protease Fragment

The amplification system was as follows: 10 μM 100 k-F primer 1 ul; 10 μM Protease-R primer 1 ul; Template 100 k recovered product (50 ng/ul) 1 ul template E4 ORF6/7 expression framer recovered product (50 ng/ul) 1 ul template E4 ORF6/7 expression framer recovered product (50 ng/uL) 1 uL; Q5 high-fidelity enzyme 25 ul; Replenish water to 50 ul.

The PCR procedure was as follows: initial denaturation at 98° C., 10 sec, 1 cycle; Denaturation 98° C., 5 sec; Annealing at 60° C. for 30 sec; Extension at 72° C., 50 sec, 35 cycle; Extension at 72° C. for 5 min and 1 cycle; Maintain 4° C. The amplification results were shown in FIG. 12, where Lane 1 was the product of fragment 100 k, E4 ORF6/7 expression frame and protease fusion PCR, demonstrating the correct amplification result.

The fragments were purified by an Axygen™ gel extraction kit.

8. connection

Using Gibson of NEB, the 100 k, E4 ORF6/7 expression frame and protease fusion PCR gel recovery products were connected to the vector after E2a was knocking out in Step 4. The connection system was as follows: 100 ng of the vector fragment after knocking out E2a with the gel recovery product, 100k of the gel recovery product, 50 ng of the E4 ORF6/7 expression frame and protease fusion PCR fragment, 10 ul of Gibson premix solution, and water supplementing to 20 uL. Incubate at 50° C. for 40 minutes.

### 9. Conversion

Taking out the Kana resistance culture medium plate, putting the prepared NEB 10β competent cells on ice for melting, adding 10 ul of connecting product, gently sucking and uniformly beating by a pipette, and putting on ice for 30 minutes; The centrifuge tube was placed in a 42° C. water bath and hot-struck for 90 seconds to screen transformants by kanamycin resistance.

### 10. Performing Transformant Screening by Colony PCR

The transformants were subjected to colony PCR.

### Design Downstream Primers for Colony PCR

DBP-upsteam-F:
(SEQ ID NO: 72)
GTTGGGCTCGCATGTGCCG
DBP-downsteam-R:
(SEQ ID NO: 73)
ACTCCCATGGATCACAACCC

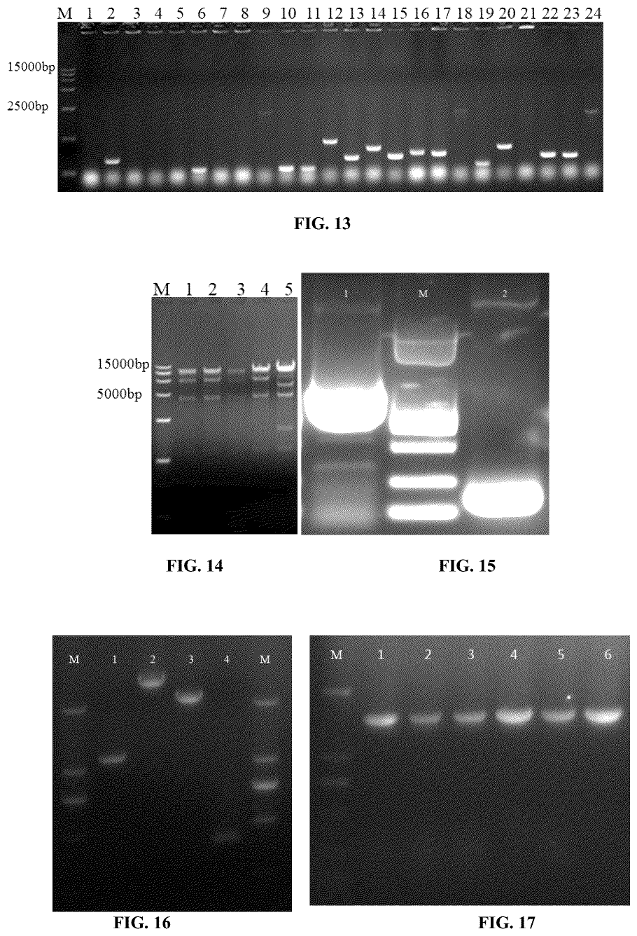

The reaction system was as follows: 1 ul of 10 μM DBP-upstream-F primer, 1 ul of 10 μM DBP-downstream-R primer, 10 ul of Q5 high-fidelity enzyme, and supplementing water to 20 ul. Monoclonal colonies were selected into the reaction system. The PCR procedure was as follows: initial denaturation at 98° C., 10 sec, 1 cycle; Denaturation 98° C., 5 sec; Annealing at 60° C. for 30 sec; Extension at 72° C., 20 sec, 35 cycle; Extension at 72° C. for 5 min and 1 cycle; Maintain 4° C. Agarose gel electrophoresis verification was performed, and as shown in FIG. 13, positive bands appeared at 9, 18, 21, and 24.

### 11. Plasmid Restriction Enzyme Digestion Verification

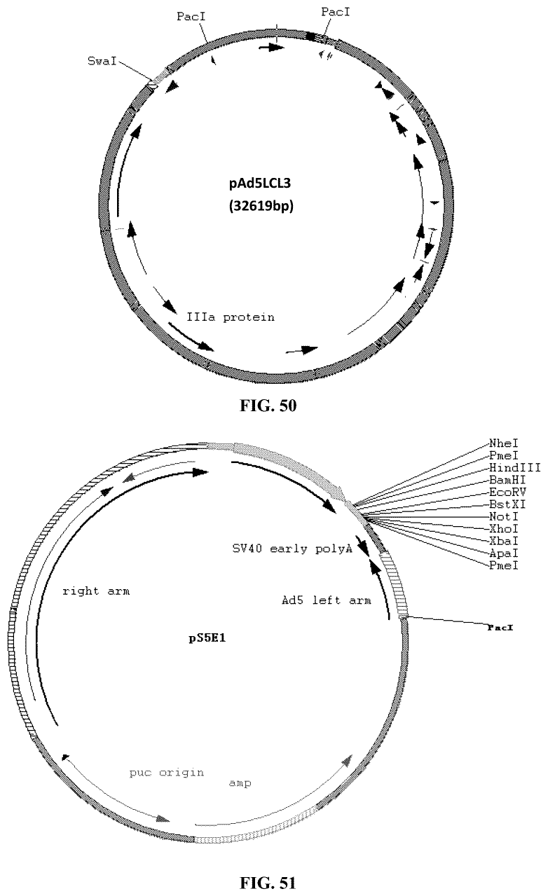

Four positive clone colonies of No. 9, No. 18, No. 21, and No. 24 were picked out, and the plasmid was extracted for XhoI restriction endonuclease verification. The restriction endonuclease results were shown in FIG. 14, where Lane 1 was the XhoI restriction endonuclease of No. 9 positive clone, Lane 2 is the XhoI restriction endonuclease of No. 18 positive clone, Lane 3 was the XhoI restriction endonuclease of No. 21 positive clone, Lane 4 was the XhoI restriction endonuclease of No. 24 positive clone, and Lane 5 was the XhoI restriction endonuclease of the control plasmid pAd5LCL3. As shown in FIG. 14, the digestion results of plasmid XhoI were all correct, while the sequencing results were correct. That is, the pAd5LCL3 plasmid, with the deleted E1, E3, E4 and E2a genes and the ORF6/7 expression frame of E4 region placed at the sequence position that was deleted in E2a region, was obtained. The vector map was shown in FIG. 50.

### Example 4: Construction of the African Swine Fever Human Adenovirus Type 5 Vector E1 Region Shuttle Plasmid pS5E-P72-IRES-B602L

### 1. Construction of Human Adenovirus Type 5 Vector E1 Region Shuttle Plasmid

The skeleton of shuttle plasmid pS5E1 was composed of puc origin, amp and other basic elements (2796 bp) (the pS5E1 skeleton was synthesized by Beijing BioMed Gene Technology Co., Ltd.), HAd5 partial sequence of ITR in the left arm (355 bp), PIX in the right arm and PIVa2 partial sequence (2100 bp), and CMV-MCS (Seq ID No. 12) (944 bp) SV40 Early Polya (Seq ID No. 13) (160 bp).

### 1) Primer Design

puc-Ad5-right arm-F:
(SEQ ID NO: 74)
TAATGCAGCTGGCTTATCGAAACGTGGAATGCGAGACCGTCT
Ad5-right arm-CMV-R:
(SEQ ID NO: 168)
ACACACAAGCAGGGAGCAGATACAAGGGTGGGAAAGAATATATAAG
CMV-F:
(SEQ ID NO: 75)
GTATCTGCTCCCTGCTTGTG
CMV-SV40-R:
(SEQ ID NO: 169)
TAAACAAGTTGGGGTGGGCGAAGTGATCAGCGGGTTTAAACGGG
SV40-F:
(SEQ ID NO: 76)
CTTCGCCCACCCCAACTTGT
SV40-R:
(SEQ ID NO: 77)
AGAGGTCGACGGTATACAGAC
SV40-Ad5-left arm-F:
(SEQ ID NO: 78)
TGTCTGTATACCGTCGACCTCTCCGAAAAACACCTGGGCGAGTCTC
C
Ad5-left arm-puc-R:
(SEQ ID NO: 79)
ACACTATAGAATACACGGAATTCTTAATTAAATCATCAATAATATA
CCTTATTTTG
puc-F:
(SEQ ID NO: 80)
GAATTCCGTGTATTCTATAGTGT
puc-R:
(SEQ ID NO: 81)
TTTCGATAAGCCAGCTGCATTA

### 2) Amplification of the Target Fragment

\textcircled{1} The MCS fragment of CMV promoter of pSSE1 shuttle plasmid was amplified using pCDNA3.1(+) as the template (the plasmid was purchased from Thermo Fisher Scientific Co., Ltd.) and CMV-F and CMV-SV40-R as the primers; Amplification system: pCDNA3.1(+) plasmid 50 ng, 10 uM CMV-F primer 1 ul, 10 uM CMV-SV40-R primer 1 ul, Q5 high-fidelity enzyme 20 ul; Replenish water to 40 ul; PCR program: 98° C., 10 s; 98° C., 5s, 60° C., 30s, 72° C., 1 min, 35 cycles; 72° C., 5 min.

\textcircled{2} The SV40-earlypolyA fragment of the pSSE1 shuttle plasmid was amplified using pCDNA3.1(+) as the template (the plasmid was purchased from Thermo Fisher Scientific Co., Ltd.) and SV40-F and SV40-R as the primers; Amplification system: pCDNA3.1(+) plasmid 50 ng, 10 uM SV40-F primer 1 ul, 10 uM SV40-R primer 1 ul, Q5 high-fidelity enzyme 20 ul; Replenish water to 40 ul; PCR program: 98° C., 10 s; 98° C., 5s, 60° C., 30s, 72° C., 10 sec, 35 cycles; 72° C., 5 min.

Agarose validation of that amplification product was shown in FIG. 15, with lane 1 bee the CMV-MCS fragment, lane 2 being the SV40 earlypolyA fragment, and M being the DL 2,000 DNA Marker. As shown in FIG. 15, the amplification result was correct.

\textcircled{3} Purification was performed by an Axygen™ gel extraction kit.

\textcircled{4} pSSE1 shuttle plasmid backbone was amplified by PCR using pSSE1 backbone plasmid synthesized by BoMed Co. as the template and puc-F and puc-R as the primers. The amplification system consisted of pSSE1 backbone plasmid of 50 μg, 10 μM PUC-F primer of 1 ul, 10 μM PUC-R primer of 1 ul, and Q5 high-fidelity enzyme of 20 ul; Replenish water to 40 ul; PCR program: 98° C., 10 s; 98° C., 5s, 60° C., 30s, 72° C., 1 min 20 sec, 35 cycles; 72° C., 5 min.

\textcircled{5} The left arm of pSSE1 shuttle plasmid was amplified using pAd5LCL3 plasmid as a template and SV40-Ad5-left arm-F and Ad5-left arm-puc-R as primers. The amplification system: pAd5LCL3 plasmid 50 ng, 10 uM SV40-Ad5-left arm-F primer 1 ul, 10 uM Ad5-left arm-puc-R primer 1 ul, Q5 high-fidelity enzyme 20 ul, and water replenishment to 40 ul. PCR program: 98° C., 10 s; 98° C., 5s, 60° C., 30s, 72° C., 20s, 35 cycles; 72° C., 5 min.

\textcircled{6} The right arm of pSSE1 shuttle plasmid was amplified using pAd5LCL3 plasmid as the template and puc-Ad5-right arm-F and Ad5-right arm-CMV-R as the primers. The amplification system: pAd5LCL3 plasmid 50 ng, 10 uM puc-Ad5-right arm-F primer 1 ul, 10 uM Ad5-right arm-CMV-R primer 1 ul, and Q5 high-fidelity enzyme 20 ul. The water was replenished to 40 uL. PCR program: 98° C., 10 s; 98° C., 5s, 60° C., 30s, 72° C., 15s, 35 cycles; 72° C., 5 min.

\textcircled{7} CMV-MCS-SV40 Early Polya fragment of pSSE1 shuttle plasmid was amplified using CMV-MCS, a recovered gel product, as a template and CMV-F and SV40-R as primers. The amplification system consisted of pAd5LCL3 plasmid of 50 ng, 10 μM CMV-F primer of 1 ul, 10 μM SV40-R primer of 1 ul, and Q5 high-fidelity enzyme of 20 ul, supplemented with water to 40 ul. PCR program: 98° C., 10 s; 98° C., 5s, 60° C., 30s, 72° C., 40s, 35 cycles; 72° C., 5 min.

Agarose validation of that amplification product was shown in FIG. 16, with lane 1 being the CMV-MCS-SV40 earlypolyA fusion fragment, lane 2 being PUC, lane 3 bee HAd5 right arm and lane 4 being HAd5 left arm.

### 3) Ligation Transformation of Fragments

The fragments were purified by an Axygen™ gel extraction kit, and the four fragments, pSSE1 skeleton, HAd5 left arm, HAd5 right arm, and CMV-MCS-SV40 earlypolyA, were connected by a BMD seamless cloning kit. The ligation system was 2×Smealess Cloning Mix 10 ul, pSSE1 skeletal fragment 50 ng, HAd5 left arm 50 ng, HAd5 right arm 50 ng, CMV-MCS-SV40 polyA 50 ng, supplemented water to 20 ul, and incubated at 50 C for 40 minutes to obtain the ligated product plasmid pS5E1. The ligated products were transformed into DH5α competent cells, plated on plates containing ampicillin resistance and incubated at 37° C. for 12-16 hours.

### 4) Plasmid Validation

\textcircled{1} Colony PCR

Colonies were selected for agarose gel validation and shown in FIG. 17 as positive bands.

\textcircled{2} Enzyme Digestion Verification

The selected positive clones were cultured in 5 mL LB liquid medium containing ampicillin resistance for 12-15 hours, and the plasmid was extracted for restriction endonuclease validation. The electrophoresis results were shown in FIG. 18, where Left 1-6 showed single restriction endonuclease of plasmid pS5E1 NcoI, and Right 1-6 showed single restriction endonuclease of plasmid pS5E1 PacI, with M being 15000 bp Marker. The restriction endonuclease result was correct, and the shuttle plasmid pS5E1 of human adenovirus type 5 vector region was successfully constructed, with the vector map shown in FIG. 51.

2. Construction of Shuttle Plasmid pS5E1-P72-IRES-B602L of African Swine Fever Human Adenovirus Type 5 Vector.

1) Ligation of pS5E1 Fragment to the IRES Fragment

\textcircled{1} Primer synthesis

IRES-EcoRV-F:
(SEQ ID NO: 82)
ccg GATATC TGTCGTCATCATCCTTATAGTCC
IRES-NotI-R:
(SEQ ID NO: 83)
aaatat GCGGCCGC GGTTGTGGCCATTATCATCGTG

\textcircled{2} amplifying IRES fragment

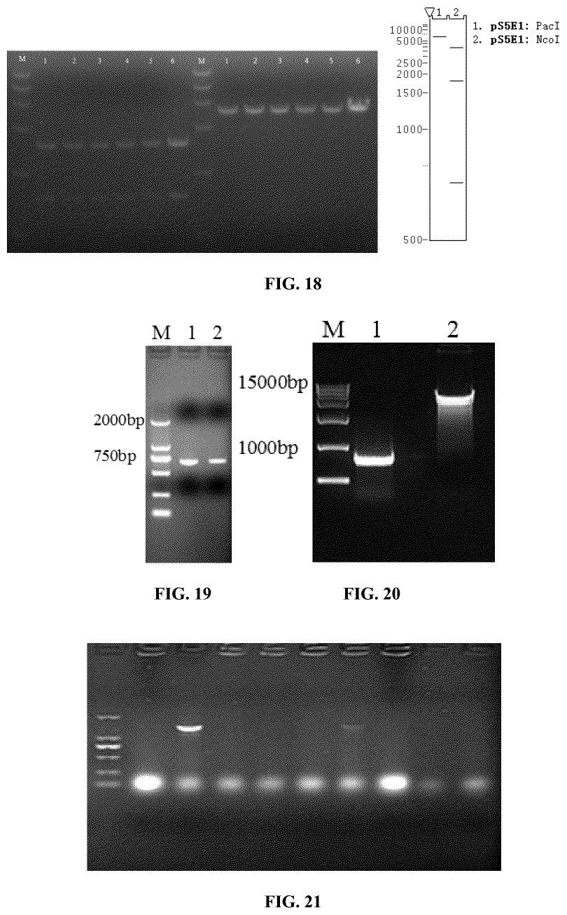

Amplification system: 25 ul of Q5 enzyme, 1 uL of 10 uM primer IRES-ECORV-F, 1 uL of 10 uM primer IRES-NOTI-R, 2 ul of template IRES, and water supplementing to 50 ul; PCR program: 98° C., 10 s; 98° C., 5s, 60° C., 30s, 72° C., 20s, 35 cycles; 72° C., 5 min. The electrophoretic detection of amplification results was shown in FIG. 19, where lanes 1 and 2 were the PCR amplification products of IRES fragment and M was DL 15,000 DNA Marker, so the amplification results were correct.

\textcircled{3} IRES fragment was purified by Axygen™ PCR purification kit.

\textcircled{4} Excision of the target fragment IRES with the pSSE1 vector

Enzyme digestion reaction system: the vector pS5E1, IRES fragment ˜2 ug, and each of EcoRV and NotI was 1 uL; 10×cut smart buffer 5 ul; Replenish water to 50 ul; Reaction condition: 37° C., 30 min; Inactivated at 65° C. for 20 min; Recovery and purification of gum. The electrophoresis detection of the enzyme-cleaved product was shown in FIG. 20, where lane 1 was the fragment IRES EcoRV, NotI enzyme-cleaved, lane 2 was pSSE1 EcoRV, NotI enzyme-cleaved, and M was DL 15,000 DNA Marker.

\textcircled{5} pSSE1 vector was connected with IRES fragment

Ligation system: pSSE1 (100 ng); IRES fragment (vector:fragment=1:5, molar ratio); T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

Amplification system: 10 ul of Q5 enzyme, 10 uM primer IRES-EcoRV-F 1 ul, and 10 uM primer IRES-NotI-R 1 ul, and supplementing water to 20 ul; PCR program: 98° C., 10 s; 98° C., 5s, 60° C., 30s, 72° C., 20s, 35 cycles; 72° C., 5 min. Electrophoresis verification was performed as shown in FIG. 21, in which No. 1-9 was a colony and M was DL 2,000 DNA Marker, and as shown in FIG. 21, positive bands appeared in No. 2 and No. 6.

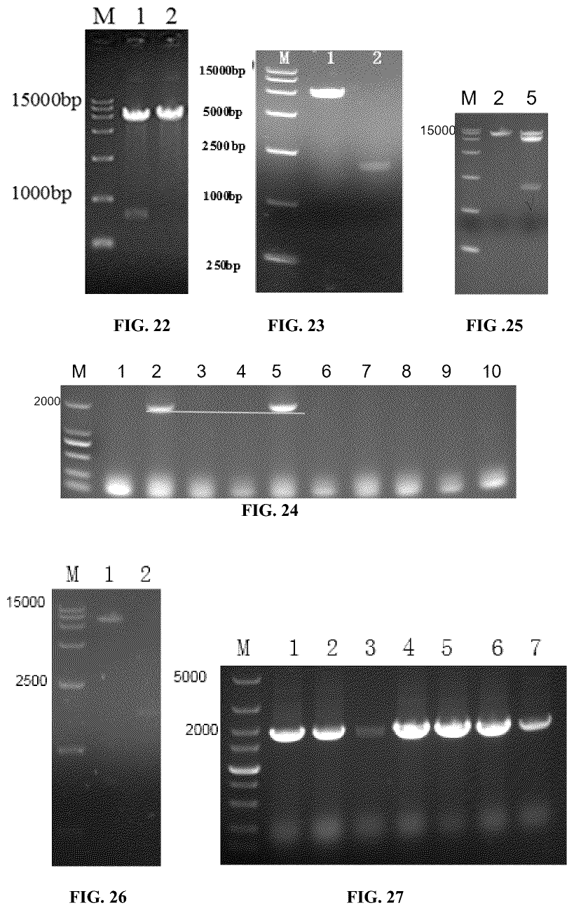

\textcircled{7} Plasmid NotI and EcoRV restriction enzyme digestion verification: 2 and 6 were selected for plasmid extraction and restriction enzyme digestion verification, and the results were shown in FIG. 22. The restriction enzyme digestion results of plasmid No. 2 NotI and EcoRV and plasmid No. 6 NotI and EcoRV were shown to be correct.

2) Ligation of pS5E1-IRES to P72 Fragment 0 primer synthesis

P72-his-EcoRV-R:
(SEQ ID NO: 84)
cgGATATCTCAGTGGTGGTGGTGATGGTGGGTGCTGTATCTCAGCACGG
P72-BamHI-F:
(SEQ ID NO: 85)
cgcGGATCCgccaccATGGCCAGCGGCGGAGCTTT

\textcircled{2} PCR amplification of P72 fragment

Amplification system: 25 ul of Q5 enzyme, 1 uL of 10 uM primer P72-BamHI-F, 1 uL of 10 uM primer P72-His-ECORV-R, 1 uL of template P72, and water supplementing to 50 ul; PCR program: 98° C., 10 s; 98° C., 5s, 60° C., 30s, 72° C., 40s, 35 cycles; 72° C., 5 min.

\textcircled{3} The P72 fragment was purified by Axygen™ PCR purification kit.

\textcircled{4} The target fragment P72 was digested with pS5E1-IRES vector

Enzyme digestion reaction system: the vector pS5E1-IRES, P72 fragment −2 ug, and each of EcoRV and BamHI was 1 μL; 10×cutsmart buffer 5 ul; Replenish water to 50 ul. Reaction condition: 37° C., 30 min; Inactivated at 65° C. for 20 min. Recovery and purification of gum. The electrophoresis detection of the enzyme digestion product was shown in FIG. 23, where lane 1 was for fragment pS5E1-IRES and NotI enzyme digestion, lane 2 was for P72 and NotI enzyme digestion, and M was DL 15,000 DNA Marker.

\textcircled{5} The target fragment P72 was connected with pS5E1-IRES

Ligation system: PS5E1-IRES (100 ng); P72 fragment (vector:fragment=1:5, molar ratio); T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

Amplification system: Q5 enzyme 10 ul, 10 uM primer P72-BamHI-F 1 ul, 10 uM primer P72-his-EcoRV-R 1 ul, and water supplement to 20 ul; PCR program: 98° C., 10 s; 98° C., 5s, 60° C., 30s, 72° C., 20s, 35 cycles; 72° C., 5 min. Electrophoresis verification was performed as shown in FIG. 24, in which No. 1-10 was the colony and M was DL 15,000 DNA Marker, and as shown in FIG. 24, positive bands appeared in No. 2 and No. 5.

\textcircled{7} Plasmid restriction endonuclease assay (BamHI&EcoRV), select 2 and 5 for plasmid extraction and restriction endonuclease assay. The result was shown in FIG. 25, where plasmid No. 5 was a positive plasmid.

3) Ligation of pS5E1-P72-IRES to Fragment B602L

\textcircled{1} primer synthesis

B602L-NotI-F:
(SEQ ID NO: 86)
aaatatGCGGCCGCATGGCCGAATTCAATATTGATGAA
B602L-XhoI-R:
(SEQ ID NO: 87)
cggCTCGAGTCAGTGGTGGTGGTGATGGTGGGCGTAATCGGGCACGTCGT

\textcircled{2} PCR amplification of B602L fragment

Amplification system: 25 ul of Q5 enzyme, B602L-NotI-F 1 ul of 10 uM primers, B602L-XhoI-R 1 ul of 10 uM primers, and P72 1 ul of template and water supplementing to 50 ul; PCR program: 98° C., 10 s; 98° C., 5s, 60° C., 30s, 72° C., 40s, 35 cycles; 72° C., 5 min.

\textcircled{3} The B602L fragment was purified by Axygen™ PCR purification kit.

\textcircled{4} The target fragment B602L was digested with pS5E1-P72-IRES vector

Enzyme digestion reaction system: vectors pS5E1-P72-IRES, B602L fragment 2 ug, NotI and XhoI 1 uL each; 10×cutsmart buffer 5 ul; Replenish water to 50 ul. Reaction condition: 37° C., 30 min; Inactivated at 65° C. for 20 min. Recovery and purification of gum. The electrophoresis detection of the enzyme digestion product was shown in FIG. 26, where lane 1 was subjected to enzyme digestion with pS5E1-P72-IRES, NotI and XhoI, and lane 2 was subjected to enzyme digestion with B602L fragment, NotI and XhoI, and M was DL 15,000 DNA Marker.

\textcircled{5} ligation of pS5E1-P72-IRES vector to B602L fragment

Linkage system: pS5E1-P72-IRES 100 ng; B602L fragment 50 ng; T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

Amplification system: Q5 enzyme 10 ul, 10 uM primer B602L-NotI-F 1 ul, 10 uM primer B602L-XhoI-R 1 ul, supplementing water to 20 ul; PCR program: 98° C., 10 s; 98° C., 5s, 60° C., 30s, 72° C., 20s, 35 cycles; 72° C., 5 min; Electrophoresis verification was performed, as shown in FIG. 27, where No. 1-7 was a colony and M was DL 5,000 DNA Marker, and as shown in FIG. 27, positive bands appeared.

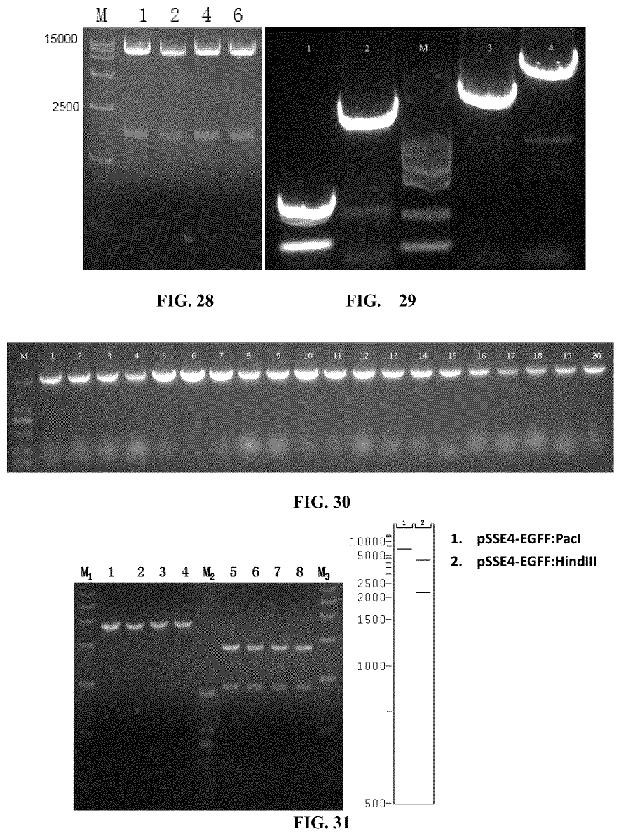

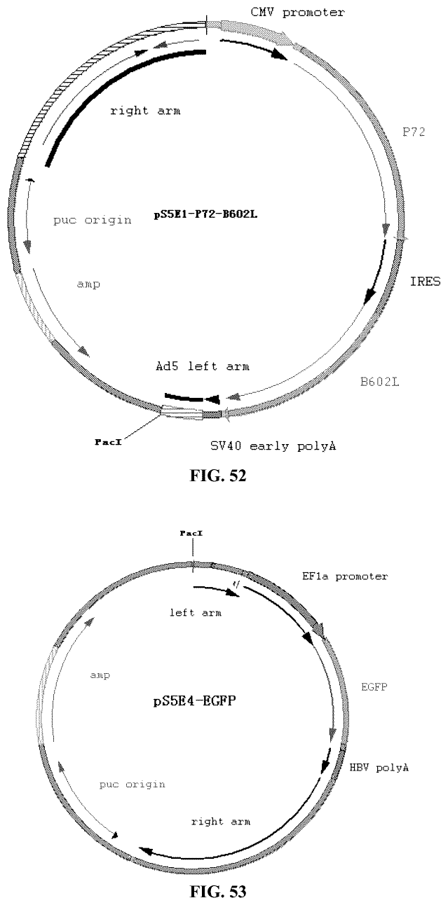

\textcircled{7} Plasmid NotI and XhoI enzyme digestion verification: 1, 2, 4 and 6 were selected for plasmid extraction and enzyme digestion verification. The results were shown in FIG. 28, where the enzyme digestion identification of plasmids NotI and XhoI in lanes 1, 2, 4 and 6 was conducted, and M was DL 15,000 DNA Marker. As shown in FIG. 28, the enzyme digestion result was correct, and the African swine fever human adenovirus type 5 vector E1 region shuttle plasmid pSSE1-P72-IRES-B602L was successfully constructed, and the vector map was shown in FIG. 52.

### Example 5: Construction of African Swine Fever Human Adenovirus Type 5 Vector E4 Region Shuttle Plasmid pS5E4-P30-2A-P54

### 1. Human Adenovirus Type 5 Vector E4 Region Shuttle Plasmid Construction

The skeleton of the shuttle plasmid pS5E4 was composed of basic elements such as puc origin and amp, the ITR sequence of the left arm (370 bp), the partial fiber gene sequence of the right arm (1746 bp) in the Ad5E4 region, and the EF1α-EGFP-HBV polyA gene.

1) Gene Synthesis: The EF1α-EGFP-HBV polyA Gene was Synthesized by BoMed.

### 2) Primer Design

puc-Ad5E4-left armF:
(SEQ ID NO: 88)
AGGTGACACTATAGAATACACGTTAATTAAATCATCAATAATATACC
TTATTTTG
Ad5E4-left arm-EF1a-R:
(SEQ ID NO: 89)
caatccccccttttcttttaaaaAACACCACTCGACACGGCAC
EF1α-F:
(SEQ ID NO: 90)
ttttaaaagaaaaggggggattg
EF1α-R:
(SEQ ID NO: 91)
TAGAGCCCCAGCTGGTTCTTT
EF1α-Ad5E4-right arm-F:
(SEQ ID NO: 92)
GGAAAGAACCAGCTGGGGCTCTAGCAATTGAAAAATAAACACGTTGA
Ad5E4-right arm-puc-R:
(SEQ ID NO: 93)
TAATACGACTCACTATAGGGAGACCCAAAATGTAACCACTGTGAG
puc-F:
(SEQ ID NO: 94)
TCTCCCTATAGTGAGTCGTATT
puc-R:
(SEQ ID NO: 95)
CGTGTATTCTATAGTGTCACCT
ORF6/7-Protease-F:
(SEQ ID NO: 96)
CGTTGAAACATAACACAAACGATACGGCGCAGACGGCAAGGGTGGG

### 3) Amplification of the Target Fragment

\textcircled{1} A Using the synthetic fragment of EF1α-EGFP-HBV gene as the template and EF1α-F and EF1α-R as the primers, the EF1α-EGFP-HBV polyA fragment of the pS5E4-EGFP shuttle plasmid was amplified; Amplification system: the synthetic fragment of EF1α-EGFP-HBV gene was 50 μg, 10 μM EF1α-F primer was 1 ul, 10 μM EF1α-R primer was 1 ul, and Q5 high-fidelity enzyme was 20 ul; Replenish water to 40 ul. PCR procedure: 98° C., 10 sec; 98° C., 5 sec, 60° C., 30 sec, 72° C., 40 sec, 35 cycles; 72° C., 5 min.

\textcircled{2} The left arm fragment of pS5E1 shuttle plasmid was amplified using pAd5LCL3 as the template and puc-Ad5E4-left arm-F and Ad5E4-left arm-EF1α-R as the primers. Amplification system: pAd5LCL3 plasmid 50 ng, 10 uM puc-Ad5E4-left arm-F primer 1 ul, 10 uM Ad5E4-left arm-EF1α-R primer 1 ul, Q5 high-fidelity enzyme 20 ul; Replenish water to 40 ul. PCR program: 98° C., 10 s; 98° C., 5s, 60° C., 30s, 72° C., 10 sec, 35 cycles; 72° C., 5 min.

\textcircled{3} The right arm fragment of the pS5E4-EGFP shuttle plasmid was amplified using pAd5LCL3 as the template and EF1α-Ad5E4-right arm-F and Ad5E4-right arm-puc-R as the primers; Amplification system: pAd5LCL3 plasmid 50 ng, 10 uM EF1α-Ad5E4-right arm-F primer 1 ul, 10 uM Ad5E4-right arm-puc-R primer 1 ul, Q5 high-fidelity enzyme 20 ul; Replenish water to 40 ul.

PCR program: 98° C., 10 s; 98° C., 5s, 60° C., 30s, 72° C., 40 sec, 35 cycles; 72° C., 5 min.

\textcircled{4} PCR amplification of the pS5E4-EGFP shuttle plasmid backbone using pS5E1 plasmid as the template and puc-F and puc-R as the primers; Amplification system: pS5E1 framework plasmid of 50 ng, 10 uM puc-F primer of 1 ul, 10 uM puc-R primer of 1 ul, Q5 high-fidelity enzyme of 20 ul; Replenish water to 40 ul. PCR program: 98° C., 10 s; 98° C., 5s, 60° C., 30s, 72° C., 1 min 20 sec, 35 cycles; 72° C., 5 min. Agarose validation of that amplification product was shown in FIG. 29, where lane 1 was the left arm of the pS5E4-EGFP shuttle plasmid, lane 2 was the right arm of the pS5E4-EGFP shuttle plasmid, lane 3 was EF1α-EGFP-HBV, lane 4 was the pS5E4-EGFP shuttle plasmid backbone, and M was DL 2,000 DNA Marker. As shown in FIG. 29, the amplification result was correct.

### 4) The Target Fragment was Purified by an Axygen™ Gel Extraction Kit.

### 5) Ligation Transformation of Fragments

The four fragment of pS5E4-EGFP shuttle plasmid left arm, pS5E4-EGFP shuttle plasmid right arm, EF1α-EGFP-HBV, and pS5E4-EGFP shuttle plasmid skeleton were connected by use that bode seamless cloning kit. The ligation system consisted of 2×Smealess Cloning Mix 10 ul, pS5E4-EGFP shuttle plasmid left arm segment 50 ng, pS5E4-EGFP shuttle plasmid right arm segment 50 ng, EF1α-EGFP-HBV segment 50 ng, pS5E4-EGFP shuttle plasmid backbone segment 50 ng, water replenishment to 20 ul, and incubation at 50 C for 40 minutes. The ligated products were transformed into DH5α competent cells, plated on plates containing ampicillin resistance and incubated at 37° C. for 12-16 hours.

### 6) Plasmid Validation

\textcircled{1} colony PCR

The target fragment was amplified by PCR using the primer puc-Ad5E4-left arm-F/ER1α-R as the primer colony, and validated by agarose gel assay. The results were shown in FIG. 30, and a positive band appeared.

\textcircled{2} enzyme digestion verification

The positive clones No. 3, 4, 5 and 6 were picked and placed in 5 mL LB liquid medium containing ampicillin resistance for culture for 12-15 hours, and the plasmids were extracted for restriction enzyme digestion verification. The electrophoresis results were shown in FIG. 31, where lane1-4 were the single restriction enzyme digestion of the positive clones No. 3, 4, 5 and 6 PacI, lane 5-8 were the single restriction enzyme digestion of the positive clones No. 3, 4, 5 and 6 HindIII, M₁ and M₃were M₃: 15000 bp Marker. M₂: 2000 bp Marker; The results of enzyme digestion were correct and the sequencing was correct. The human adenovirus type 5 vector E4 region shuttle plasmid pSSE4-EGFP was successfully constructed, and the vector map was shown in FIG. 53.

2. Construction of African Swine Fever Human Adenovirus Type 5 Vector E4 Region Shuttle Plasmid pSSE4-P30-2A-P54

### 1) Primer Design

P30-BamHI-F:
(SEQ ID NO: 97)
cgcGGATCCGCCACCATGGACTTCATCCTGAACATCA
P30-2A-R:
(SEQ ID NO: 98)
CTCCGCTTCC GGCGTAGTCGGGCACGTCGTA
P2A-F:
(SEQ ID NO: 99)
ACGACGTGCCCGACTACGCC GGAAGCGGAGCTACTAACTTC
P2A-R:
(SEQ ID NO: 100)
CTGGAAGAACTCGCTGTCCAT AGGTCCAGGGTTCTCCTCCACGT
2A-P54-F:
(SEQ ID NO: 101)
CCCTGGACCT ATGGACAGCGAGTTCTTCCAG
P54-XhoI-R:
(SEQ ID NO: 102)
ccg CTCGAG TTAGAGGGAGTTTTCCAGGTC

### 2) Amplifying the Target Fragments P30, P54 and 2A

\textcircled{1} The P30 fragment was amplified using the P30 gene synthetic fragment as the template and P30-BamHI-F and P30-2A-R as the primers; Amplification system: P30 gene synthetic fragment 50 μg, 10 μM p30-BamHI-F primer 1 ul, 10 μM p30-2A-R primer 1 ul, Q5 high-fidelity enzyme 20 ul; Replenish water to 40 ul; PCR procedure: 98° C., 10 sec; 98° C., 5 sec, 60° C., 30 sec, 72° C., 20 sec, 35 cycles; 72° C., 5 min.

\textcircled{2} The P54 fragment was amplified using the P54 synthetic fragment as the template and 2A-P54-F and P54-XhoI-R as the primers; Amplification system: P54 gene synthesis fragment 50 ng, 10 uM 2A-P54-F primer 1 ul, 10 uM P54-XhoI-R primer 1 ul, Q5 high-fidelity enzyme 20 ul; Replenish water to 40 ul; PCR procedure: 98° C., 10 sec; 98° C., 5 sec, 60° C., 30 sec, 72° C., 20 sec, 35 cycles; 72° C., 5 min.

\textcircled{3} Using the synthetic fragment of 2A gene as a template and P2A-F and P2A-R as primers, we amplified the 2A fragment; Amplification system: synthetic fragment of 2A gene (50 μg), 10 μM P2A-F primer (1 ul), 10 μM P2A-R primer (1 ul), and Q5 high-fidelity enzyme (20 ul); Replenish water to 40 ul; PCR procedure: 98° C., 10 sec; 98° C., 5 sec, 60° C., 30 sec, 72° C., 20 sec, 35 cycles; 72° C., 5 min.

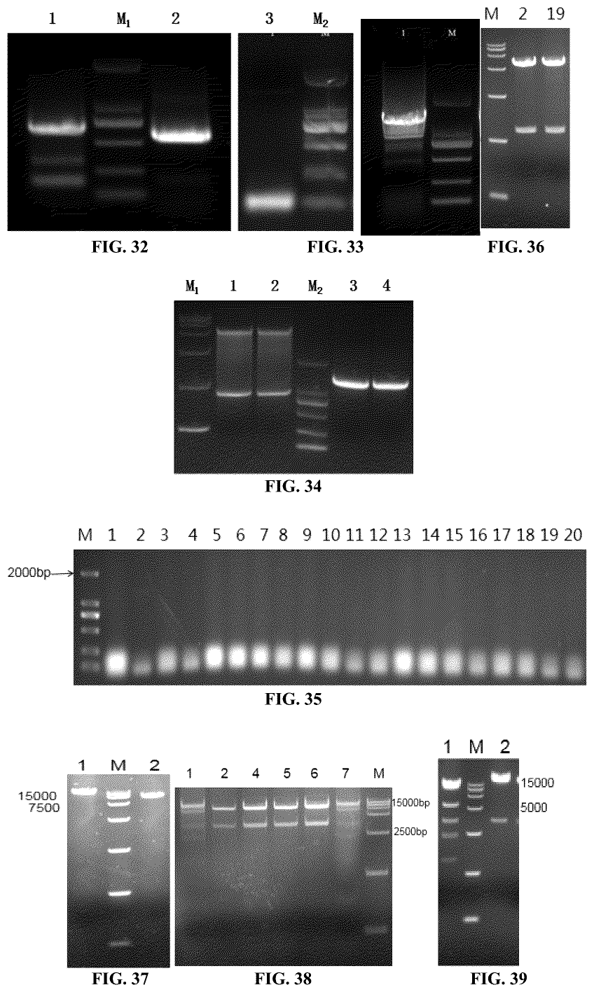

The amplification results were shown in FIG. 32, in which lane 1 was the P30 amplified fragment, lane 2 was the P54 amplified fragment, lane 3 was the 2A amplified fragment, and M1 and M2 were 2000 bp ladder.

### 3) The Target Fragment was Purified by an Axygen™ Gel Extraction Kit.

### 4) Amplifying P30-2A-P54 Fragments by Fusion PCR

Amplification system: 50 ng recovered fragment of P30 gel, 50 ng recovered fragment of P54, 50 ng recovered fragment of P2A, 1 ul of 10 uM P30-BamHI-F primer, 1 ul of 10 uM P54-XhoI-R primer, and 25 ul; of Q5 high-fidelity enzyme; Replenish water to 50 ul; PCR procedure: 98° C., 10 sec; 98° C., 5 sec, 60° C., 30 sec, 72° C., 50 sec, 35 cycles; 72° C., 5 min. The fusion results were shown in FIG. 33, where lane 1 was the P30-2A-P54 fragment and M was DL 2,000 DNA Marker.

5) Excision of the Target Fragment P30-2A-P54 with the pS5E4-EGFP Vector

Enzyme digestion reaction system: the vectors were pS5E4-EGFP, P30-2A-P54 fragment (2 ug), BamHI and XhoI (1 ul; each). 10×cutsmart buffer 5 ul; Replenish water to 50 ul. Reaction condition: 37° C., 30 min; Inactivated at 65° C. for 20 min. Recovery and purification. The enzyme digestion results were shown in FIG. 34, where lanes 1 and 2 were pS5E4-EGFP, BamHI and XhoI were recovered by double enzyme digestion, lanes 3 and 4 were fragments P30-2A-P54, BamHI and XhoI were recovered by double enzyme digestion, and M was DL 15,000 DNA Marker.

6) Ligation and Transformation of pSSE4 Vector to P30-2A-P54 Fragment

Ligation system: pSSE4 (100 ng), P30-2A-P54 fragment (50 ng), T4 DNA ligase 1 ul, 10×ligase buffer 1 ul, supplemented to 10 ul. Reaction condition: room temperature, 30 min. The ligated products were transformed into DH5α competent cells, plated on plates containing ampicillin resistance and incubated at 37° C. for 12-16 hours.

### 7) Plasmid Validation

\textcircled{2} colony PCR

Using primers P30-BamHI-F and P54-XhoI-R as primers, the target fragment was amplified by colony PCR and verified by agarose gel assay, the results were shown in FIG. 35, where No. lane1-20 were the colonies and M was DL 2,000 DNA Marker, as shown in FIG. 35, positive bands appeared on No. 2 and No. 19.

\textcircled{2} enzyme digestion verification

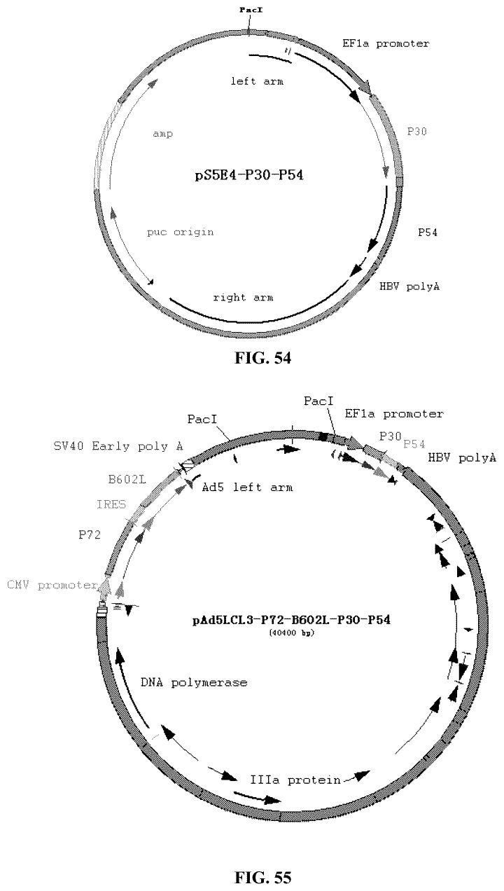

Selecte positive clones No. 2 and No. 19, culture in 5 mL LB liquid medium containing ampicillin resistance for 12-15 hours, extracting plasmid for double enzyme digestion verification of BmHI and XhoI; The enzyme digestion results were shown in FIG. 36, where lane 2 was the double enzyme digestion verification of the No. 2 positive clones BamHI and XhoI, and lane 19 was the double enzyme digestion verification of the No. 19 positive clones BamHI and XhoI, with M being 15000 bp Marker. The enzyme digestion result was correct and the sequencing was correct. The African swine fever human adenovirus type 5 vector E4 region shuttle plasmid pS5E4-P30-2A-P54 was successfully constructed, and the vector map was shown in FIG. 54.

### Example 6:Shuttle Plasmid pAd5LCL3-P72-IRES-B602L, pS5E4-P30-2A-P54 and pAd5LCL3 to Recombinating the pAd5LCL3-P72-B602L-P30-P54 Plasmid

1. Autologous Recombination of Shuttle Plasmid pS5E1-P72-IRES-B602L and Adenovirus Vector Plasmid pAd5LCL3

1) PacI and SwaI perform enzyme digestion on shuttle plasmid pS5E1-P72-IRES-B602L and adenovirus vector plasmid pAd5LCL3, and the enzyme digestion reaction system was as follows:
A, shuttle plasmid pS5E1-P72-IRES-B602L3p g; PacI 2 ul; buffer cutsmart 4 ul; and adding water to 40 ul.
B, adenovirus vector plasmid pAd5LCL3 3 ug; SwaI 2 ul; Buffer 3.1 4 ul; and adding water to 40 ul.

Reaction condition was 37° C. for 1 h; Inactivated at 65° C. for 20 min.

Two uL of agarose gel were verified as shown in FIG. 37, with lane 1 being pAd5LCL3 and lane 2 being pS5E1-P72-IRES-B602L.

2) Dephosphorylation of that enzyme digestion product

Reaction system: 37.5 ul of enzyme digestion reaction solution; Dephosphorylase 1 uL; Dephosphorylated buffer 5 ul; Replenish water to 50 ul. Reaction condition were 37° C. for 1 h; Inactivated at 65° C. for 5 min.

3) Use OMEGA Ultra-Sep Gel Extraction Kit to recover the vectors and fragments.

4) 100 ng of the purified shuttle plasmid pS5E1-P72-IRES-B602L and 100 ng of the purified adenovirus vector pAd5LCL3were co-transformed into BJ5183 competent cells, and the transformed product was coated with an LB plate containing Kan and cultured at 37° C. for 12-16 h.

5) The colonies were selected and cultured in 5 mL LB liquid medium containing Kan under shaking at 37° C. for 12-16 h, and the plasmids were extracted for XhoI enzyme digestion verification. The results were shown in FIG. 38, where Lane 1-7 was a clone of pAd5LCL3-P72-IRES-B602L, M was DL 15,000 DNA Marker. As shown in FIG. 38, Clones 1 and 7 were correctly digested.

6) The No. 1 positive plasmid was converted into DH5α competent state, a colony was selected and cultured in 5 mL LB liquid medium containing Kan under shaking at 37° C. for 12-16 h, and the plasmid was extracted for XhoI restriction enzyme digestion verification again. The restriction enzyme digestion result was shown in FIG. 39, where the No. 1 lane was subjected to XhoI restriction enzyme digestion of pAd5LCL3-P72-IRES-B602L plasmid. Lane 2 was the pAd5LCL3-P72-IRES-B602L plasmid PacI restriction endonuclease, and M was DL 15,000 DNA Marker. As shown in FIG. 39, the restriction endonuclease result was correct, and the adenovirus vector plasmid pAd5LCL3-P72-IRES-B602L was successfully constructed.

2. Autologous recombination of shuttle plasmid pS5E4-P30-2A-P54 and adenovirus vector plasmid pAd5LCL3-P72-IRES-B602L to obtain pAd5LCL3-P72-B602L-P54

1) PacI and I-sceI perform enzyme digestion on shuttle plasmid pS5E4-P30-2A-P54 and adenovirus vector plasmid pAd5LCL3-P72-IRES-B602L, and the enzyme digestion reaction system was as follows:

A. Shuttle plasmid PSSE4-P30-2A-P54 3 s g; PacI 2 ul; 10×cutsmart buffer 4 ul; Replenish water to 40 ul.

B. Adenovirus vector plasmid pAd5LCL3-P72-IRES-b602L3 ug; I-sceI 2 ul; Buffer cutsmart 4 ul; Replenish water to 40 ul.

Reaction condition were 37° C. for 1 h; Inactivated at 65° C. for 20 min.

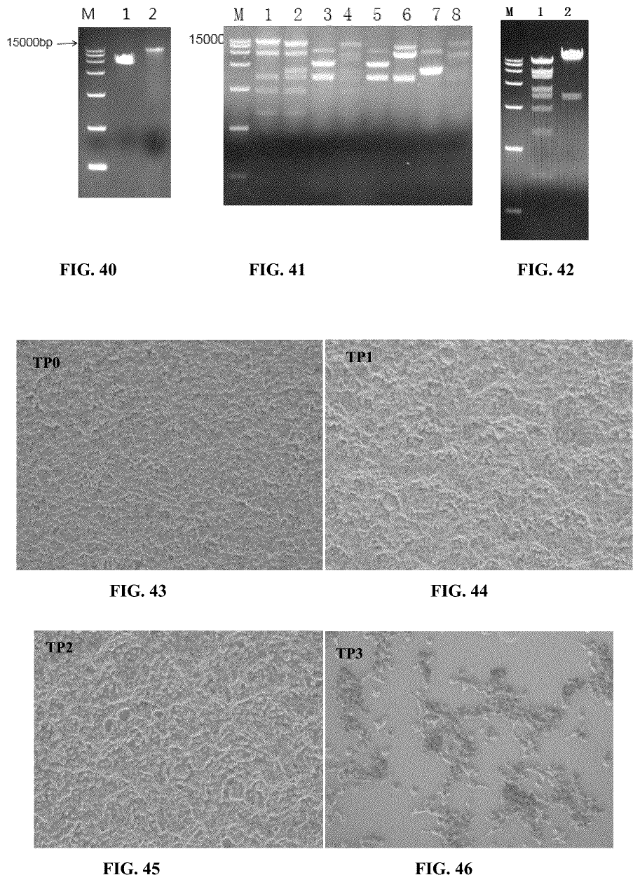

2 ul of agarose gel was used for validation and the results were shown in FIG. 40, with lane 1 being pAd5LCL3 and lane 2 being pSSE1-P72-IRES-B602L.

2) Dephosphorylation of that enzyme digestion product

Reaction system: 37.5 ul of enzyme digestion reaction solution; Dephosphorylase 1 uL; Dephosphorylated buffer 5 ul; Replenish water to 50 ul. Reaction condition were 37° C. for 1 h; Inactivated at 65° C. for 5 min.

3) Use OMEGA Ultra-Sep Gel Extraction Kit to recover the vectors and fragments.

4) 100 ng of the purified shuttle plasmid and 100 ng of the purified adenovirus vector were co-transformed into BJS183 competent cells, and the transformed product was coated with an LB plate containing Kan and cultured at 37° C. for 12-16 h.

5) Eight colonies were selected and cultured in 5 mL LB liquid medium containing Kan under shaking at 37° C. for 12-16 h, and the plasmids were extracted for XhoI enzyme digestion verification. The results were shown in FIG. 41, with lanes 1-8 as colonies and M as DL 15,000 DNA Marker. It can be seen from FIG. 41 that plasmid No. 4 was correct.

6) transform that No. 4 positive plasmid into DH5a competence; One colony was selected and cultured in 5 mL LB liquid medium containing Kan under shaking at 37° C. for 12-16 h, and the plasmid was extracted for XhoI restriction enzyme digestion verification again. The results of enzyme digestion were shown in FIG. 42, among which, lane No. 1 was for the enzyme digestion of plasmid pAd5LCL3-P72-B602L-P30-P54 XhoI, and lane No. 2 was for the enzyme digestion of plasmid pAd5LCL3-P72-B602L-P30-P54 PacI, with the M of DL 15,000 DNA Marker. As shown in FIG. 42, the enzyme digestion result was correct. The adenovirus vector plasmid pAd5LCL3-P72-B602L-P30-P54 was successfully constructed, and the vector map was shown in FIG. 55.

### Example 7:Packaging of Recombinant Adenovirus

Packaging the pAd5LCL3-P72-B602L-P30-P54 plasmid with 293TD37 cells as follows steps:

Preparation of 293TD37 cells: The cells were prepared one day before transfection. The 293TD37 cells to be transfected were inoculated into a 6-well plate with 0.5×10⁶ viable cells/well and incubated at 37° C. with 5% CO₂ for 24 hours. The cells showed 40-50% confluency on the day of transfection.

Linearization of plasmid pAd5LCL3-P72-B602L-P30-P54: The plasmid to be transfected was digested with PacI enzyme, incubated at 37° C. for 40 min, and inactivated at 65° C. for 20 min.

Transfection: The linearized 2 pg plasmid and PEI were diluted with 100 ul serum-free medium, respectively. The plasmid diluent was added into the PEI diluent, and repeatedly aspirated for 5 times or vortexed for 10 seconds to be mixed evenly, and incubated for 10 minutes at room temperature to form a transfection complex. During incubation, cell culture medium was gently aspirated from the plates, 2 mL of fresh growth medium was added, and after 10 minutes the transfection complex was added to the cells in fresh medium.

Cell culture: the transfected 293TD37 cells were incubated at 37° C. for 72-96 hours in a 5% CO₂ incubator; 6-well plate cell suspensions were collected in 1.5 ml centrifuge tubes, TP0, 72-96 hours after viral plasmid transfection.

Continuous inoculation: The collected cell suspension was repeatedly frozen and thawed at −80° C. for 3 times, centrifuged at 2000 g at 4 C for 10 minutes, 500 ul of supernatant was collected to infect 293TD37 cells (293TD37 cells need to be prepared one day in advance), incubated at 37° C. with 5% CO₂ for 60 minutes, supplemented with 2 mL of FBS medium, and cultured at 37° C. with 5% CO₂ for 72 hours, to collect cell suspension, namely TP1. The previous steps were repeated again and the cell suspension, namely TP2, was collected. The exposure was continued until the TP4 cells became diseased.

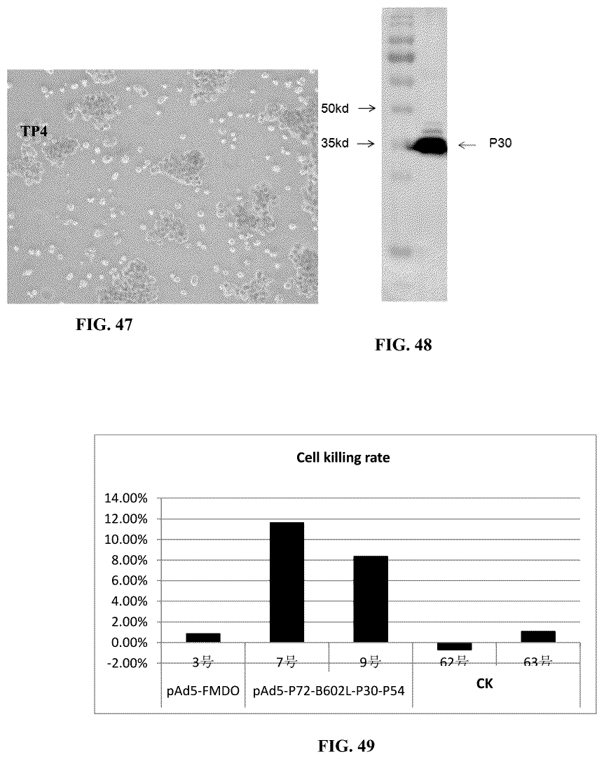

Cytopathic effect: When the 293TD37 cells were cultured from TP0 to TP4, the cells gradually became diseased until the 293TD37 cells were completely diseased at TP4. The cytopathic effects caused by TP0 to TP4 were shown in FIG. 43-47, respectively. TP4 is completely diseased.

### Example 8: Detection of Titer of African Swine Fever Multiantigen Recombinant Adenovirus Vaccine

The 293TD37 cells were prepared. The cells with good growth in T75 culture flask were taken, the supernatant was discarded, the cells were washed with PBS, and digested with 0.25% trypsin, and then 10 mL of fresh DMEM medium containing 10% fetal bovine serum was added to stop the digestion, which was then blown, mixed, inoculated into 6-well plates (5×10⁵/mL, 2 ml per well), and allowed to stand for culture in a 37° C. 5% CO₂ incubator. After 24 hours, when the cells adhered to grow into single-layer cells, the culture medium was discarded, and the recombinant adenovirus was continuously diluted 10-3 to 10-6 times with serum-free DMEM maintenance solution, and two wells were inoculated with each dilution degree at 250 uL per well. After one hour of infection, the supernatant was discarded to supplement the complete culture medium, and then the medium was allowed to stand for culture in a 5% carbon dioxide incubator at 37° C. After 24 h, the supernatant was discarded and the cells were washed with PBS (1 mL per well). After PBS was discarded, 1 mL of cold formaldehyde was added into each well for fixation, and formaldehyde was discarded at room temperature for 10 min. Then the cells were washed with PBS (1 mL per well), followed by adenovirus antibody-FITC (1 ml per well). After 1 h at room temperature, the cells were washed with PBS again (1 mL per well). After two times, 1 mL of PBS was added into each well and counted under fluorescence microscope (200 times, 10 continuous fields). Calculation: Virus titer (FFU/mL)=Mean×1013×4×10⁽⁻ⁿ⁾. The FFU of the pAd5LCL3P72-B602L-P30-P54 virus was 2×10⁸ FFU/mL with a high titer.

### Example 9: Detection of Stability of African Swine Fever Multiantigen Recombinant Adenovirus Vaccine pAd5LCL3-P72-B602L-P30-P54

The 293TD37 cells were prepared. The cells that grew well in T75 culture flask were taken, the supernatant was discarded, the cells were washed with PBS, and digested with 0.25% trypsin. Then 10 mL of fresh DMEM medium containing 10% fetal bovine serum was added to stop the digestion, and then the cells were blown and mixed evenly. The 293TD37 cells were planted into 6-well plates (5×10⁵ cells/mL, 2 mL/well), incubated for 1 hour at room temperature to adhere to the wall, and incubated for microscopic observation of its attachment degree. pAd5LCL3-P72-B602L-P30-P54 virus particles were used for infection, and the titer of infection was 5 MOI/well. After the 293TD37 cells developed lesions 48 hours later, the cells were collected, repeatedly frozen and thawed for three times, and then centrifuged at 2000 g; the collected supernatant was detected for FFU, and then new 293TD37 cells were reinfected until the 30th generation. The collected virus solutions of passages 5, 10, 15, 20, 25 and 30 were tested and found that the genome of the virus was still intact, indicating that the replication-defective pAd5LCL3-P72-B602L-P30-P54 virus could be stably packaged in 293TD37 cells.

### Example 10: Detection of African Swine Fever Multiantigen Recombinant Adenovirus Vaccine pAd5LCL3-P72-B602L-P30-P54 recovery mutation (RCA)

PAd5LCL3-P72-B602L-P30-P54 virus RCA detection, detection method was as follows:

1. Prepare pAd5LCL3-P72-B602L-P30-P54 virus solution, measure its virus titer and determine the concentration of virus particles. The DNA of the host cell is digested in the virus solution with 1% Benzonase (Benzonase 7.5-15 units/mL virus solution) in a water bath of 37° C. for 40 min. The virus particles were collected using a 300Kd ultrafiltration centrifuge tube after centrifugation at 1000 g for 30 min, followed by elution with 1×PBS. A260 was measured as the particle concentration=A260×1.1×10¹² VP/mL.

2. For virus infection, 12-well plates of A549 cells were prepared, with each well cell being 2.5×10⁵/well, the culture medium was discarded and PBS was used for one time. Adenovirus was inoculated according to 1×10⁹ VP/well/0.5 ml to infect A549 cells. Wild-type human adenovirus type 5 was used as the positive control at 37° C. and 5% CO₂. After 1 h, the virus solution was discarded and made up of 5% complete culture medium and cultured at 37° C. and 5% CO₂ for 48 h.

3. Immunostaining was performed, and the cell supernatant was discarded. The cells were surface washing cells in PBS, fixed with ice formaldehyde, placed at −20 C for 20 min, and washed with 1×PBS for three times, each time for 5 min. Then 2 ml 1% BSA-PBS solution was added into each well, placed in a shaker, and incubated for 1 h. After the supernatant was discarded, human adenovirus type 5 fluorescent antibody (1:500 dilution) was added and incubated for 1 h, followed by washing with 1×PBS for three times, 5 min each time.

RCA was calculated using the equation as observed under a 10-fold fluorescence microscope

$$RCA = ( average positive cell field ) \times ( 374 field / well ) \times ( dilution factor ) ) / Total VPs in 0.5 ml viral sample$$

The judging standard was that the level of RCA was less than 1RCA/3×10¹⁰ vp. Through statistics, the RCA level of the pAd5LCL3-P72-B602L-P30-P54 is less than 1 RCA/3*10¹⁰ VP, which indicates that the replication-defective pAd5LCL3-P72-B602L-P30-P54 virus prepared by the invention can be stably packaged in 293TD37 cells and has low probability of not being converted into wild type or wild type.

### Example 11: Detection of Expression of pAd5LCL3-P72-B602L-P30-P54 Protein in African Swine Fever Multiantigen Recombinant Adenovirus Vaccine

The 293TD37 cells were prepared one day in advance and placed in a 12-well cell culture plate. The 293TD37 cells were infected with the African swine fever multiantigen recombinant adenovirus vaccine pAd5LCL3-P72-B602L-P30-P54 virus, and the cells became diseased 48 hours later. All 1 ml of cells were collected, washed with PBS, and prepared for Western Blot detection. The target protein was detected by antibodies of P30, which were rabbit serum immunized with prokaryotic expression of P30 protein. The experimental results were shown in FIG. 48, and P30 protein could be clearly seen in the vaccine. Rabbit serum also immunized with P54 and P72 proteins were shown in FIG. 56: M, preincubated with Makker; Lane 1, P54 antibody serum; Lane 2, P72 antibody serum; Lane 3: 293 TD37 cell control. Thus, the target protein of the African swine fever multi-antigen recombinant adenovirus vaccine pAd5LCL3-P72-B602L-P30-P54 was significantly expressed.

### Example 12: Immunological Evaluation of African Swine Fever Multi-Antigen Recombinant Adenovirus Vaccine pAd5LCL3-P72-B602L-P30-P54 in Mouse Model

### 12.1 Detection of Vaccine Humoral Immune Response

Twenty SPF-grade mice (6-8 weeks of age) were randomly divided into four groups, five for each group. Mice were immunized with pAd5LCL3-P72-B602L-P30-P54 according to the groupings shown in Table 1. The injection method was as follows: intramuscular injection was performed on the medial aspect of the posterior thigh. Injection dose: 100 ul.

TABLE 1
Groups of Vaccinated Mice

| | | | Immune | Number |
|---|---|---|---|---|
| group | Vector vaccine | dosage | mode | of mice |
| High | pAd5LCL3-P72- | 1*10\hat{ }8 FFU | intramuscular | 5 |
| dose | B602L-P30-P54 | | injection | |
| Medium | pAd5LCL3-P72- | 1*10\hat{ }7 FFU | intramuscular | 5 |
| dose | B602L-P30-P54 | | injection | |
| Low | pAd5LCL3-P72- | 1*10\hat{ }6 FFU | intramuscular | 5 |
| dose | B602L-P30-P54 | | injection | |
| Control | pAd5LCL3 | 1*10\hat{ }7 FFU | intramuscular | 5 |
| | | | injection | |

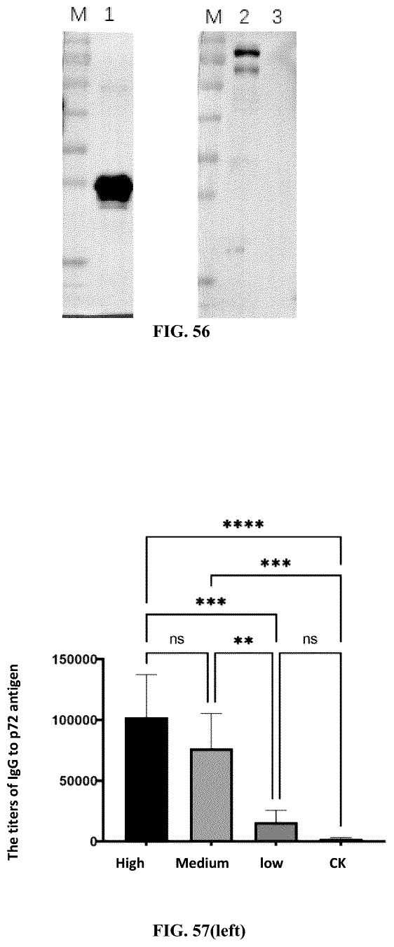

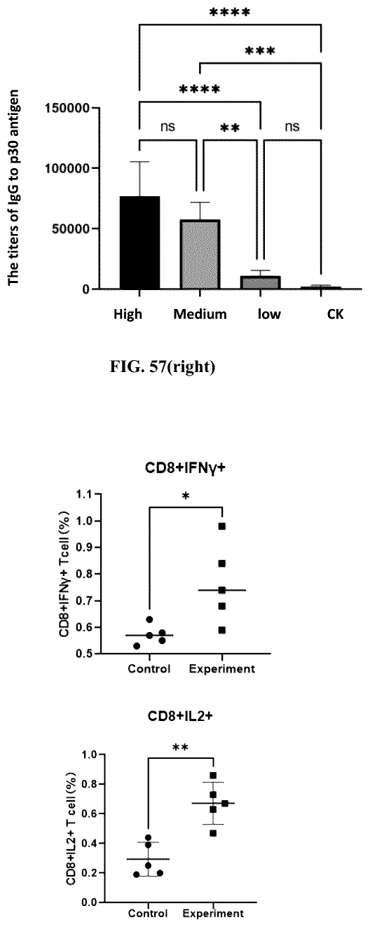

The blood was collected 14 days after immunization, and the serum was isolated. The IgG antibody titers against the African swine fever target proteins P72 and P30 in the serum were detected by indirect ELISA. The test results were shown in FIG. 57 (ns, P≥0.05; *:P<0 0.05; **:P<0.01; ***:P<0.001; ****:P<0.0001), where the left was IgG antibody titer of P72 and the right is IgG antibody titer of P30.

As shown in FIG. 57, after intramuscular injection of pAd5LCL3-P72-B602L-P30-P54, mice were able to produce high concentrations of IgG antibodies to both p72 and p30 proteins. Among the P72 antibodies, the average titer of antibodies in the high-dose group was more than 105, and the average titer in the medium-dose group was 70000, showing a significant difference from that in the control group. High-dose group and medium-dose group could induce high titer of P30 antibody.

### 12.2 Detection of Cellular Immune Response

Ten SPF-grade mice (6-8 weeks of age) were randomly divided into two groups, five for each group. Mice were immunized with pAd5LCL3-P72-B602L-P30-P54 according to the groupings shown in Table 2. The injection method was as follows: intramuscular injection was performed on the medial aspect of the posterior thigh. Injection dose: 100 ul.

TABLE 2
Groups of Vaccinated Mice

| | | | Immune | Number |
|---|---|---|---|---|
| group | Vector vaccine | dosage | mode | of mice |
| Experimental | pAd5LCL3-P72- | 1*10\hat{ }7 FFU | intramuscular | 5 |
| | B602L-P30-P54 | | injection | |
| Control | pAd5LCL3 | 1*10\hat{ }7 FFU | intramuscular | 5 |
| | | | injection | |

The mice were sacrificed 14 days after immunization, and the splenic lymphocytes were isolated. PK15 cells transfected with the shuttle plasmids pS5E1-P72-IRES-B602L and pS5E4-P30-2A-P54 were stimulated and cultured for 6 hours, while protein secretion blockers were added to block cytokine secretion. After 6 hours, Fc receptors were blocked, dead cells and cell surface molecular markers were stained, and intracellular cytokines were stained after the cells were fixed and perforated. Cell surface markers included CD4 and CD8, and intracellular cytokines included IFNγ and IL2. The expression levels of IFNγ and IL2 in CD4+T cells and CD8+T cells stimulated by the target protein were analyzed by flow cytometry (CyExpert).

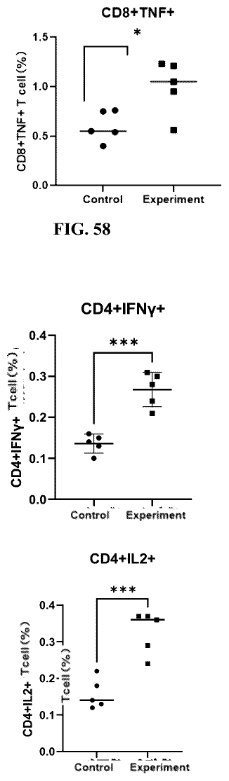

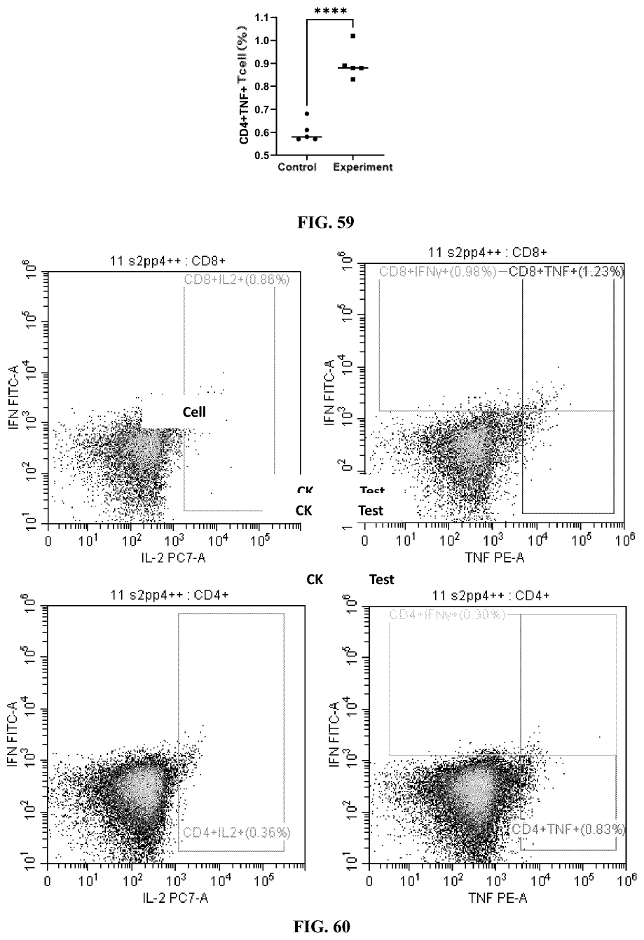

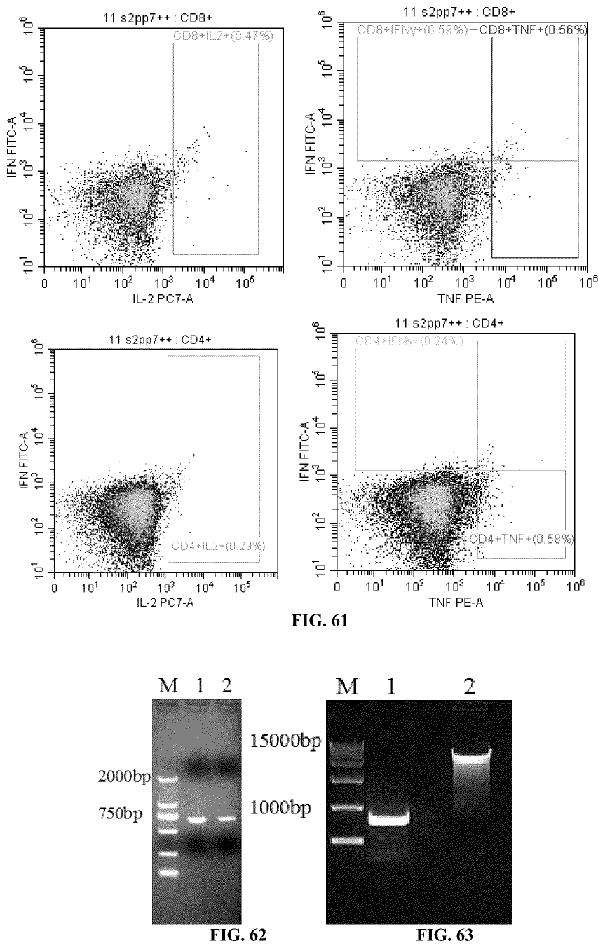

The CD8+T cell and CD4+T cell immune responses induced by pAd5LCL3-P72-B602L-P30-P54 were shown in FIG. 58 and FIG. 59, and the representative results were shown in FIG. 60 and FIG. 61. Among them, FIG. 60 was the representative diagram of cellular immune response after intramuscular injection of pAd5LCL3-P72-B602L-P30-P54, and FIG. 61 is the representative diagram of blank control immune response. The results showed that, 14 days after immunization, after the spleen cells were stimulated by the target protein, the levels of IFNγ, TNFα and IL2 expressed in CD8+T cells were significantly higher than those in the HAd5 vector control group (Control) (P<0 0.05). After stimulation, the expression levels of IFNγ, TNFα and IL2 in CD4+T cells were significantly higher than those in the HAd5 vector control group (P<0.05).

### 12.3 Summary of Immunogenicity Evaluation of Mouse Model

pAd5LCL3-P72-B602L-P30-P54 recombinant adenovirus has strong immunogenicity and can induce mice to produce high levels of serum IgG antibodies. High doses of 1*10⁸ FFU and medium doses of 1*10⁷ FFU resulted in high immunologically induced titers. Since the P72 and B602L antigens, P30 and P54 antigens were respectively regulated and expressed by the same expression elements, the serum IgG antibodies of P72 and P30 can represent that all four antigens have high immunogenicity. The results of cellular immune response showed that intramuscular injection of the adenovirus vector vaccine of 1*10⁷ FFU could induce specific cellular immune response in the immunized mice.

### Example 13: Immunological Evaluation of African Swine Fever Multiantigen Recombinant Adenovirus Vaccine pAd5LCL3-P72-B602L-P30-P54 on Target Animals (Ternary Piglets)

### 13.1 Target Animal (Sanyuan Pig) Vaccine Humoral Immune Response Detection

Animal immunization with the African swine fever multiantigen recombinant adenovirus pAd5LCL3-P72-B602L-P30-P54 vaccine: Three-yuan pigs were immunized with the pAd5LCL3-P72-B602L-P30-P54 vaccine of 1*10⁹ FFU. Four weeks later, blood samples were collected and serum was isolated from the pigs. The immunized serum samples were tested by the IDVET African Swine Fever Assay Kit. The specific immune mode was shown in Table 3:

TABLE 3
Groups of Vaccinated Ternary Piglets

| | | | | The number |
|---|---|---|---|---|
| | | | Immune | of Ternary |
| group | Vector vaccine | dosage | mode | Piglets |
| Experimenta | pAd5LCL3-P72- | 1*10\hat{ }9 FFU | intramuscular | 5 |
| | B602L-P30-P54 | | injection | |
| Control | pAd5LCL3 | 1*10\hat{ }9 FFU | intramuscular | 2 |
| | | | injection | |

There were five immune experimental groups and two blank control groups in total. The immune experimental results were shown in Table 4.

TABLE 4
experimental test results

| sample name | OD450 | S/P % | result |
|---|---|---|---|
| 1# | 0.87185 | 0.64717 | sun |
| 2# | 0.57005 | 0.409588 | sun |
| 3# | 0.7095 | 0.519366 | sun |
| 4# | 0.66055 | 0.480831 | sun |
| 5# | 0.64565 | 0.469102 | sun |
| 6# (blank) | 0.0855 | 0.028143 | cloudy |
| 7# (blank) | 0.0777 | 0.022003 | cloudy |

Where for each sample the percent S/P (S/P %), S/P %=(OdSample−ODNC)/(ODPC−ODNC)*100 was calculated, S/P % was calculated for each sample, when S/P %≤30 was negative, 30%<S/P %<40% was suspected, and S/P %≥40% was positive.

Experimental validity determination: the experiment was valid under the following conditions:

(1) The mean net OD of the positive control was greater than 0.350; ODPC >0.350

(2) The average net OD value ratio of the positive control to the negative control is greater than 3; OD PC/ODNC >3

The experimental results showed that the recombinant adenovirus pAd5LCL3-P72-B602L-P30-P54 vaccine could induce sufficient immune response in the Sanyuan pig immune test.

13.2 Cytotoxic t Cell (CTL) Killing Experiment Induced by African Swine Fever Multiantigen Recombinant Adenovirus pAd5LCL3-P72-B602L-P30-P54 Vaccine

Animal immunization with African swine fever multiantigen recombinant adenovirus pAd5LCL3-P72-B602L-P30-P54 vaccine: Three-yuan pigs were immunized with 1×10⁸ FFU of pAd5LCL3-P72-B602L-P30-P54 vaccine. After four weeks, blood samples were collected. Porcine peripheral blood lymphocyte separation: The porcine peripheral blood lymphocyte separation kit of Tianjin HaoyangHuake Biotechnology Co., Ltd. was used for lymphocyte separation of the collected porcine blood sample, and the effector cells were counted by a counter. Cytotoxic T cell (CTL) killing assay: A lactate dehydrogenase cytotoxicity assay kit (purchased from Beyotime) was used to detect the cytotoxic T cell (CTL) killing assay. Specific steps: 1. PK15 cells (purchased from the Cell Resource Center of Institute of Basic Medical Science, China Academy of Medical Sciences) were prepared one night in advance and infected with African swine fever pAd5LCL3-P72-B602L-P30-P54 vaccine and adenovirus vector control vaccine (25MOI, 18 hours in advance).

2. Before the experiment, the infected PK15 cells were digested with trypsin and diluted to 1×10⁵/ml by suspension culture in serum-free medium as the target cells. At the bottom of the 96 holes cell culture Target cells were added to the plates, with 100 ul added to each well. The natural release control wells for the three effector cells were treated with 100 ul of culture medium without adding target cells.

3. Add 100 ul effector cells to each well, and the ratio of effector cells to target cells was 50:1. Only 100 ul of culture medium was added to the natural release wells without adding effector cells. Meanwhile, a maximum release control hole was arranged, and a cell release reagent is added.

4. The samples were incubated at 37° C. for 4 hours in a carbon dioxide incubator with 5% CO₂.

5. The plates were centrifuged to 250 g for 10 minutes. One hundred and forty 140 ul of supernatant was aspirated from each well and correspondingly added to another 96-well ELISA plate prepared according to the instructions of the lactate dehydrogenase cytotoxicity test kit and sixty μ 0 was added. The absorbance value of OD490 was detect.

$$Killing activity ( \% ) = [ ( OD test group - total OD natural release ) / ( OD maximum release group - total OD natural release ) ] \times 100 \%$$

The experimental results were shown in FIG. 49. After multiple trials, statistical analysis showed that compared with the group with unrelated antigen (equivalent to blank) adenovirus pAd5LCL3, the group with African swine fever vaccine pAd5LCL3-P72-B602L-P30-P54 had higher CTL killing level with significant difference (P<0.05). The normal saline group showed essentially no levels of killing and the small amounts of data may have been due to error or natural kill. The higher the killing level to the cytotoxic T cells (CTLs), the stronger the specific immune response to the African swine fever virus was proved. Therefore, the African swine fever vaccine pAd5LCL3-P72-B602L-P30-P54 of the present embodiment can significantly enhance the strong specific immune response to the African swine fever virus.

### Example 14: Construction of the African Swine Fever Human Adenovirus Type 5 Vector E1 Region Shuttle Plasmid pS5E1-CP129R Ubiqutin-IRES-MGF5L6L

1. Construction of the shuttle plasmid in the E1 region of the human adenovirus type 5 vector according to the construction method described in Example 4.

2. Construction of shuttle plasmid pS5E1-P72-IRES-B602L of African swine fever human adenovirus type 5 vector

1) Ligation of pS5E1 Fragment to the IRES Fragment

\textcircled{1} primer synthesis

IRES-EcoRV-F:
(SEQ ID NO: 103)
ccg GATATC TGTCGTCATCATCCTTATAGTCC
IRES-NotI-R:
(SEQ ID NO: 104)
aaatat GCGGCCGC GGTTGTGGCCATTATCATCGTG

\textcircled{2} amplifying IRES fragment

Amplification system: 25 ul of Q5 enzyme, 1 uL of 10 uM primer IRES-EcoRV-F, 1 uL of 10 uM primer IRES-NOTI-R, 2 ul of template IRES, and water supplementing to 50 ul; PCR program: 98° C., 10 s; 98° C., 5s, 60° C., 30s, 72° C., 20s, 35 cycles; 72° C., 5 min. The electrophoretic detection of amplification results was shown in FIG. 62, where lanes 1 and 2 were the PCR amplification products of IRES fragment and M was DL 15,000 DNA Marker, so the amplification results were correct.

\textcircled{3} IRES fragment was purified by Axygen™ PCR purification kit.

\textcircled{4} Excision of the target fragment IRES with the pS5E1 vector

Enzyme digestion reaction system: the vector pS5E1, IRES fragment ˜2 ug, and each of EcoRV and NotI was 1 uL; 10×cutsmart buffer 5 ul; Replenish water to 50 ul; Reaction condition: 37° C., 30 min; Inactivated at 65° C. for 20 min; Recovery and purification of gum. The electrophoretic detection of the digested products was shown in FIG. 63, where lane 1 is the enzyme digestion of fragments IRES EcoRV and NotI, lane 2 was the enzyme digestion of pS5E1 EcoRV and NotI, and M was DL 15,000 DNA Marker.

\textcircled{5} pS5E1 vector was connected with IRES fragment

Ligation system: pS5E1 (100 ng); IRES fragment (vector:fragment=1:5, molar ratio); T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

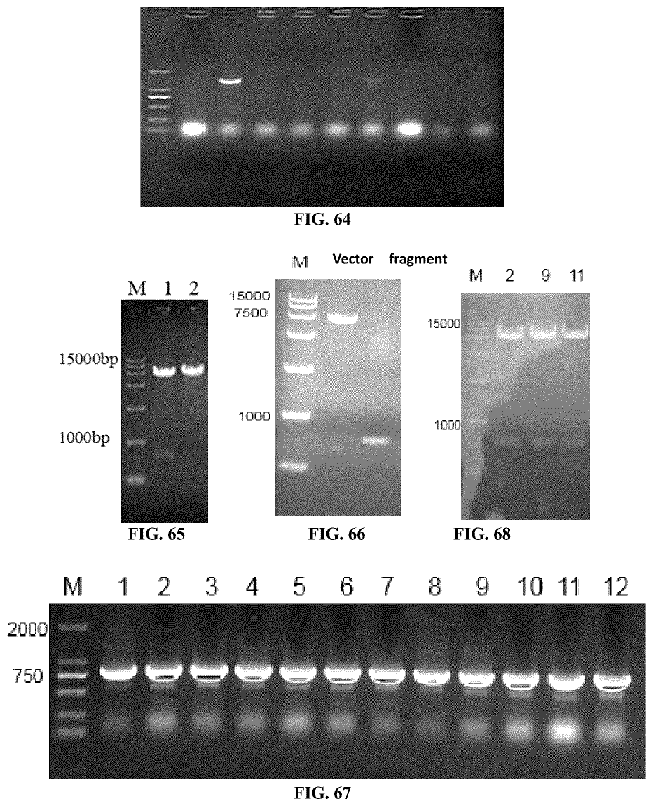

Amplification system: 10 ul of Q5 enzyme, 10 uM primer IRES-EcoRV-F 1 ul, and 10 uM primer IRES-NotI-R 1 ul, and supplementing water to 20 ul; PCR program: 98° C., 10 s; 98° C., 5s, 60° C., 30s, 72° C., 20s, 35 cycles; 72° C., 5 min. Electrophoresis verification was performed as shown in FIG. 64, where No. 1-9 was a colony and M was a Marker, and as shown in FIG. 64, positive bands appeared in No. 2 and No. 6.

\textcircled{7} Plasmid NotI and EcoRV restriction enzyme digestion verification: 2 and 6 were selected for plasmid extraction and restriction enzyme digestion verification, and the results were shown in FIG. 65. The restriction enzyme digestion results of plasmid No. 2 NotI and EcoRV and plasmid No. 6 NotI and EcoRV were shown to be correct.

2) Ligation of pS5E1-IRES to the MGF5L6L Fragment

\textcircled{1} primer synthesis

MGF5L6L-NotI-F:
(SEQ ID NO: 105)
aaggaaaaaaGCGGCCGCgccaccATGCTGGTGATCTTCCTGGG
MGF5L6L-XhoI-R:
(SEQ ID NO: 106)
catgCTCGAG TCAGGCGTAGTCAGGCACAT

\textcircled{2} PCR amplification of MGF5L6L fragment

Amplification system: 25 ul of Q5 enzyme, 10 uM primer MGF5L6L-NotI-F 1 ul, 10 uM primer MGF5L6L-XhoI-R 1 ul, and template MGF5L6L 1 ul, supplemented with water to 50 ul; PCR program: 98° C., 10 s; 98° C., 5 s, 60° C., 30s, 72° C., 40s, 35 cycles; 72° C., 5 min.

\textcircled{3} The MGF5L6L fragment was purified by Axygen™ PCR purification kit.

\textcircled{4} The target fragment MGF5L6L was digested with pS5E1-IRES vector

Enzyme digestion reaction system: vectors pS5E1-IRES, MGF5L6L fragment ˜2 ug, NotI and XhoI 1 uL each; 10×cutsmart buffer 5 ul; Replenish water to 50 ul. Reaction condition: 37° C., 30 min; Inactivated at 65° C. for 20 min. Recovery and purification of gum. The electrophoresis detection of the digested product was shown in FIG. 66, where lane 1 is pS5E1-IRES, NotI and XhoI double-digestion, lane 2 was fragment MGF5L6L, NotI and XhoI double-digestion, and M was DL 15,000 DNA Marker.

\textcircled{5} The target fragment MGF5L6L was connected with pS5E1-IRES

Ligation system: pSSE1-IRES (100 ng); MGF5L6L fragment (vector:fragment=1: 3 molar ratio); T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

Amplification system: 10 ul of Q5 enzyme, 10 uM primer MGF5L6L-NotI-F 1 ul, and 10 uM primer MGF5L6L-XhoI-R 1 ul were replenished to 20 ul; PCR program: 98° C., 10 s; 98° C., 5s, 60° C., 30s, 72° C., 30s, 35 cycles; 72° C., 5 min. Electrophoresis verification was performed as shown in FIG. 67, in which No. 1-12 was a colony and M was DL 2,000 DNA Marker.

\textcircled{7} Plasmid restriction endonuclease assay (NotI and XhoI), colonies 2, 9 and 11 were selected for plasmid extraction and restriction endonuclease assay. The results were as shown in FIG. 68, all of which were positive plasmids.

3) Ligation of pS5E1-IRES-MGF5L6L to Fragment CP129Rubiqutin

\textcircled{1} primer synthesis

CP129R-BamHI-F:
(SEQ ID NO: 107)
cgcGGATCCgccaccATGGAGCACCCCAGCACAAA
CP129R-ubiqutin-R:
(SEQ ID NO: 108)
GGGTTTTCACGAAAATCTGCATGGCGTAATCGGGCACGTCGTAA
ubiqutin-F:
(SEQ ID NO: 109)
ATGCAGATTTTCGTGAAAACCC
ubiqutin-EcoRV-R:
(SEQ ID NO: 110)
ccgGATATC TTACTTGTCTTCTGGTTTGTTGA

\textcircled{2} PCR amplification of CP129Rubiqutin fragment

### Amplified CP129R Fragment

Amplification system: 25 ul of Q5 enzyme, 1 uL of primer CP 129R-BamHI-F, 1 uL of primer CP129R-ubiquitin-R, and 2 uL of template CP129R; the water supplementing amount was up to 50 ul; Reaction condition: 98° C. for 30 s; 98° C. for 10 s, 68° C. for 30 s, 72° C. for 15 s, 35 cycles; 72° C. 5 min.

### Amplified Ubiqutin Fragment

Amplification system: 25 ul of Q5 enzyme, 1 uL of primer ubiqutin-F, 1 uL of primer ubiqutin-EcoRV-R, and 2 uL of template ubiqutin, and supplementing water to 50 ul; Reaction condition: 98° C. for 30 s; 98° C. for 10 s, 68° C. for 30 s, 72° C. for 15 s, 35 cycles; 72° C. 5 min.

### Fusion PCR Amplification of CP129Rubiqutin Fragment

Amplification system: 25 ul of Q5 enzyme, upstream primer CP129R-BamHI-F, downstream primer ubiqutin-EcoRV-R, template fragment CP129R and fragment ubiqutin 50 ng each, and supplementing water to 50 ul; Reaction condition: 98° C.; 98° C. for 5 s, 68° C. for 30 s, 72° C. for 30 s, 35 cycles; 72° C. 7 min.

\textcircled{3} The CP129Rubiqutin fragment was purified by an Axygen™ PCR purification kit.

\textcircled{4} The target fragment CP129Rubiqutin was digested with pSSE1-IRES-MGF5L6L vector

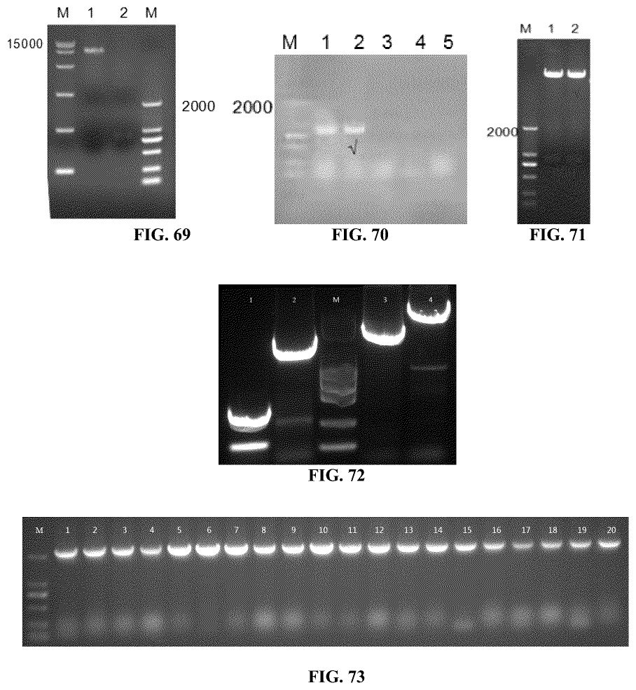

Enzyme digestion reaction system: the vector pSSE1-IRES-MGF5L6L, CP129Rubiqutin fragment 2 ug, and each of EcoRV and BamHI 1 ul; 10×cutsmart buffer 5 ul; Replenish water to 50 ul. Reaction condition: 37° C., 30 min; Inactivated at 65° C. for 20 min. Recovery and purification of gum. The electrophoresis detection of the enzyme-cleaved product was shown in FIG. 69, with lane 1 being pS5E1-IRES-MGF5L6L, EcoRV and BamHI enzyme-cleaved; Lane 2 is the CP129Rubiqutin fragment, EcoRV and BamHI restriction endonucleases, M 15000 bp Marker, 2000 bp Marker.

\textcircled{5} The pS5E1-IRES-MGF 5L6L vector was connected with the CP129Rubiqutin fragment

Ligation system: pS5E1-IRES-MGF 5L6L100N g; CP129Rubiqutin fragment 50 ng; T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

Amplification system: 10 ul of Q5 enzyme, 10 uM primer CP 129R-BAMH-F 1 uL, and 10 uM primer ubiqutin-EcoRV-R 1 ul were replenished to 20 ul; PCR program: 98° C., 10 s; 98° C., 5s, 60° C., 30s, 72° C., 30s, 35 cycles; 72° C., 5 min; Electrophoresis verification was performed as shown in FIG. 70, in which No. 1-5 was a colony and M was DL 2,000 DNA Marker. As seen in FIG. 70, positive bands were present for No. 1 and 2.

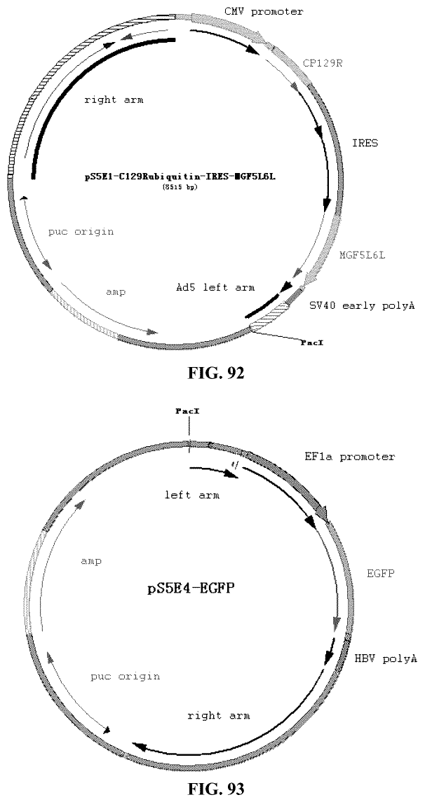

\textcircled{7} Plasmid BamHI and EcoRV restriction enzyme digestion verification: The No. 1 and No. 2 colonies were selected for plasmid extraction and restriction enzyme digestion verification. The results were shown in FIG. 70. The plasmids BamHI and EcoRV restricted to No. 1 and No. 2 colonies in lanes 1 and 2 were identified by restriction enzyme digestion, and M was DL 2,000 DNA Marker. As shown in FIG. 71, the restriction enzyme digestion result was correct, and the African swine fever human adenovirus type 5 vector E1 regional shuttle plasmid pS5E1-CP129Rubiqutin-IRES-MGF5L6L was successfully constructed. The vector map was shown in FIG. 92.

### Example 15 Construction of African Swine Fever Human Adenovirus Type 5 Vector E4 Region Shuttle Plasmid pS5E4-CP312R-2A-MGF5L6L

### 1. Shuttle Plasmid Construction of Human Adenovirus Type 5 Vector E4 Region

The skeleton of the shuttle plasmid pS5E4 was composed of basic elements such as puc origin and amp, the ITR sequence of the left arm (370 bp), the partial fiber gene sequence of the right arm (1746 bp) in the hAd5E4 region, and the EF1α-EGFP-HBV polyA gene.

### 1) Gene Synthesis

The EF1α-EGFP-HBV polyA gene was synthesized by BoMed.

### 2) Primer Design

puc-AdSE4-left arm-F:
(SEQ ID NO: 111)
AGGTGACACTATAGAATACACGTTAATTAAATCATCAATAATA
TACCTTATTTTG
Ad5E4-left arm-EF1α-R:
(SEQ ID NO: 112)
caatccccccttttcttttaaaaAACACCACTCGACACGGCAC
EF1α-F:
(SEQ ID NO: 113)
ttttaaaagaaaaggggggattg
EF1α-R:
(SEQ ID NO: 114)
TAGAGCCCCAGCTGGTTCTTT
EF1α-AdSE4-right arm-F:
(SEQ ID NO: 115)
GGAAAGAACCAGCTGGGGCTCTAGCAATTGAAAAATAAAC
ACGTTGA
Ad5E4-right arm-puc-R:
(SEQ ID NO: 116)
TAATACGACTCACTATAGGGAGACCCAAAATGTAACCACTGTGAG
puc-F:
(SEQ ID NO: 117)
TCTCCCTATAGTGAGTCGTATT
puc-R:
(SEQ ID NO: 118)
CGTGTATTCTATAGTGTCACCT
ORF6/7-Protease-F:
(SEQ ID NO: 119)
CGTTGAAACATAACACAAACGATACGGCGCAGACGGCAAGGGGGG

### 3) Amplification of the Target Fragment

\textcircled{1} Using the synthetic fragment of EF1α-EGFP-HBV gene as the template and EF1α-F and EF1α-R as the primers, the EF1α-EGFP-HBV polyA fragment of the pS5E4-EGFP shuttle plasmid was amplified; Amplification system: the synthetic fragment of EF1α-EGFP-HBV gene was 50 μg, 10 μM EF1α-F primer was 1 ul, 10 μM EF1α-R primer was 1 ul, and Q5 high-fidelity enzyme was 20 ul; Replenish water to 40 ul. PCR procedure: 98° C., 10 sec; 98° C., 5 sec, 60° C., 30 sec, 72° C., 40 sec, 35 cycles; 72° C., 5 min.

\textcircled{2} The left arm fragment of pS5E1 shuttle plasmid was amplified using pAd5LCL3 as the template and puc-Ad5E4-left arm-F and Ad5E4-left arm-EF1α-R as the primers. Amplification system: pAd5LCL3 plasmid 50 ng, 10 uM puc-Ad5E4-left arm-F primer 1 ul, 10 uM Ad5E4-left arm-EF1α-R primer 1 ul, Q5 high-fidelity enzyme 20 ul; Replenish water to 40 ul. PCR program: 98° C., 10 s; 98° C., 5s, 60° C., 30s, 72° C., 10 sec, 35 cycles; 72° C., 5 min.

\textcircled{3} The right arm fragment of the pS5E4-EGFP shuttle plasmid was amplified using pAd5LCL3 as the template and EF1α-Ad5E4-right arm-F and Ad5E4-right arm-puc-R as the primers; Amplification system: pAd5LCL3 plasmid 50 ng, 10 uM EF1α-Ad5E4-right arm-F primer 1 ul, 10 uM Ad5E4-right arm-puc-R primer 1 ul, Q5 high-fidelity enzyme 20 ul; Replenish water to 40 ul.

PCR program: 98° C., 10 s; 98° C., 5s, 60° C., 30s, 72° C., 40 sec, 35 cycles; 72° C., 5 min.

\textcircled{4} QPCR amplification of the pS5E4-EGFP shuttle plasmid backbone using pS5E1 plasmid as the template and puc-F and puc-R as the primers; Amplification system: pS5E1 skeletal plasmid 50 ng, 10 uM puc-F primer 1 ul, 10 uM puc-R primer 1 ul, Q5 high-fidelity enzyme 20 ul; Replenish water to 40 ul. PCR program: 98° C., 10 s; 98° C., 5s, 60° C., 30s, 72° C., 1 min 20 sec, 35 cycles; 72° C., 5 min. Agarose validation of that amplification product was shown in FIG. 72, with lane 1 bee the left arm of the pS5E4-EGFP shuttle plasmid, lane 2 being the right arm of the pS5E4-EGFP shuttle plasmid, lane 3 bee EF1α-EGFP-HBV, lane 4 being the pS5E4-EGFP shuttle plasmid backbone, and M being DL 2,000 DNA Marker. As shown in FIG. 72, the amplification result was correct.

### 4) The Target Fragment was Purified by an Axygen™ Gel Extraction Kit.

### 5) Ligation and Transformation

The four fragments of pS5E4-EGFP shuttle plasmid left arm, pS5E4-EGFP shuttle plasmid right arm, EF1α-EGFP-HBV, and pS5E4-EGFP shuttle plasmid skeleton were connected by use that bode seamless cloning kit. The ligation system consisted of 10 μL of 2×Smealess Cloning Mix, 50 ng of the pS5E4-EGFP shuttle plasmid left arm fragment, 50 ng of the pS5E4-EGFP shuttle plasmid right arm fragment, 50 ng of the EF1α-EGFP-HBV fragment, 50 ng of the pS5E4-EGFP shuttle plasmid backbone fragment, and 20 μl of water replenishment, incubated at 50° C. for 40 minutes. The ligated products were transformed into DH5α competent cells, plated on plates containing ampicillin resistance and incubated at 37° C. for 12-16 hours.

### 6) Plasmid Validation

\textcircled{1} colony PCR

Colony PCR amplification of the target fragment using the primer puc-Ad5E4-left arm-F/EF1α-R as the primer and agarose gel verification revealed a positive band as shown in FIG. 73.

\textcircled{2} enzyme digestion verification

The positive clones No. 3, 4, 5 and 6 were picked and placed in 5 mL LB liquid medium containing ampicillin resistance for culture for 12-15 hours, and the plasmids were extracted for restriction endonuclease verification.

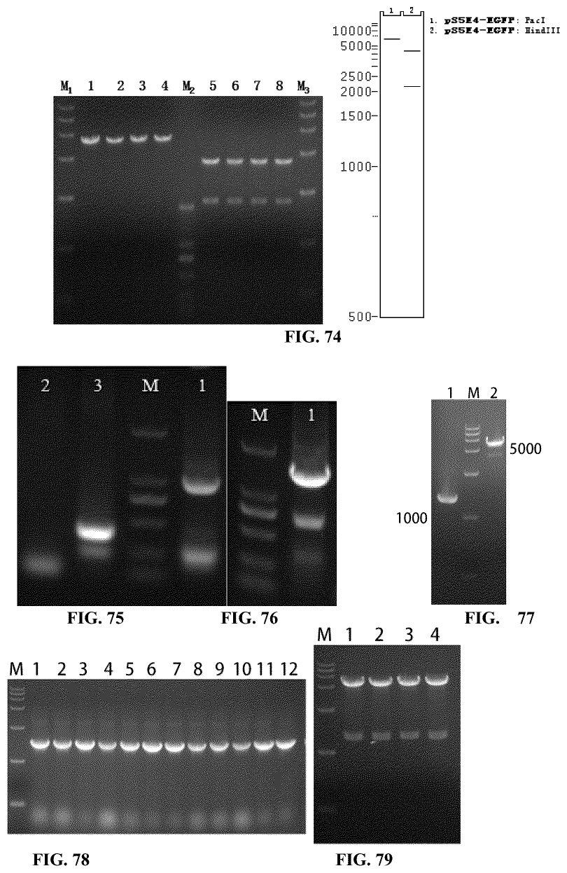

The electrophoresis results were shown in FIG. 74, where 1-4 was the single restriction endonuclease of the positive clones No. 3, 4, 5 and 6 PacI, 5-8 was the single restriction endonuclease of the positive clones No. 3, 4, 5 and 6 HindIII, M₁ and M₃ were DL15,000 bp DNA Marker, M2 was DL 2,000 bp DNA Marker; The results of enzyme digestion were correct and the sequencing was correct. The human adenovirus type 5 vector E4 region shuttle plasmid pS5E4-EGFP was successfully constructed, and the vector map was shown in FIG. 93.

2. Construction of the E4 Region Shuttle Plasmid pS5E4-CP312R-2A-MGF5L6L of African Classical Swine Fever Human Adenovirus Type 5 Vector

### 1) Primer Design

PS5E4-CP312R-HamHI-F:
(SEQ ID NO: 120)
ccaagctgtgaccggcgcctacGGATCCGCCACCATGACAACCCACAT
CP312R-2A-R:
(SEQ ID NO: 121)
GAAGTTAGTAGCTCCGCTTCCGGCGTAATCAGGCACGTCGTA
CP312R-2A-F:
(SEQ ID NO: 122)
TACGACGTGCCTGATTACGCCGGAAGCGGAGCTACTAACTTC
2A-MGF110-4L-R:
(SEQ ID NO: 123)
GCCCAGAAACACCACCAGCATAGGTCCAGGGTTCTCCTCCA
MGF110-4L-F:
(SEQ ID NO: 124)
ATGCTGGTGGTGTTTCTGGG
MGF110-4L-XhoI-R:
(SEQ ID NO: 125)
CGGGTTTAAACGGGCCCTCTAGACTCGAGTCACAGGTCCTTCT
EF1α2 (jd)-F:
(SEQ ID NO: 126)
tggtgcctcctgaactgcgt
HBV (jd)-R:
(SEQ ID NO: 127)
TAAGGGTCAATGTCCATGCC

### 2) Amplification of the Target Fragments CP312R, MGF110-4L, 2A

\textcircled{1} The synthetic fragment of CP312R gene was used as the template and PS5E4-CP312R-HamHI-F and CP312R-2A-R as the primers to amplify the CP312R fragment; Amplification system: synthetic fragment of CP312R gene (50 μg, 10 μM PS5E4-CP312R-Hamhi-F primer (1 ul), 10 μM CP312R-2A-R primer (1 ul), Q5 high-fidelity enzyme (20 ul); Replenish water to 40 ul; PCR procedure: 98° C., 10 sec; 98° C., 5 sec, 60° C., 30 sec, 72° C., 20 sec, 35 cycles; 72° C., 5 min.

\textcircled{2} Using the synthetic fragment of MGF110-4L gene as a template and MGF110-4L-F and MGF110-4L-XhoI-R as primers to amplify the MGF110-4L fragment; Amplification system: MGF110-4L gene synthetic fragment 50 ng, 10 uM MGF110-4L-F primer 1 ul, 10 uM MGF110-4L-XhoI-R primer 1 ul, Q5 high-fidelity enzyme 20 ul; Replenish water to 40 ul; PCR procedure: 98° C., 10 sec; 98° C., 5 sec, 60° C., 30 sec, 72° C., 20 sec, 35 cycles; 72° C., 5 min.

\textcircled{3} The synthetic fragment of 2A gene was used as the template and CP312R-2A-F and 2A-MGF110-4L-R as the primers to amplify the 2A fragment; Amplification system: synthetic fragment of 2A gene (50 ug), 10 μM CP 312R-2A-F primer (1 ul), 10 μM 2A-MGF 110-4 L-R primer (1 ul), and Q5 high-fidelity enzyme (20 ul); Replenish water to 40 ul; PCR procedure: 98° C., 10 sec; 98° C., 5 sec, 60° C., 30 sec, 72° C., 20 sec, 35 cycles; 72° C., 5 min.

The amplification results were shown in FIG. 75, where lane 1 was the CP312R amplification fragment, lane 2 was the 2A amplification fragment, lane 3 was the MGF110-4L amplification fragment, and M was DL 15,000 DNA Marker.

### 3) The Target Fragment was Purified by an Axygen™ Gel Extraction Kit.

4) amplifying the CP312R-2A-MGF110-4L fragment by fusion PCR

Amplification system: 50 ng of CP312R gel recovery fragment, 50 ng of 2A gel recovery fragment, 50 ng of MGF110-4L gel recovery fragment, 1 ul of 10 uM PS5E4-CP312R-HamHI-F primer, 1 ul of 10 uM MGF110-4L-XhoI-R primer, and 25 ul; of Q5 high-fidelity enzyme; Replenish water to 50 ul; PCR procedure: 98° C., 10 sec; 98° C., 5 sec, 60° C., 30 sec, 72° C., 50 sec, 35 cycles; 72° C., 5 min. The fusion results were shown in FIG. 76, where lane 1 was the fragment CP312R-2A-MGF110-4L, and M was DL 15,000 DNA Marker.

5) Enzyme Digestion with pS5E4-EGFP Vector

Enzyme digestion reaction system: vector pS5E4-EGFP 2 ug, BamHI and XhoI 1 uL each; 10×cutsmart buffer 5 ul; Replenish water to 50 ul. Reaction condition: 37° C., 30 min; Inactivated at 65° C. for 20 min. Recovery and purification. The gel recovery results were shown in FIG. 77. In lane 1, the gel was recovered from fragment CP312R-2A-MGF110-4L, and lane 2, the vector of pS5E4-EGFP. The gel was recovered by double enzyme digestion with BamHI and XhoI, and M was DL 15,000 DNA Marker.

6) Seamless Clone Connection and Transformation of pS5E4 Vector and CP312R-2A-MGF110-4L Fragment

Ligation system: recovery products of pS5E4-EGFP vector(100 ng), CP312R-2A-MGF110-4L fragment (50 ng), 2×Smealess Cloning Mix 5 ul, replenished to 10 ul. Reaction condition: 50° C., 40 min. The ligated products were transformed into DH5α competent cells, plated on plates containing ampicillin resistance and incubated at 37° C. for 12-16 hours.

### 7) Plasmid Validation

\textcircled{2} colony PCR

Using EF1α2(d)-F and HBV(jd)-R as primers, the target fragment was amplified by colony PCR and verified by agarose gel assay as shown in FIG. 78, where No. 1-12 was a colony and M was DL 15,000 DNA Marker, all of which were positive bands.

\textcircled{2} enzyme digestion verification

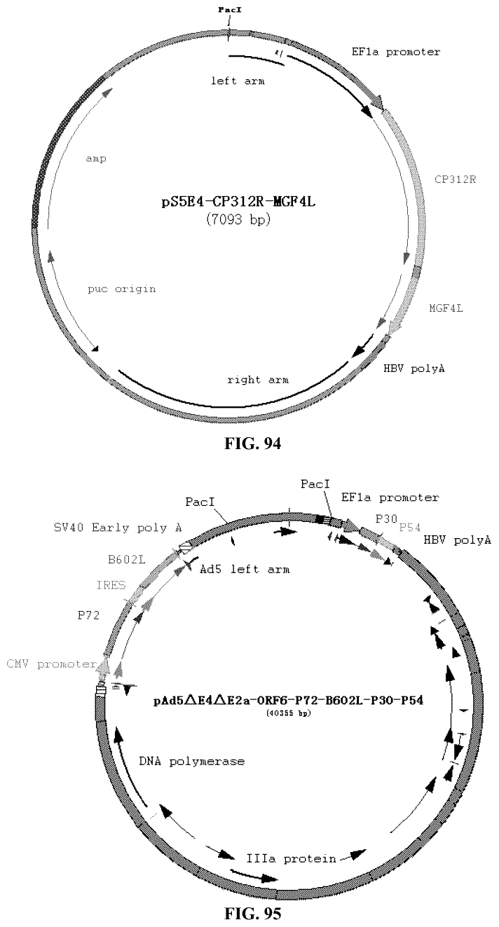

The No. 1, No. 2, No. 3 and No. 4 positive clone were picked and place in 5 mL LB liquid medium containing ampicillin resistance for culture for 12-15 hours, and plasmids were extract for double enzyme digestion verification by BmHI and XhoI; The enzyme digestion results were shown in FIG. 79, where lanes 1, 2, 3 and 4 were the double enzyme digestion verification of positive clones BamHI and XhoI, and M was DL 15,000 DNA Marker. The enzyme digestion result was correct and the sequencing was correct. The African swine fever human adenovirus type 5 vector E4 region shuttle plasmid pSSE4-CP312R-2A-MGF110-4L was successfully constructed, and the vector map was shown in FIG. 94.

### Example 16: construction of plasmid pAd5LCL3-CP129R-ubiqutin-MGF5161-CP312R-MGF110-4L Recombinantly with pAd5LCL3

1. Homologous Recombination of Shuttle Plasmid pSSE1-CP129Rubiqutin-IRES-MGF5L6L and adenovirus vector plasmid pAd5LCL3

1) PacI and SwaI perform enzyme digestion on the shuttle plasmid pSSE1-CP129Rubiqutin-IRES-MGF 5L6L and the adenovirus vector plasmid pAd5LCL3, and the enzyme digestion reaction system was as follows: A. Shuttle plasmid pSSE1-CP129Rubiqutin-IRES-MGF 5L6L3 μg; PacI 2 μl; buffer cutsmart 4 μl; Replenish water to 40 μl.

B, adenovirus vector plasmid pAd5LCL3 3 μg; SwaI 2 μl; Buffer 3.1 4 μl; Replenish water to 40 μl.

Reaction condition were 37° C. for 1 h; Inactivated at 65° C. for 20 min.

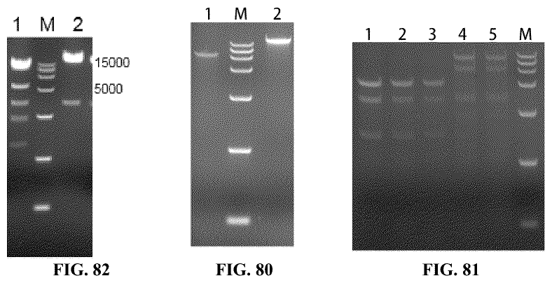

2 ul of agarose gel was used for validation and the results were shown in FIG. 80, with lane 1 being pSSE1-CP129Rubiqutin-IRES-MGF5L6L and lane 2 being pAd5LCL3.

2) Dephosphorylation of that enzyme digestion product

Reaction system: 37.5 μL enzyme digestion reaction solution; 1 μL dephosphorylase; Dephosphorylated buffer 5 μL; Refill water to 50 μL. Reaction condition were 37° C. for 1 h; Inactivated at 65° C. for 5 min.

3) Use OMEGA Ultra-Sep Gel Extraction Kit to recover the vectors and fragments.

4) 100 ng of the purified shuttle plasmid and 100 ng of the purified adenovirus vector were co-transformed into BJ5183 competent cells, and the transformed product was coated with an LB plate containing Kan and cultured at 37° C. for 12-16 h.

5) The colonies were selected and cultured in 5 mL LB liquid medium containing Kan under shaking at 37° C. for 12-16 h, and the plasmids were extracted for XhoI enzyme digestion verification. The results were shown in FIG. 81, where lanes 1-5 were clones of pAd5LCL3-CP 129 Rubiqutin-IRES-MGF 5L6L and M: 15000 bp marker. It can be seen from FIG. 81 that Clones 4 and 5 were correctly digested.

6) The No. 4 positive plasmid was converted to DH5a competent state, one colony was picked out and cultured in 5 mL LB liquid medium containing Kan under shaking at 37° C. for 12-16 h, and the plasmid was extracted for XhoI restriction enzyme digestion verification again. The restriction enzyme digestion results were shown in FIG. 82, where lanes 1 and 2 were subjected to XhoI restriction enzyme digestion of plasmid ppAd5LCL3-CP129Rubiqutin-IRES-MGF5L6L. M was DL 15,000 DNA Marker. As shown in FIG. 82, the enzyme digestion result was correct, and the adenovirus vector plasmid pAd5LCL3-CP129Rubiqutin-IRES-MGF5L6L was successfully constructed.

2. The shuttle plasmid pS5E4-CP312R-2A-MGF110-4L was homologously recombined with the adenovirus vector plasmid pAd5LCL3-C19Rubiqutin-IRES-MGF5L6L to obtain pAd5LCL3-C19Rubiqutin-MGF5L6L-CP312R-MGF110-4L.

1) PacI and I-sceI perform enzyme digestion on the shuttle plasmid pS5E4-CP312R-2A-MGF110-4L and the adenovirus vector plasmid pAd5LCL3-CP129Rubiqutin-IRES-MGF 5L6L, and the enzyme digestion reaction system was as follows:

A. Shuttle plasmid pS5E4-CP312R-2A-MGF110-4L3p g; PacI 2 μl; 10×cutsmart buffer 4 μl; Replenish water to 40 μl.

B. Adenovirus vector plasmid pAd5LCL3-CP129rubiqutin-IRES-MGF5L6L3 ug; I-sceI 2 μl; Buffer cutsmart 4 μl; Replenish water to 40 μl.

Reaction condition were 37° C. for 1 h; Inactivated at 65° C. for 20 min.

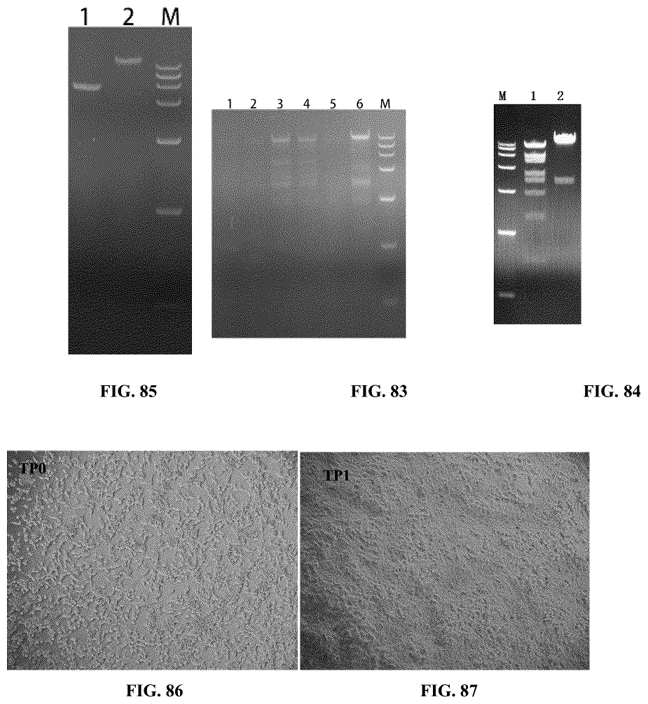

2 ul of agarose gel was used for validation and the results were shown in FIG. 83, with lane 1 being pS5E4-CP312R-2A-MGF110-4L and lane 2 being pAd5LCL3-CP129Rubiqutin-IRES-MGF5L6L.

2) Dephosphorylation of that enzyme digestion product

Reaction system: 37.5 μL enzyme digestion reaction solution; 1 μL dephosphorylase; Dephosphorylated buffer 5 μL; Refill water to 50 μL. Reaction condition were 37° C. for 1 h; Inactivated at 65° C. for 5 min.

3) Use OMEGA Ultra-Sep Gel Extraction Kit to recover the vectors and fragments.

4) 100 ng of the purified shuttle plasmid and 100 ng of the purified adenovirus vector were co-transformed into BJS183 competent cells, and the transformed product was coated with an LB plate containing Kan and cultured at 37° C. for 12-16 h.

5) Six colonies were selected and cultured in 5 mL LB liquid medium containing Kan under shaking at 37° C. for 12-16 h, and the plasmids were extracted for XhoI enzyme digestion verification. The results were shown in FIG. 84, where lanes 1-6 were plasmids, and M was DL 15,000 DNA Marker. It can be seen that plasmids No. 3 and 4 were correct.

6) transform that No. 3 positive plasmid into DH5a competence; One colony was selected and cultured in 5 mL LB liquid medium containing Kan under shaking at 37° C. for 12-16 h, and the plasmid was extracted for XhoI restriction enzyme digestion verification again. The enzyme digestion results were shown in FIG. 85, where lane 1 was an XhoI enzyme digestion of the plasmid pAd5LCL3-CP129Rubiqutin-MGF5L6L-CP312R-MGF110-4L, lane 2 was an PacI enzyme digestion of the plasmid pAd5LCL3-CP129Rubiqutin-MGF5L6L-CP312R-MGF110-4L, and M was DL 15,000 DNA Marker. As shown in FIG. 85, the enzyme digestion result was correct, and the adenovirus vector plasmid pAd5LCL3-CP129Rubiqutin-MGF5L6L-CP312R-MGF110-4L was successfully constructed. The vector map was shown in FIG. 95.

### Example 17: Packaging of Recombinant Adenovirus

Wrap the plasmid pAd5LCL3-CP129Rubiqutin-MGF 5L6L-CP312R-MGF 110-4L with 293TD37 cells as follows:

Preparation of 293TD37 cells: The cells were prepared one day before transfection. The 293TD37 cells to be transfected were inoculated into a 6-well plate at 0.5×10⁶/well, and incubated at 37° C. with 5% CO₂ for 24 hours. On the day of transfection, the cells showed 40-50% confluency.

Linearization of plasmid pAd5LCL3-C129Rubiqutin-MGF5L6L-CP312R-MGF110-4L: The plasmid to be transfected was digested with PacI enzyme, incubated at 37° C. for 40 min, and inactivated at 65° C. for 20 min.

Transfection: The linearized 2 pg plasmid and PEI were diluted with 100 μl serum-free medium, respectively. The plasmid diluent was added into the PEI diluent, and repeatedly aspirated for 5 times or vortexed for 10 seconds to be mixed evenly, and incubated for 10 minutes at room temperature to form a transfection complex. During incubation, cell culture medium was gently aspirated from the plates, 2 mL of fresh growth medium was added, and after 10 minutes the transfection complex was added to the cells in fresh medium.

Cell culture: the transfected 293TD37 cells were incubated at 37° C. for 72-96 hours in a 5% CO₂ incubator; 6-well plate cell suspensions were collected in 1.5 ml centrifuge tubes, TP0, 72-96 hours after viral plasmid transfection.

Continuous inoculation: The collected cell suspension was repeatedly frozen and thawed at −80° C. for 3 times, centrifuged at 2000 g for 10 minutes at 4 C, and 500 μl of supernatant was collected to infect 293TD37 cells (293TD37 cells need to be prepared one day in advance). The cells were incubated at 37° C. with 5% CO₂ for 60 minutes, followed by the addition of 2 mL of FBS medium, followed by culture at 37° C. with 5% CO₂ for 72 hours, and the cell suspension, namely TP1; was collected. The previous steps were repeated and the cell suspension, TP2, was collected. The drug was continued until the cells became diseased.

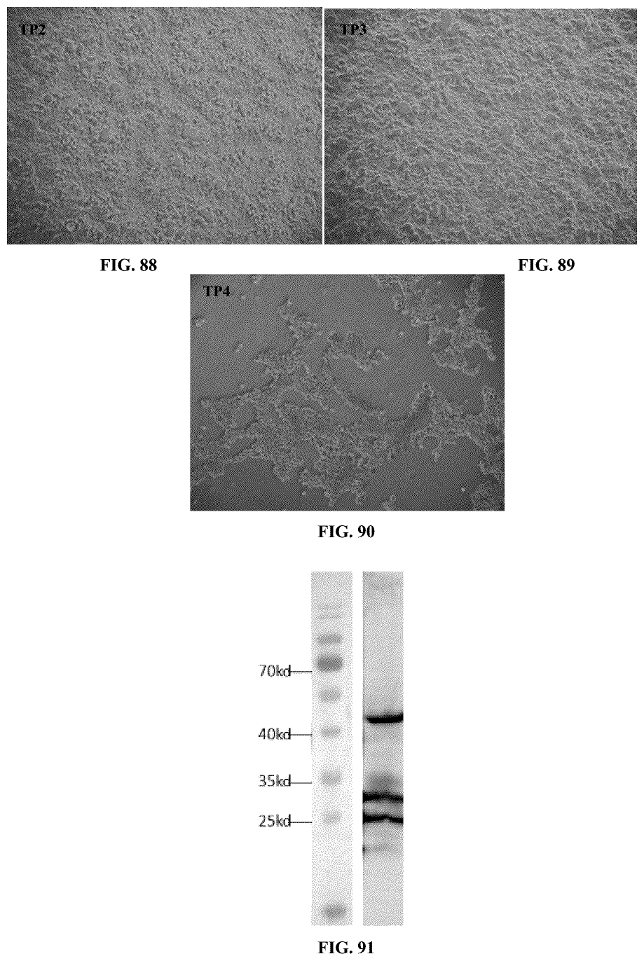

Cytopathic effect: When the 293TD37 cells were cultured from TP0 to TP4, the cells gradually became diseased until the 293TD37 cells became completely diseased. The cytopathic effects caused by TP0 to TP4 were shown in FIG. 86-90, respectively. TP4 was completely diseased.

### Example 18: Detection of Titer of African Swine Fever Multiantigen Recombinant Adenovirus Vaccine

The 293TD37 cells were prepared. The cells with good growth in T75 culture flask were taken, the supernatant was discarded, the cells were washed with PBS, and digested with 0.25% trypsin, and then 10 mL of fresh DMEM medium containing 10% fetal bovine serum was added to stop the digestion, which was then blown, mixed, inoculated into 6-well plates (5×10⁵/mL, 2 ml per well), and allowed to stand for culture in a 37° C. 5% CO₂ carbon dioxide incubator. After 24 hours, when the cells adhered to grow into single-layer cells, the culture medium was discarded, and the recombinant adenovirus was continuously diluted 10-3 to 10-6 times with serum-free DMEM maintenance solution, and two wells were inoculated with each dilution degree at 250 uL per well. After one hour of infection, the supernatant was discarded to supplement the complete culture medium, and then the medium was allowed to stand for culture in a 5% carbon dioxide incubator at 37° C. After 24 h, the supernatant was discarded and the cells were washed with PBS (1 mL per well). After PBS was discarded, 1 mL of cold formaldehyde was added into each well for fixation, and formaldehyde was discarded at room temperature for 10 min. Then the cells were washed with PBS (1 mL per well), followed by adenovirus antibody-FITC (1 ml per well). After 1 h at room temperature, the cells were washed with PBS again (1 mL per well). After two times, 1 mL of PBS was added into each well and counted under fluorescence microscope (200 times, 10 continuous fields). Calculation: Virus titer (FFU/mL)=Mean×1013×4×10(−n). The FFU of the pAd5LCL3-CP129Rubiqutin-MGF5L6L-CP312R-MGF110-4L virus was 1.9×10⁸FFU/mL, with a high titer.

### Example 19: Detection of Stability of African Swine Fever Multiantigen Recombinant Adenovirus

Vaccine pAd5LCL3-CP129Rubiqutin-MGF5L6L-CP312R-MGF110-4L

The 293TD37 cells were prepared. The cells that grew well in T75 culture flask were taken, the supernatant was discarded, the cells were washed with PBS, and digested with 0.25% trypsin. Then 10 mL of fresh DMEM medium containing 10% fetal bovine serum was added to stop the digestion, and then the cells were blown and mixed evenly. The 293TD37 cells were planted into 6-well plates (5×10⁵ cells/mL, 2 mL/well), incubated for 1 hour at room temperature to adhere to the wall, and incubated for microscopic observation of its attachment degree. The infection was carried out with pAd5LCL3-CP129Rubiqutin-MGF5L6L-CP312R-MGF110-4L viral particles and the titer of infection was 5 MOI/well. After the 293TD37 cells developed lesions 48 hours later, the cells were collected, repeatedly frozen and thawed for three times, and then centrifuged at 2000 g; the collected supernatant was detected for FFU, and then new 293TD37 cells were reinfected until the 30th generation. The collected virus solutions of passages 5, 10, 15, 20, 25 and 30 were tested, and the genome of the virus was found to be still intact, indicating that the replication-defective pAd5LCL3-CP129Rubiqutin-MGF5L6L-CP312R-MGF110-4L virus could be stably packaged in 293TD37 cells.

### Example 20: Detection of African Swine Fever Multiantigen Recombinant Adenovirus Vaccine pAd5LCL3-CP129Rubiqutin-MGF5L6L-CP312R-MGF110-4L Recovery Mutation (RCA)

pAd5LCL3-CP129Rubiqutin-MGF5L6L-CP312R-MGF110-4L virus RCA detection, detection method was as follows:

1. Prepare pAd5LCL3-CP129 Rubiqutin-MGF5L6L-CP312R-MGF110-4L virus solution, and measure the virus titer and determine the concentration of virus particles. The DNA of the host cell shall be digested with 1% Universal Nuclease (7.5-15 units/mL virus solution) in the virus solution and water bath at 37° C. for 40 min. Using a 300Kd ultrafiltration centrifuge tube, the virus particles were collected after centrifugation at 1000 g for 30 min, followed by elution with 1×PBS. A260 was measured, and the particle concentration=A260*1.1*10 12 VP/mL.

2. Virus infection: A 6-well plate of A549 cells was prepared, with each well cell being 2.5×10⁵/well. The medium was discarded and washed with PBS once. Adenovirus was inoculated with virus at 1×10⁹ vp/well to infect A549 cells. Wild-type human adenovirus type 5 was used as the control at 37° C. and 5% CO₂. After 1 h, the virus solution was discarded and supplemented with 5% complete medium. The cells were cultured at 37° C. and 5% CO₂ for 48 h.

3. Immunostaining was performed, and the cell supernatant was discarded. The cells were surface washing cells in PBS, fixed with ice formaldehyde, placed at −20 C for 20 min, and washed with 1×PBS for three times, each time for 5 min. Then 2 ml 1% BSA-PBS solution was added into each well, placed in a shaker, and incubated for 1 h. After the supernatant was discarded, human adenovirus type 5 fluorescent antibody (1:500 dilution) was added and incubated for 1 h, followed by washing with 1×PBS for three times, 5 min each time.

RCA was calculated using the equation as observed under a 10-fold fluorescence microscope

$$RCA = ( average positive cell field ) \times ( 374 field / well ) \times ( dilution factor ) ) / Total VPs in 0.5 ml viral sample$$

The judging standard was that the level of RCA was less than 1RCA/3×10¹⁰ vp. According to statistics, the RCA level of the pAd5LCL3-CP129Rubiqutin-MGF5L6L-CP312R-MGF110-4L was less than 1RCA/3×10¹⁰ vp, which indicates that the replication-defective pAd5LCL3-CP129Rubiqutin-MGF5L6L-CP312R-MGF110-4L virus prepared by the invention can be stably packaged in 293TD37 cells and has low probability of not being converted into wild type or wild type.

### Example 21: Detection of Protein Expression of African Swine Fever Multiantigen Recombinant Adenovirus Vaccine pAd5LCL3-CP129 Rubiqutin-MGF5L6L-CP312R-MGF110-4L

The 293TD37 cells were prepared one day in advance and placed in a 12-well cell culture plate. The 293TD37 cells were infected with the African swine fever multiantigen recombinant adenovirus vaccine pAd5LCL3-CP129Rubiqutin-MGF5L6L-CP312R-MGF110-4L virus. After 48 hours, the cells became diseased, and 1 ml of cells were collected. The cells were washed with PBS, and samples were prepared for Western Blot detection. The antibody of HA was used to detect the target protein, and the antibody of HA was purchased from Abcam. Wherein, CP129Rubiquitin, MGF5L6L and CP312R have HA tags, wherein the size of the CP129Rubiquitin fusion protein was 34kda, the size of the MGF5L6L protein was 25kda, and the size of the CP312R protein was 35kda. The experimental results were shown in FIG. 91. MGF5L6L protein can be clearly seen in this vaccine, and thus the protein expression level of pAd5LCL3-CP129Rubiqutin-MGF5L6L-CP312R-MGF110-4L was high.

### Example 22: Immunological Evaluation of African Swine Fever Multiantigen Recombinant Adenovirus Vaccine pAd5LCL3-CP129Rubiqutin-MGF5L6L-CP312R-MGF110-4L on Mouse Model

### 22.1 Cell Immune Response Detection

Ten SPF-grade mice (6-8 weeks of age) were randomly divided into two groups, five for each group. Mice were immunized with pAd5LCL3-CP129Rubiqutin-MGF5L6L-CP312R-MGF110-4L according to the groupings shown in Table 5. The injection method was as follows: intramuscular injection was performed on the medial aspect of the posterior thigh. Injection dose: 100u1.

TABLE 5
Groups of Vaccinated Mice

| | | | Immune | Number |
|---|---|---|---|---|
| group | Vector vaccine | dosage | mode | of mice |
| Experiment | pAd5LCL3- | 1*10\hat{ }7 FFU | intramuscular | 5 |
| | CP129Rubiqutin- | | injection | |
| | MGF5L6L-CP312R- | | | |
| | MGF110-4L | | | |
| Control | pAd5LCL3 | 1*10\hat{ }7 FFU | intramuscular | 5 |
| | | | injection | |

The mice were sacrificed 14 days after immunization, and the splenic lymphocytes were isolated. PK15 cells transfected with the shuttle plasmids pS5E1-CP129Rubiqutin-MGF5L6L and pS5E4-CP312R-MGF110-4L were stimulated and cultured for 6 hours, while protein secretion blockers were added to block cytokine secretion. After 6 hours, Fc receptors were blocked, dead cells and cell surface molecular markers were stained, and intracellular cytokines were stained after the cells were fixed and perforated. Cell surface markers included CD4 and CD8, and intracellular cytokines included IFNγ and IL2. The expression levels of IFNγ and IL2 in CD4+T cells and CD8+T cells stimulated by the target protein were analyzed by flow cytometry (CyExpert).

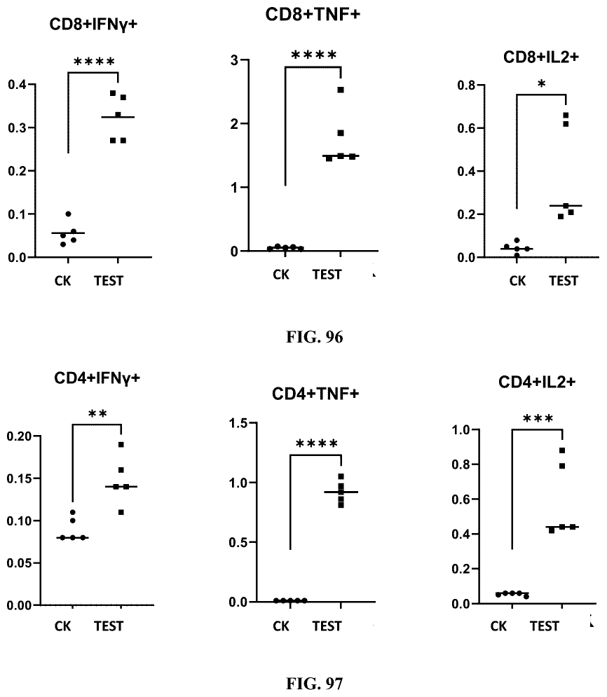

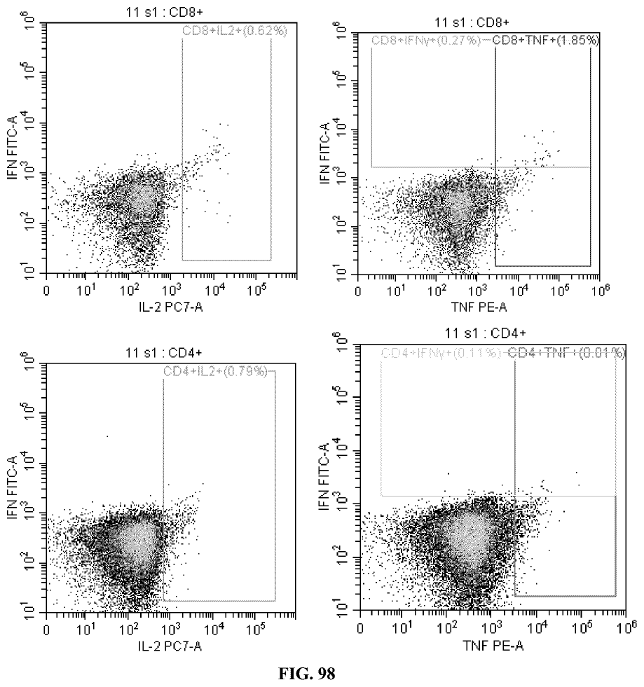

The immune responses of CD8+T cells and CD4+T cells induced by pAd5LCL3-CP129Rubiqutin-MGF5L6L-CP312R-MGF110-4Lwere shown in FIG. 96 and FIG. 97, and the representative results were shown in FIGS. 98 to 99. FIG. 98 was the representative cellular immune response after intramuscular injection of pAd5LCL3-CP129Rubiqutin-MGF5L6L-CP312R-MGF110-4L, and FIG. 99 is the representative blank control immune response. The results showed that, 14 days after immunization, after the spleen cells were stimulated by the target protein, the levels of IFNγ, TNFα and IL2 expressed in CD8+T cells were significantly higher than those in the HAd5 vector control group (Control)(P<0 0.05). After stimulation, the expression levels of IFNγ, TNFα and IL2 in CD4+T cells were significantly higher than those in the HAd5 vector control group (P<0.05). The results of cellular immune response showed that intramuscular injection of the adenovirus vector vaccine of 1*10⁷ FFU could induce specific cellular immune response in the immunized mice.

### Example 23: Construction of the African Swine Fever Human Adenovirus Type 5 Vector E1 Region Shuttle Plasmid pS5E1-L8Lubiqutin-IRES-I215L

1. Construction of shuttle plasmid in E1 region of human adenovirus type 5 vector. The construction was conducted in the same way as in Example 4 to obtain shuttle plasmid pS5E1.

2. Construction of African swine fever human adenovirus type 5 vector shuttle plasmid pS5E1-L8Lubiqutin-IRES-I215L

1) Ligation of pS5E1 to the IRES fragment

The ligation of pS5E1 and IRES fragments was performed according to the method provided in Example 4 to construct pS5E1-IRES.

2) Ligation of pS5E1-IRES to the I215L fragment

\textcircled{1} primer synthesis

I215L-NotI-F:
(SEQ ID NO: 128)
aaggaaaaaaGCGGCCGCgccaccATGGTGAGCAGGTTTCTGATC
I215L-XhoI-R:
(SEQ ID NO: 129)
catgCTCGAGTCAGGCGTAATCGGGCACAT

\textcircled{2} PCR amplification of I215L fragment

Amplification system: 25 ul of Q5 enzyme, I215L-NotI-F 1 ul of 10 μM primer, I215L-XhoI-R 1 ul of 10 μM primer, and I215L 1 ul of template, and water supplementing to 50 ul. PCR program: 98° C., 10 s; 98° C., 5s, 60° C., 30s, 72° C., 40s, 35 cycles; 72° C., 5 min.

\textcircled{3} The I215L fragment was purified by Axygen™ PCR purification kit.

\textcircled{4} The target fragment I215L was digested with pS5E1-IRES vector

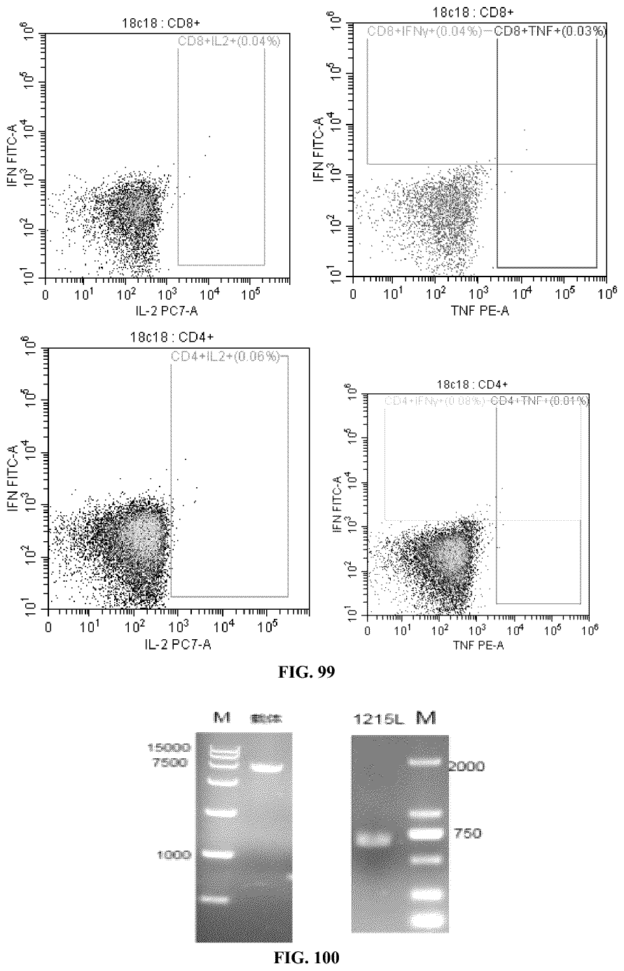

Enzyme digestion reaction system: vectors pS5E1-IRES, I215L fragment −2 ug, NotI and XhoI 1 uL each; 10×cutsmart buffer 5 ul; Replenish water to 50 ul. Reaction condition: 37° C., 30 min; Inactivated at 65° C. for 20 min. Recovery and purification of gum. The electrophoresis detection of the enzyme digestion product was shown in FIG. 100, where the lane vector is pS5E1-IRES NotI and XhoI double-digestion, and lane I215L was the fragment I215L NotI and XhoI double-digestion, and M was DL 15,000 DNA Markeror DL 2,000 DNA Marker.

\textcircled{5} The target fragment I215L was connected with pS5E1-IRES

Ligation system: PS5E1-IRES (100 ng); An I215L fragment (vector:fragment=1: 3 molar ratio); T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

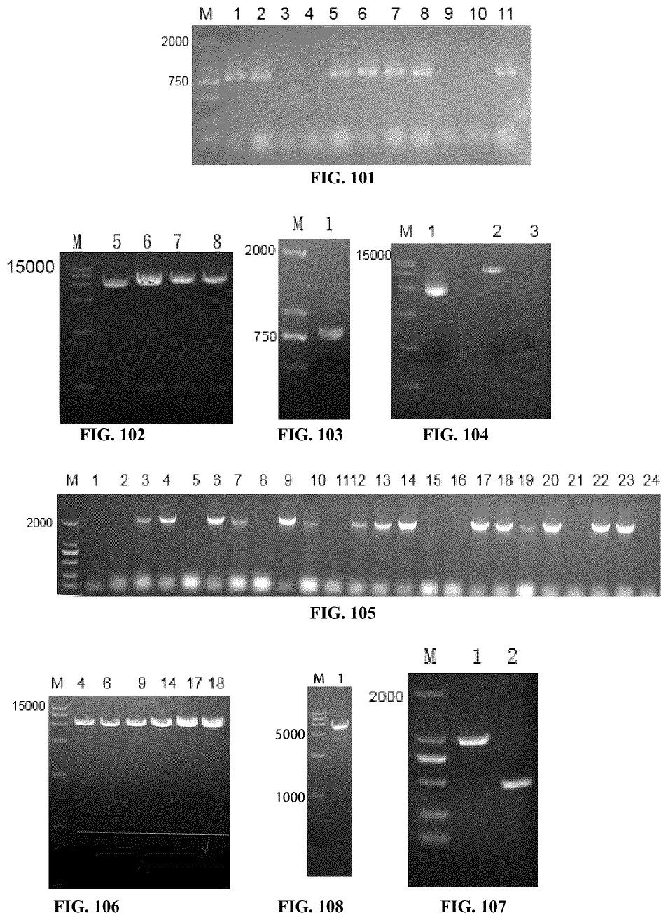

Amplification system: 10 ul of Q5 enzyme, 10 uM universal primer CMV-F 1 ul, and 10 uM primer I215L-XhoI-R 1 ul, and supplementing water to 20 ul; PCR program: 98° C., 10 s; 98° C., 5s, 60° C., 30s, 72° C., 30s, 35 cycles; 72° C., 5 min. Electrophoresis verification was performed as shown in FIG. 101, in which No. 1-11 was a colony and M was DL 2,000 DNA Marker. As shown in FIG. 101, colonies 1, 2, 5, 6, 7, 8, and 11 were positive.

\textcircled{7} Plasmid restriction endonuclease assay (NotI and XhoI), colonies 5, 6, 7 and 8 were selected for plasmid extraction and restriction endonuclease assay. The results were as shown in FIG. 102, all of which were positive plasmids.

3) Ligation of pS5E1-IRES-MGF5L6L to fragment L8Lubiqutin

\textcircled{1} primer synthesis

L8L-BamHI-F:
(SEQ ID NO: 130)
cgcGATCCgccaccATGGGCAACAGACTGATCAAG
L8L-ubiqutin-R:
(SEQ ID NO: 131)
AAGGGTTTTCACGAAAATCTGCATGGCGTAGTCGGGCACGTCGT
ubiqutin-F:
(SEQ ID NO: 132)
ATGCAGATTTTCGTGAAAACCC
ubiqutin-EcoRV-R:
(SEQ ID NO: 133)
ccgGATATCTTACTTGTCTTCTGGTTTGTTGA

\textcircled{2} PCR amplification of L8Lubiqutin fragment

### Amplified L8L Fragment:

Amplification system: 25 ul of Q5 enzyme, 1 uL of primer L8L-BAMHI-F, 1 uL of primer L8L-ubiquitin-R, and 2 uL of template L8L; water supplementation was conducted to 50 ul; Reaction condition: 98° C. for 30 s; 98° C. for 10 s, 68° C. for 30 s, 72° C. for 15 s, 35 cycles; 72° C., 5 min.

### Amplified Ubiqutin Fragment:

Amplification system: 25 ul of Q5 enzyme, 1 uL of primer UBIQUTIN-F, 1 uL of primer ubiqutin—EcoRV-R, and 2 uL of template UBIQUTIN, and supplementing water to 50 ul; Reaction condition: 98° C. for 30 s; 98° C. for 10 s, 68° C. for 30 s, 72° C. for 15 s, 35 cycles; 72° C. 5 min.

The L8L and ubiqutin fragments were purified by an Axygen™ gel extraction and purification kit.

### Amplification of L8Lubiqutin Fragment by Fusion PCR

Amplification system: 25 ul of Q5 enzyme, 50 ng of upstream primer L8L-BamHI-F, downstream primer ubiqutin-EcoRV-R template fragment L8L and fragment ubiqutin respectively, and water supplementing to 50 ul; Reaction condition: 98° C.; 98° C. for 5 s, 68° C. for 30 s, 72° C. for 30 s, 35 cycles; 72° C. 7 min.

\textcircled{3} The L8Lubiqutin fragment was purified by Axygen™ PCR purification kit, and the fused L8Lubiqutin fragment was shown in FIG. 103.

\textcircled{4} The target fragment L8Lubiqutin was digested with pS5E1-IRES-I215L vector

Enzyme digestion reaction system: the vectors were pS5E1-IRES-I215L, L8Lubiqutin fragment −2 ug, and each of EcoRV and BamHI was 1 uL; l0 xcutsmart buffer 5 ul; Replenish water to 50 ul. Reaction condition: 37° C., 30 min; Inactivated at 65° C. for 20 min. Recovery and purification of gum. The electrophoresis detection of the enzyme digestion product was shown in FIG. 104, wherein lane 1 is the pS5E1-IRES-I215L plasmid; Lane 2 was the pS7E1-IRES-I215L plasmid EcoRV and BamHI restriction enzyme digestion; Lane 3 was the EcoRV and BamHI restriction endonucleases of the L8Lubiqutin fragment, and M was DL 15,000 DNA Marker.

\textcircled{5} pS5E1-IRES-I215L vector was connected with L8Lubiqutin fragment

Ligation system: pS5E1-IRES-I215L 100 ng; L8Lubiqutin fragment 50 ng; T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

Amplification system: 10 ul of Q5 enzyme, 10 uM universal primer CMV-F 1 ul, and 10 uM primer ubiqutin-EcoRV-R 1 ul were replenished to 20 ul; PCR program: 98° C., 10 s; 98° C., 5s, 60° C., 30s, 72° C., 40s, 35 cycles; 72° C., 5 min; Electrophoresis verification was performed as shown in FIG. 105, in which No. 1-24 was a colony and M was DL 2,000 DNA Marker.

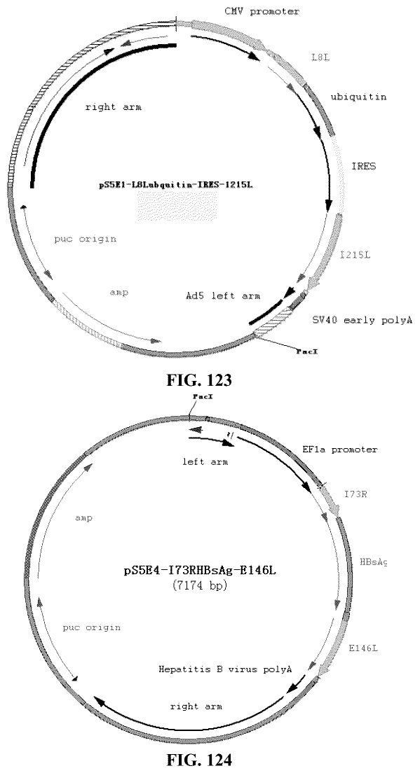

\textcircled{7} Plasmid digestion verification of BamHI and EcoRV: The colonies of 4, 6, 9, 14, 17 and 18 were selected for plasmid extraction and digestion verification. The results were shown in FIG. 106. The enzyme digestion identification of BamHI and EcoRV shows that M was DL 15,000 DNA Marker. As shown in FIG. 106, the enzyme digestion result was correct, and the African swine fever human adenovirus type 5 vector E1 region shuttle plasmid pS5E1-L8Lubiqutin-IRES-I215L was successfully constructed. The vector map is shown in FIG. 123.

### Example 24: Construction of the African Swine Fever Human Adenovirus Type 5 Vector E4 Region Shuttle Plasmid pS5E4-I73RHBsAg-2A-E146L

1, human adenovirus type 5 vector E4 region shuttle plasmid construction

The human adenovirus type 5 vector E4 region shuttle plasmid pS5E4-EGFP was successfully constructed according to the method provided in Example 5.

2. Construction of African swine fever human adenovirus type 5 vector E4 region shuttle plasmid pS5E4-I73RHBsAg-2A-E146L

1) Primer design

pS5E4-173R-BamHI-F:
(SEQ ID NO: 134)
ccaagctgtgaccggcgcctacGGATCCgccaccATGGAGACA
CAGAAG
I73R-HBsAg-R:
(SEQ ID NO: 135)
AGCCGCTGGTGGTGTTCTCCATGGCGTAGTCAGGCACATCGTA
HBsAg-F:
(SEQ ID NO: 136)
ATGGAGAACACCACCAGCGGC
HBsAg-2A-R:
(SEQ ID NO: 137)
TGAAGTTAGTAGCTCCGCTTCCGATGTACACCCAGAGGCAGAA
2A-F:
(SEQ ID NO: 138)
GGAAGCGGAGCTACTAACTTC
2A-E146L-R:
(SEQ ID NO: 139)
ACAAAGTCTGTTGTTCCGCCCATAGGTCCAGGGTTCTCCTCCA
E146L-F:
(SEQ ID NO: 140)
ATGGGCGGAACAACAGACTTT
E146L-pS5E4-XhoI-R:
(SEQ ID NO: 141)
CGGGTTTAAACGGGCCCTCTAGACTCGAGTTAGATGATTCTCTGC

2) Amplification of target fragments I73R, HBsAg, 2A, E146L

\textcircled{1} The I73R fragment was amplified using the I73R gene synthetic fragment as the template and pS5E4-I73R-BamHI-F and I73-HBsAg-R as the primers; Amplification system: synthetic fragment of 173 gene (50 μg, 10 μMpS5E4-173R-BamHI-F primer (1 ul), 10 μM I73-HBsAg-R primer (1 ul), Q5 high-fidelity enzyme (20 ul); Replenish water to 40 ul; PCR procedure: 98° C., 10 sec; 98° C., 5 sec, 60° C., 30 sec, 72° C., 20 sec, 35 cycles; 72° C., 5 min.

\textcircled{2} Using the HBsAg gene synthetic fragment as a template and HBsAg-F and HBsAg-2A-R as primers, amplifying the HBsAg fragment; Amplification system: HBsAg gene synthetic fragment 50 ng, 10 uM HBsAg-F primer 1 ul, 10 uM HBsAg-2A-R primer 1 ul, Q5 high-fidelity enzyme 20 ul; Replenish water to 40 ul; PCR procedure: 98° C., 10 sec; 98° C., 5 sec, 60° C., 30 sec, 72° C., 20 sec, 35 cycles; 72° C., 5 min.

\textcircled{3} Using the synthetic fragment of 2A gene as a template and 2A-F and 2A-E146L-R as primers, the 2A fragment was amplified; Amplification system: synthetic fragment of 2A gene (50 μg), 10 μM 2A-F primer (1 ul), 10 μM 2A-E146L-R primer (1 ul), and Q5 high-fidelity enzyme (20 ul); Replenish water to 40 ul; PCR procedure: 98° C., 10 sec; 98° C., 5 sec, 60° C., 30 sec, 72° C., 20 sec, 35 cycles; 72° C., 5 min.

\textcircled{4} Using the synthetic fragment of E146L gene as the template and E146L-F and E146L-pS5E4-XhoI-R as the primers, the E146L fragment was amplified. Amplification system: synthetic fragment of E146L gene (50 μg), 10 μM E146L-F primer (1 ul), 10 μM E146L-pSSE4-XhoI-R primer (1 ul), and high-fidelity enzyme (Q5) (20 ul). Replenish water to 40 ul; PCR procedure: 98° C., 10 sec; 98° C., 5 sec, 60° C., 30 sec, 72° C., 30 sec, 35 cycles; 72° C., 5 min.

The fragments of interest were purified by an Axygen™ gel extraction kit.

4) Amplifying the I73RHBsAg fragment by fusion PCR Amplification system: 50 μg of recovered I73R gel fragment, 50 μg of recovered HBsAg gel fragment, 1 ul of 10 μM pS5E4-I73R-Hamhi-F primer, 1 ul of 10 μM HBsAg-2A-R primer, and 25 ul; of Q5 high-fidelity enzyme; Replenish water to 50 ul; PCR procedure: 98° C., 10 sec; 98° C., 5 sec, 60° C., 30 sec, 72° C., 20 sec, 35 cycles; 72° C., 5 min.

5) Amplifying the 2A-E146L fragment by fusion PCR

Amplification system: 50 ng of recovered 2A gel fragment and 50 ng of recovered E146L gel fragment, 1 ul of 10 uM 2A-F primer, 1 ul of 10 uM E146L-pSSE4-XhoI-R primer, and 25 ul; of Q5 high-fidelity enzyme; Replenish water to 50 ul; PCR procedure: 98° C., 10 sec; 98° C., 5 sec, 60° C., 30 sec, 72° C., 30 sec, 35 cycles; 72° C., 5 min. The fusion results were shown in FIG. 107, with lane 1 being the I73RHBsAg fragment, lane 2 being the 2A-E146L fragment, and M being the 2000 bp Marker.

6) Enzyme digestion with pSSE4-EGFP vector

Enzyme digestion reaction system: vector pS5E4-EGFP 2 ug, BamHI and XhoI 1 uL each; 10×cutsmart buffer 5 ul; Replenish water to 50 ul. Reaction condition: 37° C., 30 min; Inactivated at 65° C. for 20 min. Recovery and purification.

7) Purifying the vector fragment by an Axygen™ gel extraction kit;

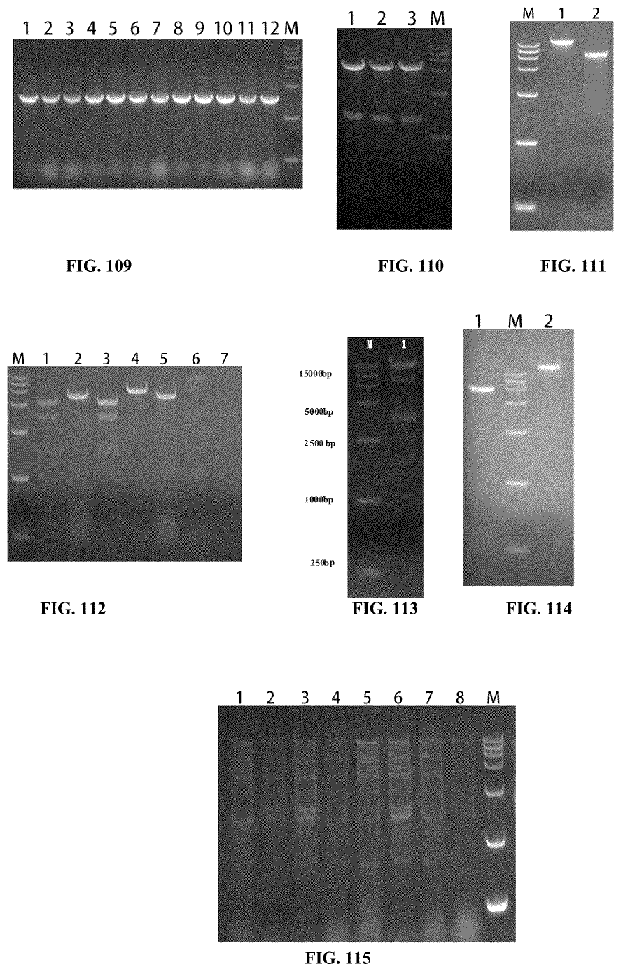

The recovery products were shown in FIG. 108, where lane 1 was the double enzyme digestion recovery of fragments pS5E4-EGFPBamHI and XhoI, and M was DL 15,000 DNA Marker.

8) recovery products of pS5E4-EGFP vector and I73RHBsAg fragment, 2A-E146L seamless clone connection and transformation

Ligation system: pS5E4-EGFP gel recovery (100 ng), I73RHBsAg fragment (50 ng), 2A-E146L fragment (50 ng), 2×Smealess Cloning Mix 5 ul, replenished to 10 ul. Reaction condition: 50° C., 40 min. The ligated products were transformed into DH5α competent cells, plated on plates containing ampicillin resistance and incubated at 37° C. for 12-16 hours.

9) Plasmid validation

\textcircled{1} colony PCR

Using EF1α2(d)-F and HBV(jd)-R as primers, the target fragment was amplified by colony PCR and verified by agarose gel assay as shown in FIG. 109, where No. 1-12 was a colony and M was DL 15,000 DNA Marker, all of which were positive bands.

\textcircled{2} enzyme digestion verification

The No. 1, No. 2 and No. 3 positive clone were picked and place in 5 mL LB liquid medium containing ampicillin resistance for culture for 12-15 hours, and that plasmids were extract for double enzyme digestion verification of BamHI and XhoI; The enzyme digestion results were shown in FIG. 110, where lanes 1, 2 and 3 were for the double enzyme digestion verification of the positive clones BamHI and XhoI, and M was DL 15,000 DNA Marker. The enzyme digestion result was correct and the sequencing was correct. The African swine fever human adenovirus type 5 vector E4 region shuttle plasmid pS5E4-I73RHBsAg-2A-E146L was successfully constructed, and the vector map was shown in FIG. 124.

### Example 25: pAd5LCL3-L8Lubiqutin-IRES-I215L, pS5E4-I73RHBsAg-2A-E146L Reconstructed with pAd5LCL3 Plasmid pAd5LCL3-L8Lubiqutin-I215L-I73RHBsAg-E146L

1. Autologous recombination of shuttle plasmid pS5E1-L8Lubiqutin-IRES-I215L and adenovirus vector plasmid pAd5LCL3

1) PacI and SwaI perform enzyme digestion on the shuttle plasmid pS5E1-L8Lubiqutin-IRES-I215L and the adenovirus vector plasmid pAd5LCL3, and the enzyme digestion reaction system was as follows:

A. Shuttle plasmid pS5E1-L8Lubiqutin-IRES-I215L3 μg; PacI 2 ul; buffer cutsmart 4 ul; Replenish water to 40 ul.

B, adenovirus vector plasmid pAd5LCL3 3 ug; SwaI 2 ul; Buffer 3.1 4 ul; Replenish water to 40 ul.

Reaction condition were 37° C. for 1 h; Inactivated at 65° C. for 20 min.

Two uL of agarose gel were used for validation, and the results were shown in FIG. 111, where lane 1 was pAd5LCL3 and lane 2 is pS5E1-L8Lubiqutin-IRES-I215L.

2) Dephosphorylation of that enzyme digestion product

Reaction system: 37.5 ul; of enzyme digestion reaction solution; Dephosphorylase 1 uL; Dephosphorylated buffer 5 ul; Replenish water to 50 ul. Reaction condition were 37° C. for 1 h; Inactivated at 65° C. for 5 min.

3) Use OMEGA Ultra-Sep Gel Extraction Kit to recover the vectors and fragments.

4) 100 ng of the purified shuttle plasmid and 100 ng of the purified adenovirus vector were co-transformed into BJ5183 competent cells, and the transformed product was coated with an LB plate containing Kan and cultured at 37° C. for 12-16 h.

5) The colonies were selected and cultured in 5 mL LB liquid medium containing Kan under shaking at 37° C. for 12-16 h, and the plasmids were extracted for XhoI enzyme digestion verification. The results were shown in FIG. 112, where Lanes 1-7 were clones of pAd5LCL3-L8Lubiqutin-IRES-I215L and M: 15000 bp MARKER. As shown in FIG. 112, Clones 6 and 7 were correctly digested.

6) The No. 6 positive plasmid was converted into DH5a competent state, a colony was selected and cultured in 5 mL LB liquid medium containing Kan under shaking at 37° C. for 12-16 h, and the extracted plasmid was subjected to XhoI restriction endonuclease verification again. The restriction endonuclease result was shown in FIG. 113, where lane 1 was subjected to XhoI restriction endonuclease of pAd5LCL3-L8Lubiqutin-IRES-I215L plasmid. M was DL 15,000 DNA Marker. As can be seen from FIG. 113, the enzyme digestion result was correct, and the adenovirus vector plasmid pAd5LCL3-L8Lubiqutin-IRES-I215L was successfully constructed.

2. Autologous recombination of shuttle plasmid pS5E4-I73RHBsAg-2A-E146L and adenovirus vector plasmid pAd5LCL3-L8Lubiqutin-IRES-I215L to obtain pAd5LCL3-L8Lubiqutin-I215L-I73RHBsAg-E146L.

1) PacI and I-sceI perform enzyme digestion on the shuttle plasmid pS5E4-I73RHBsAg-2A-E146L and the adenovirus vector plasmid pAd5LCL3-L8Lubiqutin-IRES-I215L, and the enzyme digestion reaction system was as follows:

A. Shuttle plasmid pS5E4-I73RHBsAg-2A-E146L3p g; PacI 2 ul; 10×cutsmart buffer 4 ul; Replenish water to 40 ul.

B. Adenovirus vector plasmid pAd5LCL3-L8Lubiqutin-IRES-I215L3 ug; I-sceI 2 ul; Buffer cutsmart 4 ul; Replenish water to 40 ul.

Reaction condition were 37° C. for 1 h; Inactivated at 65° C. for 20 min.

2 ul of agarose gel was used for validation, and the results were shown in FIG. 114, with lane 1 being pS5E4-I73RHBsAg-2A-E146L and lane 2 being pAd5LCL3-L8Lubiqutin-IRES-I215L.

2) Dephosphorylation of that enzyme digestion product

Reaction system: 37.5 ul; of enzyme digestion reaction solution; Dephosphorylase 1 uL; Dephosphorylated buffer 5 ul; Replenish water to 50 ul. Reaction condition were 37.0° C. for 1 h; Inactivated at 65° C. for 5 min.

3) Use OMEGA Ultra-Sep Gel Extraction Kit to recover the vectors and fragments.

4) 100 ng of the purified shuttle plasmid and 100 ng of the purified adenovirus vector were co-transformed into BJS183 competent cells, and the transformed product was coated with an LB plate containing Kan and cultured at 37° C. for 12-16 h.

5) Six colonies were selected and cultured in 5 mL LB liquid medium containing Kan under shaking at 37° C. for 12-16 h, and the plasmids were extracted for XhoI enzyme digestion verification. The results were shown in FIG. 115, where lanes 1-8 were plasmids, and M was DL 15,000 DNA Marker. It can be seen that plasmids 1-8 were correct.

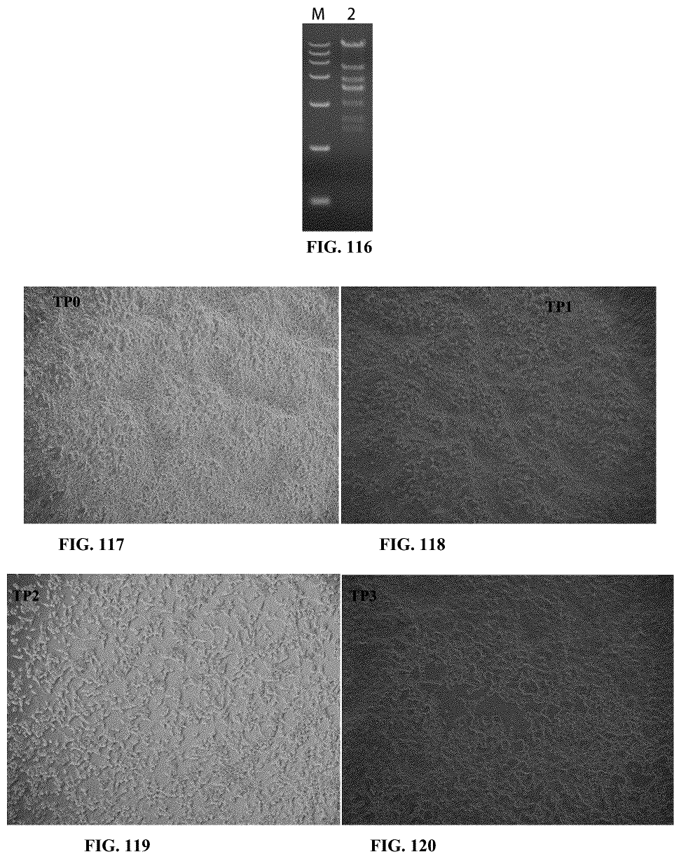

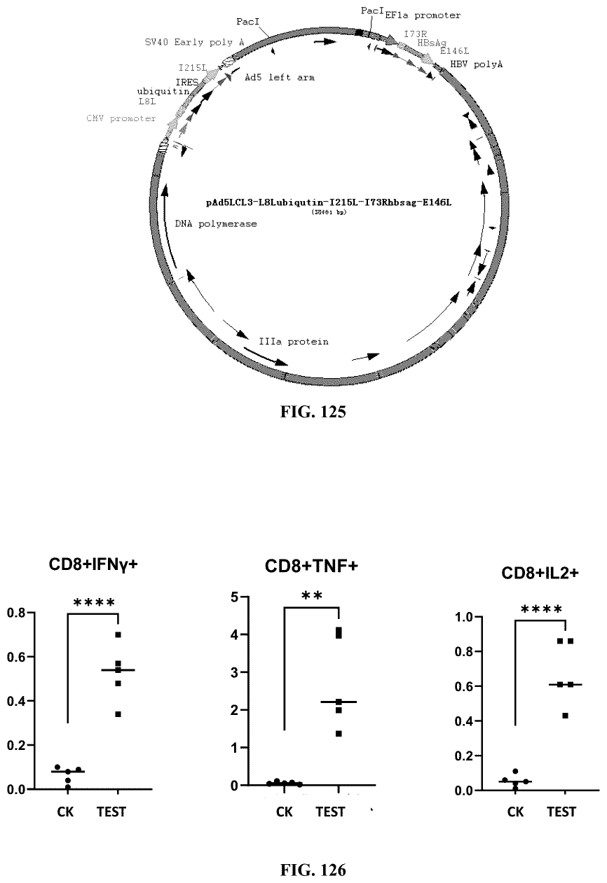

6) transform that No. 2 positive plasmid into DH5a competence; One colony was selected and cultured in 5 mL LB liquid medium containing Kan under shaking at 37° C. for 12-16 h, and the plasmid was extracted for XhoI restriction enzyme digestion verification again. The enzyme digestion result was shown in FIG. 116. The plasmid XhoI in lane 2 was pAd5LCL3-L8Lubiqutin-I215L-I73RHBsAg-E146L plasmid, and M was DL 15,000 DNA Marker. As shown in FIG. 116, pAd5LCL3-L8Lubiqutin-I215L-I73RHBsAg-E146L was successfully constructed. The enzyme digestion result was correct, and its vector map was shown in FIG. 125.

### Example 26: Packaging of Recombinant Adenovirus

Wrap the pAd5LCL3-L8Lubiqutin-I215L-I73RHBsAg-E146L plasmid in 293TD37 cells as follows: Preparation of 293TD37 cells: The cells were prepared one day before transfection. The 293TD37 cells to be transfected were inoculated into a 6-well plate at 0.5×10⁶/well, and incubated at 37° C. with 5% CO₂ for 24 hours. On the day of transfection, the cells showed 40-50% confluency.

Linearization of plasmid pAd5LCL3-L8Lubiqutin-I215L-I73RHBsAg-E146L: The plasmid to be transfected was digested with PacI enzyme, incubated for 40 min at 37° C., and inactivated for 20 min at 65° C.

Transfection: The linearized 2 pg plasmid and PEI were diluted with 100 ul serum-free medium, respectively. The plasmid diluent was added into the PEI diluent, and repeatedly aspirated for 5 times or vortexed for 10 seconds to be mixed evenly, and incubated for 10 minutes at room temperature to form a transfection complex. During incubation, cell culture medium was gently aspirated from the plates, 2 mL of fresh growth medium was added, and after 10 minutes the transfection complex was added to the cells in fresh medium.

Cell culture: the transfected 293TD37 cells were incubated at 37° C. for 72-96 hours in a 5% CO₂ incubator; 6-well plate cell suspensions were collected in 1.5 ml centrifuge tubes, TP0, 72-96 hours after viral plasmid transfection.

Continuous inoculation: The collected cell suspension was repeatedly frozen and thawed at −80° C. for 3 times, centrifuged at 2000 g at 4 C for 10 minutes, 500 ul of supernatant was collected to infect 293TD37 cells (293TD37 cells need to be prepared one day in advance), incubated at 37° C. with 5% CO₂ for 60 minutes, supplemented with 2 mL of FBS medium, and cultured at 37° C. with 5% CO₂ for 72 hours, to collect cell suspension, namely TP1. The previous steps were repeated and the cell suspension, TP2, was collected. The drug was continued until the cells became diseased.

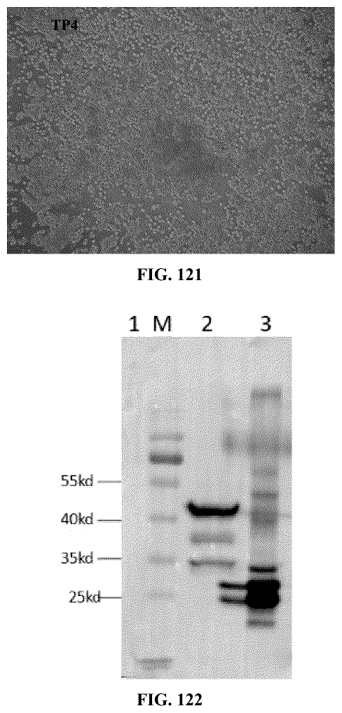

Cytopathic effect: When the 293TD37 cells were cultured from TP0 to TP4, the cells gradually became diseased until the 293TD37 cells were completely diseased at TP4. The cytopathic effects caused by TP0 to TP4 were shown in FIG. 117-121.

### Example 27: Detection of Titer of African Swine Fever Multiantigen Recombinant Adenovirus Vaccine

The 293TD37 cells were prepared. The cells with good growth in T75 culture flask were taken, the supernatant was discarded, the cells were washed with PBS, and digested with 0.25% trypsin, and then 10 mL of fresh DMEM medium containing 10% fetal bovine serum was added to stop the digestion, which was then blown, mixed, inoculated into 6-well plates (5×10⁵/mL, 2 ml per well), and allowed to stand for culture in a 37° C. 5% CO₂ incubator. After 24 hours, when the cells adhered to grow into single-layer cells, the culture medium was discarded, and the recombinant adenovirus was continuously diluted 10-3 to 10-6 times with serum-free DMEM maintenance solution, and two wells were inoculated with each dilution degree at 250 uL per well. After one hour of infection, the supernatant was discarded to supplement the complete culture medium, and then the medium was allowed to stand for culture in a 5% carbon dioxide incubator at 37° C. After 24 h, the supernatant was discarded and the cells were washed with PBS (1 mL per well). After PBS was discarded, 1 mL of cold formaldehyde was added into each well for fixation, and formaldehyde was discarded at room temperature for 10 min. Then the cells were washed with PBS (1 mL per well), followed by adenovirus antibody-FITC (1 ml per well). After 1 h at room temperature, the cells were washed with PBS again (1 mL per well). After two times, 1 mL of PBS was added into each well and counted under fluorescence microscope (200 times, 10 continuous fields). Calculation: Virus titer (FFU/mL)=Mean×1013×4×10⁽⁻ⁿ⁾. The FFU for the pAd5LCL3-L8Lubiqutin-I215L-I73RHBsAg-E146L virus was 2.2×10⁸FFU/mL, with a high titer.

### Example 28: Detection of Stability of African Swine Fever Multiantigen Recombinant Adenovirus Vaccine pAd5LCL3-L8Lubiqutin-I215L-I73RHBsAg-E146L

The 293TD37 cells were prepared. The cells that grew well in T75 culture flask were taken, the supernatant was discarded, the cells were washed with PBS, and digested with 0.25% trypsin. Then 10 mL of fresh DMEM medium containing 10% fetal bovine serum was added to stop the digestion, and then the cells were blown and mixed evenly. The 293TD37 cells were planted into 6-well plates (5×10⁵ cells/mL, 2 mL/well), incubated for 1 hour at room temperature to adhere to the wall, and incubated for microscopic observation of its attachment degree. The infection was carried out with the pAd5LCL3-L8Lubiqutin-I215L-I73RHBsAg-E146 viral particle and the titer of infection was 5MOI/well. After the 293TD37 cells developed lesions 48 hours later, the cells were collected, repeatedly frozen and thawed for three times, and then centrifuged at 2000 g; the collected supernatant was detected for FFU, and then new 293TD37 cells were reinfected until the 30th generation. The collected virus solutions of passages 5, 10, 15, 20, 25 and 30 were tested, and the genome of the virus was found to be still intact, indicating that the replication-defective pAd5LCL3-L8Lubiqutin-I215L-I73RHBsAg-E146 virus could be stably packaged in 293TD37 cells.

### Example 29: Detection of Recovery Mutation (RCA) in the African Swine Fever Multiantigen Recombinant Adenovirus Vaccine pAd5LCL3-L8Lubiqutin-I215L-I73RHBsAg-E146

pAd5LCL3-L8Lubiqutin-I215L-I73RHBsAg-E146L virus RCA detection method was as follows:

1. Prepare a solution of pAd5LCL3-L8Lubiqutin-I215L-I73RHBsAg-E146 virus, and measure the virus titer and determine the concentration of virus particles. The DNA of the host cell shall be digested with 1% Universal Nuclease (7.5-15 units/mL virus solution) in the virus solution and water bath at 37° C. for 40 min. Using a 300Kd ultrafiltration centrifuge tube, the virus particles were collected after centrifugation at 1000 g for 30 min, followed by elution with 1×PBS. A260 was measured, and the particle concentration=A260 *1.1*10¹² VP/mL.

2. Virus infection: A 6-well plate of A549 cells was prepared, with each well cell being 2.5×10⁵/well. The medium was discarded and washed with PBS once. Adenovirus was inoculated with virus at 1×10⁹ vp/well to infect A549 cells. Wild-type human adenovirus type 5 was used as the control at 37° C. and 5% CO₂. After 1 h, the virus solution was discarded and supplemented with 5% complete medium. The cells were cultured at 37° C. and 5% CO₂ for 48 h.

3. Immunostaining was performed, and the cell supernatant was discarded. The cells were surface washing cells in PBS, fixed with ice formaldehyde, placed at −20 C for 20 min, and washed with 1×PBS for three times, each time for 5 min. Then 2 ml 1% BSA-PBS solution was added into each well, placed in a shaker, and incubated for 1 h. After the supernatant was discarded, human adenovirus type 5 fluorescent antibody (1:500 dilution) was added and incubated for 1 h, followed by washing with 1×PBS for three times, 5 min each time.

RCA was calculated using the equation as observed under a 10-fold fluorescence microscope

$$RCA = ( average positive cell field ) \times ( 374 field / well ) \times ( dilution factor ) ) / Total VPs in 0.5 ml viral sample$$

The judging standard was the level of RCA <1 RCA/3×10¹⁰ VP. Through counting that the RCA level of the pAd5LCL3-L8Lubiqutin-I215L-I73RHBsAg-E146L was less than 1 RCA/3*10¹⁰ VP, the replication-defective pAd5LCL3-L8Lubiqutin-I215L-I73RHBsAg-E146L virus prepared by the invention can be stably packaged in 293TD37 cells and cannot be converted into wild type or has low probability of being converted into wild type.

### Example 30: Detection of Protein Expression of African Swine Fever Multiantigen Recombinant Adenovirus Vaccine pAd5LC3-L8Lubiqutin-I215L-I73RHBsAg-E146L

The 293TD37 cells were prepared one day in advance and placed in a 12-well cell culture plate. The 293TD37 cells were infected with the African swine fever multiantigen recombinant adenovirus vaccine pAd5LCL3-L8Lubiqutin-I215L-173R HBsAg-E146L virus. After 48 hours, the cells became diseased, and 1 ml of cells were collected. The cells were washed with PBS, and samples were prepared for Western Blot detection. The antibody of HA was used to detect the target protein, and the antibody of HA was purchased from Abcam. Among them, L8Lubiquitin fusion protein, and I215L protein have HA tag, and the protein size was 32kda and 26kda. The experimental results were shown in FIG. 122. The cells in lanes 1 and 2 and 3 were the 293 blank cells and the cells in lanes 2 and 3 were the samples infected with pAd5LC3-L8Lubiqutin-I215L-I73RHBsAg-E146L. It could be clearly seen that the L8Lubiquitin fusion protein and I215L protein were normally expressed, so that thepAd5LC3-L8Lubiqutin-I215L-I73RHBsAg-E146L vaccine could normally express the target protein in 293 cells.

### Example 31: immunological evaluation on African swine fever multiantigen recombinant adenovirus Vaccine pAd5LCL3-L8Lubiqutin-I215L-I73RHBsAg-E146L Mouse Model

### Detection of Vaccine Cell Immune Response

Ten SPF-grade mice (6-8 weeks of age) were randomly divided into two groups, five for each group. Mice were immunized with pAd5LCL3-L8Lubiqutin-I215L-I73RHBsAg-E146L according to the groupings shown in Table 6. The injection method was as follows: intramuscular injection was performed on the medial aspect of the posterior thigh. Injection dose: 100 ul.

TABLE 6
Groups of Vaccinated Mice

| | | | Immune | Number |
|---|---|---|---|---|
| group | Vector vaccine | dosage | mode | of mice |
| Experiment | pAd5LCL3- | 1*10\hat{ }7 FFU | intramuscular | 5 |
| | L8Lubiqutin- | | injection | |
| | I215L-I73RHBsAg- | | | |
| | E146L | | | |
| control | pAd5LCL3 | 1*10\hat{ }7 FFU | intramuscular | 5 |
| | | | injection | |

The mice were sacrificed 14 days after immunization, and the splenic lymphocytes were isolated. PK15 cells transfected with the shuttle plasmids pS5E1-L8Lubiqutin-I215L and pS5E4-I73RHBsAg-E146L were stimulated and cultured for 6 hours, while protein secretion blockers were added to block cytokine secretion. After 6 hours, Fc receptors were blocked, dead cells and cell surface molecular markers were stained, and intracellular cytokines were stained after the cells were fixed and perforated. Cell surface markers included CD4 and CD8, and intracellular cytokines included IFNγ and IL2. The expression levels of IFNγ and IL2 in CD4+T cells and CD8+T cells stimulated by the target protein were analyzed by flow cytometry (CyExpert).

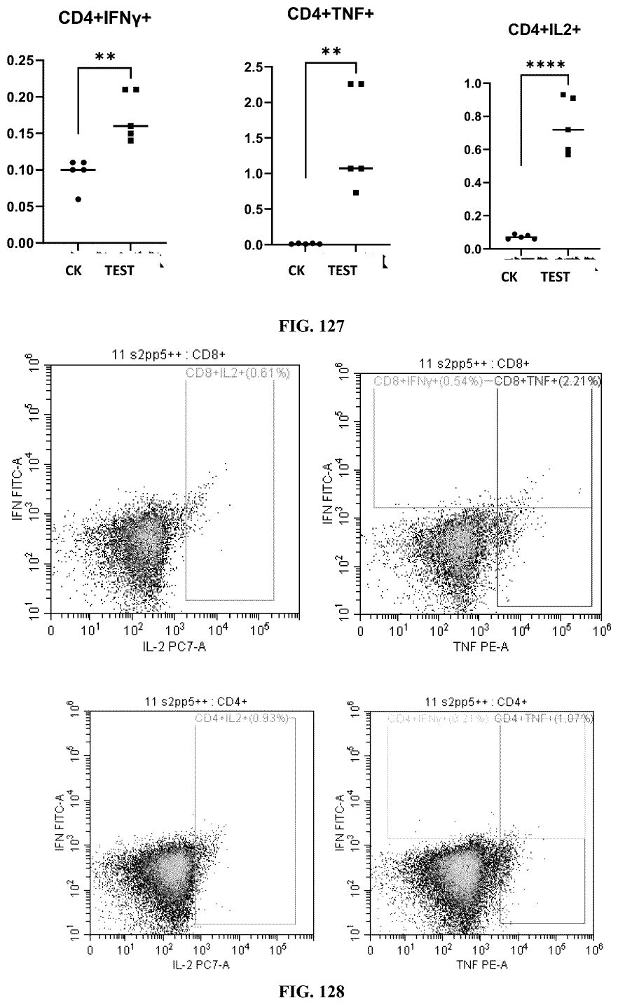

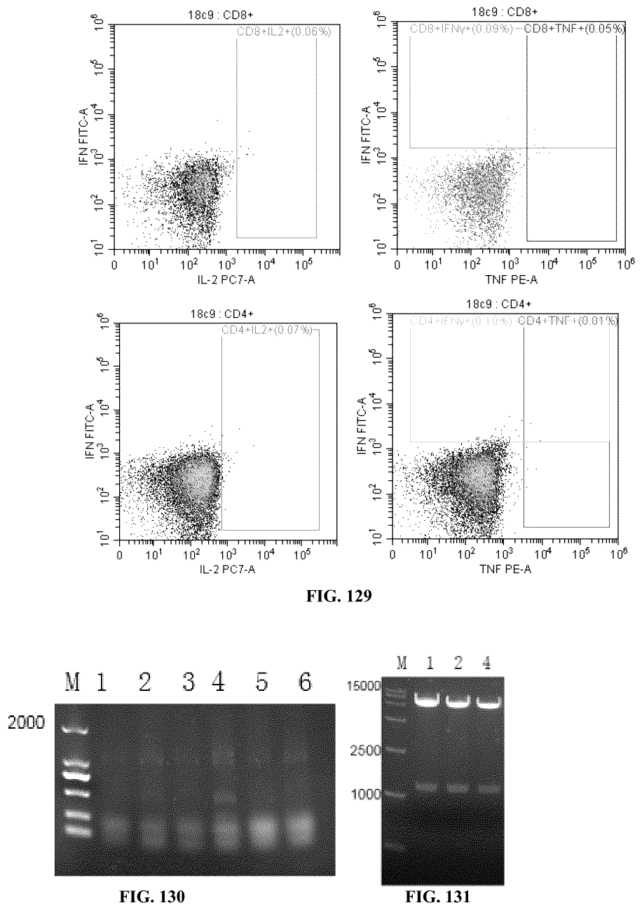

pAd5LCL3-L8Lubiqutin-I215L-I73RHBsAg-E146L-induced CD8+T cell and CD4+T cell immune response was shown in FIG. 126 and FIG. 127, and the representative results were shown in FIG. 128 to FIG. 129. FIG. 128 was the representative cellular immune response after intramuscular injection of pAd5LCL3-L8Lubiqutin-I215L-I73RHBsAg-E146L, and FIG. 129 was the representative blank control immune response. The results showed that, 14 days after immunization, after the spleen cells were stimulated by the target protein, the levels of IFNγ, TNFα and IL2 expressed in CD8+T cells were significantly higher than those in the HAd5 vector control group (Control)(P<0 0.05). After stimulation, the expression levels of IFNγ, TNFα and IL2 in CD4+T cells were significantly higher than those in the HAd5 vector control group (P<0.05). The results of cellular immune response test showed that intramuscular injection of the adenovirus vector vaccine of 1*10 7 FFU could induce specific cellular immune response in the immunized mice.

### Example 32: Construction of the African Swine Fever Human Adenovirus Type 5 vector E1 Region Shuttle Plasmid pS5E1-EP402R-IRES-EP153R

1. Construction of human adenovirus type 5 vector E1 region shuttle plasmid

The human adenovirus type 5 vector E1 region shuttle plasmid pS5E1 was successfully constructed according to the method provided in Example 4.

2. Construction of shuttle plasmid pSSE1-EP402R-IRES-EP153R of African swine fever human adenovirus type 5 vector

1) ligation of pSSE1 to the IRES fragment

pS5E1-IRES was successfully constructed according to the method provided in Example 4.

2) ligation of pSSE1-IRES to the EP402R fragment

\textcircled{1} primer synthesis

EP402R-BamHI-F:
(SEQ ID NO: 142)
cgcGGATCCgccaccATGATCATCATCGTGATCTTCC
EP402R-EcoRV-R:
(SEQ ID NO: 143)
ccgGATATCttaAGCGTAGTCTGGGACGTCGT

\textcircled{2} PCR amplification of EP402R fragment

Amplification system: 25 ul of Q5 enzyme, 10 μM primer EP402R-BamHI-F 1 ul, 10 μM primer EP402R-EcoRV 1 ul, template EP402R 1 ul, and water supplementing to 50 ul; PCR program: 98° C., 10 s; 98° C., 5s, 60° C., 30s, 72° C., 45s, 35 cycles; 72° C., 5 min.

\textcircled{3} The EP402R fragment was purified by an Axygen™ PCR purification kit.

\textcircled{4} The target fragment EP402R was digested with pS5E1-IRES vector

Enzyme digestion reaction system: the vector pS5E1-IRES, EP402R fragment −2 ug, and each of EcoRV and BamHI was 1 ul; 10×cutsmart buffer 5 ul; Replenish water to 50 ul. Reaction condition: 37° C., 30 min; Inactivated at 65° C. for 20 min. Gum is recovered and predicated. The electrophoretic detection of the digested products was shown in FIG. 23, where lane 1 was the double digestion of fragments EP402R, BamHI and EcoRV, and lane 2 was the double digestion of pS5E1-IRES, BamHI and EcoRV, and M was DL 15,000 DNA Marker.

\textcircled{5} The target fragment EP402R was connected with pS5E1-IRES

Ligation system: PS5E1-IRES (100 ng); Fragment of EP402R (vector:fragment=1: 3 molar ratio); T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

Amplification system: 10 ul of Q5 enzyme, 10 μM primer EP 402R-BAMH-F 1 uL, and 10 μM primer EP402R-EcoRV-R 1 ul were replenished to 20 ul; PCR program: 98° C., 10 s; 98° C., 5s, 60° C., 30s, 72° C., 1 min, 35 cycles; 72° C., 5 min. Electrophoresis verification was performed as shown in FIG. 130, where No. 1-6 was a colony and M was DL 2,000 DNA Marker.

\textcircled{7} Plasmid restriction endonuclease assay (BamHI&EcoRV), colony 1, 2 and 4 were selected for plasmid extraction and restriction endonuclease assay. The results were as shown in FIG. 131, all of which were positive plasmids.

3) ligation of pS5E1-EP402R-IRES to fragment EP153R

\textcircled{1} primer synthesis

EP153R-NotI-F:
(SEQ ID NO: 144)
ATAAGAAT GCGGCCGCgccaccATGTTCAGCAACAAGAAGTAC
AT
EP153R-XhoI-R:
(SEQ ID NO: 145)
AAACTCGAGTCACTTGCTACAGATGTACAG

\textcircled{1} PCR amplification of EP153R fragment

Amplification system: 25 ul of Q5 enzyme, 10 μM primer EP153R-NotI-F 1 ul, 10 μM primer EP153R-XhoI-R 1 ul, and template EP153R 1 ul were replenished to 50 ul; PCR program: 98° C., 10 s; 98° C., 5s, 60° C., 30s, 72° C., 20s, 35 cycles; 72° C., 5 min.

\textcircled{3} the EP153R fragment was purified by an Axygen™ PCR purification kit.

\textcircled{4} The target fragment EP153R was digested with pS5E1-EP402R-IRES vector

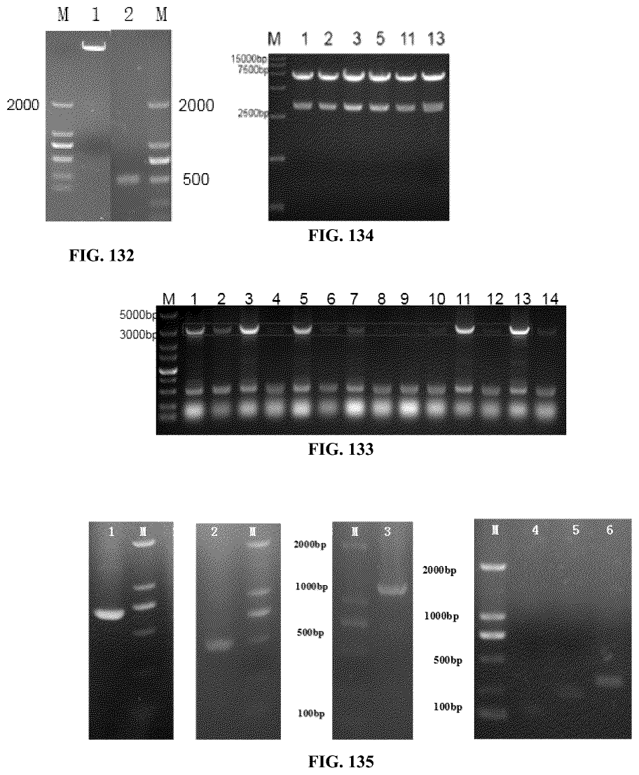

Enzyme digestion reaction system: vectors pS5E1-EP402R-IRES, EP153R fragment ˜2 ug, and 1 uL for NotI and XhoI respectively; 10×cutsmart buffer 5 ul; Replenish water to 50 ul. Reaction condition: 37° C., 30 min; Inactivated at 65° C. for 20 min. Recovery and purification of gum. The electrophoresis detection of the enzyme digestion product was shown in FIG. 132, where lane 1 was pS5E1-EP402R-IRES, NotI and XhoI enzyme digestion, lane 2 was EP153R fragment, NotI and XhoI enzyme digestion, and M was DL 2,000 DNA Marker.

\textcircled{5} pS5E1-EP402R-IRES vector was connected with EP153R fragment

Linkage system: PS5E1-EP 402R-IRES 100 ng; EP153R fragment 50 ng; T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

Amplification system: 10 ul of Q5 enzyme, 10 uM universal primer CMV-F 1 ul, and 10 uM primer EP153R-XhoI-R 1 ul were replenished to 20 ul; PCR program: 98° C., 10 s; 98° C., 5s, 60° C., 30s, 72° C., 2 min 30 s, 35 cycles; 72° C., 5 min; Electrophoresis verification was performed as shown in FIG. 133, where No. 1-14 was the colony and M was DL 5,000 DNA Marker.

\textcircled{7} Plasmid BamHI and XhoI restriction enzyme digestion verification: 1, 2, 3, 11 and 13 were selected for plasmid extraction and restriction enzyme digestion verification. The results were shown in FIG. 134, where lanes 1, 2, 4 and 6 were used for plasmid BamHI and XhoI restriction enzyme digestion identification, and M was DL 15,000 DNA Marker. As shown in FIG. 134, the enzyme digestion result was correct, and the African swine fever human adenovirus type 5 vector E1 region shuttle plasmid pS5E1-EP402R-IRES-EP153R was successfully constructed, and the vector map was shown in FIG. 150.

### Example 33: Construction of African Swine Fever Human Adenovirus Type 5 Vector E4 Region Shuttle Plasmid pS5E4-I177L-2A-K205Rubiqutin

1. Construction of human adenovirus type 5 vector E4 region shuttle plasmid

The human adenovirus type 5 vector E4 region shuttle plasmid pS5E4-EGFP was successfully constructed according to the method of Example 5.

2. Construction of African swine fever human adenovirus type 5 vector E4 region shuttle plasmid pSSE4-I177L-2A-K205Rubiqutin

1) Primer design

EF1α-BamHI-1177L-F:
(SEQ ID NO: 146)
ccaagctgtgaccggcgcctacGGATCCGCCACCATGTG
GAAGGTGAA
I177L-R:
(SEQ ID NO: 147)
GGCGTAATCGGGCACGTCG
I177L-2A-F:
(SEQ ID NO: 148)
CTACGACGTGCCCGATTACGCCGGAAGCGGAGCTACTAACTTC
2A-K205R-R:
(SEQ ID NO: 149)
AACTGCTCTCTGGGCTCCACCATAGGTCCAGGGTTCTCCTCCA
K205R-F:
(SEQ ID NO: 150)
ATGGTGGAGCCCAGAGAGCA
K205R-ubiqutin-R:
(SEQ ID NO: 151)
GGGTTTTCACGAAAATCTGCATGGCGTAATCGGGCACATCGT
ubiqutin-F:
(SEQ ID NO: 152)
ATGCAGATTTTCGTGAAAACCC
ubiquitin-XhoI-HBV-R:
(SEQ ID NO: 153)
GGGTTTAAACGGGCCCTCTAGACTCGAGTTACTTGTCTTCTGGT
TTGTTGA

2) Amplification of the target fragment I177L-K205Rubiqutin

\textcircled{1} The I177L fragment was amplified using the I177L gene synthesis fragment as the template and EF1α-BamHI-II77L-F and I177L-R as the primers; Amplification system: synthetic fragment of I177L gene of 50 ng, 10 μM EF1α-BamHI-I177L-F primer of 1 ul, 10 μM I177L-R primer of 1 ul, and Q5 high-fidelity enzyme of 20 ul; Replenish water to 40 ul; PCR procedure: 98° C., 10 sec; 98° C., 5 sec, 60° C., 30 sec, 72° C., 30 sec, 35 cycles; 72° C., 5 min.

\textcircled{2} Using the synthetic fragment of 2A gene as a template and I177L-2A-F and 2A-K205R-R as primers, we amplified the 2A fragment; Amplification system: synthetic fragment of 2A gene (50 μg), 10 μM I177L-2A-F primer (1 ul), 10 μM 2A-K205R-R primer (1 ul), and Q5 high-fidelity enzyme (20 ul); Replenish water to 40 ul; PCR procedure: 98° C., 10 sec; 98° C., 5 sec, 60° C., 30 sec, 72° C., 20 sec, 35 cycles; 72° C., 5 min.

\textcircled{3} The K205R fragment was amplified using the K205R gene synthesis fragment as the template and K205R-F and K205R-ubiquitin-R as the primers; Amplification system: synthetic fragment of K205R gene (50 g, 10 μM K205R-F primer (1 ul), 10 μM K205R-ubiquitin-R primer (1 ul), and high-fidelity enzyme Q5 (20 ul). Replenish water to 40 ul; PCR procedure: 98° C., 10 sec; 98° C., 5 sec, 60° C., 30 sec, 72° C., 30 sec, 35 cycles; 72° C., 5 min.

\textcircled{4} Using the synthetic fragment of ubiquitin gene as the template and ubiquitin-F and ubiquitin-XhoI-HBV-R as the primers, the ubiquitin fragment was amplified. Amplification system: ubiqutin gene synthetic fragment 50 ng, 10 uM ubiqutin-F primer 1 ul, 10 uM ubiqutin-XhoI-HBV-R primer 1 ul, Q5 high-fidelity enzyme 20 ul; Replenish water to 40 ul; PCR procedure: 98° C., 10 sec; 98° C., 5 sec, 60° C., 30 sec, 72° C., 40 sec, 35 cycles; 72° C., 5 min.

3) The target fragment was purified by an Axygen™ gel extraction kit.

4) Amplifying the I177L-2A fragment and the K205Rubiqutin fragment by fusion PCR

Amplification system: 50 ng of I177L gel recovery fragment, 50 ng of 2A gel recovery fragment, 1 ul of 10 μM EF1α-BAMHI-I177L-F primer, 1 ul of 10 μM 2A-K205R-R primer, and 25 ul; of Q5 high-fidelity enzyme; Replenish water to 50 ul; PCR procedure: 98° C., 10 sec; 98° C., 5 sec, 60° C., 30 sec, 72° C., 40 sec, 35 cycles; 72° C., 5 min.

Amplification system: 50 ng of K205R recovered fragment, 50 ng of ubiqutin recovered fragment, 1 ul of 10 uM K205R-F primer, 1 ul of 10 uM 2A-K205R-R primer, and 25 ul; of Q5 high-fidelity enzyme; Replenish water to 50 ul; PCR procedure: 98° C., 10 sec; 98° C., 5 sec, 60° C., 30 sec, 72° C., 40 sec, 35 cycles; 72° C., 5 min.

The electrophoretic detection of the PCR product was shown in FIG. 135, wherein lane 1 was fragment K205R; Lane 2 was fragment ubiqutin; Lane 3 was fragment K205Rubiqutin, and M was DL 2,000 DNA Marker. Lane 4 was fragment 2a; Lane 5 was fragment I177L; Lane 6 was fragment I177L-2A, and M was DL 2,000 DNA Marker.

5) Enzyme digestion with pSSE4-EGFP vector

Enzyme digestion reaction system: vector pSSE4-EGFP 2 ug, BamHI and XhoI 1 uL each; 10×cutsmart buffer 5 ul; Replenish water to 50 ul. Reaction condition: 37° C., 30 min; Inactivated at 65° C. for 20 min. Recovery and purification.

6) Purifying the Vector Fragment by an Axygen™ Gel Extraction Kit

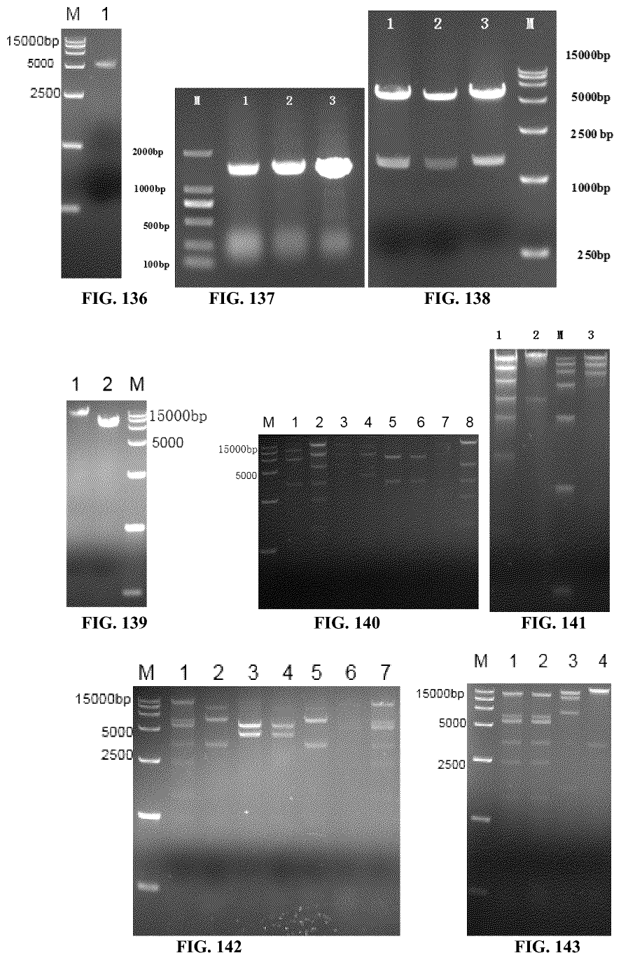

The gel recovery results were shown in FIG. 136, where lane 1 was the fragment pS5E4-EGFP, BamHI and XhoI double enzyme digestion for gel recovery, and M was DL 15,000 DNA Marker.

7) seamless clonal connection and transformation of pS5E4-EGFP gel recovery vector with I177L-2A fragment and K205Rubiqutin

Ligation system: pS5E4-EGFP gel recovery product (100 ng), I177L-2A fragment (50 ng), K205Rubiqutin fragment (50 ng), 2×Smealess Cloning Mix 5 ul, replenished to 10 ul. Reaction condition: 50° C., 40 min. The ligated products were transformed into DH5α competent cells, plated on plates containing ampicillin resistance and incubated at 37° C. for 12-16 hours.

8) plasmid validation

\textcircled{1} colony PCR

Using EF1α2(d)-F and HBV(jd)-R as primers, the target fragment was amplified by colony PCR and verified by agarose gel assay. The results were shown in FIG. 137, where lane 1-3 was the colonies and M was DL 15,000 DNA Marker.

\textcircled{2} enzyme digestion verification

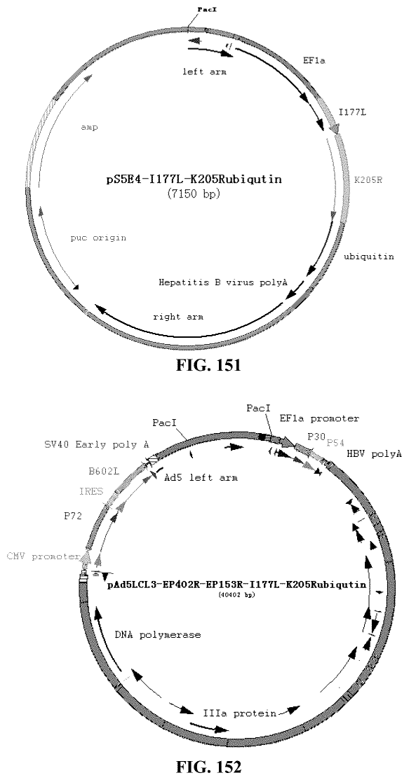

The No. 1, No. 2 and No. 3 positive clone were picked and place in 5 mL LB liquid medium containing ampicillin resistance for culture for 12-15 hours, and that plasmids were extract for double enzyme digestion verification of BmHI and XhoI; The enzyme digestion results were shown in FIG. 138, where lanes 1, 2 and 3 were the double enzyme digestion verification of the positive clones BamHI and XhoI, and M was DL 15,000 DNA Marker. The enzyme digestion result was correct and the sequencing was correct. The African swine fever human adenovirus type 5 vector E4 region shuttle plasmid pS5E4-I177L-2A-K205Rubiqutin was successfully constructed and the sequencing was correct. The vector map was shown in FIG. 151.

### Example 34: pAd5LCL3-EP402R-IRES-EP153R, pS5E4-I177L-2A-K205Rubiqutin Recombinant with pAd5LCL3 Construction of pAd5LCL3-EP402R-EP153R-I177L-K205Rubiqutin Plasmid

1. Homologous recombination of shuttle plasmid pS5E1-EP402R-IRES-EP153R with adenovirus vector plasmid pAd5LCL3

1) PacI and SwaI perform enzyme digestion on the shuttle plasmid pS5E1-EP402R-IRES-EP153R and the adenovirus vector plasmid pAd5LCL3, and the enzyme digestion reaction system was as follows:

A. Shuttle plasmid pS5E1-EP 402R-IRES-EP 153R 3p g; PacI 2 μl; buffer cutsmart 4 μl; Replenish water to 40 μl.

B, adenovirus vector plasmid pAd5LCL3 3 ug; SwaI 2 μl; Buffer 3.1 4 μl; Replenish water to 40 μl.

Reaction condition were 37° C. for 1 h; Inactivated at 65° C. for 20 min.

Two uL of agarose gel were verified as shown in FIG. 139, with lane 1 being pAd5LCL3 and lane 2 being pS5E1-EP402R-IRES-EP153R.

2) Dephosphorylation of that enzyme digestion product

Reaction system: 37.5 μL enzyme digestion reaction solution; 1 μL dephosphorylase; Dephosphorylated buffer 5 μL; Refill water to 50 μL. Reaction condition were 37° C. for 1 h; Inactivated at 65° C. for 5 min.

3) Use OMEGA Ultra-Sep Gel Extraction Kit to recover the vectors and fragments.

4) 100 ng of the purified shuttle plasmid and 100 ng of the purified adenovirus vector were co-transformed into BJ5183 competent cells, and the transformed product was coated with an LB plate containing Kan and cultured at 37° C. for 12-16 h.

5) The colonies were selected and cultured in 5 mL LB liquid medium containing Kan under shaking at 37° C. for 12-16 h, and the plasmids were extracted for XhoI enzyme digestion verification. The results were shown in FIG. 140, where lanes 1-8 were clones of PAD5LCL3-EP 402R-IRES-EP153R and M: 15000 bp marker. It can be seen from FIG. 140 that Clones 2, 3 and 8 were correctly digested.

6) The No. 2 positive plasmid was converted into DH5α competent state, a colony was selected and cultured in 5 mL LB liquid medium containing Kan under shaking at 37° C. for 12-16 h, and the plasmid was extracted for XhoI restriction enzyme digestion verification again. The restriction enzyme digestion result was shown in FIG. 141, which shows that lane 1 was plasmid XhoI restriction enzyme digestion of pAd5LCL3-EP402R-IRES-EP153R; The plasmid PacI digestion of pAd5LCL3-EP402R-IRES-EP153R in lane 2 and the plasmid BamHI digestion of pAd5LCL3-EP402R-IRES-EP153R in lane 3 showed the correct digestion results. The adenovirus vector pAd5LCL3-EP402R-IRES-EP153R was successfully constructed.

2. Autologous recombination of shuttle plasmid pS5E4-I177L-2A-K205Rubiqutin with adenovirus vector plasmid pAd5LCL3-EP402R-IRES-EP153R to obtain pAd5LCL3-EP402R-EP153R-I177L-K205Rubiqutin

1) Enzymatic cleavage by 1) PacI and I-sceI of the shuttle plasmid pS5E4-I177L-2A-K205Rubiqutin and the adenovirus vector plasmid pAd5LCL3-EP402R-IRES-EP153R in the following reaction system:

A. The shuttle plasmid pS5E4-I177L-2A-K205Rubiqutin 3 pg; PacI 2 μl; 10×cutsmart buffer 4 μl; Replenish water to 40 μl.

B. Adenovirus vector plasmid pAd5LCL3-EP402R-IRES-EP153R 3 ug; I-sceI 2 μl; Buffer cutsmart 4 μl; Replenish water to 40 μl.

Reaction condition were 37° C. for 1 h; Inactivated at 65° C. for 20 min.

2) Dephosphorylation of that enzyme digestion product

Reaction system: 37.5 μL enzyme digestion reaction solution; 1 μL dephosphorylase; Dephosphorylated buffer 5 μL; Refill water to 50 μL. Reaction condition were 37° C. for 1 h; Inactivated at 65° C. for 5 min.

3) Use OMEGA Ultra-Sep Gel Extraction Kit to recover the vectors and fragments.

4) 100 ng of the purified shuttle plasmid and 100 ng of the purified adenovirus vector were co-transformed into BJ5183 competent cells, and the transformed product was coated with an LB plate containing Kan and cultured at 37° C. for 12-16 h.

5) Six colonies were selected and cultured in 5 mL LB liquid medium containing Kan under shaking at 37° C. for 12-16 h, and the plasmids were extracted for XhoI enzyme digestion verification. The results were shown in FIG. 142, where lanes 1-7 were plasmids, and M was DL 15,000 DNA Marker. It can be seen that plasmids No. 1 and 7 were correct.

6) transform that No. 1 positive plasmid into DH5α competence; One colony was selected and cultured in 5 mL LB liquid medium containing Kan under shaking at 37° C. for 12-16 h, and the plasmid was extracted for XhoI restriction enzyme digestion verification again. The digestion results were shown in FIG. 143, with lanes 1 and 2 for the digestion of plasmid XhoI of pAd5LCL3-EP402R-EP 153R-I177L-K205Rubiqutin, and lane 3 for the digestion of plasmid BamHI of pAd5LCL3-EP402R-EP153R-I177L-K205Rubiqutin. Lane 4 was the PacI restriction endonuclease of the plasmid pAd5LCL3-EP402R-EP153R-I177L-K205Rubiqutin, and M was DL 15,000 DNA Marker. As shown in FIG. 143, the restriction endonuclease result was correct. The adenovirus vector pAd5LCL3-EP402R-EP153R-I177L-K205Rubiqutin has been successfully constructed. The restriction endonuclease result was correct, and the vector map was shown in FIG. 152.

### Example 35: Packaging of Recombinant Adenovirus

Wrap the plasmid pAd5LCL3-EP402R-EP153R-I177L-K205Rubiqutin in 293TD37 cells as follows:

Preparation of 293TD37 cells: The cells were prepared one day before transfection. The 293TD37 cells to be transfected were inoculated into a 6-well plate at 0.5 ×106/well, and incubated at 37° C. with 5% CO₂ for 24 hours. On the day of transfection, the cells showed 40-50% confluency.

Linearization of plasmid pAd5LCL3-EP402R-EP153R-I177L-K205Rubiqutin: The plasmid to be transfected was digested with PacI enzyme, incubated for 40 min at 37 C, and inactivated for 20 min at 65° C.

Transfection: The linearized 2 pg plasmid and PEI were diluted with 100 μl serum-free medium, respectively. The plasmid diluent was added into the PEI diluent, and repeatedly aspirated for 5 times or vortexed for 10 seconds to be mixed evenly, and incubated for 10 minutes at room temperature to form a transfection complex. During incubation, cell culture medium was gently aspirated from the plates, 2 mL of fresh growth medium was added, and after 10 minutes the transfection complex was added to the cells in fresh medium.

Cell culture: the transfected 293TD37 cells were incubated at 37° C. for 72-96 hours in a 5% CO₂ incubator; 6-well plate cell suspensions were collected in 1.5 ml centrifuge tubes, TP0, 72-96 hours after viral plasmid transfection.

Continuous inoculation: The collected cell suspension was repeatedly frozen and thawed at −80° C. for 3 times, centrifuged at 2000 g for 10 minutes at 4 C, and 500 μl of supernatant was collected to infect 293TD37 cells (293TD37 cells need to be prepared one day in advance). The cells were incubated at 37° C. with 5% CO₂ for 60 minutes, followed by the addition of 2 mL of FBS medium, followed by culture at 37° C. with 5% CO₂ for 72 hours, and the cell suspension, namely TP1, was collected. The previous steps were repeated and the cell suspension, TP2, was collected. The drug was continued until the cells became diseased.

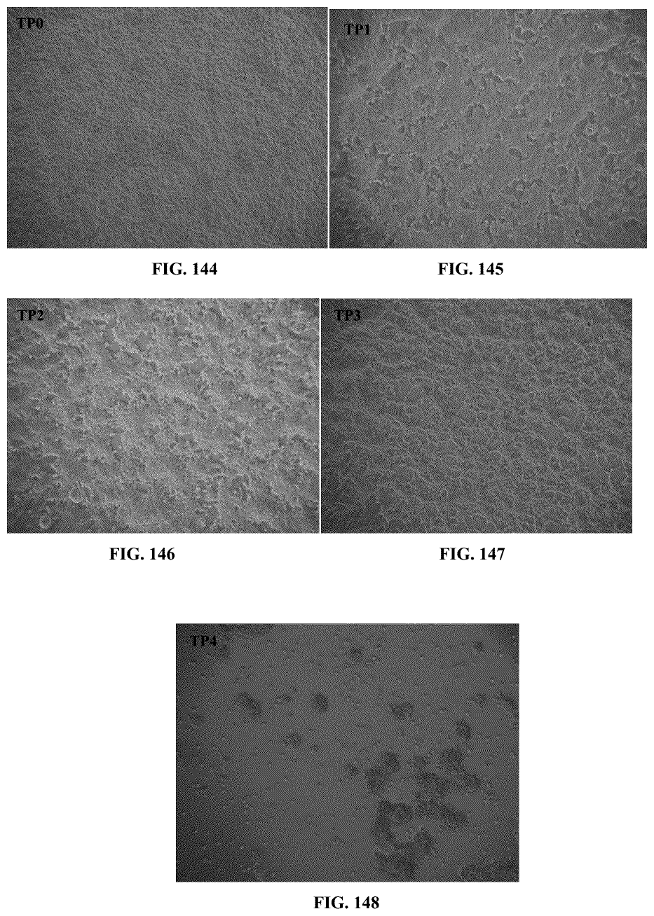

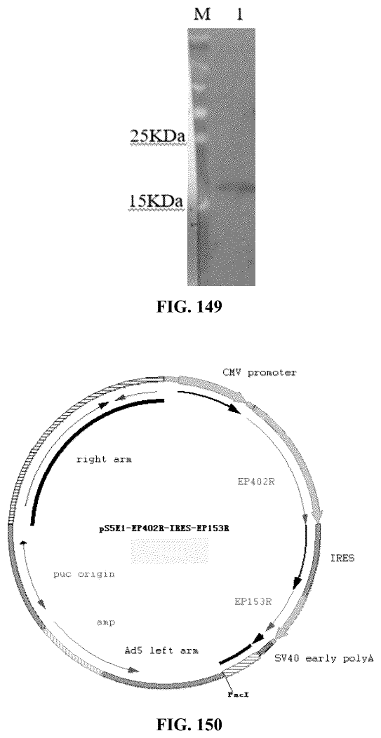

Cytopathic effect: When the 293TD37 cells were cultured from TP0 to TP4, the cells gradually became diseased until the TP4293TD37 cells became completely diseased. The cytopathic effects caused by TP0 to TP4 were shown in FIG. 144-148, respectively. TP4 was completely diseased.

### Example 36: Detection of Titer of African Swine Fever Multiantigen Recombinant Adenovirus Vaccine

The 293TD37 cells were prepared. The cells with good growth in T75 culture flask were taken, the supernatant was discarded, the cells were washed with PBS, and digested with 0.25% trypsin, and then 10 mL of fresh DMEM medium containing 10% fetal bovine serum was added to stop the digestion, which was then blown, mixed, inoculated into 6-well plates (5×10⁵/mL, 2 ml per well), and allowed to stand for culture in a 37° C. 5% CO₂ incubator. After 24 hours, when the cells adhered to grow into single-layer cells, the culture medium was discarded, and the recombinant adenovirus was continuously diluted 10-3 to 10-6 times with serum-free DMEM maintenance solution, and two wells were inoculated with each dilution degree at 250 uL per well. After one hour of infection, the supernatant was discarded to supplement the complete culture medium, and then the medium was allowed to stand for culture in a 5% carbon dioxide incubator at 37° C. After 24 h, the supernatant was discarded and the cells were washed with PBS (1 mL per well). After PBS was discarded, 1 mL of cold formaldehyde was added into each well for fixation, and formaldehyde was discarded at room temperature for 10 min. Then the cells were washed with PBS (1 mL per well), followed by adenovirus antibody-FITC (1 ml per well). After 1 h at room temperature, the cells were washed with PBS again (1 mL per well). After two times, 1 mL of PBS was added into each well and counted under fluorescence microscope (200 times, 10 continuous fields). Calculation: Virus titer (FFU/mL)=Mean×1013×4×10⁽⁻ⁿ⁾. The FFU of the pAd5LCL3-EP402R-EP153R-I177L-K205Rubiqutin virus was 1.8×10⁸FFU/mL, with a high titer.

### Example 37: Stability Test of African Swine Fever Multiantigen Recombinant Adenovirus Vaccine pAd5LCL3-EP402R-EP153R-I177L-K205Rubiqutin

The 293TD37 cells were prepared. The cells that grew well in T75 culture flask were taken, the supernatant was discarded, the cells were washed with PBS, and digested with 0.25% trypsin. Then 10 mL of fresh DMEM medium containing 10% fetal bovine serum was added to stop the digestion, and then the cells were blown and mixed evenly. The 293TD37 cells were planted into 6-well plates (5×10⁵cells/mL, 2 mL/well), incubated for 1 hour at room temperature to adhere to the wall, and incubated for microscopic observation of its attachment degree. The infection was carried out with the pAd5LCL3-EP402R-EP153R-I177L-K205Rubiqutin virus particle and the titer of infection was 5 MOI/well. After the 293TD37 cells developed lesions 48 hours later, the cells were collected, repeatedly frozen and thawed for three times, and then centrifuged at 2000 g; the collected supernatant was detected for FFU, and then new 293TD37 cells were reinfected until the 30th generation. The collected virus solutions from passages 5, 10, 15, 20, 25, and 30 were tested and the virus genome was found to be still intact, indicating that the replication-defective pAd5LCL3-EP402R-EP153R-I177L-K205Rubiqutin virus could be stably packaged in 293TD37 cells.

### Example 38: Detection of the African Swine Fever Multiantigen Recombinant Adenovirus Vaccine

pAd5LCL3-EP402R-EP153R-I177L-K205Rubiqutin recovery mutation (RCA) pAd5LCL3-EP402R-EP153R-I177L-K205Rubiqutin virus RCA detection method is as follows:

1. Prepare pAd5LCL3-EP402R-EP153R-I177L-K205Rubiqutin virus solution, test the virus titer and determine the concentration of virus particles. The DNA of the host cell shall be digested with 1% Universal Nuclease (7.5-15 units/mL virus solution) in the virus solution and water bath at 37° C. for 40 min. Using a 300Kd ultrafiltration centrifuge tube, the virus particles were collected after centrifugation at 1000 g for 30 min, followed by elution with 1×PBS. A260 was measured, and the particle concentration=A260*1.1*10 12 VP/mL. 2. Virus infection: A 6-well plate of A549 cells was prepared, with each well cell being 2.5×10⁵/well. The medium was discarded and washed with PBS once. Adenovirus was inoculated with virus at 1×10⁹ vp/well to infect A549 cells. Wild-type human adenovirus type 5 was used as the control at 37° C. and 5% CO₂. After 1 h, the virus solution was discarded and supplemented with 5% complete medium. The cells were cultured at 37° C. and 5% CO₂ for 48 h.

3. Immunostaining was performed, and the cell supernatant was discarded. The cells were surface washing cells in PBS, fixed with ice formaldehyde, placed at −20° C. for 20 min, and washed with 1×PBS for three times, each time for 5 min. Then 2 ml 1% BSA-PBS solution was added into each well, placed in a shaker, and incubated for 1 h. After the supernatant was discarded, human adenovirus type 5 fluorescent antibody (1:500 dilution) was added and incubated for 1 h, followed by washing with 1×PBS for three times, 5 min each time.

RCA was calculated using the equation as observed under a 10-fold fluorescence microscope

$$RCA = ( average positive cell field ) \times ( 374 field / well ) \times ( dilution factor ) ) / Total VPs in 0.5 ml viral sample$$

The judging standard was that the level of RCA was less than 1RCA/3×10¹⁰ vp. According to statistics, the RCA level of pAd5LCL3-EP402R-EP153R-I177L-K205Rubiqutin was less than 1RCA/3×10¹⁰ vp, which indicates that the replication-defective pAd5LCL3-EP402R-EP153R-I177L-K205Rubiqutin virus prepared by the invention can be stably packaged in 293TD37 cells and cannot be converted into wild type or the probability of conversion into wild type was low.

### Example 39: Detection of Protein Expression of African Swine Fever Multiantigen Recombinant Adenovirus Vaccine pAd5LCL3-EP402R-EP153R-I177L-K205Rubiqutin

The 293TD37 cells were prepared one day in advance and placed in a 12-well cell culture plate. The 293TD37 cells were infected with the African swine fever multiantigen recombinant adenovirus vaccine pAd5LCL3-EP402R-EP153R-I177L-K205Rubiqutin virus. The cells became diseased 48 hours later, and 1 ml of cells was collected. The cells were washed with PBS, and samples were prepared for Western Blot detection. The target protein was detected using our EP153R mouse antiserum, which was obtained by immunizing mice with EP153R protein expressed systemically in E. coli. The size of the EP153R protein was 15kda. The experimental results were shown in FIG. 148, lane 1 was the sample of pAd5LCL3-EP402R-EP153R-I177L-K205Rubiqutin infected with 293TD37 cells; It could be clearly seen that the normal expression of the EP153R protein was present, and thus it could be seen that the pAd5LCL3-EP402R-EP153R-I177L-K205Rubiqutin vaccine was able to express the target protein in 293 cells.

At the same time, the target protein was detected by our EP153R mouse antiserum, which was obtained by immunizing mice with EP153R protein expressed systematically in E. coli. The size of the EP153R protein was 15 kda.

### Example 40: Immunologic Evaluation of African Swine Fever Multiantigen Recombinant Adenovirus Vaccine pAd5LCL3-EP402R-EP153R-I177L-K205Rubiqutin in a Mouse Model

### 40.1 Detection of Humoral Immune Response of Vaccine

Twenty SPF-grade mice (6-8 weeks of age) were randomly divided into four groups, five for each group. Mice were immunized with pAd5LCL3-EP 402R-EP 153R-I177L-K205Rubiqutin according to the groupings shown in Table 7. The injection method was as follows: intramuscular injection was performed on the medial aspect of the posterior thigh. Injection dose: 100 ul.

TABLE 7
Groups of Vaccinated Mice

| | | | Immune | Number |
|---|---|---|---|---|
| group | Vector vaccine | dosage | mode | of mice |
| High | pAd5LCL3-EP402R- | 1*10\hat{ }8 FFU | intramuscular | 5 |
| dose | EP153R-I177L- | | injection | |
| | K205Rubiqutin | | | |
| Medium | pAd5LCL3-EP402R- | 1*10\hat{ }7 FFU | intramuscular | 5 |
| dose | EP153R-I177L- | | injection | |
| | K205Rubiqutin | | | |
| Low | pAd5LCL3-EP402R- | 1*10\hat{ }6 FFU | intramuscular | 5 |
| dose | EP153R-I177L- | | injection | |
| | K205Rubiqutin | | | |
| control | pAd5LCL3 | 1*10\hat{ }7 FFU | intramuscular | 5 |
| | | | injection | |

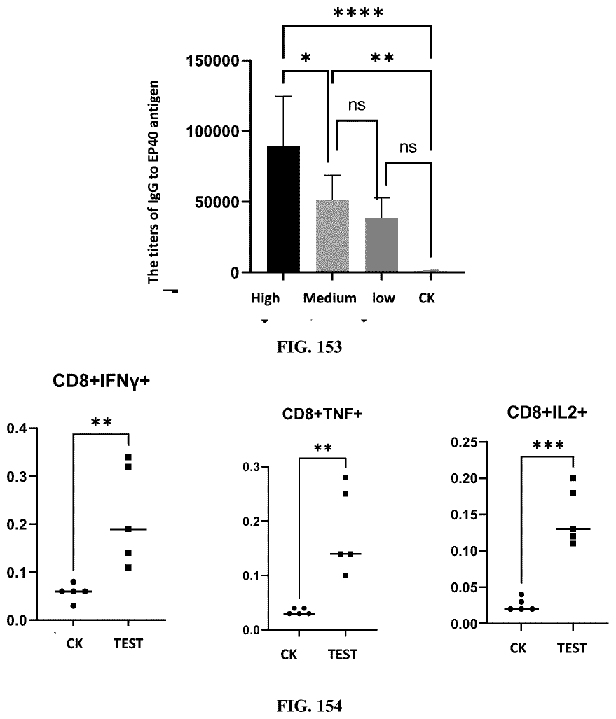

Blood was collected from the mice 14 days after immunization, serum was isolated, and IgG antibody titers against the African swine fever target protein EP402R (obtained after our company prepared and immunized the mice in insect cells) in serum were detected by indirect ELISA. The test results were shown in FIG. 153 (ns, P>0 0.05; *, P<0 0.05; **, P<0.01; ***, P<0.001; ****, P<0.0001). Mice were able to produce higher concentrations of IgG antibodies to the EP402R protein after intramuscular injection of pAd5LCL3-EP402R-EP153R-I177L-K205Rubiqutin. The mean titer of antibodies in the high-dose group was more than 70000, and that in the medium-dose group was also 50000, showing significant difference from that in the control group.

### 40.2 Detection of Cellular Immune Response

Ten SPF-grade mice (6-8 weeks of age) were randomly divided into two groups, five for each group. Mice were immunized with pAd5LCL3-EP402R-EP153R-I177L-K205Rubiqutin according to the groupings shown in Table 8. The injection method was as follows: intramuscular injection was performed on the medial aspect of the posterior thigh. Injection dose: 100 ul.

TABLE 8
Groups of Vaccinated Mice

| | | | Immune | Number |
|---|---|---|---|---|
| group | Vector vaccine | dosage | mode | of mice |
| Experiment | pAd5LCL3-EP402R- | 1*10\hat{ }7 FFU | intramuscular | 5 |
| | EP153R-I177L- | | injection | |
| | K205Rubiqutin | | | |
| Control | pAd5LCL3 | 1*10\hat{ }7 FFU | intramuscular | 5 |
| | | | injection | |

The mice were sacrificed 14 days after immunization, and the splenic lymphocytes were isolated. PK15 cells transfected with the shuttle plasmids pSE1-EP402R-IRES-EP153R and pSE4-I177L-2A-K205Rubiqutin were stimulated for 6 hours and protein secretion blockers were added to block cytokine secretion. After 6 hours, Fc receptors were blocked, dead cells and cell surface molecular markers were stained, and intracellular cytokines were stained after the cells were fixed and perforated. Cell surface markers included CD4 and CD8, and intracellular cytokines included IFNγ and IL2. The expression levels of IFNγ and IL2 in CD4+T cells and CD8+T cells stimulated by the target protein were analyzed by flow cytometry (CyExpert).

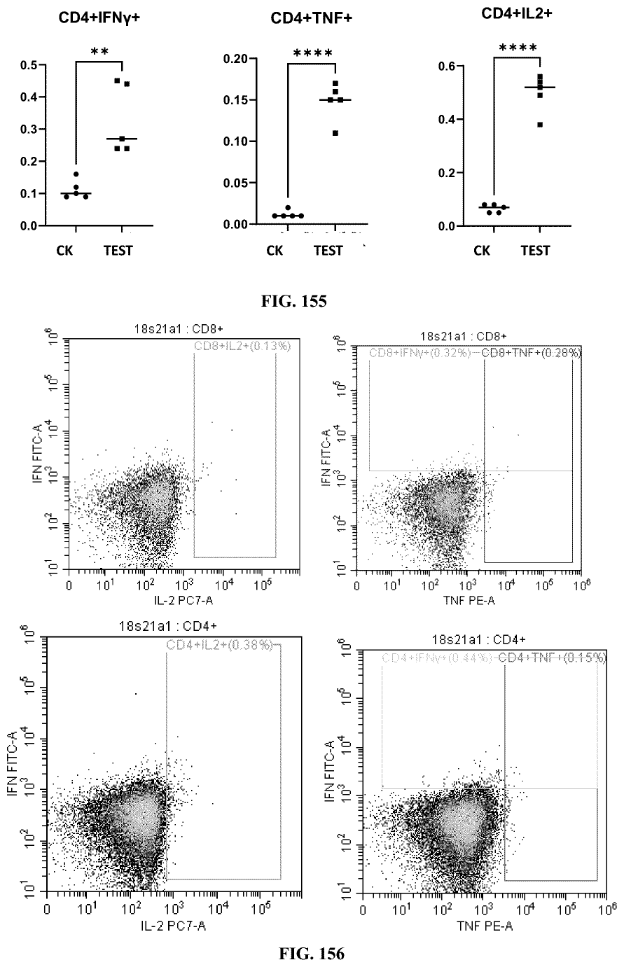

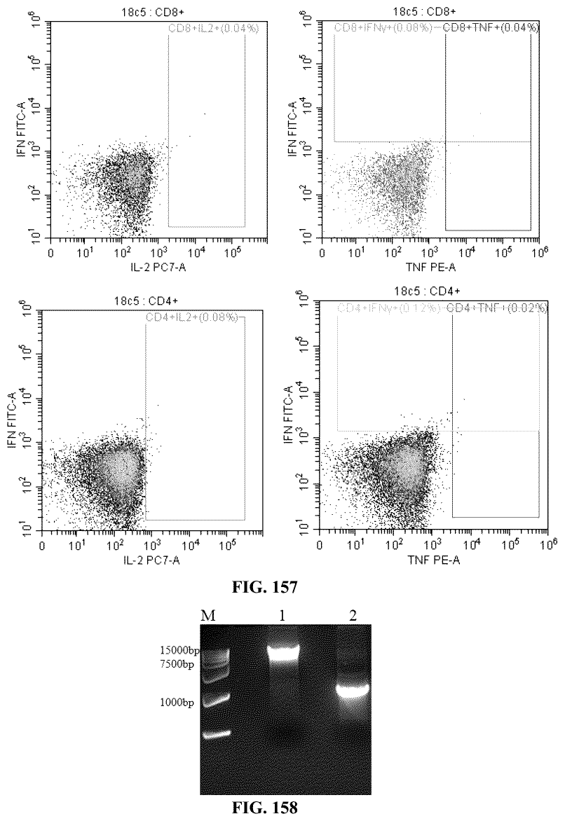

pAd5LCL3-EP402R-EP153R-I177L-K205Rubiqutin induced CD8+T cell and CD4+T cell immune response was shown in FIG. 154 and FIG. 155. The representative results were shown in FIG. 156 to FIG. 157. FIG. 156 was the representative diagram of cellular immune response after intramuscular injection of pAd5LCL3-EP402R-EP153R-I177L-K205Rubiqutin, and FIG. 157 was the representative diagram of blank control immune response. The results showed that, 14 days after immunization, after the spleen cells were stimulated by the target protein, the levels of IFNγ, TNFα and IL2 expressed in CD8+T cells were significantly higher than those in the HAd5 vector control group (Control)(P<0 0.05). After stimulation, the expression levels of IFNγ, TNFα and IL2 in CD4+T cells were significantly higher than those in the HAd5 vector control group (P<0.05).

### 40.3 Summary of Immunogenicity Evaluation of Mouse Model

The recombinant adenovirus pAd5LCL3-EP402R-EP153R-I177L-K205Rubiqutin was immunogenic and induced high levels of serum IgG antibodies in mice. High doses of 1*10⁸ FFU and medium doses of 1*10⁷ FFU resulted in high immunologically induced titers. The results of cellular immune response showed that intramuscular injection of the adenovirus vector vaccine of 1*10⁷FFU could induce specific cellular immune response in the immunized mice.

### Example 41: Construction of the African Swine Fever Human Adenovirus Type 5 Vector E1 Region Shuttle Plasmid pS5E1-F317L-IRES-A151R

1. Construction of human adenovirus type 5 vector E1 region shuttle plasmid

The human adenovirus type 5 vector E1 region shuttle plasmid pS5E1 was successfully constructed according to the method provided in Example 4.

2. Construction of shuttle plasmid pSSE1-F317L-IRES-A151R of African swine fever human adenovirus type 5 vector

1) ligation of pS5E1 to the IRES fragment

pS5E1-IRES was successfully constructed according to the method provided in Example 4.

2) ligation of pS5E1-IRES to F317L fragment

\textcircled{1} primer synthesis

F317L-BamHI-F:
(SEQ ID NO: 154)
cgc GGATCC gccaccATGGTGGAGACCCAGATGGACA
F317L-EcoRV-R:
(SEQ ID NO: 155)
ccg GATATC TCAGTGGTGGTGGTGGTGGTG

\textcircled{2} PCR amplification of F317L fragment

Amplification system: 25 ul of Q5 enzyme, 1 uL of 10 uM primer F317L-BamHI-F, 1 uL of 10 uM primer F317L-EcoRVR, 1 uL of template F317L, and water supplementing to 50 ul; PCR program: 98° C., 10 s; 98° C., 5s, 60° C., 30s, 72° C., 20s, 35 cycles; 72° C., 5 min.

\textcircled{3} The F317L fragment was purified by Axygen™ PCR purification kit.

\textcircled{4} The target fragment F317L was digested with pSSE1-IRES vector

Enzyme digestion reaction system: vectors pSSE1-IRES, F317L fragment ˜2 ug, BamHI and EcoRV 1 uL each; 10×cutsmart buffer 5 ul; Replenish water to 50 ul. Reaction condition: 37° C., 30 min; Inactivated at 65° C. for 20 min. Recovery and purification of gum. The electrophoresis detection of the digested product was shown in FIG. 158, where lane 1 was pSSE1-IRES, BamHI and EcoRV double-digestion, lane 2 was fragment F317L, BamHI and EcoRV double-digestion, and M was DL 15,000 DNA Marker.

\textcircled{5} The target fragment F317L was connected with pSSE1-IRES

Ligation system: PSSE1-IRES (100 ng); F317L fragment (vector:fragment=1: 3 molar ratio); T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

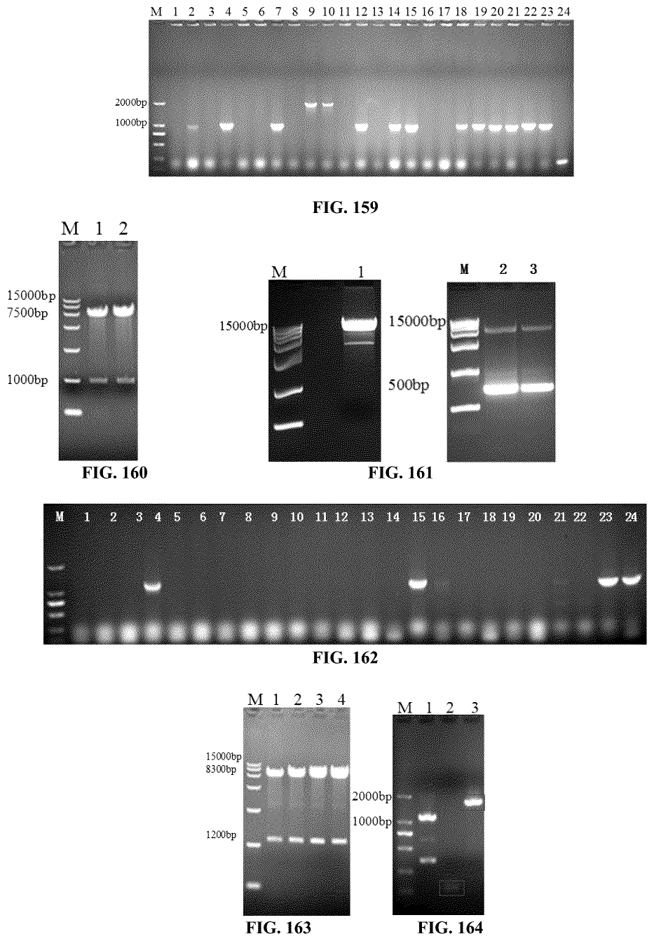

Amplification system: Q5 enzyme 10 ul, 10 uM primer F317L-BamHI-F 1 uL, 10 uM primer IRES-NotI-R 1 ul, and supplementing water to 20 ul. PCR program: 98° C., 10 s; 98° C., 5s, 60° C., 30s, 72° C., 20s, 35 cycles; 72° C., 5 min. Electrophoresis verification was performed as shown in FIG. 159, in which No. 1-24 was a colony and M was DL 2,000 DNA Marker. As shown in FIG. 159, No. 9 and No. 10 were positive colonies.

\textcircled{7} Plasmid digestion verification (BamHI and EcoRV), 9 and 10 colonies were selected for plasmid extraction and digestion verification. The results were as shown in FIG. 160, all of which were positive plasmids.

3) ligation of pS5E1-F317L-IRES to fragment A151R

\textcircled{1} primer synthesis

A151R-NotI-F:
(SEQ ID NO: 156)
aaatat GCGGCCGC ATGAACAAGAAGATCATCGTGATG
A151R-6His-XhoI-R:
(SEQ ID NO: 157)
cggCTCGAGTCAGTGGTGGTGGTGATGGTGCTGGAAGATG
TTGGGGGACATGA

\textcircled{3} PCR amplification of A151R fragment

Amplification system: 25 ul of Q5 enzyme, 1 μL of primer A151R-NotI-F, 1 μL of primer A151R-6 His-XHOI-R, and 50 ul; of template A151R; Reaction condition: 98° C. for 30 s; 98° C. for 10 s, 68° C. for 30 s, 72° C. for 30 s, 35 cycles; 72° C. 5 min.

\textcircled{3} The A151R fragment was purified by an Axygen™ PCR purification kit.

\textcircled{4} The target fragment A15IR was digested with pSSE1-F317L-IRES vector

Enzyme digestion reaction system: vectors pSSE1-F317L-IRES, A151R fragment ˜2 ug, NotI and XhoI 1 uL each; 10×cutsmart buffer 5 ul; Replenish water to 50 ul. Reaction condition: 37° C., 30 min; Inactivated at 65° C. for 20 min. Recovery and purification of gum. The electrophoresis detection of the enzyme digestion product was shown in FIG. 161, where lane 1 was subjected to enzyme digestion with pS5E1-F317L-IRES, NotI and XhoI; Lanes 2 and 3 show the A151R fragment, digested by NotI and XhoI, and M was DL 15,000 DNA Marker.

\textcircled{5} pS5E1-F317L-IRES vector was connected with A151R fragment

Linkage system: pS5E1-F317L-IRES 100 ng; A151R fragment 50 ng; T4 DNA ligase 1 ul; 10×ligase buffer 1 ul; Replenish water to 10 ul. Reaction condition: room temperature, 30 min. The ligated products were transformed into DH5α competent cells, plated on plates containing ampicillin resistance and incubated at 37° C. for 12-16 hours.

\textcircled{6} colony PCR

Amplification system: Q5 enzyme 10 ul, 10 uM primer IRES-EcoRV-F 1 ul, 10 uM primer A151R-6His-XhoI-R 1 ul, and water supplement to 20 ul; PCR program: 98° C., 10 s; 98° C., 5s, 60° C., 30s, 72° C., 20s, 35 cycles; 72° C., 5 min; Electrophoresis verification was performed as shown in FIG. 162, whereNo. 1-24 was the colony, and M was DL 2,000 DNA Marker.

\textcircled{7} Plasmid digestion verification of BamHI and XhoI: The colonies of 4, 15, 23 and 24 were selected for plasmid extraction and digestion verification. The results were shown in FIG. 28. For the digestion identification of BamHI and EcoRV, M was DL 2,000 DNA Marker. As shown in FIG. 163, the enzyme digestion result was correct, and the successful construction of the African swine fever human adenovirus type 5 vector E1 region shuttle plasmid pS5E1-F317L-IRES-A151R was sequenced correctly, and the vector map was shown in FIG. 51.

### Example 42: Construction of African Swine Fever Human Adenovirus Type 5 Vector E4 Region Shuttle Plasmid pS5E4-P34-2A-pp62

1. human adenovirus type 5 vector E4 region shuttle plasmid construction

The human adenovirus type 5 vector E4 region shuttle plasmid pS5E4-EGFP was successfully constructed according to the method provided in Example 5.

2. Construction of African swine fever human adenovirus type 5 vector E4 region shuttle plasmid pS5E4-P34-2A-pp62

1) primer design

EF1a-BamHI-P34-F:
(SEQ ID NO: 158)
tccaagctgtgaccggcgcctacGGATCCGCCACCATGGGGAAT
CGCGGGTCTTCT
P34-2A-R:
(SEQ ID NO: 159)
GCCCTTTTTGGCGCAGCTGTT
P34-2A-F:
(SEQ ID NO: 160)
AGAACAGCTGCGCCAAAAAGGGCGGAAGCGGAGCTACTAACTTC
2A-pp62-R:
(SEQ ID NO: 161)
AACTGCTTCATGTTGCTGGGCATAGGTCCAGGGTTCTCCTCCA
pp62-F:
(SEQ ID NO: 162)
ATGCCCAGCAACATGAAGCAG
pp62-XhoI-pS5E4-R:
(SEQ ID NO: 163)
GGGTTTAAACGGGCCCTCTAGACTCGAGttaCAGCAGCTTCAG
GATCTCGTT

2) amplifying the target fragments P34, 2A and pp62

\textcircled{1} A The P34 fragment was amplified using the P34 gene synthetic fragment as the template and EF1α-BamHI-P34-F and P34-2A-R as the primers; Amplification system: P34 gene synthetic fragment 50 ng, 10 uM EF1α-BamHI-P34-F primer 1 ul, 10 uM P34-2A-R primer 1 ul, Q5 high-fidelity enzyme 20 ul; Replenish water to 40 ul; PCR procedure: 98° C., 10 sec; 98° C., 5 sec, 60° C., 30 sec, 72° C., 40 sec, 35 cycles; 72° C., 5 min.

\textcircled{2} The synthetic fragment of 2A gene was used as the template, and P34-2A-F and 2A-pp62-R were used as the primers to amplify the 2A fragment; Amplification system: synthetic fragment of 2A gene (50 μg), 10 μM p34-2A-F primer (1 ul), 10 μM 2A-PP62-R primer (1 ul), and Q5 high-fidelity enzyme (20 ul); Replenish water to 40 ul; PCR procedure: 98° C., 10 sec; 98° C., 5 sec, 60° C., 30 sec, 72° C., 20 sec, 35 cycles; 72° C., 5 min.

\textcircled{3} The synthetic fragment of pp62 gene was used as the template and pp62-F and pp62-XhoI-pS5E4-R as the primers to amplify fragment 2A; Amplification system: synthetic fragment of pp62 gene (50 μg), 10 μM PP62-F primer (1 ul), 10 μM PP62-XhoI-pS5E4-R primer (1 ul), and Q5 high-fidelity enzyme (20 ul). Replenish water to 40 ul; PCR procedure: 98° C., 10 sec; 98° C., 5 sec, 60° C., 30 sec, 72° C., 40 sec, 35 cycles; 72° C., 5 min.

The electrophoretic detection of the PCR product was shown in FIG. 164, wherein lane 1 was fragment P34; lane2 was fragment 2A; lane 3 was fragment pp62, and M was DL 2,000 DNA Marker.

3) The target fragment was purified by an Axygen™ gel extraction kit.

4) Amplifying P34-2A fragments by fusion PCR

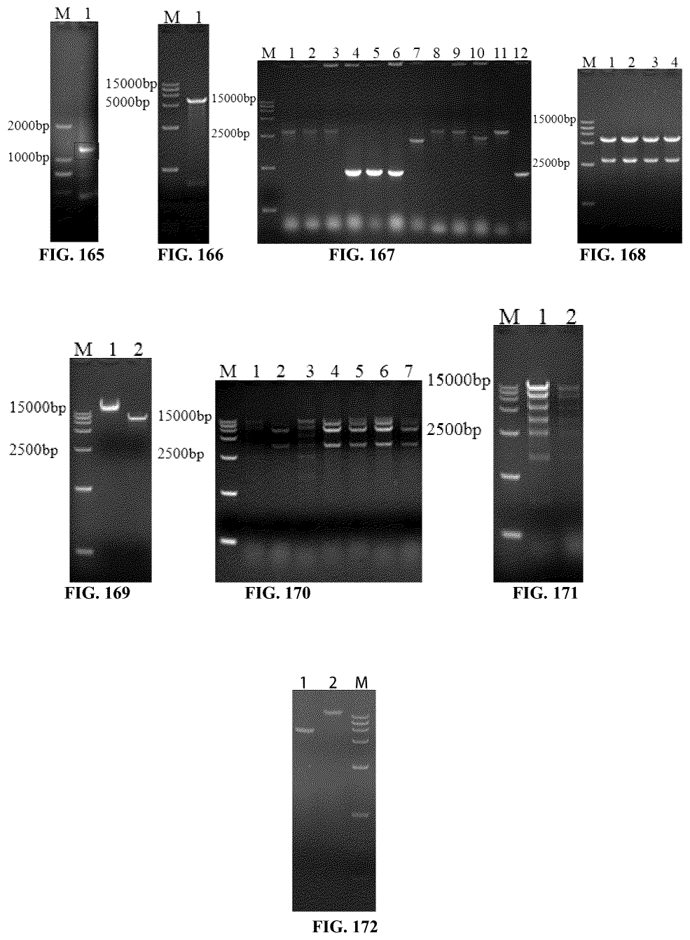

Amplification system: 50 ng of recovered fragment of P34 gel, 50 ng of recovered fragment of 2A gel, 1 ul of 10 μM EF1α-BamHI-p34-F primer, 1 ul of 10 μM 2A-PP62-R primer, and 25 ul; of Q5 high-fidelity enzyme; Replenish water to 50 ul; PCR procedure: 98° C., 10 sec; 98° C., 5 sec, 60° C., 30 sec, 72° C., 40 sec, 35 cycles; 72° C., 5 min. The fusion results were shown in FIG. 165, where lane 1 was the P34-2A fragment and M was DL 2,000 DNA Marker.

5) Enzyme digestion with pS5E4-EGFP vector

Enzyme digestion reaction system: vector pS5E4-EGFP 2 ug, BamHI and XhoI 1 uL each; 10×cutsmart buffer 5 ul; Replenish water to 50 ul. Reaction condition: 37° C., 30 min; Inactivated at 65° C. for 20 min. Recovery and purification.

6) purifying the vector fragment by an Axygen™ gel extraction kit;

The gel recovery results were shown in FIG. 166, where lane 1 was the fragment pS5E4-EGFP, BamHI and XhoI double enzyme digestion for gel recovery, and M was DL 15,000 DNA Marker.

7) seamless clone connection and transformation of pS5E4-EGFP glue recovery vector with P34-2A fragment and pp62

Ligation system: pS5E4-EGFP gel recovery (100 ng), P34-2A fragment (50 ng), pp62 fragment (50 ng), 2×Smealess Cloning Mix 5 ul, replenished to 10 ul. Reaction condition: 50° C., 40 min. The ligated products were transformed into DH5α competent cells, plated on plates containing ampicillin resistance and incubated at 37° C. for 12-16 hours.

8) plasmid validation

\textcircled{1} colony PCR

Using EF1α2(d)-F and HBV(jd)-R as primers, the target fragment was amplified by colony PCR and verified by agarose gel assay, as shown in FIG. 167, where No. 1-12 was the colony and M was DL 15,000 DNA Marker.

\textcircled{2} enzyme digestion verification

The No. 1, No. 2, No. 9 and No. 11 positive clone were picked and place in 5 mL LB liquid medium containing ampicillin resistance for culture for 12-15 hours, and plasmids were extract for double enzyme digestion verification of BmHI and XhoI; The enzyme digestion results were shown in FIG. 168, where lanes 1, 2, 9 and 11 were the double enzyme digestion verification of positive clones BamHI and XhoI, and M was DL 15,000 DNA Marker. The enzyme digestion result was correct and the sequencing was correct. The African swine fever human adenovirus type 5 vector E4 region shuttle plasmid pSSE4-P34-2A-pp62 was successfully constructed, and the vector map was shown in FIG. 53.

### Example 43: pAd5LCL3-F317L-A151R-p34-2A-PP62 Plasmid Constructed Recombinantly with pAd5LCL3

1. Autologous recombination of shuttle plasmid pSSE1-F317L-IRES-A151R and adenovirus vector plasmid pAd5LCL3

1) PacI and SwaI perform enzyme digestion on the shuttle plasmid pS5E1-F317L-IRES-A151R and the adenovirus vector plasmid pAd5LCL3, and the enzyme digestion reaction system was as follows:

A, the shuttle plasmid pSSE1-F317L-IRES-A151R3 s g; PacI 2 μl; buffer cutsmart 4 μl; Replenish water to 40 μl.

B, adenovirus vector plasmid pAd5LCL3 3 ug; SwaI 2 μl; Buffer 3.1 4 μl; Replenish water to 40 μl.

Reaction condition were 37° C. for 1 h; Inactivated at 65° C. for 20 min.

2 ul of agarose gel was used for validation and the results were shown in FIG. 169, with lane 1 being pAd5LCL3 and lane 2 being pS5E1-F317L-IRES-A151R.

2) dephosphorylation of that enzyme digestion product

Reaction system: 37.5 μL enzyme digestion reaction solution; 1 μL dephosphorylase; Dephosphorylated buffer 5 μL; Refill water to 50 μL. Reaction condition were 37° C. for 1 h; Inactivated at 65° C. for 5 min.

3) Use OMEGA Ultra-Sep Gel Extraction Kit to recover the vectors and fragments.

4) 100 ng of the purified shuttle plasmid and 100 ng of the purified adenovirus vector were co-transformed into BJ5183 competent cells, and the transformed product was coated with an LB plate containing Kan and cultured at 37° C. for 12-16 h.

5) The colonies were selected and cultured in 5 mL LB liquid medium containing Kan under shaking at 37° C. for 12-16 h, and the plasmids were extracted for XhoI enzyme digestion verification. The results were shown in FIG. 170, where Lane 1-7 was the clone of pAd5LCL3-F317L-IRES-A151R, m: 15000 bp marker. As shown in FIG. 38, the enzyme cut of Clone 3 was correct.

6) The No. 3 positive plasmid was converted into DH5α competent state, a colony was selected and cultured in 5 mL LB liquid medium containing Kan under shaking at 37° C. for 12-16 h, and the plasmid was extracted for XhoI enzyme digestion verification again. The enzyme digestion result was shown in FIG. 171. As shown in FIG. 171, the enzyme digestion result was correct, and the adenovirus vector plasmid pAd5LCL3-F317L-IRES-A151R was successfully constructed.

2. Autologous recombination of shuttle plasmid pS5E4-P34-2A-pp62 and adenovirus vector plasmid pAd5LCL3-F317L-IRES-A151R to obtain pAd5LCL3-F317L-A151R-P34-PP62

1) PacI and I-sceI perform enzyme digestion on shuttle plasmid pS5E4-P34-2A-pp62 and adenovirus vector plasmid pAd5LCL3-F317L-IRES-A151R, and the enzyme digestion reaction system was as follows:

A. Shuttle plasmid PS5E4-P34-2A-PP623p g; PacI 2 μl; 10×cutsmart buffer 4 μl; Replenish water to 40 μl.

B, adenovirus vector plasmid pAd5LCL3-F317L-IRES-A151R3 ug; I-sceI 2 μl; Buffer cutsmart 4 μl; Replenish water to 40 μl.

Reaction condition were 37° C. for 1 h; Inactivated at 65° C. for 20 min.

2 ul of agarose gel was used for validation and the results were shown in FIG. 172, with lane 1 being pS5E4-P34-2A-pp62 and lane 2 being pAd5LCL3-F317L-IRES-A151R.

2) dephosphorylation of that enzyme digestion product

Reaction system: 37.5 μL enzyme digestion reaction solution; 1 μL dephosphorylase; Dephosphorylated buffer 5 μL; Refill water to 50 μL. Reaction condition were 37° C. for 1 h; Inactivated at 65° C. for 5 min.

3) Use OMEGA Ultra-Sep Gel Extraction Kit to recover the vectors and fragments.

4) 100 ng of the purified shuttle plasmid and 100 ng of the purified adenovirus vector were co-transformed into BJ5183 competent cells, and the transformed product was coated with an LB plate containing Kan and cultured at 37° C. for 12-16 h.

5) Six colonies were selected and cultured in 5 mL LB liquid medium containing Kan under shaking at 37° C. for 12-16 h, and the plasmids were extracted for XhoI enzyme digestion verification. The results were shown in FIG. 173, where lanes 1-4 were plasmids, and M was DL 15,000 DNA Marker. It can be seen that plasmid No. 2 was correct.

6) transform that No. 2 positive plasmid into DH5α competence; One colony was selected and cultured in 5 mL LB liquid medium containing Kan under shaking at 37° C. for 12-16 h, and the plasmid was extracted for XhoI restriction enzyme digestion verification again. The enzyme digestion result was shown in FIG. 174, where the plasmid XhoI in lane 1 was pAd5LCL3-F317L-A151R-P34-2A-PP62, and the plasmid M was DL 15,000 DNA Marker. As shown in FIG. 174, the enzyme digestion result was correct. The adenovirus vector plasmid pAd5LCL3-F317L-A151R-P34-pp62 was successfully constructed, and the vector map was shown in FIG. 54.

### Example 44: Package of Recombinant Adenovirus

Package the pAd5LCL3-F317L-A151R-P34-pp62 plasmid with 293TD37 cells as follows:

Preparation of 293TD37 cells: The cells were prepared one day before transfection. The 293TD37 cells to be transfected were inoculated into a 6-well plate at 0.5×10⁶/well, and incubated at 37° C. with 5% CO₂ for 24 hours. On the day of transfection, the cells showed 40-50% confluency.

Linearization of plasmid pAd5LCL3-F317L-A151R-P34-pp62: The plasmid to be transfected was digested with PacI enzyme, incubated at 37° C. for 40 min, and inactivated at 65° C. for 20 min.

Transfection: The linearized 2 pg plasmid and PEI were diluted with 100 μl serum-free medium, respectively. The plasmid diluent was added into the PEI diluent, and repeatedly aspirated for 5 times or vortexed for 10 seconds to be mixed evenly, and incubated for 10 minutes at room temperature to form a transfection complex. During incubation, cell culture medium was gently aspirated from the plates, 2 mL of fresh growth medium was added, and after 10 minutes the transfection complex was added to the cells in fresh medium.

Cell culture: the transfected 293TD37 cells were incubated at 37° C. for 72-96 hours in a 5% CO₂ incubator; 6-well plate cell suspensions were collected in 1.5 ml centrifuge tubes, TP0, 72-96 hours after viral plasmid transfection.

Continuous inoculation: The collected cell suspension was repeatedly frozen and thawed at −80° C. for 3 times, centrifuged at 2000 g for 10 minutes at 4 C, and 500 μl of supernatant was collected to infect 293TD37 cells (293TD37 cells need to be prepared one day in advance). The cells were incubated at 37° C. with 5% CO₂for 60 minutes, followed by the addition of 2 mL of FBS medium, followed by culture at 37° C. with 5% CO₂ for 72 hours, and the cell suspension, namely TP1, was collected. The previous steps were repeated and the cell suspension, TP2, was collected. The drug was continued until the cells became diseased.

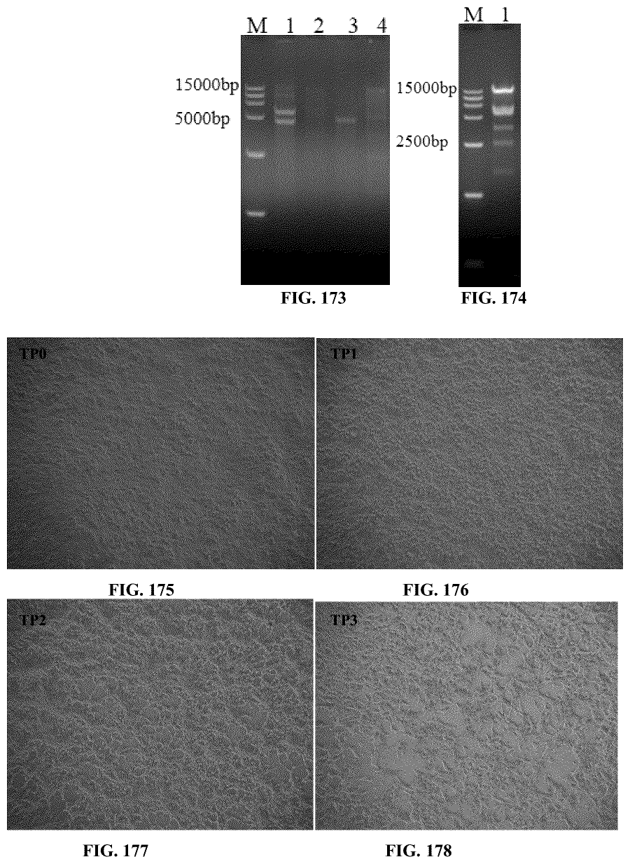

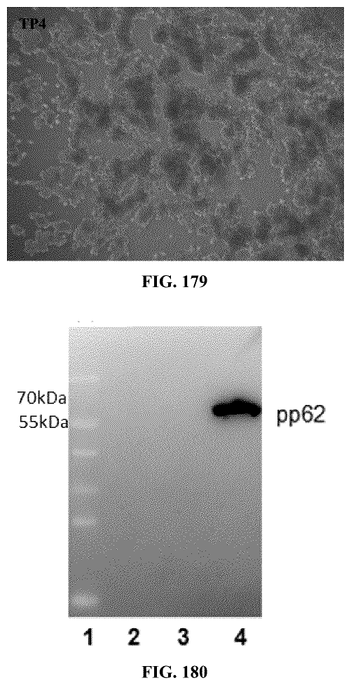

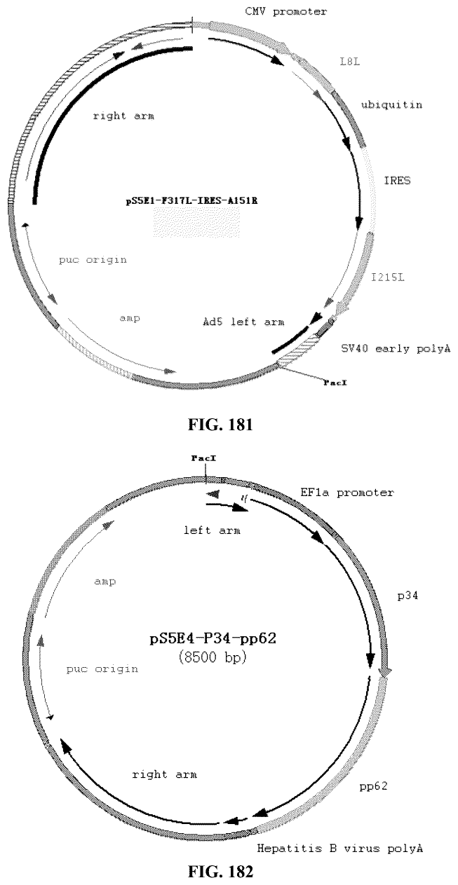

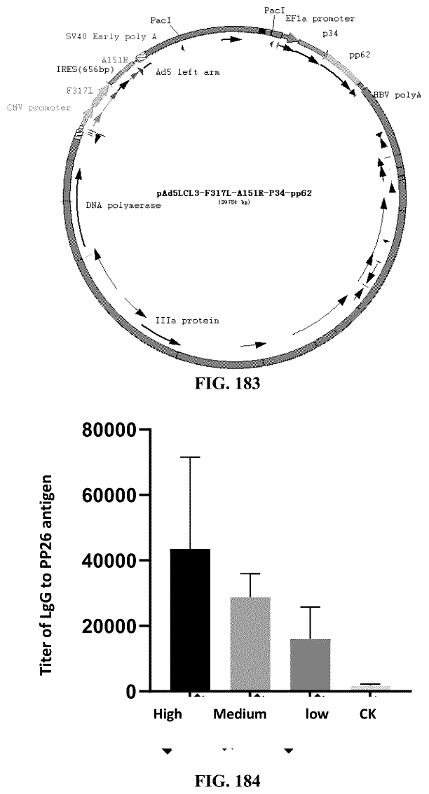

Cytopathic effect: When the 293TD37 cells were cultured from TP0 to TP4, the cells gradually became diseased until the 293TD37 cells were completely diseased at TP4. The cytopathic effects caused by TP0 to TP4 were shown in FIG. 175-179, respectively. TP4 was completely diseased.

### Example 45: Detection of Titer of African Swine Fever Multiantigen Recombinant Adenovirus Vaccine

The 293TD37 cells were prepared. The cells with good growth in T75 culture flask were taken, the supernatant was discarded, the cells were washed with PBS, and digested with 0.25% trypsin, and then 10 mL of fresh DMEM medium containing 10% fetal bovine serum was added to stop the digestion, which was then blown, mixed, inoculated into 6-well plates (5×10⁵/mL, 2 ml per well), and allowed to stand for culture in a 37° C. 5% CO₂ incubator. After 24 hours, when the cells adhered to grow into single-layer cells, the culture medium was discarded, and the recombinant adenovirus was continuously diluted 10³ to 10⁶ times with serum-free DMEM maintenance solution, and two wells were inoculated with each dilution degree at 250 uL per well. After one hour of infection, the supernatant was discarded to supplement the complete culture medium, and then the medium was allowed to stand for culture in a 5% carbon dioxide incubator at 37° C. After 24 h, the supernatant was discarded and the cells were washed with PBS (1 mL per well). After PBS was discarded, 1 mL of cold formaldehyde was added into each well for fixation, and formaldehyde was discarded at room temperature for 10 min. Then the cells were washed with PBS (1 mL per well), followed by adenovirus antibody-FITC (1 ml per well). After 1 h at room temperature, the cells were washed with PBS again (1 mL per well). After two times, 1 mL of PBS was added into each well and counted under fluorescence microscope (200 times, 10 continuous fields). Calculation: Virus titer (FFU/mL)=Mean×1013×4×10⁽⁻ⁿ⁾. The FFU of the pAd5LCL3-F317L-A151R-P34-PP62 virus was 2.4×10⁸FFU/mL, with a high titer.

### Example 46: Detection of Stability of African Swine Fever Multiantigen Recombinant Adenovirus Vaccine pAd5LCL3-F317L-A151R-P34-pp62

The 293TD37 cells were prepared. The cells that grew well in T75 culture flask were taken, the supernatant was discarded, the cells were washed with PBS, and digested with 0.25% trypsin. Then 10 mL of fresh DMEM medium containing 10% fetal bovine serum was added to stop the digestion, and then the cells were blown and mixed evenly. The 293TD37 cells were planted into 6-well plates (5×10⁵ cells/mL, 2 mL/well), incubated for 1 hour at room temperature to adhere to the wall, and incubated for microscopic observation of its attachment degree. pAd5LCL3-F317L-A151R-P34-pp62 virus particles were used for infection, and the titer of infection was 5 MOI/well. After the 293TD37 cells developed lesions 48 hours later, the cells were collected, repeatedly frozen and thawed for three times, and then centrifuged at 2000 g; the collected supernatant was detected for FFU, and then new 293TD37 cells were reinfected until the 30th generation. The collected virus solutions of passages 5, 10, 15, 20, 25 and 30 were tested, and the genome of the virus was found to be still intact, indicating that the replication-defective pAd5LCL3-F317L-A151R-P34-pp62 virus could be stably packaged in 293TD37 cells.

### Example 47: Detection of African Swine Fever Multiantigen Recombinant Adenovirus Vaccine pAd5LCL3-F317L-A151R-P34-pp62 Recovery Mutation (RCA)

PAd5LCL3-F317L-A151R-P34-pp62 virus RCA detection, detection method was as follows:

1. Prepare pAd5LCL3-F317L-A151R-P34-pp62 virus solution, measure its virus titer and determine the concentration of virus particles. The virus solution was mixed with 1% Universal Nuclease (7.5-15 units/mL virus solution) to digest the DNA of the host cell, and water-bath was conducted at 37° C. for 40 min. Using a 300Kd ultrafiltration centrifuge tube, the virus particles were collected after centrifugation at 1000 g for 30 min, followed by elution with 1×PBS. A260 was measured, and the particle concentration=A260*1.1*10¹² VP/mL.

2. Virus infection: A 6-well plate of A549 cells was prepared, with each well cell being 2.5×10⁵/well. The medium was discarded and washed with PBS once. Adenovirus was inoculated with virus at 1×10⁹ vp/well to infect A549 cells. Wild-type human adenovirus type 5 was used as the control at 37° C. and 5% CO₂. After 1 h, the virus solution was discarded and supplemented with 5% complete medium. The cells were cultured at 37° C. and 5% CO₂ for 48 h.

3. Immunostaining was performed, and the cell supernatant was discarded. The cells were surface washing-plated with PBS and fixed with iced methanol. The cells were placed at −20° C. for 20 min and washed by 1×PBS for three times, each time for 5 min. Then 2 ml 1% BSA-PBS solution was added into each well, and the cells were placed in a shaking table for incubation for 1 h. After the supernatant was discarded, human adenovirus type 5 fluorescent antibody (1:500 dilution) was added and incubated for 1 h, followed by washing with 1×PBS for three times, 5 min each time.

RCA was calculated using the equation as observed under a 10-fold fluorescence microscope

$$RCA = ( average positive cell field ) \times ( 374 field / well ) \times ( dilution factor ) ) / Total VPs in 0.5 ml viral sample$$

The judging standard was that the level of RCA was less than 1RCA/3×10¹⁰ vp. Through counting that the RCA level of the pAd5LCL3-F317L-A151R-P34-PP62 was less than 1 RCA/3*10¹⁰ VP, the replication-defective pAd5LCL3-F317L-A151R-P34-PP62 virus prepared by the invention can be stably packaged in 293TD37 cells and was not converted into wild type or has low probability of being converted into wild type.

### Example 48: Detection of Protein Expression of African Swine Fever Multiantigen Recombinant Adenovirus Vaccine pAd5LCL3-F317L-A151R-P34-pp62

The 293TD37 cells were prepared one day in advance and placed in a 12-well cell culture plate. The 293TD37 cells were infected with the African swine fever multiantigen recombinant adenovirus vaccine pAd5LCL3-F317L-A151R-P34-pp62 virus, and the cells became diseased 48 hours later. All 1 ml of cells were collected, washed with PBS, and prepared for Western Blot detection. The target protein was detected by self-made pp62 mouse antiserum. The pp62 mouse antiserum was obtained by immunizing mice with pp62 protein expressed by the insect SF9 system. The size of pp62 protein was 60 kda.

The experimental results were shown in FIG. 180. Lane 4 was the sample of pAd5LCL3-F317L-A151R-P34-PP62 infected with 293TD37 cells. It could be clearly seen that the pp62 protein was normally expressed, so that the protein of the pAd5LCL3-F317L-A151R-P34-pp62 vaccine could be normally expressed in 293 cells.

Example 49 immunologic evaluation of african swine fever multiantigen recombinant adenovirus vaccine pAd5LCL3-F317L-A151R-P34-pp62 in a murine model

### 49.1 Detection of Humoral Immune Response of Vaccine

Twenty SPF-grade mice (6-8 weeks of age) were randomly divided into four groups, five for each group.

Mice were immunized with pAd5LCL3-F317L-A151R-P34-PP62 according to the groupings shown in Table 9. The injection method was as follows: intramuscular injection was performed on the medial aspect of the posterior thigh. Injection dose: 100 ul.

TABLE 9
Groups of Vaccinated Mice

| | | | Immune | Number |
|---|---|---|---|---|
| group | Vector vaccine | dosage | mode | of mice |
| High | pAd5LCL3-F317L- | 1*10\hat{ }⁸ FFU | intramuscular | 5 |
| dose | A151R-P34-pp62 | | injection | |
| Medium | pAd5LCL3-F317L- | 1*10\hat{ }⁷ FFU | intramuscular | 5 |
| dose | A151R-P34- pp62 | | injection | |
| Low | pAd5LCL3-F317L- | 1*10\hat{ }⁶ FFU | intramuscular | 5 |
| dose | A151R-P34-pp62 | | injection | |
| control | pAd5LCL3 | 1*10\hat{ }⁷ FFU | intramuscular | 5 |
| | | | injection | |

Blood was collected 14 days after immunization, and the serum was isolated. The titer of pp62 antibody against the African swine fever target protein (pp62 protein prepared by us and expressed in insect cells) was detected by indirect ELISA. The test results were shown in FIG. 55: After intramuscular injection of pAd5LCL3-F317L-A151R-P34-pp62, mice were able to produce high concentrations of IgG antibodies against pp62 protein. The average titer of antibodies in the high-dose group was over 105, and that in the medium-dose group was 70000, showing significant difference from that in the control group.

### 49.2 Cell Immune Response Detection

Ten SPF-grade mice (6-8 weeks of age) were randomly divided into two groups, five for each group. Mice were immunized with pAd5LCL3-F317L-A151R-P34-PP62 according to the groupings shown in Table 10. The injection method was as follows: intramuscular injection was performed on the medial aspect of the posterior thigh. Injection dose: 100 ul.

TABLE 10
Groups of Vaccinated Mice

| | | | Immune | Number |
|---|---|---|---|---|
| group | Vector vaccine | dosage | mode | of mice |
| Experiment | pAd5LCL3-F317L- | 1*10\hat{ }⁷ FFU | intramuscular | 5 |
| | A151R-P34-pp62 | | injection | |
| control | pAd5LCL3 | 1*10\hat{ }⁷ FFU | intramuscular | 5 |
| | | | injection | |

The mice were sacrificed 14 days after immunization, and the splenic lymphocytes were isolated. PK15 cells transfected with the shuttle plasmids pS5E1-P72-IRES-B602L and pS5E4-P30-2A-P54 were stimulated and cultured for 6 hours, while protein secretion blockers were added to block cytokine secretion. After 6 hours, Fc receptors were blocked, dead cells and cell surface molecular markers were stained, and intracellular cytokines were stained after the cells were fixed and perforated. Cell surface markers included CD4 and CD8, and intracellular cytokines included IFNγ and IL2. The expression levels of IFNγ and IL2 in CD4+T cells and CD8+T cells stimulated by the target protein were analyzed by flow cytometry (CyExpert).

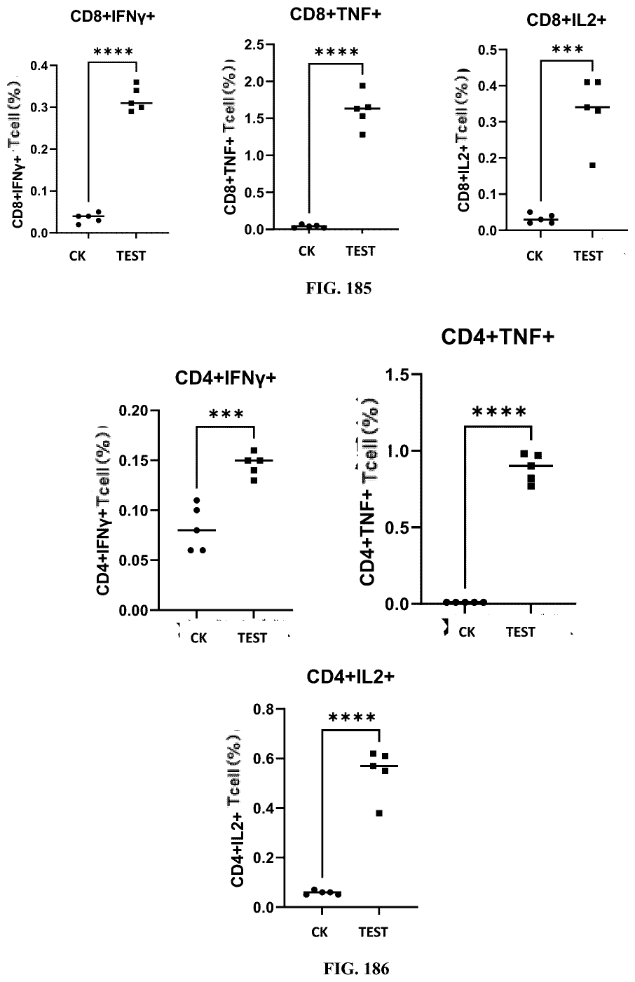

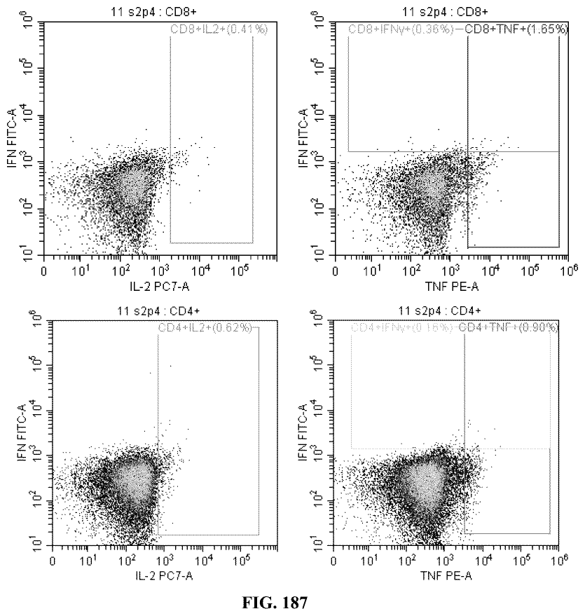

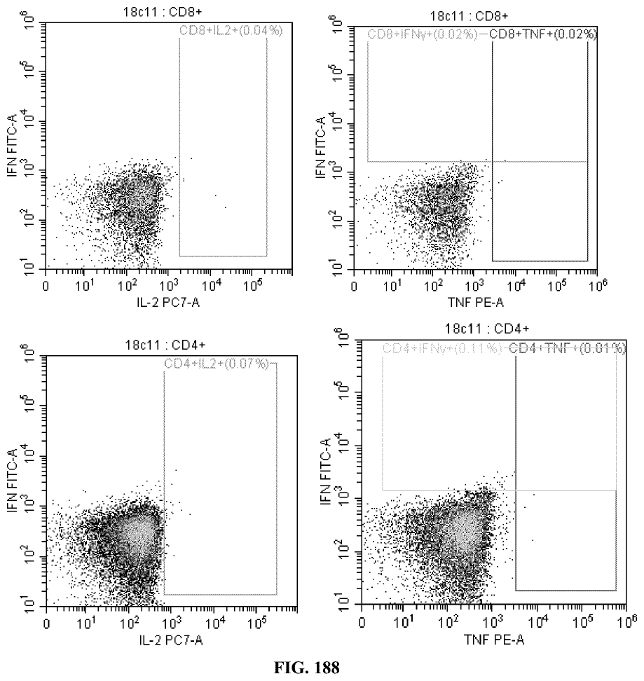

The CD8+T cell and CD4+T cell immune responses induced by pAd5LCL3-P72-B602L-P30-P54 were shown in FIG. 184 and FIG. 185, and the representative results were shown in FIG. 186 to FIG. 187, where FIG. 186 was the representative diagram of cellular immune response after intramuscular injection of pAd5LCL3-P72-B602L-P30-P54, and FIG. 187 was the representative diagram of blank control immune response. The results showed that, 14 days after immunization, after the spleen cells were stimulated by the target protein, the levels of IFNγ, TNFα and IL2 expressed in CD8+T cells were significantly higher than those in the HAd5 vector control group (Control)(P<0 0.05). After stimulation, the expression levels of IFNγ, TNFα and IL2 in CD4+T cells were significantly higher than those in the HAd5 vector control group (P<0.05).

### 12.3 Summary of Immunogenicity Evaluation of Mouse Model

PAd5LCL3-F317L-A151R-P34-pp62 recombinant adenovirus has strong immunogenicity and can induce mice to produce high levels of serum IgG antibodies. High doses of 1*10⁸ FFU and medium doses of 1*10⁷ FFU resulted in high immunologically induced titers. The results of cellular immune response test showed that intramuscular injection of the adenovirus vector vaccine of 1*10⁷FFU could induce specific cellular immune response in the immunized mice.

Although the present invention has been disclosed as above, the present invention was not limited thereto. Various changes and modifications may be made by those skilled in the art without departing from the spirit and scope of the present invention, and the scope of the present invention should be determined by the scope of the appended claims.

1. A recombinant adenovirus vector pAd5LCL3 comprising: E1, E3, E4 and E2a genes are deleted in the regions of E1, E3, E4 and E2a, wherein one or more exogenous antigen gene can be simultaneously expressed in the E1 region and E4 region.

2. The recombinant adenovirus vector pAd5LCL3 according to claim 1, wherein an ORF6/7 deleted from the E4 gene is located in the E2a region, and the ORF6/7 has a nucleotide sequence presented by SEQ ID NO.7.

3. The recombinant adenovirus vector pAd5LCL3 according to claim 2, wherein the E1 region includes a Swa I enzyme cleavage site; and an I-sceI enzyme cleavage site is disposed in the E4 region.

4. The recombinant adenovirus vector pAd5LCL3 of claim 3, wherein the pAd5LCL3 has a nucleotide sequence presented by SEQ ID NO. 5.

5. A method for constructing a recombinant adenovirus vector pAd5LCL3 comprising knocking out of E1, E3, E4, and E2a genes in the regions of E1, E3, E4 and E2a from a adenovirus cyclic vector plasmid by CRISPR/cas9, and placing an ORF6/7 expression frame of the E4 region in the E2a region.

6. The construction method according to claim 5, comprising the steps of:
1) knocking out the E1 gene of the adenovirus annular vector plasmid by using CRISPR/cas9, introducing a Swa I enzyme cutting site, seamlessly cloning the fused fragment with a vector, knocking out the E3 gene by using CRISPR/cas9, and connecting in a seamless cloning mode to obtain the adenovirus vector plasmid pAd5 without E1 and E3 genes;
2) knocking out the E4 gene of the adenovirus annular vector plasmid pAd5 by using CRISPR/cas9, amplifying by using PCR and introducing an I-sceI enzyme cutting site, and obtaining the adenovirus vector plasmid pad5 delta E4 without E1, E3 and e4 genes by using a seamless cloning method;
3) knocking out the E2a gene of the adenovirus annular vector plasmid pad5 delta E4 by using CRISPR/cas9, placing an ORF6/7 expression frame of an E4 region at the position where the E2a region is knocked out, and then using a seamless cloning method to obtain the adenovirus vector plasmid pAd5LCL3 without E1, E3, e4 and E2a genes.

7. (canceled)

8. (canceled)

9. (canceled)

10. An african swine fever virus vaccine, wherein the vaccine is obtained by constructing a recombinant adenovirus vector co-expressing four antigen genes of the african swine fever virus and packaging by 293TD37 cells.

11. The vaccine according to claim 10, wherein the recombinant adenovirus vector co-expressing four antigen genes of the African swine fever virus is packaged by a recombinant adenovirus of 293TD37 cells constructed by pcDNA3.1+(hyg)-ORF6-IRES-DBP, and the cell strain storage number of the 293TD37 cells is CCTCC NO:C201996, which is deposited in the China Type Culture Collection.

12. The vaccine of claim 10, wherein the four antigen genes are each any one of the following five groups of antigen genes: a first group: P72, B602L, P30, and P54; a second group: CP129Rubiqutin, MGF5L6L, CP312R, and MGF110-4L; a third group: L8Lubiqutin, I215L, I73Rhbsag, and E146L; a fourth group: EP402R, EP153R, I177L, and K205Rubiqutin; or a fifth group 5: F317L, A151R, P34, and pp62.

13. The vaccine of claim 12, wherein:
in the first group, P72 and B602L are expressed in the E1 region and P30 and P54 are expressed in the E4 region, constituting a recombinant adenovirus vector pAd5LCL3-P72-B602L-P30-P54 in which four antigen genes are co-expressed;
in the second group, the CP129Rubiqutin is obtain by adding the molecular adjuvant ubiquitin on the CP129R, the CP129Rubiqutin and the MGF5L6L are express in an E1 region, the CP312R and the MGF110-4L are expressed in an E4 region, and a recombinant adenovirus vector pAd5LCL3-CP129R ubiqutin-MGF5L6L-CP312R-MGF110-4L for co-expression of four antigen genes is formed;
in the third group, L8Lubiqutin is obtain by adding the molecular adjuvant ubiquitin to L8L, I73Rhbsag is obtain by adding the molecular adjuvant hbsag to 173R, L8Lubiqutin and I215L are expressed in an E1 region, I73Rhbsag and E146L are expressed in an E4 region, and a recombinant adenovirus vector pAd5LCL3-L8 lubiqutin-I215L-I73RHBsAg-E146L for co-expression of four antigen gene is formed;
in the fourth group, the K205Rubiqutin is obtain by adding the molecular adjuvant ubiqutin on the K205R, the EP402R and the EP153R are express in the E1 region, the I177L and the K205Rubiqutin are expressed in the E4 region, and a recombinant adenovirus vector pAd5LCL3-EP402R-EP153R-I177L-K205rubiqutin for co-expression of four antigen genes is for; or
in the fifth group, F317L and A151R are expressed in the E1 region, and P34 and pp62 are expressed in the E4 region, forming a recombinant adenovirus vector pAd5LCL3-F317L-A151R-P34-PP62 in which four antigen genes are co-expressed.

14. The vaccine of claim 12, wherein
the P72, B602L, P30, P54 and pAd5LCL3-P72-B602L-P30-P54 have nucleotide sequences shown by SEQ ID NO.1, Seq ID NO.2, Seq ID NO.3, Seq ID NO.4 and Seq ID NO.6, respectively,
the CP129R, ubiqutin, MGF5L6L, CP312R, MGF110-4L, pAd5LCL3-CP129R ubiqutin-MGF5L6L-CP312R-MGF110-4L respectively have nucleotide sequences shown in Seq ID NO.14, Seq ID NO.15, Seq ID NO.16, Seq ID NO.17, Seq ID NO.18 and Seq ID NO.19 in a sequence table;
the L8L, the ubiqutin, the I215L, the I73R, the hbsag, the E146L and the pAd5LCL3-L8Lubiqutin-I215L-I73R HBsAg-E146L respectively have nucleotide sequences shown in Seq ID NO.20, Seq ID NO.21, Seq ID NO.22, Seq ID NO.23, Seq ID NO.24, Seq ID NO.25 and Seq ID NO.26 in a sequence table;
the EP402R, the EP153R, the I177L, the K205R, the ubiqutin, and the pAd5LCL3-EP402R-EP153R-I177L-K205Rubiqutin respectively have nucleotide sequences shown in Seq ID NO.27, Seq ID NO.28, Seq ID NO.29, Seq ID NO.30, Seq ID NO.31 and Seq ID NO.32 in a sequence table; or
the F317L, A151R, P34, pp62, and pAd5LCL3-F317L-A151R-P34-pp62 have nucleotide sequences shown in Seq ID NO.33, Seq ID NO.34, Seq ID NO.36, Seq ID NO.36 and Seq ID NO.37, respectively, in a sequence table.

15. A construction method of an african swine fever virus vaccine comprising the steps:
1) knocking out an E1 gene of an adenovirus annular vector plasmid by using CRISPR/cas9, introducing a Swa I enzyme cutting site, seamlessly cloning a fused fragment with a vector, knocking out an E3 gene by using CRISPR/cas9, and connecting by using a seamless cloning mode to obtain an adenovirus vector plasmid pAd5; without E1 and E3 genes;
2) knocking out an E4 gene of the adenovirus annular vector plasmid pAd5 by using CRISPR/cas9, amplifying by using PCR and introducing an I-sceI enzyme cutting site, and then obtaining the adenovirus vector plasmid pad5 delta E4 without E1, E3 and e4 genes by using a seamless cloning method;
3) knocking out an E2a gene of the adenovirus annular vector plasmid pad5 delta E4 by using CRISPR/cas9, placing an ORF6/7 expression frame of an E4 region at a sequence position in which the E2a region is knocked out, and then using a seamless cloning method to obtain the adenovirus vector plasmid pAd5LCL3; without E1, E3, E4 and E2a genes;
4) construct an adenovirus E1 region shuttle plasmid, pS5E1 was connected to P72, IRES, B602L of a first group; CP129Rubiqutin, IRES, MGF5L6L of a second group; or L8Lubiqutin, IRES, I215L of a third group; or EP402R, IRES, EP153R of a fourth group; or F317L, IRES, A151R gene fragments of a e fifth group by DNA ligases to construct an African swine fever adenovirus type 5 vector E1 region shuttle plasmid, respectively, the first group: pS5E1-P72-IRE2 Group II: pS5E1-CP129Rubiqutin-IRES-MGF 5L6L; Group III, pS5E1-L8Lubiqutin-IRES-I215L; IRES-I215L; Group 4: pS5E1-EP 402R-IRES-EP 153R; Group 5: pS5E1-F317L-IRES-A151R; or
5) constructing an adenovirus E4 region shuttle plasmid which is respectively connected with P30, 2A and P54; of the first group; Or a second group of CP312R, 2A, MGF5L6L; Or group III I73Rhbsag, 2A, E146L; Or I177L, 2A, K205Rubiqutin; of the fourth group; Or the P34, 2A and pp62 genes of the fifth group are respectively P30-2A-P54, CP312R-2A-MGF5L6L, I73Rhbsag-2A-E146L, I177L-2A-K205Rubiqutin and P34-2A-pp62 through fusion PCR technology, and the EGFP is knocked out through enzyme digestion on a shuttle plasmid pS5E4-EGFP, And connecte with that gene fragment by a DNA ligase to construct an E4 region shuttle plasmid of an african swine fever adenovirus type 5 vector, namely Group I: pS5E4-P30-2A-P54; Group II: pS5E4-CP312R-2A-MGF 5L6L; Group III: pS5E4-I73Rhbsag-2A-E146L; Group IV: PS5E4-I177L-2A-K205Rubiqutin; Group V: pS5E4-P34-2A-pp62; and
6) the E1 region shuttle plasmid pS5E1-P72-IRES-B602L, or pS5E1-CP129R ubiqutin-IRES-MGF5L6L, orpS5E1-L8lubiqutin-IRES-I215L, or pS5E1-EP402R-IRES-EP153R, Or pS5E1-F317L-IRES-A151R is homologously recombined with an adenovirus vector plasmid pAd5LCL3 to obtain a first group of adenovirus vector plasmids: Group I: pAd5LCL3-P72-IRES-B602L; Group II: pAd5LCL3-CP129Rubiqutin-IRES-MGF5L6L; Group III: pAd5LCL3-L8lubiqutin-IRES-I215L; Group IV: pAd5LCL3-I177L-2A-K205Rubiqutin; Group V: pAd5LCL3-F317L-IRES-A151R; or
7) shuttle that E4 region plasmid first group: pS5E4-P30-2A-P54; Group II: pS5E4-CP312R-2A-MGF5L6L; Group III: pS5E4-I73Rhbsag-2A-E146L; Group IV: pS5E4-I177L-2A-K205Rubiqutin; Group V: pS5E4-P34-2A-pp62 and adenovirus vector plasmid Group I: pAd5LCL3-P72-IRES-B602L; Group II; pAd5LCL3-CP129Rubiqutin-IRES-MGF5L6L; Group III: pAd5LCL3-L8LUBIQUTIN-IRES-I215L; Group IV: pAd5LCL3-I177L-2A-K205Rubiqutin; And fifth group, performing homologous recombination of pAd5LCL3-F317L-IRES-A151R to obtain a recombinant adenovirus vaccine co-expressing four antigen genes, wherein the first group comprises pAd5LCL3-P72-B602L-P30-P54; Group II: pAd5LCL3-CP129Rubiqutin-MGF5L6L-CP312R-MGF110-4L; Group III: pAd5LCL3-L8Lubiqutin-I215L-I73RHBsAg-E146L; Group IV: pAd5LCL3-EP402R-EP153R-I177L-K205Rubiqutin; Group V: pAd5LCL3-F317L-A151R-P34-PP62.

16. The method according to claim 15, wherein the adenovirus annular vector plasmid described in step 1 is derived from wild-type human adenovirus type 5 virus amplified in A549 cells, virus liquid is collected and concentrated, an adenovirus type 5 genome is extracted by using HirtViral DNA Extract method, and a linear adenovirus type 5 genome is constructed into an annular adenovirus annular vector plasmid by using cosmid method.

17. The method according to claim 16, wherein the ORF6/7 expression frame gene in step 3) has a nucleotide sequence shown in Seq ID NO.7 in a sequence table; in the Step 4) the IRES has a nucleotide sequence shown in Seq ID NO.8 in a sequence table; in step 5) has the nucleotide sequence shown in Seq ID NO.9 in a sequence table.

18. The method according to claim 17, wherein the shuttle plasmid pS5E1 skeleton of step 4) employs puc origin, amp basic element, Ad5 left arm ITR partial sequence, right arm PIX, PIVa2 partial sequence, and CMV-MCS-SV40 early polyA; The skeleton of the E4 region shuttle plasmid pS5E4-EGFP in step 5) adopts puc origin, amp basic elements, a left arm ITR sequence in the Ad5E4 region, a right arm partial fiber gene sequence and an EF1α-EGFP-HBV polyA gene; Wherein that basic element of puc origin and amp have the nucleotide sequence shown in Seq ID NO.10 in the sequence table, and the EF1α-EGFP-HBV polyA gene has the nucleotide sequence shown in Seq ID NO.11 in the sequence table.

19. The method according to claim 18, wherein in step 6), the E1 region shuttle plasmid and the adenovirus vector plasmid pAd5LCL3 are homologously recombined, and the shuttle plasmid and the adenovirus vector plasmid pAd5LCL3 are subjected to enzyme digestion by PacI and SwaI, the enzyme digestion product is dephosphorylated, the OMEGA Ultra-Sep Gel Extraction Kit carries out gel recovery carrier and fragment, the conversion product is coated on a plate, colonies are picked, and XhoI enzyme digestion verification is carried out.

20. The method according to claim 19, wherein in step 7), the E4 region shuttle plasmid and the adenovirus vector plasmid are homologously recombined by performing enzyme digestion on the E4 region shuttle plasmid and the adenovirus vector plasmid by PacI and I-sceI, dephosphorylating the enzyme digestion product, recovering the carrier and the fragment from omega ultra-sepgel extract kit, coating the plate with the conversion product, picking colonies, and performing XhoI enzyme digestion verification.

21. (canceled)

22. (canceled)

23. (canceled)

24. The recombinant adenovirus vector pAd5LCL3 according to claim 1, wherein the exogenous antigen gene comprises a gene or gene fragment of a virus, bacterium, or tumor.

25. The recombinant adenovirus vector pAd5LCL3 according to claim 24, wherein the exogenous antigen gene comprises an african swine fever virus gene.


SEQUENCE LISTING (169 sequences)

SEQ ID NO: 1 - DNA, 1938 bp, Artificial Sequence
  Synthetic P72
atggccagcggcggagctttttgcctgatcgccaatgatggcaaggccgacaagatcatcctggcccaggacctgctgaattccaggatcagcaatatcaagaacgtgaacaagagctacggcaagcctgaccccgagcctacactgagccagatcgaggagacacacctggtgcactttaacgcccacttcaagccctacgtgcccgtgggcttcgagtacaataaggtgagaccccacaccggcacacctaccctgggcaataagctgacatttggcatccctcagtacggcgacttctttcacgacatggtgggccaccacatcctgggcgcctgccacagctcctggcaggatgcccctatccagggcacaagccagatgggcgcccacggccagctgcagaccttcccaagaaacggctacgactgggacaatcagacccctctggagggcgccgtgtacaccctggtggaccctttcggcagacctatcgtgcctggcacaaagaacgcctacagaaatctggtgtactactgcgagtaccccggcgagaggctgtacgagaatgtgaggtttgacgtgaacggcaactccctggatgagtactccagcgatgtgaccacactggtgaggaagttttgtatccctggcgataagatgacaggctacaagcacctggtgggccaggaggtgtccgtggagggcacctccggccctctgctgtgtaatatccacgatctgcacaagcctcaccagagcaagcccatcctgaccgatgagaatgacacacagaggacctgcagccacaccaatcctaagttcctgtcccagcacttccctgagaacagccacaatatccagacagccggcaagcaggacatcacacccatcacagacgccacatacctggacatcaggagaaatgtgcactactcctgcaatggcccccagacacctaagtactaccagccccccctggccctgtggatcaagctgaggttctggttcaatgagaacgtgaatctggccatcccctccgtgtccatccctttcggcgagagattcatcaccatcaagctggcctcccagaaggacctggtgaatgagtttcctggcctgttcgtgaggcagagcaggttcatcgccggcagaccttccagaaggaacatcagattcaagccttggtttatccctggcgtgatcaacgagatctccctgaccaacaacgagctgtacatcaataacctgtttgtgacccctgagatccacaatctgttcgtgaagagggtgagattctccctgatcagagtgcacaagacccaggtgacacacaccaataacaatcaccacgacgagaagctgatgtccgccctgaagtggcctatcgagtacatgttcatcggcctgaagcctacctggaacatctccgaccagaacccccaccagcacagagactggcacaagttcggccacgtggtgaatgccatcatgcagcctacccaccacgccgagatcagcttccaggatagggatacagccctgcctgatgcctgctcctccatcagcgatattagccctgtgacctaccccatcaccctgcctatcatcaagaacatcagcgtgacagcccacggcatcaacctgatcgacaagtttcccagcaagttttgctcctcctacatcccttttcactacggcggcaatgccatcaagacacccgacgaccccggcgccatgatgatcacattcgccctgaagcctagagaggagtaccagccctccggccacatcaacgtgagcagggccagggagttctacatcagctgggatacagactacgtgggcagcatcaccaccgccgatctggtggtgagcgcctccgccatcaatttcctgctgctgcagaacggcagcgccgtgctgagatacagcacc

SEQ ID NO: 2 - DNA, 1617 bp, Artificial Sequence
  Synthetic B602L
atggccgaattcaatattgatgaactgctgaaaaacgtgctggaagatccctctaccgagatctctgaagagaccctgaaacagctgtatcagcgcaccaacccctataaacagttcaagaatgactcccgggtggccttttgctccttcaccaatctgagggagcagtacatccgccgcctgatcatgacctccttcatcggctacgtcttcaaggccctccaggaatggatgcccagctatagcaagcctacccacaccaccaagaccctgctcagcgaactcatcaccctcgtggacaccctgaagcaggagaccaacgacgtgcccagcgaatccgtcgtgaacaccatcctgagcatcgccgacagctgcaaaacccagacccagaaaagcaaggaagccaaaaccaccatcgacagcttcctgagagaacacttcgtgttcgaccccaacctgcatgcccagagcgcctacacctgcgccgacaccaacgtcgacacctgcgccagcatgtgcgccgacacaaacgtggacacctgcgctagcatgtgcgctgataccaacgtcgatacctgcgcctccacctgcacctccaccgagtacaccgacctggccgaccccgagcgcatccctctgcacatcatgcagaaaaccctgaacgtgcccaacgaactgcaggccgatattgacgccatcacccagaccccccagggctaccgcgccgctgctcatatcctgcagaacatcgaactccaccagagcatcaagcacatgctggagaacccccgcgccttcaagcccatcctgtttaacaccaagatcacacgctatctgtcccagcacatccccccccaggacaccttctacaaatggaactactacatcgaagacaactacgaagaactgcgcgccgccacagagagcatctaccccgaaaagcccgacctggagttcgccttcatcatctacgacgtggtggacagcagcaaccagcagaaggtcgacgagttctactataagtacaaggaccagatctttagcgaggtgagcagcatccagctgggcaactggaccctgctgggcagctttaaggccaaccgcgagcgctacaactacttcaaccagaacaacgaaatcatcaagcggatcttggacaggcacgaggaggacctgaagatcggcaaagagatcctccgcaacaccatctaccacaaaaaggccaaaaacatccaggagaccggacccgacgcccccggactgtctatctacaacagcacattccacaccgacagcggcatcaaaggactgctcagcttcaaagagctcaaaaacctggaaaaagccagcggcaacatcaagaaagcccgcgaatacgatttcatcgacgactgcgaagagaaaatcaagcagctgctgagcaaagagaacctgacccccgacgaggaaagcgagctgatcaagaccaaaaagcagctggacaacgccctggagatgctgaacgtgcctgacgacaccatccgcgtggacatgtgggtgaacaacaacaacaaactcgaaaaggagatcctgtacaccaaggccgagctgtacccctacgacgtgcccgattacgcc

SEQ ID NO: 3 - DNA, 630 bp, Artificial Sequence
  Synthetic P30
atggacttcatcctgaacatcagcatgaagatggaggtgatcttcaagaccgacctgcggagcagcagccaggtggtgttccacgccgggagcctgtacaactggttcagcgtggagatcattaacagcggccggattgtgaccaccgccattaagaccctcctgtccaccgtgaagtacgacatcgtcaaaagcgccaggatttatgccggccagggctacaccgaacaccaggcccaggaggagtggaacatgatcctgcacgtgctgttcgaggaagagaccgagagcagcgccagcagcgagaacatccacgagaagaacgataatgagacaaacgaatgcaccagcagcttcgagacactgttcgaacaggaaccctctagcgaggtgccaaaggacagcaagctgtacatgctggcccagaagaccgtgcagcacattgagcagtacggcaaagcccccgacttcaacaaggtgatccgcgcccacaacttcatccagaccatctacggcacccccctgaaagaggaagaaaaagaagtggtgaggctcatggtgatcaaactgctgaagaagattagcttttacctgacctacatctacccctacgacgtgcccgactacgcc

SEQ ID NO: 4 - DNA, 555 bp, Artificial Sequence
  Synthetic P54
atggacagcgagttcttccagcccgtgtacccccgccactacggagagtgcctgagccccgtgaccacccccagcttcttcagcacacacatgtacaccatcctgatcgccatcgtggtgctggtgattatcatcatcgtcctgatctacctgttcagctctcgcaagaaaaaggccgctgccatcgaagaggaggacatccagttcatcaacccctaccaggaccagcagtgggtggaagtgaccccccagcccggcaccagcaagcctgctggagccaccaccgccagcgtgggaaaacccgtgaccggccgccccgctaccaacagacccgctaccaataagcccgtgaccgacaaccccgtcaccgaccgcctcgtgatggccaccggcggaccagctgccgctcctgctgctgctagcgcccctgctcaccccgctgagccatacaccaccgtgaccacccagaacaccgccagccagaccatgagcgccattgagaatctccgccagcgcaatacatatacccacaaagacctggaaaactccctctaa

SEQ ID NO: 5 - DNA, 32619 bp, Artificial Sequence
  Synthetic pAd5LCL3
agcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacacgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgatcgattaattaacgacccatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttattaccctgttatccctagcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatctttgagaccgcacagggtcttaataagggtgcagagatcctcaggtccttgacaaggtgagtgaatgcagccttcggtttctaccgagtgctgagttatggtaatgggcttttctcccaccatgaccaccaatttctgacgcttggttggcaacttgtagctaaggcggtgtccggtggtattactgtcgtaggtgactttggcctgctttaccagacaaaagataccccttttgcactggtgcaagttaaccatgtcttggagctcttgattcatgcgctgttgctcggccgctgccctgcgtctttctagcaggcgctgctctgtaataattccgtccatttctagctagagaaacctgaattagaatagcccgtagagttgcttgaattgttcataaaccccacagtagctgcgcctttggcctaataccctaagggttttctaagctcacctcctgttctggtaaacagagttattgaggtctgtccggaaaaagtctggtttacggtcaggcggtaggtgtggtgcagcggccggtgacgcactcgtacgttcccggcaggtaaggagggtggtgttttttctgatggagtagctgagctcggagaggttctctcgtagactcactccgtctgggttgaaactgttgtaaatcacagagggagagatgttaaaagtaccaggtaaggttcgccttggtttgcttgggcgggtgaagacggtggcgtttacaggatggcgataggagccccagtatattttaatttctgtatttattatactcagcacagagatggcaacaaagatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagactagggttctgtgagtttgattaaggtacggtgatctgtataagctatgtggtggtggggctatactactgaatgaaaaatgacttgaaattttctgcaattgaaaaataaacacgttgaaacataacacaaacgatacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggctggtgcacacggaccacgtcaaagacttcaaacaaaacataaagaagggtgggctcgtccatgggatccacctcaaaagtcatgtctagcgcgtgggcggagttggcgtagagaaggttttggcccaggtctgtgagtgcgcccatggacataaagttactggagaatgggatgcgccaaagggtgcgatcgcaaagaaactttttctgggtaatgctgtcaactgcggtcttgcctataagcggataggggaagttagcagggtaggcctgtccttcgcgcatggtgggggcaaggtagccaacaaatccagagttgttgtgttggtgtaggatgcccacctgttggtagtccttgtatttagtatcatccaccacctgacggctcatgggctggaagtttctaaagaaggagtacatgcggtccttgtagctctctgggatatagaagccctggtagccaatgttatagttagctagcatttgtaccaggaaccagtctttggtcatgttacactgggcaacgttgtaaccctccccgtcaactgagcgcttaatttcaaactcgttgggggtaagcaggcggtcattgccaggccagctgacagaagagtcaaaggtaatggccaccttcttaaaggtgtggttgaggtaaaaggttccatctaggtagggtatagagccagagtaggtgtaataagggtcgtagcccgagcccagtgatggggtttccttagtcttaaggcgcgtgaaggcccagccgcggaaagccgcccagttgcgggaggggatggatatgggcacgttggtagcgttggcgggtatagggtagagcatgttggcggcggagagatagtcgttaaaggactggtcgttggtgtcgtttctaagcatggcctcaagcgtggaggcggtgttgtgggccatggggaagaaggtggcgtaaaggcaaatgctatcaaacttaatgctggctccgtcaacccttaggtcatttcctagggagctctgcagaaccatgttaacatccttcctgaagttccactcgtaggtgtatgagcccggcaggagaaggaggtttttaatggcaaagaacttctgaggcacctggatgtggaagggcacatagcgaccattgcccagcaacattgagcggtagcgcaggccagcattgcggtggtggttaaatgggttgacgttgtccatatagtcaagggaccagcgtgctccaaggttaatgtagcagtccactagcccgggagccaccactcgcttgttcatgtagtcgtaggtgtttgggttatcagaaatttttacgttggaaggactgtactttagcttgtcgggcaaatacagcgctatgttggagtacaggaaatttctccacaggttggcatttagattgatttccatggcaaaattatttccaactcttatttcatttttatctgaaaattctgtagcatctttttcccatccattttcctgacctgttttaggttttaccttggtaagagtctctgtattaatcacacctcccagtggaaagcagtaatttggaagttcatcttcagttccatgattttcaataattctaacatctggatcatagctgtcaacagcctgattccacatagaaaagtacctggttctatcaccaatggaatcaagcaaaagctggtatgaaagctctgtgtttctgtcttgcaaatctacaacagcattcaactgcgatgcttggcccgccagaacacccatattacccgtgctgttgtaatacattagaccaataaaattgtccctaaaagcaatgtaattaggcctgttgggcatagattgttggcccattagttctcgtgagttaccttccttaatagtgggcatgtaagaaatatgagtgtctggggtttctatatctacatcttcactgtacaataccactttaggagtcaagttatcaccattgcctgcggctgcctcagtagttgagaaaaattgcatttccacttgactttctagctttccattttgttgctttacaagaatgccttgccctccattttcatttgtgggttttgcatatgaaccgtaacatggtttcattggggtagtcttttttaggactctcccagctgcatgattaatttctgtttcgtaccactgagattctcctatttgaggttcaggttgaaatgttttatcggcatatttaggtgtttgaccttcgacacctatttgaataccctcctttgtaatatttataccagaataaggcgcctgcccaaatacgtgagttttttgctgctcagcttgctcgtctacttcgtcttcgttgtcatcgtcctcttcttctaggtttatttcaagagcagtagcagcttcatcccattcgcaaggatttggggcacccttgggagccagggcgttgtaggcagtgccagagtagggcttaaaagtagggcccctgtccagcacgccgcggatgtcaaagtacgtggaagccatgtccagcacacggttatcacccacagctagggtgaaccgcgccttgtacgagtacgcagtatcctcacggtccacagggatgaaccgcagcgtcaaacgctgggaccggtctgtggtcacgtcgtgcgtaggcgccaccgtggggtttctaaacttgttattcaggctgaagtacgtctcggtggcgcgggcaaactgcaccagcccggggctcaggtactccgaggcgtcctggcccgagatgtgcatgtaagaccactgcggcatcatcgaaggggtagccatcttggaaagcgggcgcgcggcggctcagcagctcctctggcggcgacatggacgcatacatgacacacatacgacacgttagctatcagaagcatcgtcggcgcttcagggattgcacccccagacccacgatgctgttcagtgtgctttgccagttgccactggctacgggccgcaacgatcgcggaccgctggcggcgcggcgcagggacgcgcggctaggacgggttacaacaacggcggtcgggcctggcagcacaggtttctgctgggtgtcggcggggggaggcaggtccagcgttacgggtgtgtgctggcccagcactccggtagccatgggcgcgatgggacgggtggtgggcaggccttgctttagtgcctcctcgtacgagggaggctcgtctatttgcgtcaccagagtttcttccctgtcggggcgcggacgcttttcgccacgcccctctggagacactgtctccacggccggtggaggctcctctacgggagggcggggatcaagcttactgttaatcttattttgcactgcctggttggccaggtccaccaccccgctaatgccagaggccaggccatctaccaccttttgttggaaattttgctctttcaacttatccctcagcatctggcctgtgctgctgttccaggccttgctgccatagttcttaacggtggaaccgaaatttttaatgccgctccacagcgagccccagctgaaggcgccaccgctcatattgctggtgccgatatcttgccagtttcccatgaacgggcgcgagccgtgtcgcggggccagagacgcaaagttgatgtcttccattctacaaaatagttacaggaccaagcgagcgtgagagtccagactttttattttgatttttccacatgcaacttgtttttaatcagtgtctctgcgcctgcaaggccacggatgcaattccgggcacggcgccaatcgccgcggcgatcagtggaataaggaggggcaggataccgccgcgcatgcgacggtgcgacgcgcgccgccgccggtggtgcgcacgacgcatgccgcccgtcaggccgtggccggccatgcccctcctacggtgcattcttcctcggaatcccggcaccgggaaacggaggcggcaggtgagggccatatctgcaagaaccacaaagaccggcttttaaacgatgctggggtggtagcgcgctgttggcagcaccagggtcctgcctccttcgcgagccaccctgcgcacggaaatcggggccagcacgggctggcgacggcgacggcggcggcgggttccagtggtggttcggcgtcgggtagttgctcgtcttctggggcggtaggtgtagccacgatagccgggggtaggcgcaatggaaggatgtagggcatattcgggcagtagcgcgctggcggcgccgtacttcctcgaacggcgcgggcgccggggggctgaaacgcgaaacatccacgggtccgtttgcacctccgtagaggtcttggacgcggccgcagcgaccgcctgcaccgcggcatccgccaccgctgaggcaaccggggacgtttgtgtctccatgccctctgtggcggtggcaatactggtgctactggtagtgggtatctgaacgtccacggtctgcacgcccagtcccggcgccacctgcttgattggccgcacgcggacctcgggctccagcccaggttccacggtcattttttccaagacatcttccagtcgctggcgcttgggtaccatcagctgcacggtgggtgccaagtcaccagactcgcgctttaggccgcgcttttcttcggacggtgcaagcgcgggcagcacctgctgcagtgttacgggctttaggctaggtgttgggttgccctcgtccagcggcaacgccagcatgtccttatgccgctttccgtaggcaaactccccgaggcgctcgttggcctgctcaagcaggtcctcgtcgccgtacacctcatcatacacgcgcttgtaggtgcgggtggagcgctcaccgggcgtaaagactacggtggtgccgggtcgcaaaacacgttttacgcgtcgacctttccactgtacccgtcgcctgggcgcggtagcgtgcagcagttccacctcgtcgtcaagttcatcatcatcatctttctttttctttttgacccgctttagctttcggggcttgtaatcctgctcttccttcttcggggggccatagatctccggcgcgatgacctggagcatctcttctttgattttgcgcttggacatagcttcgttgcgcgccgccgccgctggatacatacaacagtacgagtctaagtagttttttcttgcaatctagttgcgcggggggcgggtgcgcacgggcacgcgcaggccgctaaccgagtcgcgcacccaatacacgttgcccctgcgaccctgagtcatagcactaatggccgcggctgctgcggcggccgctcgtcgcctggacctggggggcacagtgacaatacccgcggccagccttcgagcggcccgcatggccgcccgtcggccggtgcgacgtgcgcggttaagcagggccgccgccgcgcgttgggcggcagtgccgggtcggcggcggtggcgacgtgctacgcgcctccgccgtctcttcattttagcatagcgccgggctccgcgcaccacggtctgaatggccgcgtccactgtggacactggtggcggcgtgggcgtgtagttgcgcgcctcctccaccaccgcgtcgatggcgtcatcgacggtggtgcgcccagtgcggccgcgtttgtgcgcgccccagggcgcgcggtagtgcccgcgcacgcgcactgggtgttggtcggagcgcttcttggccccgccaaacatcttgcttgggaagcgcaggccccagcctgtgttattgctgggcgatataaggatggacatgcttgctcaaaaagtgcggctcgataggacgcgcggcgagactatgcccagggccttgtaaacgtaggggcaggtgcggcgtctggcgtcagtaatggtcactcgctggactcctccgatgctgttgcgcagcggtagcgtcccgtgatctgtgagagcaggaacgttttcactgacggtggtgatggtgggggctggcgggcgcgccaaaatctggttctcgggaaagcgattgaacacgtgggtcagagaggtaaactggcggatgagttgggagtagacggcctggtcgttgtagaagctcttggagtgcacgggcaacagctcggcgcccaccaccggaaagttgctgatctggcgcgtggagcggaaggtcacggggtcttgcatcatgtctggcaacgaccagtagacctgctccgagccgcaggttacgtcaggagtgcaaagcagggtccatgagcggattccggtctgagggtcgccgtagttgtatgcaaggtaccagctgcggtactgggtgaaggtgctgtcattgcttattaggttgtaactgcgtttcttgctgtcctctgtcaggggtttgatcaccggtttcttctgaggcttctcgacctcgggttgcgcagcgggggcggcagcttcggccgctgcttcggcctcagcgcgcttctcctcagcccgtgtggcaaaggtgtcgccgcgaatggcatgatcgttcatgtcctccaccggctgcattgccgcggctgccgcgttggagttctcttccgcgccgctgccactgctgttgctgccgcctgcgccacccccgccctgttcggtgtcatctttcaagctcgcctggtaggcgtccacatccaacagtgcgggaatgttaccaccctccagatcatcgtaggtgatcctaaagccctcctggaagggttgccgcttgcggatgcccaacaagttgctcaggcggctgtgggtgaagtccaccccgcatcctggcagcaaaatgatgtctggatggaaggcttcgtttgtatataccccaggcatgacaagaccagtgacggggtcaaaccccagtctgaagttgcgggtgtcaaactttaccccgatgtcgctttccagaaccccgttctgtctgcccactttcaagtagtgctccacgatcgcgttgttcataaggtctatggtcatggtctcggagtagttgccctcgggcagcgtgaactccacccactcgtatttcagctccacctgattgtccttagtaggcaagcgcgacaccatcacccgcgccttaaacttattggtaaacatgaactcgttcacatttggcatgttggtatgcaggatggttttcaggtcgccgccccagtgcgaccggtcgtcaagattgatggtctgtgtgcttgcctcccccgggctgtagtcattgttttgaatgaccgtggtcagaaagttgctgtggtcgttctggtagttcagggatgccacatccgttgacttgttgtccaccaggtacacacgggtggtgtcgaataggggtgccaactcagagtaacggatgctgtttctccccccggtaggccgcaggtaccgcggaggcacaaacggcgggtccaggggagcatcgaagggagaacccagcgccgccgccactggcgccgcgctcaccacactctcgtaggagggaggaggaccttcctcatacatcgccgcgcgccgcatactaaggggaatacaagaaaaccaacgctcggtgccatggccttggtgagttttttattttgcatcatgctttttttttttttaaaacattctccccagcctggggcgaaggtgcgcaaacgggttgccactccctcccaaatccaggacgctgctgtcgtctgccgagtcatcgtcctcccacaccagaccccgctgacggtcgtgcctttgacgacgggtgggcgggcgcgggcctggcacgtccctgtgctcctgcgcgtacgtcttccatctactcatcttgtccactaggctctctatcccgttgttgggaaatgccggaggcaggtttttttcgcgctgcggctgcagcagcgagttgtttaggtactcctcctcgcccagcaggcgcgggcgggtggtgcgagtgctggtaagagaccctatcaagcttggaaatgggctactagcatctgaccgcggggccgcagcgcctagatcggacaagctgcttggcctgcggaagctttcctttcgcagcgccgcctctgcctgctcgcgctgttgcaactctagcagggtctgcggttgcggggaaaacacgctgtcgtctatgtcgtcccagaggaatccatcgttaccctcgggcacctcgaatcccccggtgtagaaaccagggggcggtagccagtgcgggttcaagatggcattggtgaaatactcggggttcacggcggccgcgcgatgcaagtagtccattaggcggttgataaacggccggtttgaggcatacatgcccggttccatgttgcgcgcggtcatgtccagcgccacgctgggcgttaccccgtcgcgcatcaggttaaggctcacgctctgctgcacgtagcgcaaaatgcgctcctcctcgctgtttaaactgtgcaacgaggggatcttctgccgccggttggtcagcaggtagtttagggttgcctccaggctgcccgtgtcctcctgccccagcgcgcggctgacacttgtaatctcctggaaagtatgctcgtccacatgcgcctgacctatggcctcgcggtacagtgtcagcaagtgacctaggtatgtgtcccgggacacgctgccactgtccgtgaagggcgctattagcagcagcaacaggcgcgagttgggcgtcagcaagctagacacggtcgcgcggtcgcctgtgggagcccgcaccccccacagcccctgcaagtttttgaaagcctggctcaggtttacggtctgcaggccttgtctactggtctggaaaaaatagtctggcccagactggtacacctcactttgcggtgtctcagtcaccattagccgcagtgcgctcacaaagttggtgtagtcctcctgtccccgcggcacgttggcgggctgtgtactcaggaaggcgtttagtgcaaccatggagcccaggttgccctgctgctgcgcgcgctcacgctgcgccacggcctcgcgcacatcccccaccagccggtccaggttggtctgcacgttgccgctgttgtaacgagccacgcgctgaagcagcgcgtcgtagaccaggccggcctcgtcgggccggatggccctgttttcggccagcgcgtttacgatcgccagcaccttctcgtgcgtggggtttgcgcgcgccgggaccaccgcttccagaattgcggagagccggttggcctgcggctgctgccggaacgcgtcaggattgcgcgcagtcagcgacatgatgcggtccatgacctggcgccagtcgtccgtggagttaaggccggacggctggctctgcagcgccgcccgcaccgccgggtccgttgcgtcttgcatcatctgatcagaaacatcaccgcttagtactcgccgtcctctggctcgtactcatcgtcctcgtcatattcctccacgccgccgacgttgccagcgcgcgcgggtgccaccgccagcccaggtccggccccagctgcctccagggcgcgtcggcttggggcccagcgcaggtcagcgcccgcgtcaaagtaggactcggcctctctatcgccgctgcccgtgccagccagggccctttgcaggctgtgcatcagctcgcggtcgctgagctcgcgccgccggctcacgctcacggccttgtggatgcgctcgttgcgataaacgcccaggtcgtcgctcaaggtaagcaccttcagcgccatgcgcatgtagaacccctcgatctttacctccttgtctatgggaacgtaaggggtatggtatatcttgcgggcgtaaaacttgcccaggctaagcatggaatagttgatggcggccaccttgtcagccaggctcaagctgcgctcctgcaccactatgctctgcaggatgtttatcaaatcgagcagccagcggccctcgggctctactatgtttagcagcgcatccctgaatgcctcgttgtccctgctgtgctgcactataaggaacagctgcgccatgagcggcttgctatttgggttttgctccagcgcgcttacaaagtcccacagatgcatcagtcctatagccacctcctcgcgcgccacaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgggctcaaagtcctcctcgcgcagcaaccgctcgcgattcaggccatgccgcagctcgcgccctgcgtggaactttcgatcccgcatctcctcgggctcctctccctcgcggtcgcgaaacaggttctgccgcggcacgtacgcctcacgcgtatcacgcttcagctgcacccttgggtgccgctcaggagagggcgctcctagccgcgccaggccctcgccctcctccaagtccaggtagtgccgggcccggcgccgcgggggttcgtaatcaccatctgctgccgcgtcaaccgcggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttagatgcatcatccaaggacacccccgcggcccaccgcccgccgcgcggtaccgtagtcgcgccgcggggatgcggcctcttgcaagccatcgacgccgccaccaaccagcccctggaaattaggtatcacctggatctagcccgcgccctgacccgtctatgcgaggtaaacctgcaggagctcccgcctgacctgacgccgcgggagctccagaccatggacagctcccatctgcgcgatgttgtcatcaagctccgaccgccgcgcgcggacatctggactttgggctcgcgcggcgtggtggtccgatccaccgtaactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaatagccactcctccaattggtggcgggagatccagttcttcccgatcggctcgcatcctcgcaccgagcgtctctttgtcacctacgatgtagagacctatacttggatgggggcctttgggaagcagctcgtgcccttcatgctggtcatgaagttcggcggagatgagcctctagtgactgccgcgcgagacctagccgcgaaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctttcagagcacgggctcagctcccctgaagagctcacctacgaggaacttaaaaaattgccttccatcaagggcatcccgcgcttcttggaactttacattgtgggccacaacatcaacggctttgacgagatcgtgctcgccgcccaggtaattaacaaccgttccgaggtgccgggacccttccgcatcacacgcaactttatgcctcgcgcgggaaagatactcttcaacgatgtcaccttcgccctgccaaatccgcgttccaaaaagcgcacggactttttgctctgggagcagggcggatgcgacgacactgacttcaaataccagtacctcaaagtcatggtcagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagaccccctaccgccattctgctcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggatgcggccaccctcaaaggcatcaccgcgggccaggtgaatatcaaatcctcctcgtttttggaaactgacaatcttagcgcagaagtcatgcccgcttttcagagggagtactcaccccaacagctggccctcgcagacagcgatgcggaagagagtgaggacgaacgcgcccccacccccttttatagccccccttcaggaacacccggtcacgtggcctacacctacaaaccaatcaccttccttgatgccgaagagggcgacatgtgtcttcacaccctggagcgagtggaccccctagtggacaacgaccgctacccctcccacttagcctccttcgtgctggcctggacgcgagcctttgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgccctcaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagagcaggtgaccgagctgtgggaccgcctggaactgcttggtcaaacgctcaaaagcatgcctacggcggacggtctcaaaccgttgaaaaactttgcttccttgcaagaactgctatcgctgggcggcgagcgccttctggcggatttggtcagggaaaacatgcgagtcagggacatgcttaacgaagtggcccccctgctcagggatgacggcagctgcagctctcttaactaccagttgcagccggtaataggtgtgatttacgggcccaccggctgcggtaagtcgcagctgctcaggaacctgctttcttcccagctgatctcccctaccccggaaaccgttttcttcatcgccccgcaggtagacatgatccccccatctgaactcaaagcgtgggaaatgcaaatctgtgagggtaactacgcccctgggccggatggaaccattataccgcagtctggcaccctccgcccgcgctttgtaaaaatggcctatgacgatctcatcctggaacacaactatgacgttagtgatcccagaaatatcttcgcccaggccgccgcccgtgggcccattgccatcattatggacgaatgcatggaaaatcttggaggtcacaagggcgtctccaagttcttccacgcatttccttctaagctacatgacaaatttcccaagtgcaccggatacactgtgctggtggttctgcacaacatgaatccccggagggatatggctgggaacatagccaacctaaaaatacagtccaagatgcatctcatatccccacgtatgcacccatcccagcttaaccgctttgtaaacacttacaccaagggcctgcccctggcaatcagcttgctactgaaagacatttttaggcaccacgcccagcgctcctgctacgactggatcatctacaacaccaccccgcagcatgaagctctgcagtggtgctacctccaccccagagacgggcttatgcccatgtatctgaacatccagagtcacctttaccacgtcctggaaaaaatacacaggaccctcaacgaccgagaccgctggtcccgggcctaccgcgcgcgcaaaacccctaaataaagacagcaagacacttgcttgatccaaatccaaacagagtctggttttttatttatgttttaaaccgcattgggaggggaggaagccttcagggcagaaacctgctggcgcagatccaacagctgctgagaaacgacattaagttcccgggtcaaagaatttaaattctactcgctggcactcaagagtggcctcttgaggaactcaccgggtataaatacactacacgtcagctgactataataataaaacgccaactttgacccggaacgcggaaaacacctgagaaaaacacctgggcgagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgataagctatcaaacatgagaattcggcgcgccattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaacgcgcctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcacctgcacaccgcgccttaattaagaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcagcgaaccaatcagcagggtcatcgctagccagatcctctacgccggacgcatcgtggccaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgctgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcata

SEQ ID NO: 6 - DNA, 40402 bp, Artificial Sequence
  Synthetic pAd5LCL3-P72-B602L-P30-P54
agcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacacgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgatcgattaattaacgacccatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttttttaaaagaaaaggggggattggggggtacagtgcaggggaaagaatagtagacataatagcaacagacatacaaactaaagaattacaaaaacaaattacaaaattcaaaattttatcgtactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggatccgccaccatggacttcatcctgaacatcagcatgaagatggaggtgatcttcaagaccgacctgcggagcagcagccaggtggtgttccacgccgggagcctgtacaactggttcagcgtggagatcattaacagcggccggattgtgaccaccgccattaagaccctcctgtccaccgtgaagtacgacatcgtcaaaagcgccaggatttatgccggccagggctacaccgaacaccaggcccaggaggagtggaacatgatcctgcacgtgctgttcgaggaagagaccgagagcagcgccagcagcgagaacatccacgagaagaacgataatgagacaaacgaatgcaccagcagcttcgagacactgttcgaacaggaaccctctagcgaggtgccaaaggacagcaagctgtacatgctggcccagaagaccgtgcagcacattgagcagtacggcaaagcccccgacttcaacaaggtgatccgcgcccacaacttcatccagaccatctacggcacccccctgaaagaggaagaaaaagaagtggtgaggctcatggtgatcaaactgctgaagaagattagcttttacctgacctacatctacccctacgacgtgcccgactacgccggaagcggagctactaacttcagcctgctgaagcaggctggagacgtggaggagaaccctggacctatggacagcgagttcttccagcccgtgtacccccgccactacggagagtgcctgagccccgtgaccacccccagcttcttcagcacacacatgtacaccatcctgatcgccatcgtggtgctggtgattatcatcatcgtcctgatctacctgttcagctctcgcaagaaaaaggccgctgccatcgaagaggaggacatccagttcatcaacccctaccaggaccagcagtgggtggaagtgaccccccagcccggcaccagcaagcctgctggagccaccaccgccagcgtgggaaaacccgtgaccggccgccccgctaccaacagacccgctaccaataagcccgtgaccgacaaccccgtcaccgaccgcctcgtgatggccaccggcggaccagctgccgctcctgctgctgctagcgcccctgctcaccccgctgagccatacaccaccgtgaccacccagaacaccgccagccagaccatgagcgccattgagaatctccgccagcgcaatacatatacccacaaagacctggaaaactccctctaactcgagtctagagggcccgtttaaacccgctgatcagcctcgataatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgagaagccttagagtctcctcttctgaggcggaaagaaccagctggggctctagcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatctttgagaccgcacagggtcttaataagggtgcagagatcctcaggtccttgacaaggtgagtgaatgcagccttcggtttctaccgagtgctgagttatggtaatgggcttttctcccaccatgaccaccaatttctgacgcttggttggcaacttgtagctaaggcggtgtccggtggtattactgtcgtaggtgactttggcctgctttaccagacaaaagataccccttttgcactggtgcaagttaaccatgtcttggagctcttgattcatgcgctgttgctcggccgctgccctgcgtctttctagcaggcgctgctctgtaataattccgtccatttctagctagagaaacctgaattagaatagcccgtagagttgcttgaattgttcataaaccccacagtagctgcgcctttggcctaataccctaagggttttctaagctcacctcctgttctggtaaacagagttattgaggtctgtccggaaaaagtctggtttacggtcaggcggtaggtgtggtgcagcggccggtgacgcactcgtacgttcccggcaggtaaggagggtggtgttttttctgatggagtagctgagctcggagaggttctctcgtagactcactccgtctgggttgaaactgttgtaaatcacagagggagagatgttaaaagtaccaggtaaggttcgccttggtttgcttgggcgggtgaagacggtggcgtttacaggatggcgataggagccccagtatattttaatttctgtatttattatactcagcacagagatggcaacaaagatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagactagggttctgtgagtttgattaaggtacggtgatctgtataagctatgtggtggtggggctatactactgaatgaaaaatgacttgaaattttctgcaattgaaaaataaacacgttgaaacataacacaaacgatacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggctggtgcacacggaccacgtcaaagacttcaaacaaaacataaagaagggtgggctcgtccatgggatccacctcaaaagtcatgtctagcgcgtgggcggagttggcgtagagaaggttttggcccaggtctgtgagtgcgcccatggacataaagttactggagaatgggatgcgccaaagggtgcgatcgcaaagaaactttttctgggtaatgctgtcaactgcggtcttgcctataagcggataggggaagttagcagggtaggcctgtccttcgcgcatggtgggggcaaggtagccaacaaatccagagttgttgtgttggtgtaggatgcccacctgttggtagtccttgtatttagtatcatccaccacctgacggctcatgggctggaagtttctaaagaaggagtacatgcggtccttgtagctctctgggatatagaagccctggtagccaatgttatagttagctagcatttgtaccaggaaccagtctttggtcatgttacactgggcaacgttgtaaccctccccgtcaactgagcgcttaatttcaaactcgttgggggtaagcaggcggtcattgccaggccagctgacagaagagtcaaaggtaatggccaccttcttaaaggtgtggttgaggtaaaaggttccatctaggtagggtatagagccagagtaggtgtaataagggtcgtagcccgagcccagtgatggggtttccttagtcttaaggcgcgtgaaggcccagccgcggaaagccgcccagttgcgggaggggatggatatgggcacgttggtagcgttggcgggtatagggtagagcatgttggcggcggagagatagtcgttaaaggactggtcgttggtgtcgtttctaagcatggcctcaagcgtggaggcggtgttgtgggccatggggaagaaggtggcgtaaaggcaaatgctatcaaacttaatgctggctccgtcaacccttaggtcatttcctagggagctctgcagaaccatgttaacatccttcctgaagttccactcgtaggtgtatgagcccggcaggagaaggaggtttttaatggcaaagaacttctgaggcacctggatgtggaagggcacatagcgaccattgcccagcaacattgagcggtagcgcaggccagcattgcggtggtggttaaatgggttgacgttgtccatatagtcaagggaccagcgtgctccaaggttaatgtagcagtccactagcccgggagccaccactcgcttgttcatgtagtcgtaggtgtttgggttatcagaaatttttacgttggaaggactgtactttagcttgtcgggcaaatacagcgctatgttggagtacaggaaatttctccacaggttggcatttagattgatttccatggcaaaattatttccaactcttatttcatttttatctgaaaattctgtagcatctttttcccatccattttcctgacctgttttaggttttaccttggtaagagtctctgtattaatcacacctcccagtggaaagcagtaatttggaagttcatcttcagttccatgattttcaataattctaacatctggatcatagctgtcaacagcctgattccacatagaaaagtacctggttctatcaccaatggaatcaagcaaaagctggtatgaaagctctgtgtttctgtcttgcaaatctacaacagcattcaactgcgatgcttggcccgccagaacacccatattacccgtgctgttgtaatacattagaccaataaaattgtccctaaaagcaatgtaattaggcctgttgggcatagattgttggcccattagttctcgtgagttaccttccttaatagtgggcatgtaagaaatatgagtgtctggggtttctatatctacatcttcactgtacaataccactttaggagtcaagttatcaccattgcctgcggctgcctcagtagttgagaaaaattgcatttccacttgactttctagctttccattttgttgctttacaagaatgccttgccctccattttcatttgtgggttttgcatatgaaccgtaacatggtttcattggggtagtcttttttaggactctcccagctgcatgattaatttctgtttcgtaccactgagattctcctatttgaggttcaggttgaaatgttttatcggcatatttaggtgtttgaccttcgacacctatttgaataccctcctttgtaatatttataccagaataaggcgcctgcccaaatacgtgagttttttgctgctcagcttgctcgtctacttcgtcttcgttgtcatcgtcctcttcttctaggtttatttcaagagcagtagcagcttcatcccattcgcaaggatttggggcacccttgggagccagggcgttgtaggcagtgccagagtagggcttaaaagtagggcccctgtccagcacgccgcggatgtcaaagtacgtggaagccatgtccagcacacggttatcacccacagctagggtgaaccgcgccttgtacgagtacgcagtatcctcacggtccacagggatgaaccgcagcgtcaaacgctgggaccggtctgtggtcacgtcgtgcgtaggcgccaccgtggggtttctaaacttgttattcaggctgaagtacgtctcggtggcgcgggcaaactgcaccagcccggggctcaggtactccgaggcgtcctggcccgagatgtgcatgtaagaccactgcggcatcatcgaaggggtagccatcttggaaagcgggcgcgcggcggctcagcagctcctctggcggcgacatggacgcatacatgacacacatacgacacgttagctatcagaagcatcgtcggcgcttcagggattgcacccccagacccacgatgctgttcagtgtgctttgccagttgccactggctacgggccgcaacgatcgcggaccgctggcggcgcggcgcagggacgcgcggctaggacgggttacaacaacggcggtcgggcctggcagcacaggtttctgctgggtgtcggcggggggaggcaggtccagcgttacgggtgtgtgctggcccagcactccggtagccatgggcgcgatgggacgggtggtgggcaggccttgctttagtgcctcctcgtacgagggaggctcgtctatttgcgtcaccagagtttcttccctgtcggggcgcggacgcttttcgccacgcccctctggagacactgtctccacggccggtggaggctcctctacgggagggcggggatcaagcttactgttaatcttattttgcactgcctggttggccaggtccaccaccccgctaatgccagaggccaggccatctaccaccttttgttggaaattttgctctttcaacttatccctcagcatctggcctgtgctgctgttccaggccttgctgccatagttcttaacggtggaaccgaaatttttaatgccgctccacagcgagccccagctgaaggcgccaccgctcatattgctggtgccgatatcttgccagtttcccatgaacgggcgcgagccgtgtcgcggggccagagacgcaaagttgatgtcttccattctacaaaatagttacaggaccaagcgagcgtgagagtccagactttttattttgatttttccacatgcaacttgtttttaatcagtgtctctgcgcctgcaaggccacggatgcaattccgggcacggcgccaatcgccgcggcgatcagtggaataaggaggggcaggataccgccgcgcatgcgacggtgcgacgcgcgccgccgccggtggtgcgcacgacgcatgccgcccgtcaggccgtggccggccatgcccctcctacggtgcattcttcctcggaatcccggcaccgggaaacggaggcggcaggtgagggccatatctgcaagaaccacaaagaccggcttttaaacgatgctggggtggtagcgcgctgttggcagcaccagggtcctgcctccttcgcgagccaccctgcgcacggaaatcggggccagcacgggctggcgacggcgacggcggcggcgggttccagtggtggttcggcgtcgggtagttgctcgtcttctggggcggtaggtgtagccacgatagccgggggtaggcgcaatggaaggatgtagggcatattcgggcagtagcgcgctggcggcgccgtacttcctcgaacggcgcgggcgccggggggctgaaacgcgaaacatccacgggtccgtttgcacctccgtagaggtcttggacgcggccgcagcgaccgcctgcaccgcggcatccgccaccgctgaggcaaccggggacgtttgtgtctccatgccctctgtggcggtggcaatactggtgctactggtagtgggtatctgaacgtccacggtctgcacgcccagtcccggcgccacctgcttgattggccgcacgcggacctcgggctccagcccaggttccacggtcattttttccaagacatcttccagtcgctggcgcttgggtaccatcagctgcacggtgggtgccaagtcaccagactcgcgctttaggccgcgcttttcttcggacggtgcaagcgcgggcagcacctgctgcagtgttacgggctttaggctaggtgttgggttgccctcgtccagcggcaacgccagcatgtccttatgccgctttccgtaggcaaactccccgaggcgctcgttggcctgctcaagcaggtcctcgtcgccgtacacctcatcatacacgcgcttgtaggtgcgggtggagcgctcaccgggcgtaaagactacggtggtgccgggtcgcaaaacacgttttacgcgtcgacctttccactgtacccgtcgcctgggcgcggtagcgtgcagcagttccacctcgtcgtcaagttcatcatcatcatctttctttttctttttgacccgctttagctttcggggcttgtaatcctgctcttccttcttcggggggccatagatctccggcgcgatgacctggagcatctcttctttgattttgcgcttggacatagcttcgttgcgcgccgccgccgctggatacatacaacagtacgagtctaagtagttttttcttgcaatctagttgcgcggggggcgggtgcgcacgggcacgcgcaggccgctaaccgagtcgcgcacccaatacacgttgcccctgcgaccctgagtcatagcactaatggccgcggctgctgcggcggccgctcgtcgcctggacctggggggcacagtgacaatacccgcggccagccttcgagcggcccgcatggccgcccgtcggccggtgcgacgtgcgcggttaagcagggccgccgccgcgcgttgggcggcagtgccgggtcggcggcggtggcgacgtgctacgcgcctccgccgtctcttcattttagcatagcgccgggctccgcgcaccacggtctgaatggccgcgtccactgtggacactggtggcggcgtgggcgtgtagttgcgcgcctcctccaccaccgcgtcgatggcgtcatcgacggtggtgcgcccagtgcggccgcgtttgtgcgcgccccagggcgcgcggtagtgcccgcgcacgcgcactgggtgttggtcggagcgcttcttggccccgccaaacatcttgcttgggaagcgcaggccccagcctgtgttattgctgggcgatataaggatggacatgcttgctcaaaaagtgcggctcgataggacgcgcggcgagactatgcccagggccttgtaaacgtaggggcaggtgcggcgtctggcgtcagtaatggtcactcgctggactcctccgatgctgttgcgcagcggtagcgtcccgtgatctgtgagagcaggaacgttttcactgacggtggtgatggtgggggctggcgggcgcgccaaaatctggttctcgggaaagcgattgaacacgtgggtcagagaggtaaactggcggatgagttgggagtagacggcctggtcgttgtagaagctcttggagtgcacgggcaacagctcggcgcccaccaccggaaagttgctgatctggcgcgtggagcggaaggtcacggggtcttgcatcatgtctggcaacgaccagtagacctgctccgagccgcaggttacgtcaggagtgcaaagcagggtccatgagcggattccggtctgagggtcgccgtagttgtatgcaaggtaccagctgcggtactgggtgaaggtgctgtcattgcttattaggttgtaactgcgtttcttgctgtcctctgtcaggggtttgatcaccggtttcttctgaggcttctcgacctcgggttgcgcagcgggggcggcagcttcggccgctgcttcggcctcagcgcgcttctcctcagcccgtgtggcaaaggtgtcgccgcgaatggcatgatcgttcatgtcctccaccggctgcattgccgcggctgccgcgttggagttctcttccgcgccgctgccactgctgttgctgccgcctgcgccacccccgccctgttcggtgtcatctttcaagctcgcctggtaggcgtccacatccaacagtgcgggaatgttaccaccctccagatcatcgtaggtgatcctaaagccctcctggaagggttgccgcttgcggatgcccaacaagttgctcaggcggctgtgggtgaagtccaccccgcatcctggcagcaaaatgatgtctggatggaaggcttcgtttgtatataccccaggcatgacaagaccagtgacggggtcaaaccccagtctgaagttgcgggtgtcaaactttaccccgatgtcgctttccagaaccccgttctgtctgcccactttcaagtagtgctccacgatcgcgttgttcataaggtctatggtcatggtctcggagtagttgccctcgggcagcgtgaactccacccactcgtatttcagctccacctgattgtccttagtaggcaagcgcgacaccatcacccgcgccttaaacttattggtaaacatgaactcgttcacatttggcatgttggtatgcaggatggttttcaggtcgccgccccagtgcgaccggtcgtcaagattgatggtctgtgtgcttgcctcccccgggctgtagtcattgttttgaatgaccgtggtcagaaagttgctgtggtcgttctggtagttcagggatgccacatccgttgacttgttgtccaccaggtacacacgggtggtgtcgaataggggtgccaactcagagtaacggatgctgtttctccccccggtaggccgcaggtaccgcggaggcacaaacggcgggtccaggggagcatcgaagggagaacccagcgccgccgccactggcgccgcgctcaccacactctcgtaggagggaggaggaccttcctcatacatcgccgcgcgccgcatactaaggggaatacaagaaaaccaacgctcggtgccatggccttggtgagttttttattttgcatcatgctttttttttttttaaaacattctccccagcctggggcgaaggtgcgcaaacgggttgccactccctcccaaatccaggacgctgctgtcgtctgccgagtcatcgtcctcccacaccagaccccgctgacggtcgtgcctttgacgacgggtgggcgggcgcgggcctggcacgtccctgtgctcctgcgcgtacgtcttccatctactcatcttgtccactaggctctctatcccgttgttgggaaatgccggaggcaggtttttttcgcgctgcggctgcagcagcgagttgtttaggtactcctcctcgcccagcaggcgcgggcgggtggtgcgagtgctggtaagagaccctatcaagcttggaaatgggctactagcatctgaccgcggggccgcagcgcctagatcggacaagctgcttggcctgcggaagctttcctttcgcagcgccgcctctgcctgctcgcgctgttgcaactctagcagggtctgcggttgcggggaaaacacgctgtcgtctatgtcgtcccagaggaatccatcgttaccctcgggcacctcgaatcccccggtgtagaaaccagggggcggtagccagtgcgggttcaagatggcattggtgaaatactcggggttcacggcggccgcgcgatgcaagtagtccattaggcggttgataaacggccggtttgaggcatacatgcccggttccatgttgcgcgcggtcatgtccagcgccacgctgggcgttaccccgtcgcgcatcaggttaaggctcacgctctgctgcacgtagcgcaaaatgcgctcctcctcgctgtttaaactgtgcaacgaggggatcttctgccgccggttggtcagcaggtagtttagggttgcctccaggctgcccgtgtcctcctgccccagcgcgcggctgacacttgtaatctcctggaaagtatgctcgtccacatgcgcctgacctatggcctcgcggtacagtgtcagcaagtgacctaggtatgtgtcccgggacacgctgccactgtccgtgaagggcgctattagcagcagcaacaggcgcgagttgggcgtcagcaagctagacacggtcgcgcggtcgcctgtgggagcccgcaccccccacagcccctgcaagtttttgaaagcctggctcaggtttacggtctgcaggccttgtctactggtctggaaaaaatagtctggcccagactggtacacctcactttgcggtgtctcagtcaccattagccgcagtgcgctcacaaagttggtgtagtcctcctgtccccgcggcacgttggcgggctgtgtactcaggaaggcgtttagtgcaaccatggagcccaggttgccctgctgctgcgcgcgctcacgctgcgccacggcctcgcgcacatcccccaccagccggtccaggttggtctgcacgttgccgctgttgtaacgagccacgcgctgaagcagcgcgtcgtagaccaggccggcctcgtcgggccggatggccctgttttcggccagcgcgtttacgatcgccagcaccttctcgtgcgtggggtttgcgcgcgccgggaccaccgcttccagaattgcggagagccggttggcctgcggctgctgccggaacgcgtcaggattgcgcgcagtcagcgacatgatgcggtccatgacctggcgccagtcgtccgtggagttaaggccggacggctggctctgcagcgccgcccgcaccgccgggtccgttgcgtcttgcatcatctgatcagaaacatcaccgcttagtactcgccgtcctctggctcgtactcatcgtcctcgtcatattcctccacgccgccgacgttgccagcgcgcgcgggtgccaccgccagcccaggtccggccccagctgcctccagggcgcgtcggcttggggcccagcgcaggtcagcgcccgcgtcaaagtaggactcggcctctctatcgccgctgcccgtgccagccagggccctttgcaggctgtgcatcagctcgcggtcgctgagctcgcgccgccggctcacgctcacggccttgtggatgcgctcgttgcgataaacgcccaggtcgtcgctcaaggtaagcaccttcagcgccatgcgcatgtagaacccctcgatctttacctccttgtctatgggaacgtaaggggtatggtatatcttgcgggcgtaaaacttgcccaggctaagcatggaatagttgatggcggccaccttgtcagccaggctcaagctgcgctcctgcaccactatgctctgcaggatgtttatcaaatcgagcagccagcggccctcgggctctactatgtttagcagcgcatccctgaatgcctcgttgtccctgctgtgctgcactataaggaacagctgcgccatgagcggcttgctatttgggttttgctccagcgcgcttacaaagtcccacagatgcatcagtcctatagccacctcctcgcgcgccacaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgggctcaaagtcctcctcgcgcagcaaccgctcgcgattcaggccatgccgcagctcgcgccctgcgtggaactttcgatcccgcatctcctcgggctcctctccctcgcggtcgcgaaacaggttctgccgcggcacgtacgcctcacgcgtatcacgcttcagctgcacccttgggtgccgctcaggagagggcgctcctagccgcgccaggccctcgccctcctccaagtccaggtagtgccgggcccggcgccgcgggggttcgtaatcaccatctgctgccgcgtcaaccgcggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttagatgcatcatccaaggacacccccgcggcccaccgcccgccgcgcggtaccgtagtcgcgccgcggggatgcggcctcttgcaagccatcgacgccgccaccaaccagcccctggaaattaggtatcacctggatctagcccgcgccctgacccgtctatgcgaggtaaacctgcaggagctcccgcctgacctgacgccgcgggagctccagaccatggacagctcccatctgcgcgatgttgtcatcaagctccgaccgccgcgcgcggacatctggactttgggctcgcgcggcgtggtggtccgatccaccgtaactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaatagccactcctccaattggtggcgggagatccagttcttcccgatcggctcgcatcctcgcaccgagcgtctctttgtcacctacgatgtagagacctatacttggatgggggcctttgggaagcagctcgtgcccttcatgctggtcatgaagttcggcggagatgagcctctagtgactgccgcgcgagacctagccgcgaaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctttcagagcacgggctcagctcccctgaagagctcacctacgaggaacttaaaaaattgccttccatcaagggcatcccgcgcttcttggaactttacattgtgggccacaacatcaacggctttgacgagatcgtgctcgccgcccaggtaattaacaaccgttccgaggtgccgggacccttccgcatcacacgcaactttatgcctcgcgcgggaaagatactcttcaacgatgtcaccttcgccctgccaaatccgcgttccaaaaagcgcacggactttttgctctgggagcagggcggatgcgacgacactgacttcaaataccagtacctcaaagtcatggtcagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagaccccctaccgccattctgctcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggatgcggccaccctcaaaggcatcaccgcgggccaggtgaatatcaaatcctcctcgtttttggaaactgacaatcttagcgcagaagtcatgcccgcttttcagagggagtactcaccccaacagctggccctcgcagacagcgatgcggaagagagtgaggacgaacgcgcccccacccccttttatagccccccttcaggaacacccggtcacgtggcctacacctacaaaccaatcaccttccttgatgccgaagagggcgacatgtgtcttcacaccctggagcgagtggaccccctagtggacaacgaccgctacccctcccacttagcctccttcgtgctggcctggacgcgagcctttgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgccctcaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagagcaggtgaccgagctgtgggaccgcctggaactgcttggtcaaacgctcaaaagcatgcctacggcggacggtctcaaaccgttgaaaaactttgcttccttgcaagaactgctatcgctgggcggcgagcgccttctggcggatttggtcagggaaaacatgcgagtcagggacatgcttaacgaagtggcccccctgctcagggatgacggcagctgcagctctcttaactaccagttgcagccggtaataggtgtgatttacgggcccaccggctgcggtaagtcgcagctgctcaggaacctgctttcttcccagctgatctcccctaccccggaaaccgttttcttcatcgccccgcaggtagacatgatccccccatctgaactcaaagcgtgggaaatgcaaatctgtgagggtaactacgcccctgggccggatggaaccattataccgcagtctggcaccctccgcccgcgctttgtaaaaatggcctatgacgatctcatcctggaacacaactatgacgttagtgatcccagaaatatcttcgcccaggccgccgcccgtgggcccattgccatcattatggacgaatgcatggaaaatcttggaggtcacaagggcgtctccaagttcttccacgcatttccttctaagctacatgacaaatttcccaagtgcaccggatacactgtgctggtggttctgcacaacatgaatccccggagggatatggctgggaacatagccaacctaaaaatacagtccaagatgcatctcatatccccacgtatgcacccatcccagcttaaccgctttgtaaacacttacaccaagggcctgcccctggcaatcagcttgctactgaaagacatttttaggcaccacgcccagcgctcctgctacgactggatcatctacaacaccaccccgcagcatgaagctctgcagtggtgctacctccaccccagagacgggcttatgcccatgtatctgaacatccagagtcacctttaccacgtcctggaaaaaatacacaggaccctcaacgaccgagaccgctggtcccgggcctaccgcgcgcgcaaaacccctaaataaagacagcaagacacttgcttgatccaaatccaaacagagtctggttttttatttatgttttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttgtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggccagcggcggagctttttgcctgatcgccaatgatggcaaggccgacaagatcatcctggcccaggacctgctgaattccaggatcagcaatatcaagaacgtgaacaagagctacggcaagcctgaccccgagcctacactgagccagatcgaggagacacacctggtgcactttaacgcccacttcaagccctacgtgcccgtgggcttcgagtacaataaggtgagaccccacaccggcacacctaccctgggcaataagctgacatttggcatccctcagtacggcgacttctttcacgacatggtgggccaccacatcctgggcgcctgccacagctcctggcaggatgcccctatccagggcacaagccagatgggcgcccacggccagctgcagaccttcccaagaaacggctacgactgggacaatcagacccctctggagggcgccgtgtacaccctggtggaccctttcggcagacctatcgtgcctggcacaaagaacgcctacagaaatctggtgtactactgcgagtaccccggcgagaggctgtacgagaatgtgaggtttgacgtgaacggcaactccctggatgagtactccagcgatgtgaccacactggtgaggaagttttgtatccctggcgataagatgacaggctacaagcacctggtgggccaggaggtgtccgtggagggcacctccggccctctgctgtgtaatatccacgatctgcacaagcctcaccagagcaagcccatcctgaccgatgagaatgacacacagaggacctgcagccacaccaatcctaagttcctgtcccagcacttccctgagaacagccacaatatccagacagccggcaagcaggacatcacacccatcacagacgccacatacctggacatcaggagaaatgtgcactactcctgcaatggcccccagacacctaagtactaccagccccccctggccctgtggatcaagctgaggttctggttcaatgagaacgtgaatctggccatcccctccgtgtccatccctttcggcgagagattcatcaccatcaagctggcctcccagaaggacctggtgaatgagtttcctggcctgttcgtgaggcagagcaggttcatcgccggcagaccttccagaaggaacatcagattcaagccttggtttatccctggcgtgatcaacgagatctccctgaccaacaacgagctgtacatcaataacctgtttgtgacccctgagatccacaatctgttcgtgaagagggtgagattctccctgatcagagtgcacaagacccaggtgacacacaccaataacaatcaccacgacgagaagctgatgtccgccctgaagtggcctatcgagtacatgttcatcggcctgaagcctacctggaacatctccgaccagaacccccaccagcacagagactggcacaagttcggccacgtggtgaatgccatcatgcagcctacccaccacgccgagatcagcttccaggatagggatacagccctgcctgatgcctgctcctccatcagcgatattagccctgtgacctaccccatcaccctgcctatcatcaagaacatcagcgtgacagcccacggcatcaacctgatcgacaagtttcccagcaagttttgctcctcctacatcccttttcactacggcggcaatgccatcaagacacccgacgaccccggcgccatgatgatcacattcgccctgaagcctagagaggagtaccagccctccggccacatcaacgtgagcagggccagggagttctacatcagctgggatacagactacgtgggcagcatcaccaccgccgatctggtggtgagcgcctccgccatcaatttcctgctgctgcagaacggcagcgccgtgctgagatacagcacccaccatcaccaccaccactgagatatcggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaaccgcggccgcatggccgaattcaatattgatgaactgctgaaaaacgtgctggaagatccctctaccgagatctctgaagagaccctgaaacagctgtatcagcgcaccaacccctataaacagttcaagaatgactcccgggtggccttttgctccttcaccaatctgagggagcagtacatccgccgcctgatcatgacctccttcatcggctacgtcttcaaggccctccaggaatggatgcccagctatagcaagcctacccacaccaccaagaccctgctcagcgaactcatcaccctcgtggacaccctgaagcaggagaccaacgacgtgcccagcgaatccgtcgtgaacaccatcctgagcatcgccgacagctgcaaaacccagacccagaaaagcaaggaagccaaaaccaccatcgacagcttcctgagagaacacttcgtgttcgaccccaacctgcatgcccagagcgcctacacctgcgccgacaccaacgtcgacacctgcgccagcatgtgcgccgacacaaacgtggacacctgcgctagcatgtgcgctgataccaacgtcgatacctgcgcctccacctgcacctccaccgagtacaccgacctggccgaccccgagcgcatccctctgcacatcatgcagaaaaccctgaacgtgcccaacgaactgcaggccgatattgacgccatcacccagaccccccagggctaccgcgccgctgctcatatcctgcagaacatcgaactccaccagagcatcaagcacatgctggagaacccccgcgccttcaagcccatcctgtttaacaccaagatcacacgctatctgtcccagcacatccccccccaggacaccttctacaaatggaactactacatcgaagacaactacgaagaactgcgcgccgccacagagagcatctaccccgaaaagcccgacctggagttcgccttcatcatctacgacgtggtggacagcagcaaccagcagaaggtcgacgagttctactataagtacaaggaccagatctttagcgaggtgagcagcatccagctgggcaactggaccctgctgggcagctttaaggccaaccgcgagcgctacaactacttcaaccagaacaacgaaatcatcaagcggatcttggacaggcacgaggaggacctgaagatcggcaaagagatcctccgcaacaccatctaccacaaaaaggccaaaaacatccaggagaccggacccgacgcccccggactgtctatctacaacagcacattccacaccgacagcggcatcaaaggactgctcagcttcaaagagctcaaaaacctggaaaaagccagcggcaacatcaagaaagcccgcgaatacgatttcatcgacgactgcgaagagaaaatcaagcagctgctgagcaaagagaacctgacccccgacgaggaaagcgagctgatcaagaccaaaaagcagctggacaacgccctggagatgctgaacgtgcctgacgacaccatccgcgtggacatgtgggtgaacaacaacaacaaactcgaaaaggagatcctgtacaccaaggccgagctgtacccctacgacgtgcccgattacgcccaccatcaccaccaccactgactcgagtctagagggcccgtttaaacccgctgatcacttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccggaaaaacacctgggcgagtctccacgtaaacggtcaaagtccccgcgggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgataagctatcaaacatgagaattcggcgcgccattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaacgcgcctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcacctgcacaccgcgccttaattaagaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcagcgaaccaatcagcagggtcatcgctagccagatcctctacgccggacgcatcgtggccaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgctgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcata

SEQ ID NO: 7 - DNA, 1023 bp, Artificial Sequence
  Synthetic ORF6/7
atcgtttgtgttatgtttcaacgtgtttatttttcaattgcagaaaatttcaagtcatttttcattcagtagtatagccccaccaccacatagcttatacagatcaccgtaccttaatcaaactcacagaaccctagtctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcat

SEQ ID NO: 8 - DNA, 644 bp, Artificial Sequence
  Synthetic IRES
ggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaacc

SEQ ID NO: 9 - DNA, 66 bp, Artificial Sequence
  Synthetic 2A
ggaagcggagctactaacttcagcctgctgaagcaggctggagacgtggaggagaaccctggacct

SEQ ID NO: 10 - DNA, 2796 bp, Artificial Sequence
  Synthetic Skeleton of puc origin, amp and other basic elements
gaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcattcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacgtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcttatcgaaa

SEQ ID NO: 11 - DNA, 1722 bp, Artificial Sequence
  Synthetic EF1a-EGFP-HBV polyA
ttttaaaagaaaaggggggattggggggtacagtgcaggggaaagaatagtagacataatagcaacagacatacaaactaaagaattacaaaaacaaattacaaaattcaaaattttatcgtactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagctccctatcagtgatagagatctccctatcagtgatagagattcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgagtctagagggcccgtttaaacccgctgatcagcctcgataatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgagaagccttagagtctcctcttctgaggcggaaagaaccagctggggctcta

SEQ ID NO: 12 - DNA, 957 bp, Artificial Sequence
  Synthetic CMV-MCS
gtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcacttcgcccacccc

SEQ ID NO: 13 - DNA, 147 bp, Artificial Sequence
  Synthetic SV40
aacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctct

SEQ ID NO: 14 - DNA, 414 bp, Artificial Sequence
  Synthetic CP129R
atggagcaccccagcacaaactacacccccgagcagcagcacgagaagctgaagcactacgtgctgatccctaagcacctgtggtcctacatcaagtacggcacccacgtgagatactacacaacacagaacgtgttcagagtgggcggctttgtgctgcagaacccttacgaggccgtgatcaagaatgaggtgaagacagccatcagactgcagaattccttcaacaccaaggccaagggccacgtgacctgggccgtgccatacgataacatcagcaagctgtacgccaagcccgatgccatcatgctgacaatccaggagaatgtggagaaggccctgcacgccctgaatcagaatgtgctgaccctggccagcaagatcaggtacccttacgacgtgcccgattacgcc

SEQ ID NO: 15 - DNA, 471 bp, Artificial Sequence
  Synthetic Ubiqutin
atgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaa

SEQ ID NO: 16 - DNA, 645 bp, Artificial Sequence
  Synthetic MGF5L6L
atgctggtgatcttcctgggcatcctgggcctgctggccagccaggtgagcagccagctggtgggccagctgaggcccaccgaggagccacctgaggaggagctggagtactggtgtgcctacatggagagctgccagttctgctgggattgtcaggatggcacctgcatcaacaagatcgacggctccgtgatctacaagaatgagtacgtgaagagctgtctggtgagcagatggctggacaagtgcatgtacgatctggacaagggcatctaccacaccatgaactgcaaccaggtgctgggcctgcctaaccagcccgccggacagctgcaccccaccgataatcccccccaggaggagctggaatactggtgcacctacaccgagaactgcaagttctgctggaattgtcagaatggcctgtgtgagggcaagctggagaacacaaccatcctggagaatgagtacgtccagagctgcatcgtgagcagatggctcaacaagtgtatgtacgatctcggccagggcatccaccacgtgatggcctgctccgagcccaagccctggaatccttacaagatcctgaagagagagtggaaggagaataactcctacccctacgatgtgcctgactacgcctga

SEQ ID NO: 17 - DNA, 948 bp, Artificial Sequence
  Synthetic CP312R
atgacaacccacatcttccacgccgatgatctgctgcaggccctgcagcaggccaaggccgagaagaacttctccagcgtgttcagcctggattgggataagctgaggacagccaagaggaacacaaccgtgaagtacgtgacagtgaacgtgatcgtgaagggcaagaaggcccccctgatgttcaacttccagaacgagaagcacgtgggcacaatcccccctagcacagatgaggaggtgatcagaatgaatgccgagaatcccaagttcctggtgaagaagagagatagggacccctgcctgcagttcaataagtacaagatcagccctcccctggaggatgatggcctgacagtgaagaagaacgagcagggcgaggagatctaccctggcgacgaggagaagagcaagctgttccagatcatcgagctgctggaggaggcctttgaggacgccgtgcagaagggccctgaggccatgaagaccaagcacgtgatcaagctgatccagagaaagatcagcaactccgccgtgaagaacgccgataagcctctgcccaaccctatcgccaggatcagaatcaagatcaatcctgccacatccatcctgacacccatcctgctggacaagaacaagcctatcacactgcagaacggcaagaccagcttcgaggagctgaaggatgaggacggcgtgaaggccaaccctgacaacatccacaagctgatcgagtcccacagcatccacgatggcatcatcaacgccagaagcatctgcatctccaacatgggcatcagcttccccctgtgcctggagatgggcgtggtgaaggtgtttgagaagaataatggcatcgacgtgaatagcatctacggctccgacgacatctccacactggtgaaccagatcgccatcgcctacccctacgacgtgcctgattacgcc

SEQ ID NO: 18 - DNA, 375 bp, Artificial Sequence
  Synthetic MGF110-4L
atgctggtggtgtttctgggcatcctgggcctgctggccaatcaggtggtgggcctgcccacccaggccggaggacatctgagatccaccgataatcccccccaggaggagctgggctactggtgcacctacatggagaggtgcaagttctgctgggagtgcgcccacggcatctgcaagaataaggtgaacaattccatgcctctgatcatcgagaattcctacctgaccacatgtgaggtgtccaggtggtacaaccagtgtacctacggcgagggcaatggccactaccacgtgatggactgctccgatcccgtgcctcacaacagaccctaccagctgctgatgaagatctacgagaagaaggacctgtga

SEQ ID NO: 19 - DNA, 38432 bp, Artificial Sequence
  Synthetic pAd5LCL3-CP129Rubiqutin-MGF5L6L- CP312R-MGF110-4L
agcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacacgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgatcgattaattaacgacccatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttttttaaaagaaaaggggggattggggggtacagtgcaggggaaagaatagtagacataatagcaacagacatacaaactaaagaattacaaaaacaaattacaaaattcaaaattttatcgtactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggatccgccaccatgacaacccacatcttccacgccgatgatctgctgcaggccctgcagcaggccaaggccgagaagaacttctccagcgtgttcagcctggattgggataagctgaggacagccaagaggaacacaaccgtgaagtacgtgacagtgaacgtgatcgtgaagggcaagaaggcccccctgatgttcaacttccagaacgagaagcacgtgggcacaatcccccctagcacagatgaggaggtgatcagaatgaatgccgagaatcccaagttcctggtgaagaagagagatagggacccctgcctgcagttcaataagtacaagatcagccctcccctggaggatgatggcctgacagtgaagaagaacgagcagggcgaggagatctaccctggcgacgaggagaagagcaagctgttccagatcatcgagctgctggaggaggcctttgaggacgccgtgcagaagggccctgaggccatgaagaccaagcacgtgatcaagctgatccagagaaagatcagcaactccgccgtgaagaacgccgataagcctctgcccaaccctatcgccaggatcagaatcaagatcaatcctgccacatccatcctgacacccatcctgctggacaagaacaagcctatcacactgcagaacggcaagaccagcttcgaggagctgaaggatgaggacggcgtgaaggccaaccctgacaacatccacaagctgatcgagtcccacagcatccacgatggcatcatcaacgccagaagcatctgcatctccaacatgggcatcagcttccccctgtgcctggagatgggcgtggtgaaggtgtttgagaagaataatggcatcgacgtgaatagcatctacggctccgacgacatctccacactggtgaaccagatcgccatcgcctacccctacgacgtgcctgattacgccggaagcggagctactaacttcagcctgctgaagcaggctggagacgtggaggagaaccctggacctatgctggtggtgtttctgggcatcctgggcctgctggccaatcaggtggtgggcctgcccacccaggccggaggacatctgagatccaccgataatcccccccaggaggagctgggctactggtgcacctacatggagaggtgcaagttctgctgggagtgcgcccacggcatctgcaagaataaggtgaacaattccatgcctctgatcatcgagaattcctacctgaccacatgtgaggtgtccaggtggtacaaccagtgtacctacggcgagggcaatggccactaccacgtgatggactgctccgatcccgtgcctcacaacagaccctaccagctgctgatgaagatctacgagaagaaggacctgtgactcgagtctagagggcccgtttaaacccgctgatcagcctcgataatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgagaagccttagagtctcctcttctgaggcggaaagaaccagctggggctctagcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatctttgagaccgcacagggtcttaataagggtgcagagatcctcaggtccttgacaaggtgagtgaatgcagccttcggtttctaccgagtgctgagttatggtaatgggcttttctcccaccatgaccaccaatttctgacgcttggttggcaacttgtagctaaggcggtgtccggtggtattactgtcgtaggtgactttggcctgctttaccagacaaaagataccccttttgcactggtgcaagttaaccatgtcttggagctcttgattcatgcgctgttgctcggccgctgccctgcgtctttctagcaggcgctgctctgtaataattccgtccatttctagctagagaaacctgaattagaatagcccgtagagttgcttgaattgttcataaaccccacagtagctgcgcctttggcctaataccctaagggttttctaagctcacctcctgttctggtaaacagagttattgaggtctgtccggaaaaagtctggtttacggtcaggcggtaggtgtggtgcagcggccggtgacgcactcgtacgttcccggcaggtaaggagggtggtgttttttctgatggagtagctgagctcggagaggttctctcgtagactcactccgtctgggttgaaactgttgtaaatcacagagggagagatgttaaaagtaccaggtaaggttcgccttggtttgcttgggcgggtgaagacggtggcgtttacaggatggcgataggagccccagtatattttaatttctgtatttattatactcagcacagagatggcaacaaagatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagactagggttctgtgagtttgattaaggtacggtgatctgtataagctatgtggtggtggggctatactactgaatgaaaaatgacttgaaattttctgcaattgaaaaataaacacgttgaaacataacacaaacgatacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggctggtgcacacggaccacgtcaaagacttcaaacaaaacataaagaagggtgggctcgtccatgggatccacctcaaaagtcatgtctagcgcgtgggcggagttggcgtagagaaggttttggcccaggtctgtgagtgcgcccatggacataaagttactggagaatgggatgcgccaaagggtgcgatcgcaaagaaactttttctgggtaatgctgtcaactgcggtcttgcctataagcggataggggaagttagcagggtaggcctgtccttcgcgcatggtgggggcaaggtagccaacaaatccagagttgttgtgttggtgtaggatgcccacctgttggtagtccttgtatttagtatcatccaccacctgacggctcatgggctggaagtttctaaagaaggagtacatgcggtccttgtagctctctgggatatagaagccctggtagccaatgttatagttagctagcatttgtaccaggaaccagtctttggtcatgttacactgggcaacgttgtaaccctccccgtcaactgagcgcttaatttcaaactcgttgggggtaagcaggcggtcattgccaggccagctgacagaagagtcaaaggtaatggccaccttcttaaaggtgtggttgaggtaaaaggttccatctaggtagggtatagagccagagtaggtgtaataagggtcgtagcccgagcccagtgatggggtttccttagtcttaaggcgcgtgaaggcccagccgcggaaagccgcccagttgcgggaggggatggatatgggcacgttggtagcgttggcgggtatagggtagagcatgttggcggcggagagatagtcgttaaaggactggtcgttggtgtcgtttctaagcatggcctcaagcgtggaggcggtgttgtgggccatggggaagaaggtggcgtaaaggcaaatgctatcaaacttaatgctggctccgtcaacccttaggtcatttcctagggagctctgcagaaccatgttaacatccttcctgaagttccactcgtaggtgtatgagcccggcaggagaaggaggtttttaatggcaaagaacttctgaggcacctggatgtggaagggcacatagcgaccattgcccagcaacattgagcggtagcgcaggccagcattgcggtggtggttaaatgggttgacgttgtccatatagtcaagggaccagcgtgctccaaggttaatgtagcagtccactagcccgggagccaccactcgcttgttcatgtagtcgtaggtgtttgggttatcagaaatttttacgttggaaggactgtactttagcttgtcgggcaaatacagcgctatgttggagtacaggaaatttctccacaggttggcatttagattgatttccatggcaaaattatttccaactcttatttcatttttatctgaaaattctgtagcatctttttcccatccattttcctgacctgttttaggttttaccttggtaagagtctctgtattaatcacacctcccagtggaaagcagtaatttggaagttcatcttcagttccatgattttcaataattctaacatctggatcatagctgtcaacagcctgattccacatagaaaagtacctggttctatcaccaatggaatcaagcaaaagctggtatgaaagctctgtgtttctgtcttgcaaatctacaacagcattcaactgcgatgcttggcccgccagaacacccatattacccgtgctgttgtaatacattagaccaataaaattgtccctaaaagcaatgtaattaggcctgttgggcatagattgttggcccattagttctcgtgagttaccttccttaatagtgggcatgtaagaaatatgagtgtctggggtttctatatctacatcttcactgtacaataccactttaggagtcaagttatcaccattgcctgcggctgcctcagtagttgagaaaaattgcatttccacttgactttctagctttccattttgttgctttacaagaatgccttgccctccattttcatttgtgggttttgcatatgaaccgtaacatggtttcattggggtagtcttttttaggactctcccagctgcatgattaatttctgtttcgtaccactgagattctcctatttgaggttcaggttgaaatgttttatcggcatatttaggtgtttgaccttcgacacctatttgaataccctcctttgtaatatttataccagaataaggcgcctgcccaaatacgtgagttttttgctgctcagcttgctcgtctacttcgtcttcgttgtcatcgtcctcttcttctaggtttatttcaagagcagtagcagcttcatcccattcgcaaggatttggggcacccttgggagccagggcgttgtaggcagtgccagagtagggcttaaaagtagggcccctgtccagcacgccgcggatgtcaaagtacgtggaagccatgtccagcacacggttatcacccacagctagggtgaaccgcgccttgtacgagtacgcagtatcctcacggtccacagggatgaaccgcagcgtcaaacgctgggaccggtctgtggtcacgtcgtgcgtaggcgccaccgtggggtttctaaacttgttattcaggctgaagtacgtctcggtggcgcgggcaaactgcaccagcccggggctcaggtactccgaggcgtcctggcccgagatgtgcatgtaagaccactgcggcatcatcgaaggggtagccatcttggaaagcgggcgcgcggcggctcagcagctcctctggcggcgacatggacgcatacatgacacacatacgacacgttagctatcagaagcatcgtcggcgcttcagggattgcacccccagacccacgatgctgttcagtgtgctttgccagttgccactggctacgggccgcaacgatcgcggaccgctggcggcgcggcgcagggacgcgcggctaggacgggttacaacaacggcggtcgggcctggcagcacaggtttctgctgggtgtcggcggggggaggcaggtccagcgttacgggtgtgtgctggcccagcactccggtagccatgggcgcgatgggacgggtggtgggcaggccttgctttagtgcctcctcgtacgagggaggctcgtctatttgcgtcaccagagtttcttccctgtcggggcgcggacgcttttcgccacgcccctctggagacactgtctccacggccggtggaggctcctctacgggagggcggggatcaagcttactgttaatcttattttgcactgcctggttggccaggtccaccaccccgctaatgccagaggccaggccatctaccaccttttgttggaaattttgctctttcaacttatccctcagcatctggcctgtgctgctgttccaggccttgctgccatagttcttaacggtggaaccgaaatttttaatgccgctccacagcgagccccagctgaaggcgccaccgctcatattgctggtgccgatatcttgccagtttcccatgaacgggcgcgagccgtgtcgcggggccagagacgcaaagttgatgtcttccattctacaaaatagttacaggaccaagcgagcgtgagagtccagactttttattttgatttttccacatgcaacttgtttttaatcagtgtctctgcgcctgcaaggccacggatgcaattccgggcacggcgccaatcgccgcggcgatcagtggaataaggaggggcaggataccgccgcgcatgcgacggtgcgacgcgcgccgccgccggtggtgcgcacgacgcatgccgcccgtcaggccgtggccggccatgcccctcctacggtgcattcttcctcggaatcccggcaccgggaaacggaggcggcaggtgagggccatatctgcaagaaccacaaagaccggcttttaaacgatgctggggtggtagcgcgctgttggcagcaccagggtcctgcctccttcgcgagccaccctgcgcacggaaatcggggccagcacgggctggcgacggcgacggcggcggcgggttccagtggtggttcggcgtcgggtagttgctcgtcttctggggcggtaggtgtagccacgatagccgggggtaggcgcaatggaaggatgtagggcatattcgggcagtagcgcgctggcggcgccgtacttcctcgaacggcgcgggcgccggggggctgaaacgcgaaacatccacgggtccgtttgcacctccgtagaggtcttggacgcggccgcagcgaccgcctgcaccgcggcatccgccaccgctgaggcaaccggggacgtttgtgtctccatgccctctgtggcggtggcaatactggtgctactggtagtgggtatctgaacgtccacggtctgcacgcccagtcccggcgccacctgcttgattggccgcacgcggacctcgggctccagcccaggttccacggtcattttttccaagacatcttccagtcgctggcgcttgggtaccatcagctgcacggtgggtgccaagtcaccagactcgcgctttaggccgcgcttttcttcggacggtgcaagcgcgggcagcacctgctgcagtgttacgggctttaggctaggtgttgggttgccctcgtccagcggcaacgccagcatgtccttatgccgctttccgtaggcaaactccccgaggcgctcgttggcctgctcaagcaggtcctcgtcgccgtacacctcatcatacacgcgcttgtaggtgcgggtggagcgctcaccgggcgtaaagactacggtggtgccgggtcgcaaaacacgttttacgcgtcgacctttccactgtacccgtcgcctgggcgcggtagcgtgcagcagttccacctcgtcgtcaagttcatcatcatcatctttctttttctttttgacccgctttagctttcggggcttgtaatcctgctcttccttcttcggggggccatagatctccggcgcgatgacctggagcatctcttctttgattttgcgcttggacatagcttcgttgcgcgccgccgccgctggatacatacaacagtacgagtctaagtagttttttcttgcaatctagttgcgcggggggcgggtgcgcacgggcacgcgcaggccgctaaccgagtcgcgcacccaatacacgttgcccctgcgaccctgagtcatagcactaatggccgcggctgctgcggcggccgctcgtcgcctggacctggggggcacagtgacaatacccgcggccagccttcgagcggcccgcatggccgcccgtcggccggtgcgacgtgcgcggttaagcagggccgccgccgcgcgttgggcggcagtgccgggtcggcggcggtggcgacgtgctacgcgcctccgccgtctcttcattttagcatagcgccgggctccgcgcaccacggtctgaatggccgcgtccactgtggacactggtggcggcgtgggcgtgtagttgcgcgcctcctccaccaccgcgtcgatggcgtcatcgacggtggtgcgcccagtgcggccgcgtttgtgcgcgccccagggcgcgcggtagtgcccgcgcacgcgcactgggtgttggtcggagcgcttcttggccccgccaaacatcttgcttgggaagcgcaggccccagcctgtgttattgctgggcgatataaggatggacatgcttgctcaaaaagtgcggctcgataggacgcgcggcgagactatgcccagggccttgtaaacgtaggggcaggtgcggcgtctggcgtcagtaatggtcactcgctggactcctccgatgctgttgcgcagcggtagcgtcccgtgatctgtgagagcaggaacgttttcactgacggtggtgatggtgggggctggcgggcgcgccaaaatctggttctcgggaaagcgattgaacacgtgggtcagagaggtaaactggcggatgagttgggagtagacggcctggtcgttgtagaagctcttggagtgcacgggcaacagctcggcgcccaccaccggaaagttgctgatctggcgcgtggagcggaaggtcacggggtcttgcatcatgtctggcaacgaccagtagacctgctccgagccgcaggttacgtcaggagtgcaaagcagggtccatgagcggattccggtctgagggtcgccgtagttgtatgcaaggtaccagctgcggtactgggtgaaggtgctgtcattgcttattaggttgtaactgcgtttcttgctgtcctctgtcaggggtttgatcaccggtttcttctgaggcttctcgacctcgggttgcgcagcgggggcggcagcttcggccgctgcttcggcctcagcgcgcttctcctcagcccgtgtggcaaaggtgtcgccgcgaatggcatgatcgttcatgtcctccaccggctgcattgccgcggctgccgcgttggagttctcttccgcgccgctgccactgctgttgctgccgcctgcgccacccccgccctgttcggtgtcatctttcaagctcgcctggtaggcgtccacatccaacagtgcgggaatgttaccaccctccagatcatcgtaggtgatcctaaagccctcctggaagggttgccgcttgcggatgcccaacaagttgctcaggcggctgtgggtgaagtccaccccgcatcctggcagcaaaatgatgtctggatggaaggcttcgtttgtatataccccaggcatgacaagaccagtgacggggtcaaaccccagtctgaagttgcgggtgtcaaactttaccccgatgtcgctttccagaaccccgttctgtctgcccactttcaagtagtgctccacgatcgcgttgttcataaggtctatggtcatggtctcggagtagttgccctcgggcagcgtgaactccacccactcgtatttcagctccacctgattgtccttagtaggcaagcgcgacaccatcacccgcgccttaaacttattggtaaacatgaactcgttcacatttggcatgttggtatgcaggatggttttcaggtcgccgccccagtgcgaccggtcgtcaagattgatggtctgtgtgcttgcctcccccgggctgtagtcattgttttgaatgaccgtggtcagaaagttgctgtggtcgttctggtagttcagggatgccacatccgttgacttgttgtccaccaggtacacacgggtggtgtcgaataggggtgccaactcagagtaacggatgctgtttctccccccggtaggccgcaggtaccgcggaggcacaaacggcgggtccaggggagcatcgaagggagaacccagcgccgccgccactggcgccgcgctcaccacactctcgtaggagggaggaggaccttcctcatacatcgccgcgcgccgcatactaaggggaatacaagaaaaccaacgctcggtgccatggccttggtgagttttttattttgcatcatgctttttttttttttaaaacattctccccagcctggggcgaaggtgcgcaaacgggttgccactccctcccaaatccaggacgctgctgtcgtctgccgagtcatcgtcctcccacaccagaccccgctgacggtcgtgcctttgacgacgggtgggcgggcgcgggcctggcacgtccctgtgctcctgcgcgtacgtcttccatctactcatcttgtccactaggctctctatcccgttgttgggaaatgccggaggcaggtttttttcgcgctgcggctgcagcagcgagttgtttaggtactcctcctcgcccagcaggcgcgggcgggtggtgcgagtgctggtaagagaccctatcaagcttggaaatgggctactagcatctgaccgcggggccgcagcgcctagatcggacaagctgcttggcctgcggaagctttcctttcgcagcgccgcctctgcctgctcgcgctgttgcaactctagcagggtctgcggttgcggggaaaacacgctgtcgtctatgtcgtcccagaggaatccatcgttaccctcgggcacctcgaatcccccggtgtagaaaccagggggcggtagccagtgcgggttcaagatggcattggtgaaatactcggggttcacggcggccgcgcgatgcaagtagtccattaggcggttgataaacggccggtttgaggcatacatgcccggttccatgttgcgcgcggtcatgtccagcgccacgctgggcgttaccccgtcgcgcatcaggttaaggctcacgctctgctgcacgtagcgcaaaatgcgctcctcctcgctgtttaaactgtgcaacgaggggatcttctgccgccggttggtcagcaggtagtttagggttgcctccaggctgcccgtgtcctcctgccccagcgcgcggctgacacttgtaatctcctggaaagtatgctcgtccacatgcgcctgacctatggcctcgcggtacagtgtcagcaagtgacctaggtatgtgtcccgggacacgctgccactgtccgtgaagggcgctattagcagcagcaacaggcgcgagttgggcgtcagcaagctagacacggtcgcgcggtcgcctgtgggagcccgcaccccccacagcccctgcaagtttttgaaagcctggctcaggtttacggtctgcaggccttgtctactggtctggaaaaaatagtctggcccagactggtacacctcactttgcggtgtctcagtcaccattagccgcagtgcgctcacaaagttggtgtagtcctcctgtccccgcggcacgttggcgggctgtgtactcaggaaggcgtttagtgcaaccatggagcccaggttgccctgctgctgcgcgcgctcacgctgcgccacggcctcgcgcacatcccccaccagccggtccaggttggtctgcacgttgccgctgttgtaacgagccacgcgctgaagcagcgcgtcgtagaccaggccggcctcgtcgggccggatggccctgttttcggccagcgcgtttacgatcgccagcaccttctcgtgcgtggggtttgcgcgcgccgggaccaccgcttccagaattgcggagagccggttggcctgcggctgctgccggaacgcgtcaggattgcgcgcagtcagcgacatgatgcggtccatgacctggcgccagtcgtccgtggagttaaggccggacggctggctctgcagcgccgcccgcaccgccgggtccgttgcgtcttgcatcatctgatcagaaacatcaccgcttagtactcgccgtcctctggctcgtactcatcgtcctcgtcatattcctccacgccgccgacgttgccagcgcgcgcgggtgccaccgccagcccaggtccggccccagctgcctccagggcgcgtcggcttggggcccagcgcaggtcagcgcccgcgtcaaagtaggactcggcctctctatcgccgctgcccgtgccagccagggccctttgcaggctgtgcatcagctcgcggtcgctgagctcgcgccgccggctcacgctcacggccttgtggatgcgctcgttgcgataaacgcccaggtcgtcgctcaaggtaagcaccttcagcgccatgcgcatgtagaacccctcgatctttacctccttgtctatgggaacgtaaggggtatggtatatcttgcgggcgtaaaacttgcccaggctaagcatggaatagttgatggcggccaccttgtcagccaggctcaagctgcgctcctgcaccactatgctctgcaggatgtttatcaaatcgagcagccagcggccctcgggctctactatgtttagcagcgcatccctgaatgcctcgttgtccctgctgtgctgcactataaggaacagctgcgccatgagcggcttgctatttgggttttgctccagcgcgcttacaaagtcccacagatgcatcagtcctatagccacctcctcgcgcgccacaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgggctcaaagtcctcctcgcgcagcaaccgctcgcgattcaggccatgccgcagctcgcgccctgcgtggaactttcgatcccgcatctcctcgggctcctctccctcgcggtcgcgaaacaggttctgccgcggcacgtacgcctcacgcgtatcacgcttcagctgcacccttgggtgccgctcaggagagggcgctcctagccgcgccaggccctcgccctcctccaagtccaggtagtgccgggcccggcgccgcgggggttcgtaatcaccatctgctgccgcgtcaaccgcggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttagatgcatcatccaaggacacccccgcggcccaccgcccgccgcgcggtaccgtagtcgcgccgcggggatgcggcctcttgcaagccatcgacgccgccaccaaccagcccctggaaattaggtatcacctggatctagcccgcgccctgacccgtctatgcgaggtaaacctgcaggagctcccgcctgacctgacgccgcgggagctccagaccatggacagctcccatctgcgcgatgttgtcatcaagctccgaccgccgcgcgcggacatctggactttgggctcgcgcggcgtggtggtccgatccaccgtaactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaatagccactcctccaattggtggcgggagatccagttcttcccgatcggctcgcatcctcgcaccgagcgtctctttgtcacctacgatgtagagacctatacttggatgggggcctttgggaagcagctcgtgcccttcatgctggtcatgaagttcggcggagatgagcctctagtgactgccgcgcgagacctagccgcgaaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctttcagagcacgggctcagctcccctgaagagctcacctacgaggaacttaaaaaattgccttccatcaagggcatcccgcgcttcttggaactttacattgtgggccacaacatcaacggctttgacgagatcgtgctcgccgcccaggtaattaacaaccgttccgaggtgccgggacccttccgcatcacacgcaactttatgcctcgcgcgggaaagatactcttcaacgatgtcaccttcgccctgccaaatccgcgttccaaaaagcgcacggactttttgctctgggagcagggcggatgcgacgacactgacttcaaataccagtacctcaaagtcatggtcagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagaccccctaccgccattctgctcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggatgcggccaccctcaaaggcatcaccgcgggccaggtgaatatcaaatcctcctcgtttttggaaactgacaatcttagcgcagaagtcatgcccgcttttcagagggagtactcaccccaacagctggccctcgcagacagcgatgcggaagagagtgaggacgaacgcgcccccacccccttttatagccccccttcaggaacacccggtcacgtggcctacacctacaaaccaatcaccttccttgatgccgaagagggcgacatgtgtcttcacaccctggagcgagtggaccccctagtggacaacgaccgctacccctcccacttagcctccttcgtgctggcctggacgcgagcctttgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgccctcaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagagcaggtgaccgagctgtgggaccgcctggaactgcttggtcaaacgctcaaaagcatgcctacggcggacggtctcaaaccgttgaaaaactttgcttccttgcaagaactgctatcgctgggcggcgagcgccttctggcggatttggtcagggaaaacatgcgagtcagggacatgcttaacgaagtggcccccctgctcagggatgacggcagctgcagctctcttaactaccagttgcagccggtaataggtgtgatttacgggcccaccggctgcggtaagtcgcagctgctcaggaacctgctttcttcccagctgatctcccctaccccggaaaccgttttcttcatcgccccgcaggtagacatgatccccccatctgaactcaaagcgtgggaaatgcaaatctgtgagggtaactacgcccctgggccggatggaaccattataccgcagtctggcaccctccgcccgcgctttgtaaaaatggcctatgacgatctcatcctggaacacaactatgacgttagtgatcccagaaatatcttcgcccaggccgccgcccgtgggcccattgccatcattatggacgaatgcatggaaaatcttggaggtcacaagggcgtctccaagttcttccacgcatttccttctaagctacatgacaaatttcccaagtgcaccggatacactgtgctggtggttctgcacaacatgaatccccggagggatatggctgggaacatagccaacctaaaaatacagtccaagatgcatctcatatccccacgtatgcacccatcccagcttaaccgctttgtaaacacttacaccaagggcctgcccctggcaatcagcttgctactgaaagacatttttaggcaccacgcccagcgctcctgctacgactggatcatctacaacaccaccccgcagcatgaagctctgcagtggtgctacctccaccccagagacgggcttatgcccatgtatctgaacatccagagtcacctttaccacgtcctggaaaaaatacacaggaccctcaacgaccgagaccgctggtcccgggcctaccgcgcgcgcaaaacccctaaataaagacagcaagacacttgcttgatccaaatccaaacagagtctggttttttatttatgttttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttgtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggagcaccccagcacaaactacacccccgagcagcagcacgagaagctgaagcactacgtgctgatccctaagcacctgtggtcctacatcaagtacggcacccacgtgagatactacacaacacagaacgtgttcagagtgggcggctttgtgctgcagaacccttacgaggccgtgatcaagaatgaggtgaagacagccatcagactgcagaattccttcaacaccaaggccaagggccacgtgacctgggccgtgccatacgataacatcagcaagctgtacgccaagcccgatgccatcatgctgacaatccaggagaatgtggagaaggccctgcacgccctgaatcagaatgtgctgaccctggccagcaagatcaggtacccttacgacgtgcccgattacgccatgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaagatatcggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaaccgcggccgcgccaccatgctggtgatcttcctgggcatcctgggcctgctggccagccaggtgagcagccagctggtgggccagctgaggcccaccgaggagccacctgaggaggagctggagtactggtgtgcctacatggagagctgccagttctgctgggattgtcaggatggcacctgcatcaacaagatcgacggctccgtgatctacaagaatgagtacgtgaagagctgtctggtgagcagatggctggacaagtgcatgtacgatctggacaagggcatctaccacaccatgaactgcaaccaggtgctgggcctgcctaaccagcccgccggacagctgcaccccaccgataatcccccccaggaggagctggaatactggtgcacctacaccgagaactgcaagttctgctggaattgtcagaatggcctgtgtgagggcaagctggagaacacaaccatcctggagaatgagtacgtccagagctgcatcgtgagcagatggctcaacaagtgtatgtacgatctcggccagggcatccaccacgtgatggcctgctccgagcccaagccctggaatccttacaagatcctgaagagagagtggaaggagaataactcctacccctacgatgtgcctgactacgcctgactcgagtctagagggcccgtttaaacccgctgatcacttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgataagctatcaaacatgagaattcggcgcgccattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaacgcgcctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcacctgcacaccgcgccttaattaagaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcagcgaaccaatcagcagggtcatcgctagccagatcctctacgccggacgcatcgtggccaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgctgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcata

SEQ ID NO: 20 - DNA, 336 bp, Artificial Sequence
  Synthetic L8L
atgggcaacagactgatcaagaaggatctgaagaagtgtgagtactactacggcgagcagcagaacctgaagcagatctggagactgctgtttaatgagcctctgggcacctacgtggtgtccagcttcctgaagaagaactacgtggtgatctccttctcctgtcctacaaacacaagaatcatgcacctgagaatcaacatctgctacgatctgtactgtatcaacggcgagtactacgagaagatcgacgattttatcaggctgtacccccacatcttctacaggcctctgtacaggtacaagagctacccttacgacgtgcccgactacgcc

SEQ ID NO: 21 - DNA, 471 bp, Artificial Sequence; identical to SEQ ID NO: 15
  Synthetic ubiqutin

SEQ ID NO: 22 - DNA, 666 bp, Artificial Sequence
  Synthetic I215L
atggtgagcaggtttctgatcgccgagtacaggcacctgatcgagaacccctccgagaactttaagatctccgtgaacgagaaggatatgacagagtgggatgtgatcctgagaggcccccctgacaccttttacgagggcggcctgtttaaggccaagatcgcctttcctcccgagtacccttacgccccccccaggctgacatttacaagcgagatgtggcaccccaacatctacagcgatggcaagctgtgtatctccatcctgcacggcgacaatgccgaggagcagggcatgacatggagccctgcccagaagatcgacaccatcctgctgagcgtgatctccctgctgaacgagcctaaccctgactcccccgccaacgtggatgccgccaagagctacaggaagctgctgtacaaggaggacctggagtcctaccccatggaggtgaagaggaccgtgaagaagtccctggatgagtgctcccccgaggacatcgagtactttaagaacgccgccagcaatgtgcctcctatcccttccgacgcctacgaggatgagtgcgaggagatggaggatgacacctacatcctgacctacgacgatgaggacgaggaggaggaggatgacgagtacccctacgatgtgcccgattacgcctga

SEQ ID NO: 23 - DNA, 243 bp, Artificial Sequence
  Synthetic I73R
atggagacacagaagctgatctccatggtgaaggaggccctggagaagtaccagtaccctctgaccgccaagaatatcaaggtggtgatccagaaggagcacaacgtggtgctgcctaccggctccatcaattccatcctgtactccaacagcgagctgttcgagaagatcgacaagaccaacaccatctaccctcctctgtggatcaggaagaactacccctacgatgtgcctgactacgcc

SEQ ID NO: 24 - DNA, 678 bp, Artificial Sequence
  Synthetic HBsAg
atggagaacaccaccagcggcttcctgggccccctgctggtgctgcaggccggattcttcctgctgacccgcatcctgaccatcccccagagcctggacagctggtggacctccctgaactttctgggcggggcccccacctgccccggacagaactctcagagccccacctccaaccacagccccaccagctgcccccccatctgcccaggatatagatggatgtgcctccgccgcttcatcatcttcctgttcatcctgctcctgtgcctcatcttcctcctcgtgctcctggactaccagggcatgctccccgtctgccccctcctgcctggcacatccaccacctccaccggcccctgcaagacctgcaccatccccgcccagggcacctccatgttcccctcctgctgctgcaccaaaccctccgacggcaactgcacctgcatccccatccccagcagctgggccttcgccaggttcctgtgggaatgggccagcgtcaggttcagctggctctccctcctcgtgcccttcgtgcagtggtttgtgggcctgagccccaccgtctggctcagcgtgatctggatgatgtggtattgggggccccgcctgtacaacatcctctcccctttcctccccctgctgcccatcttcttctgcctctgggtgtacatc

SEQ ID NO: 25 - DNA, 441 bp, Artificial Sequence
  Synthetic E146L
atgggcggaacaacagactttgtgctgagcatcacaatcgtgctggtgatcctgatcatcatcgccttcatctggtacaattttaccggctggtccccctttaagtacagcaagggcaacaccgtgacatttaagacccctgacgagagcagcatcgcctacatgagatttaggaactgtgtgttcacattcaccgaccccaagggcagcctgcacagcatcgacgtgacagaggtgctgaacaacatggccaagggctttagggatgcccagaatccccctagctccttcacactgggcggccactgccaggcccccctgaatgctttttcctttgtgctgcctggcgtgaacgacagagccacagtggccaccgccgacgaggccaagaagtgggagaactgcgacgccaccctgaccggcctgcagagaatcatctaa

SEQ ID NO: 26 - DNA, 38461 bp, Artificial Sequence
  Synthetic pAd5LCL3-L8Lubiqutin-I215L- I73RHBsAg-E146L
agcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacacgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgatcgattaattaacgacccatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttttttaaaagaaaaggggggattggggggtacagtgcaggggaaagaatagtagacataatagcaacagacatacaaactaaagaattacaaaaacaaattacaaaattcaaaattttatcgtactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggatccgccaccatggagacacagaagctgatctccatggtgaaggaggccctggagaagtaccagtaccctctgaccgccaagaatatcaaggtggtgatccagaaggagcacaacgtggtgctgcctaccggctccatcaattccatcctgtactccaacagcgagctgttcgagaagatcgacaagaccaacaccatctaccctcctctgtggatcaggaagaactacccctacgatgtgcctgactacgccatggagaacaccaccagcggcttcctgggccccctgctggtgctgcaggccggattcttcctgctgacccgcatcctgaccatcccccagagcctggacagctggtggacctccctgaactttctgggcggggcccccacctgccccggacagaactctcagagccccacctccaaccacagccccaccagctgcccccccatctgcccaggatatagatggatgtgcctccgccgcttcatcatcttcctgttcatcctgctcctgtgcctcatcttcctcctcgtgctcctggactaccagggcatgctccccgtctgccccctcctgcctggcacatccaccacctccaccggcccctgcaagacctgcaccatccccgcccagggcacctccatgttcccctcctgctgctgcaccaaaccctccgacggcaactgcacctgcatccccatccccagcagctgggccttcgccaggttcctgtgggaatgggccagcgtcaggttcagctggctctccctcctcgtgcccttcgtgcagtggtttgtgggcctgagccccaccgtctggctcagcgtgatctggatgatgtggtattgggggccccgcctgtacaacatcctctcccctttcctccccctgctgcccatcttcttctgcctctgggtgtacatcggaagcggagctactaacttcagcctgctgaagcaggctggagacgtggaggagaaccctggacctatgggcggaacaacagactttgtgctgagcatcacaatcgtgctggtgatcctgatcatcatcgccttcatctggtacaattttaccggctggtccccctttaagtacagcaagggcaacaccgtgacatttaagacccctgacgagagcagcatcgcctacatgagatttaggaactgtgtgttcacattcaccgaccccaagggcagcctgcacagcatcgacgtgacagaggtgctgaacaacatggccaagggctttagggatgcccagaatccccctagctccttcacactgggcggccactgccaggcccccctgaatgctttttcctttgtgctgcctggcgtgaacgacagagccacagtggccaccgccgacgaggccaagaagtgggagaactgcgacgccaccctgaccggcctgcagagaatcatctaactcgagtctagagggcccgtttaaacccgctgatcagcctcgataatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgagaagccttagagtctcctcttctgaggcggaaagaaccagctggggctctagcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatctttgagaccgcacagggtcttaataagggtgcagagatcctcaggtccttgacaaggtgagtgaatgcagccttcggtttctaccgagtgctgagttatggtaatgggcttttctcccaccatgaccaccaatttctgacgcttggttggcaacttgtagctaaggcggtgtccggtggtattactgtcgtaggtgactttggcctgctttaccagacaaaagataccccttttgcactggtgcaagttaaccatgtcttggagctcttgattcatgcgctgttgctcggccgctgccctgcgtctttctagcaggcgctgctctgtaataattccgtccatttctagctagagaaacctgaattagaatagcccgtagagttgcttgaattgttcataaaccccacagtagctgcgcctttggcctaataccctaagggttttctaagctcacctcctgttctggtaaacagagttattgaggtctgtccggaaaaagtctggtttacggtcaggcggtaggtgtggtgcagcggccggtgacgcactcgtacgttcccggcaggtaaggagggtggtgttttttctgatggagtagctgagctcggagaggttctctcgtagactcactccgtctgggttgaaactgttgtaaatcacagagggagagatgttaaaagtaccaggtaaggttcgccttggtttgcttgggcgggtgaagacggtggcgtttacaggatggcgataggagccccagtatattttaatttctgtatttattatactcagcacagagatggcaacaaagatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagactagggttctgtgagtttgattaaggtacggtgatctgtataagctatgtggtggtggggctatactactgaatgaaaaatgacttgaaattttctgcaattgaaaaataaacacgttgaaacataacacaaacgatacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggctggtgcacacggaccacgtcaaagacttcaaacaaaacataaagaagggtgggctcgtccatgggatccacctcaaaagtcatgtctagcgcgtgggcggagttggcgtagagaaggttttggcccaggtctgtgagtgcgcccatggacataaagttactggagaatgggatgcgccaaagggtgcgatcgcaaagaaactttttctgggtaatgctgtcaactgcggtcttgcctataagcggataggggaagttagcagggtaggcctgtccttcgcgcatggtgggggcaaggtagccaacaaatccagagttgttgtgttggtgtaggatgcccacctgttggtagtccttgtatttagtatcatccaccacctgacggctcatgggctggaagtttctaaagaaggagtacatgcggtccttgtagctctctgggatatagaagccctggtagccaatgttatagttagctagcatttgtaccaggaaccagtctttggtcatgttacactgggcaacgttgtaaccctccccgtcaactgagcgcttaatttcaaactcgttgggggtaagcaggcggtcattgccaggccagctgacagaagagtcaaaggtaatggccaccttcttaaaggtgtggttgaggtaaaaggttccatctaggtagggtatagagccagagtaggtgtaataagggtcgtagcccgagcccagtgatggggtttccttagtcttaaggcgcgtgaaggcccagccgcggaaagccgcccagttgcgggaggggatggatatgggcacgttggtagcgttggcgggtatagggtagagcatgttggcggcggagagatagtcgttaaaggactggtcgttggtgtcgtttctaagcatggcctcaagcgtggaggcggtgttgtgggccatggggaagaaggtggcgtaaaggcaaatgctatcaaacttaatgctggctccgtcaacccttaggtcatttcctagggagctctgcagaaccatgttaacatccttcctgaagttccactcgtaggtgtatgagcccggcaggagaaggaggtttttaatggcaaagaacttctgaggcacctggatgtggaagggcacatagcgaccattgcccagcaacattgagcggtagcgcaggccagcattgcggtggtggttaaatgggttgacgttgtccatatagtcaagggaccagcgtgctccaaggttaatgtagcagtccactagcccgggagccaccactcgcttgttcatgtagtcgtaggtgtttgggttatcagaaatttttacgttggaaggactgtactttagcttgtcgggcaaatacagcgctatgttggagtacaggaaatttctccacaggttggcatttagattgatttccatggcaaaattatttccaactcttatttcatttttatctgaaaattctgtagcatctttttcccatccattttcctgacctgttttaggttttaccttggtaagagtctctgtattaatcacacctcccagtggaaagcagtaatttggaagttcatcttcagttccatgattttcaataattctaacatctggatcatagctgtcaacagcctgattccacatagaaaagtacctggttctatcaccaatggaatcaagcaaaagctggtatgaaagctctgtgtttctgtcttgcaaatctacaacagcattcaactgcgatgcttggcccgccagaacacccatattacccgtgctgttgtaatacattagaccaataaaattgtccctaaaagcaatgtaattaggcctgttgggcatagattgttggcccattagttctcgtgagttaccttccttaatagtgggcatgtaagaaatatgagtgtctggggtttctatatctacatcttcactgtacaataccactttaggagtcaagttatcaccattgcctgcggctgcctcagtagttgagaaaaattgcatttccacttgactttctagctttccattttgttgctttacaagaatgccttgccctccattttcatttgtgggttttgcatatgaaccgtaacatggtttcattggggtagtcttttttaggactctcccagctgcatgattaatttctgtttcgtaccactgagattctcctatttgaggttcaggttgaaatgttttatcggcatatttaggtgtttgaccttcgacacctatttgaataccctcctttgtaatatttataccagaataaggcgcctgcccaaatacgtgagttttttgctgctcagcttgctcgtctacttcgtcttcgttgtcatcgtcctcttcttctaggtttatttcaagagcagtagcagcttcatcccattcgcaaggatttggggcacccttgggagccagggcgttgtaggcagtgccagagtagggcttaaaagtagggcccctgtccagcacgccgcggatgtcaaagtacgtggaagccatgtccagcacacggttatcacccacagctagggtgaaccgcgccttgtacgagtacgcagtatcctcacggtccacagggatgaaccgcagcgtcaaacgctgggaccggtctgtggtcacgtcgtgcgtaggcgccaccgtggggtttctaaacttgttattcaggctgaagtacgtctcggtggcgcgggcaaactgcaccagcccggggctcaggtactccgaggcgtcctggcccgagatgtgcatgtaagaccactgcggcatcatcgaaggggtagccatcttggaaagcgggcgcgcggcggctcagcagctcctctggcggcgacatggacgcatacatgacacacatacgacacgttagctatcagaagcatcgtcggcgcttcagggattgcacccccagacccacgatgctgttcagtgtgctttgccagttgccactggctacgggccgcaacgatcgcggaccgctggcggcgcggcgcagggacgcgcggctaggacgggttacaacaacggcggtcgggcctggcagcacaggtttctgctgggtgtcggcggggggaggcaggtccagcgttacgggtgtgtgctggcccagcactccggtagccatgggcgcgatgggacgggtggtgggcaggccttgctttagtgcctcctcgtacgagggaggctcgtctatttgcgtcaccagagtttcttccctgtcggggcgcggacgcttttcgccacgcccctctggagacactgtctccacggccggtggaggctcctctacgggagggcggggatcaagcttactgttaatcttattttgcactgcctggttggccaggtccaccaccccgctaatgccagaggccaggccatctaccaccttttgttggaaattttgctctttcaacttatccctcagcatctggcctgtgctgctgttccaggccttgctgccatagttcttaacggtggaaccgaaatttttaatgccgctccacagcgagccccagctgaaggcgccaccgctcatattgctggtgccgatatcttgccagtttcccatgaacgggcgcgagccgtgtcgcggggccagagacgcaaagttgatgtcttccattctacaaaatagttacaggaccaagcgagcgtgagagtccagactttttattttgatttttccacatgcaacttgtttttaatcagtgtctctgcgcctgcaaggccacggatgcaattccgggcacggcgccaatcgccgcggcgatcagtggaataaggaggggcaggataccgccgcgcatgcgacggtgcgacgcgcgccgccgccggtggtgcgcacgacgcatgccgcccgtcaggccgtggccggccatgcccctcctacggtgcattcttcctcggaatcccggcaccgggaaacggaggcggcaggtgagggccatatctgcaagaaccacaaagaccggcttttaaacgatgctggggtggtagcgcgctgttggcagcaccagggtcctgcctccttcgcgagccaccctgcgcacggaaatcggggccagcacgggctggcgacggcgacggcggcggcgggttccagtggtggttcggcgtcgggtagttgctcgtcttctggggcggtaggtgtagccacgatagccgggggtaggcgcaatggaaggatgtagggcatattcgggcagtagcgcgctggcggcgccgtacttcctcgaacggcgcgggcgccggggggctgaaacgcgaaacatccacgggtccgtttgcacctccgtagaggtcttggacgcggccgcagcgaccgcctgcaccgcggcatccgccaccgctgaggcaaccggggacgtttgtgtctccatgccctctgtggcggtggcaatactggtgctactggtagtgggtatctgaacgtccacggtctgcacgcccagtcccggcgccacctgcttgattggccgcacgcggacctcgggctccagcccaggttccacggtcattttttccaagacatcttccagtcgctggcgcttgggtaccatcagctgcacggtgggtgccaagtcaccagactcgcgctttaggccgcgcttttcttcggacggtgcaagcgcgggcagcacctgctgcagtgttacgggctttaggctaggtgttgggttgccctcgtccagcggcaacgccagcatgtccttatgccgctttccgtaggcaaactccccgaggcgctcgttggcctgctcaagcaggtcctcgtcgccgtacacctcatcatacacgcgcttgtaggtgcgggtggagcgctcaccgggcgtaaagactacggtggtgccgggtcgcaaaacacgttttacgcgtcgacctttccactgtacccgtcgcctgggcgcggtagcgtgcagcagttccacctcgtcgtcaagttcatcatcatcatctttctttttctttttgacccgctttagctttcggggcttgtaatcctgctcttccttcttcggggggccatagatctccggcgcgatgacctggagcatctcttctttgattttgcgcttggacatagcttcgttgcgcgccgccgccgctggatacatacaacagtacgagtctaagtagttttttcttgcaatctagttgcgcggggggcgggtgcgcacgggcacgcgcaggccgctaaccgagtcgcgcacccaatacacgttgcccctgcgaccctgagtcatagcactaatggccgcggctgctgcggcggccgctcgtcgcctggacctggggggcacagtgacaatacccgcggccagccttcgagcggcccgcatggccgcccgtcggccggtgcgacgtgcgcggttaagcagggccgccgccgcgcgttgggcggcagtgccgggtcggcggcggtggcgacgtgctacgcgcctccgccgtctcttcattttagcatagcgccgggctccgcgcaccacggtctgaatggccgcgtccactgtggacactggtggcggcgtgggcgtgtagttgcgcgcctcctccaccaccgcgtcgatggcgtcatcgacggtggtgcgcccagtgcggccgcgtttgtgcgcgccccagggcgcgcggtagtgcccgcgcacgcgcactgggtgttggtcggagcgcttcttggccccgccaaacatcttgcttgggaagcgcaggccccagcctgtgttattgctgggcgatataaggatggacatgcttgctcaaaaagtgcggctcgataggacgcgcggcgagactatgcccagggccttgtaaacgtaggggcaggtgcggcgtctggcgtcagtaatggtcactcgctggactcctccgatgctgttgcgcagcggtagcgtcccgtgatctgtgagagcaggaacgttttcactgacggtggtgatggtgggggctggcgggcgcgccaaaatctggttctcgggaaagcgattgaacacgtgggtcagagaggtaaactggcggatgagttgggagtagacggcctggtcgttgtagaagctcttggagtgcacgggcaacagctcggcgcccaccaccggaaagttgctgatctggcgcgtggagcggaaggtcacggggtcttgcatcatgtctggcaacgaccagtagacctgctccgagccgcaggttacgtcaggagtgcaaagcagggtccatgagcggattccggtctgagggtcgccgtagttgtatgcaaggtaccagctgcggtactgggtgaaggtgctgtcattgcttattaggttgtaactgcgtttcttgctgtcctctgtcaggggtttgatcaccggtttcttctgaggcttctcgacctcgggttgcgcagcgggggcggcagcttcggccgctgcttcggcctcagcgcgcttctcctcagcccgtgtggcaaaggtgtcgccgcgaatggcatgatcgttcatgtcctccaccggctgcattgccgcggctgccgcgttggagttctcttccgcgccgctgccactgctgttgctgccgcctgcgccacccccgccctgttcggtgtcatctttcaagctcgcctggtaggcgtccacatccaacagtgcgggaatgttaccaccctccagatcatcgtaggtgatcctaaagccctcctggaagggttgccgcttgcggatgcccaacaagttgctcaggcggctgtgggtgaagtccaccccgcatcctggcagcaaaatgatgtctggatggaaggcttcgtttgtatataccccaggcatgacaagaccagtgacggggtcaaaccccagtctgaagttgcgggtgtcaaactttaccccgatgtcgctttccagaaccccgttctgtctgcccactttcaagtagtgctccacgatcgcgttgttcataaggtctatggtcatggtctcggagtagttgccctcgggcagcgtgaactccacccactcgtatttcagctccacctgattgtccttagtaggcaagcgcgacaccatcacccgcgccttaaacttattggtaaacatgaactcgttcacatttggcatgttggtatgcaggatggttttcaggtcgccgccccagtgcgaccggtcgtcaagattgatggtctgtgtgcttgcctcccccgggctgtagtcattgttttgaatgaccgtggtcagaaagttgctgtggtcgttctggtagttcagggatgccacatccgttgacttgttgtccaccaggtacacacgggtggtgtcgaataggggtgccaactcagagtaacggatgctgtttctccccccggtaggccgcaggtaccgcggaggcacaaacggcgggtccaggggagcatcgaagggagaacccagcgccgccgccactggcgccgcgctcaccacactctcgtaggagggaggaggaccttcctcatacatcgccgcgcgccgcatactaaggggaatacaagaaaaccaacgctcggtgccatggccttggtgagttttttattttgcatcatgctttttttttttttaaaacattctccccagcctggggcgaaggtgcgcaaacgggttgccactccctcccaaatccaggacgctgctgtcgtctgccgagtcatcgtcctcccacaccagaccccgctgacggtcgtgcctttgacgacgggtgggcgggcgcgggcctggcacgtccctgtgctcctgcgcgtacgtcttccatctactcatcttgtccactaggctctctatcccgttgttgggaaatgccggaggcaggtttttttcgcgctgcggctgcagcagcgagttgtttaggtactcctcctcgcccagcaggcgcgggcgggtggtgcgagtgctggtaagagaccctatcaagcttggaaatgggctactagcatctgaccgcggggccgcagcgcctagatcggacaagctgcttggcctgcggaagctttcctttcgcagcgccgcctctgcctgctcgcgctgttgcaactctagcagggtctgcggttgcggggaaaacacgctgtcgtctatgtcgtcccagaggaatccatcgttaccctcgggcacctcgaatcccccggtgtagaaaccagggggcggtagccagtgcgggttcaagatggcattggtgaaatactcggggttcacggcggccgcgcgatgcaagtagtccattaggcggttgataaacggccggtttgaggcatacatgcccggttccatgttgcgcgcggtcatgtccagcgccacgctgggcgttaccccgtcgcgcatcaggttaaggctcacgctctgctgcacgtagcgcaaaatgcgctcctcctcgctgtttaaactgtgcaacgaggggatcttctgccgccggttggtcagcaggtagtttagggttgcctccaggctgcccgtgtcctcctgccccagcgcgcggctgacacttgtaatctcctggaaagtatgctcgtccacatgcgcctgacctatggcctcgcggtacagtgtcagcaagtgacctaggtatgtgtcccgggacacgctgccactgtccgtgaagggcgctattagcagcagcaacaggcgcgagttgggcgtcagcaagctagacacggtcgcgcggtcgcctgtgggagcccgcaccccccacagcccctgcaagtttttgaaagcctggctcaggtttacggtctgcaggccttgtctactggtctggaaaaaatagtctggcccagactggtacacctcactttgcggtgtctcagtcaccattagccgcagtgcgctcacaaagttggtgtagtcctcctgtccccgcggcacgttggcgggctgtgtactcaggaaggcgtttagtgcaaccatggagcccaggttgccctgctgctgcgcgcgctcacgctgcgccacggcctcgcgcacatcccccaccagccggtccaggttggtctgcacgttgccgctgttgtaacgagccacgcgctgaagcagcgcgtcgtagaccaggccggcctcgtcgggccggatggccctgttttcggccagcgcgtttacgatcgccagcaccttctcgtgcgtggggtttgcgcgcgccgggaccaccgcttccagaattgcggagagccggttggcctgcggctgctgccggaacgcgtcaggattgcgcgcagtcagcgacatgatgcggtccatgacctggcgccagtcgtccgtggagttaaggccggacggctggctctgcagcgccgcccgcaccgccgggtccgttgcgtcttgcatcatctgatcagaaacatcaccgcttagtactcgccgtcctctggctcgtactcatcgtcctcgtcatattcctccacgccgccgacgttgccagcgcgcgcgggtgccaccgccagcccaggtccggccccagctgcctccagggcgcgtcggcttggggcccagcgcaggtcagcgcccgcgtcaaagtaggactcggcctctctatcgccgctgcccgtgccagccagggccctttgcaggctgtgcatcagctcgcggtcgctgagctcgcgccgccggctcacgctcacggccttgtggatgcgctcgttgcgataaacgcccaggtcgtcgctcaaggtaagcaccttcagcgccatgcgcatgtagaacccctcgatctttacctccttgtctatgggaacgtaaggggtatggtatatcttgcgggcgtaaaacttgcccaggctaagcatggaatagttgatggcggccaccttgtcagccaggctcaagctgcgctcctgcaccactatgctctgcaggatgtttatcaaatcgagcagccagcggccctcgggctctactatgtttagcagcgcatccctgaatgcctcgttgtccctgctgtgctgcactataaggaacagctgcgccatgagcggcttgctatttgggttttgctccagcgcgcttacaaagtcccacagatgcatcagtcctatagccacctcctcgcgcgccacaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgggctcaaagtcctcctcgcgcagcaaccgctcgcgattcaggccatgccgcagctcgcgccctgcgtggaactttcgatcccgcatctcctcgggctcctctccctcgcggtcgcgaaacaggttctgccgcggcacgtacgcctcacgcgtatcacgcttcagctgcacccttgggtgccgctcaggagagggcgctcctagccgcgccaggccctcgccctcctccaagtccaggtagtgccgggcccggcgccgcgggggttcgtaatcaccatctgctgccgcgtcaaccgcggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttagatgcatcatccaaggacacccccgcggcccaccgcccgccgcgcggtaccgtagtcgcgccgcggggatgcggcctcttgcaagccatcgacgccgccaccaaccagcccctggaaattaggtatcacctggatctagcccgcgccctgacccgtctatgcgaggtaaacctgcaggagctcccgcctgacctgacgccgcgggagctccagaccatggacagctcccatctgcgcgatgttgtcatcaagctccgaccgccgcgcgcggacatctggactttgggctcgcgcggcgtggtggtccgatccaccgtaactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaatagccactcctccaattggtggcgggagatccagttcttcccgatcggctcgcatcctcgcaccgagcgtctctttgtcacctacgatgtagagacctatacttggatgggggcctttgggaagcagctcgtgcccttcatgctggtcatgaagttcggcggagatgagcctctagtgactgccgcgcgagacctagccgcgaaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctttcagagcacgggctcagctcccctgaagagctcacctacgaggaacttaaaaaattgccttccatcaagggcatcccgcgcttcttggaactttacattgtgggccacaacatcaacggctttgacgagatcgtgctcgccgcccaggtaattaacaaccgttccgaggtgccgggacccttccgcatcacacgcaactttatgcctcgcgcgggaaagatactcttcaacgatgtcaccttcgccctgccaaatccgcgttccaaaaagcgcacggactttttgctctgggagcagggcggatgcgacgacactgacttcaaataccagtacctcaaagtcatggtcagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagaccccctaccgccattctgctcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggatgcggccaccctcaaaggcatcaccgcgggccaggtgaatatcaaatcctcctcgtttttggaaactgacaatcttagcgcagaagtcatgcccgcttttcagagggagtactcaccccaacagctggccctcgcagacagcgatgcggaagagagtgaggacgaacgcgcccccacccccttttatagccccccttcaggaacacccggtcacgtggcctacacctacaaaccaatcaccttccttgatgccgaagagggcgacatgtgtcttcacaccctggagcgagtggaccccctagtggacaacgaccgctacccctcccacttagcctccttcgtgctggcctggacgcgagcctttgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgccctcaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagagcaggtgaccgagctgtgggaccgcctggaactgcttggtcaaacgctcaaaagcatgcctacggcggacggtctcaaaccgttgaaaaactttgcttccttgcaagaactgctatcgctgggcggcgagcgccttctggcggatttggtcagggaaaacatgcgagtcagggacatgcttaacgaagtggcccccctgctcagggatgacggcagctgcagctctcttaactaccagttgcagccggtaataggtgtgatttacgggcccaccggctgcggtaagtcgcagctgctcaggaacctgctttcttcccagctgatctcccctaccccggaaaccgttttcttcatcgccccgcaggtagacatgatccccccatctgaactcaaagcgtgggaaatgcaaatctgtgagggtaactacgcccctgggccggatggaaccattataccgcagtctggcaccctccgcccgcgctttgtaaaaatggcctatgacgatctcatcctggaacacaactatgacgttagtgatcccagaaatatcttcgcccaggccgccgcccgtgggcccattgccatcattatggacgaatgcatggaaaatcttggaggtcacaagggcgtctccaagttcttccacgcatttccttctaagctacatgacaaatttcccaagtgcaccggatacactgtgctggtggttctgcacaacatgaatccccggagggatatggctgggaacatagccaacctaaaaatacagtccaagatgcatctcatatccccacgtatgcacccatcccagcttaaccgctttgtaaacacttacaccaagggcctgcccctggcaatcagcttgctactgaaagacatttttaggcaccacgcccagcgctcctgctacgactggatcatctacaacaccaccccgcagcatgaagctctgcagtggtgctacctccaccccagagacgggcttatgcccatgtatctgaacatccagagtcacctttaccacgtcctggaaaaaatacacaggaccctcaacgaccgagaccgctggtcccgggcctaccgcgcgcgcaaaacccctaaataaagacagcaagacacttgcttgatccaaatccaaacagagtctggttttttatttatgttttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttgtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatgggcaacagactgatcaagaaggatctgaagaagtgtgagtactactacggcgagcagcagaacctgaagcagatctggagactgctgtttaatgagcctctgggcacctacgtggtgtccagcttcctgaagaagaactacgtggtgatctccttctcctgtcctacaaacacaagaatcatgcacctgagaatcaacatctgctacgatctgtactgtatcaacggcgagtactacgagaagatcgacgattttatcaggctgtacccccacatcttctacaggcctctgtacaggtacaagagctacccttacgacgtgcccgactacgccatgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaagatatcggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaaccgcggccgcgccaccatggtgagcaggtttctgatcgccgagtacaggcacctgatcgagaacccctccgagaactttaagatctccgtgaacgagaaggatatgacagagtgggatgtgatcctgagaggcccccctgacaccttttacgagggcggcctgtttaaggccaagatcgcctttcctcccgagtacccttacgccccccccaggctgacatttacaagcgagatgtggcaccccaacatctacagcgatggcaagctgtgtatctccatcctgcacggcgacaatgccgaggagcagggcatgacatggagccctgcccagaagatcgacaccatcctgctgagcgtgatctccctgctgaacgagcctaaccctgactcccccgccaacgtggatgccgccaagagctacaggaagctgctgtacaaggaggacctggagtcctaccccatggaggtgaagaggaccgtgaagaagtccctggatgagtgctcccccgaggacatcgagtactttaagaacgccgccagcaatgtgcctcctatcccttccgacgcctacgaggatgagtgcgaggagatggaggatgacacctacatcctgacctacgacgatgaggacgaggaggaggaggatgacgagtacccctacgatgtgcccgattacgcctgactcgagtctagagggcccgtttaaacccgctgatcacttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccggaaaaacacctgggcgagtctccacgtaaacggtcaaagtccccgcgggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgataagctatcaaacatgagaattcggcgcgccattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaacgcgcctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcacctgcacaccgcgccttaattaagaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcagcgaaccaatcagcagggtcatcgctagccagatcctctacgccggacgcatcgtggccaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgctgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcata

SEQ ID NO: 27 - DNA, 1236 bp, Artificial Sequence
  Synthetic EP402R
atgatcatcatcgtgatcttcctgatgtgcctgaagatcgtgctgaacaacatcatcatctggagcaccctgaaccagaccgtgttcctgaacaacatcttcaccatcaacgacacctacggcggcctgttctggaacacctactacgacaacaacagaagcaacttcacctactgcggcatcgccggcaactactgcagctgctgcggccacaacatcagcctgtacaacaccaccaacaactgcagcctgatcatcttccccaacaacaccgagatcttcaacagaacctacgagctggtgtacctggacaagaagatcaactacaccgtgaagctgctgaagagcgtggacagccccaccatcacctacaactgcaccaacagcctgatcacctgcaagaacaacaacggcaccaacgtgaacatctacctgatcatcaacaacaccatcgtgaacgacaccaacggcgacatcctgaactactactggaacggcaacaacaacttcaccgccacctgcatgatcaacaacaccatcagcagcctgaacgagaccgagaacatcaactgcaccaaccccatcctgaagtaccagaactacctgagcaccctgttctacatcatcatcttcatcgtgagcggcctgatcatcggcatcttcatcagcatcatcagcgtgctgagcatcagaagaaagagaaagaagcacgtggaggagatcgagagcccaccacccagcgagagcaacgaggaggacatcagccacgacgacaccaccagcatccacgagccaagccccagagaaccactgctgcctaagccctacagcagataccagtacaacacccccatctactacatgagacccagcacccagcccctgaaccccttccccctgcccaagccatgcccgccacctaaaccatgccctccacccaagccttgcccgcccccaaagccatgtccaccacccaaaccttgctctccacccaagccgtgtcgtccccccaaaccatgtcctccaccaaaaccatgtcctccgccgaagccatgcccacctcctaagccatgcccccccagcaagccctgccccagccccgagagctacagcccccccaagcccctgcccagcatccccctgctgcccaacatcccccccctgagcacccagaacatcagcctgatccacgtggacagaatcatctacccatacgacgtcccagactacgcttaa

SEQ ID NO: 28 - DNA, 477 bp, Artificial Sequence
  Synthetic EP153R
atgttcagcaacaagaagtacatcggcctgatcaacaagaaggagggcctgaagaagaagatcgacgactacagcatcctgatcatcggcatcctgatcggcacaaacatcctgtccctgatcatcaacatcatcggcgagatcaataagcctatctgctaccagaacgatgacaagatcttctactgtcctaaggactgggtgggctacaacaatgtgtgctactacttcggcaacgaggagaagaattacaacaatgccagcaactactgtaagcagctgaatagcaccctgaccaataataacacaatcctggtgaacctgacaaagacactgaacctgacaaaaacatacaaccacgagagcaattactgggtgaactacagcctgatcaagaacgagagcgtgctgctgagggattccggctactacaagaagcagaagcacgtgagcctgctgtacatctgtagcaagtga

SEQ ID NO: 29 - DNA, 225 bp, Artificial Sequence
  Synthetic I177L
atgtggaaggtgaacgaccagggcttcctgaacatctccgtgacaggcaccaagtttaatctgatcgccatcacaggcaagctgggcttctacacagaccctcctagccacctgatcatcatgcccctgaagtttttccccgtgcacaagttcagcaagaacgagcctaataagaagcagaagaggttcatctacttttacccctacgacgtgcccgattacgcc

SEQ ID NO: 30 - DNA, 642 bp, Artificial Sequence
  Synthetic K205R
atggtggagcccagagagcagttttttcaggatctgctgagcgccgtggatcagcagatggacaccgtgaagaatgacatcaaggatattatgaaggagaagacatccttcatggtgagctttgagaattttatcgagagatacgatacaatggagaagaacatccaggatctgcagaacaagtacgaggagatggccgccaacctgatgaccgtgatgaccgacaccaagatccagctgggcgccatcatcgcccagctggagatcctgatgatcaatggcacacctctgcctgccaagaagacaaccatcaaggaggccatgcccctgcccagctccaataccaataacgagcagacctccccccctgcctccggcaagacaagcgagacacccaagaagaaccccaccaacgccatgttttttacaagaagcgagtgggcctcctccaatacctttagagagaagttcctgacacctgagatccaggccatcctggatgagcagtttgccaataagacaggcatcgagagactgcacgccgagggcctgtacatgtggagaacacagttcagcgacgagcagaagaagatggtgaaggagatgatgaagaagtacccttacgatgtgcccgattacgcc

SEQ ID NO: 31 - DNA, 471 bp, Artificial Sequence; identical to SEQ ID NO: 15
  Synthetic ubiqutin

SEQ ID NO: 32 - DNA, 38677 bp, Artificial Sequence
  Synthetic pAd5LCL3-EP402R-EP153R-I177L- K205Rubiqutin
agcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacacgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgatcgattaattaacgacccatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttttttaaaagaaaaggggggattggggggtacagtgcaggggaaagaatagtagacataatagcaacagacatacaaactaaagaattacaaaaacaaattacaaaattcaaaattttatcgtactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggatccgccaccatgtggaaggtgaacgaccagggcttcctgaacatctccgtgacaggcaccaagtttaatctgatcgccatcacaggcaagctgggcttctacacagaccctcctagccacctgatcatcatgcccctgaagtttttccccgtgcacaagttcagcaagaacgagcctaataagaagcagaagaggttcatctacttttacccctacgacgtgcccgattacgccggaagcggagctactaacttcagcctgctgaagcaggctggagacgtggaggagaaccctggacctatggtggagcccagagagcagttttttcaggatctgctgagcgccgtggatcagcagatggacaccgtgaagaatgacatcaaggatattatgaaggagaagacatccttcatggtgagctttgagaattttatcgagagatacgatacaatggagaagaacatccaggatctgcagaacaagtacgaggagatggccgccaacctgatgaccgtgatgaccgacaccaagatccagctgggcgccatcatcgcccagctggagatcctgatgatcaatggcacacctctgcctgccaagaagacaaccatcaaggaggccatgcccctgcccagctccaataccaataacgagcagacctccccccctgcctccggcaagacaagcgagacacccaagaagaaccccaccaacgccatgttttttacaagaagcgagtgggcctcctccaatacctttagagagaagttcctgacacctgagatccaggccatcctggatgagcagtttgccaataagacaggcatcgagagactgcacgccgagggcctgtacatgtggagaacacagttcagcgacgagcagaagaagatggtgaaggagatgatgaagaagtacccttacgatgtgcccgattacgccatgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaactcgagtctagagggcccgtttaaacccgctgatcagcctcgataatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgagaagccttagagtctcctcttctgaggcggaaagaaccagctggggctctagcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatctttgagaccgcacagggtcttaataagggtgcagagatcctcaggtccttgacaaggtgagtgaatgcagccttcggtttctaccgagtgctgagttatggtaatgggcttttctcccaccatgaccaccaatttctgacgcttggttggcaacttgtagctaaggcggtgtccggtggtattactgtcgtaggtgactttggcctgctttaccagacaaaagataccccttttgcactggtgcaagttaaccatgtcttggagctcttgattcatgcgctgttgctcggccgctgccctgcgtctttctagcaggcgctgctctgtaataattccgtccatttctagctagagaaacctgaattagaatagcccgtagagttgcttgaattgttcataaaccccacagtagctgcgcctttggcctaataccctaagggttttctaagctcacctcctgttctggtaaacagagttattgaggtctgtccggaaaaagtctggtttacggtcaggcggtaggtgtggtgcagcggccggtgacgcactcgtacgttcccggcaggtaaggagggtggtgttttttctgatggagtagctgagctcggagaggttctctcgtagactcactccgtctgggttgaaactgttgtaaatcacagagggagagatgttaaaagtaccaggtaaggttcgccttggtttgcttgggcgggtgaagacggtggcgtttacaggatggcgataggagccccagtatattttaatttctgtatttattatactcagcacagagatggcaacaaagatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagactagggttctgtgagtttgattaaggtacggtgatctgtataagctatgtggtggtggggctatactactgaatgaaaaatgacttgaaattttctgcaattgaaaaataaacacgttgaaacataacacaaacgatacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggctggtgcacacggaccacgtcaaagacttcaaacaaaacataaagaagggtgggctcgtccatgggatccacctcaaaagtcatgtctagcgcgtgggcggagttggcgtagagaaggttttggcccaggtctgtgagtgcgcccatggacataaagttactggagaatgggatgcgccaaagggtgcgatcgcaaagaaactttttctgggtaatgctgtcaactgcggtcttgcctataagcggataggggaagttagcagggtaggcctgtccttcgcgcatggtgggggcaaggtagccaacaaatccagagttgttgtgttggtgtaggatgcccacctgttggtagtccttgtatttagtatcatccaccacctgacggctcatgggctggaagtttctaaagaaggagtacatgcggtccttgtagctctctgggatatagaagccctggtagccaatgttatagttagctagcatttgtaccaggaaccagtctttggtcatgttacactgggcaacgttgtaaccctccccgtcaactgagcgcttaatttcaaactcgttgggggtaagcaggcggtcattgccaggccagctgacagaagagtcaaaggtaatggccaccttcttaaaggtgtggttgaggtaaaaggttccatctaggtagggtatagagccagagtaggtgtaataagggtcgtagcccgagcccagtgatggggtttccttagtcttaaggcgcgtgaaggcccagccgcggaaagccgcccagttgcgggaggggatggatatgggcacgttggtagcgttggcgggtatagggtagagcatgttggcggcggagagatagtcgttaaaggactggtcgttggtgtcgtttctaagcatggcctcaagcgtggaggcggtgttgtgggccatggggaagaaggtggcgtaaaggcaaatgctatcaaacttaatgctggctccgtcaacccttaggtcatttcctagggagctctgcagaaccatgttaacatccttcctgaagttccactcgtaggtgtatgagcccggcaggagaaggaggtttttaatggcaaagaacttctgaggcacctggatgtggaagggcacatagcgaccattgcccagcaacattgagcggtagcgcaggccagcattgcggtggtggttaaatgggttgacgttgtccatatagtcaagggaccagcgtgctccaaggttaatgtagcagtccactagcccgggagccaccactcgcttgttcatgtagtcgtaggtgtttgggttatcagaaatttttacgttggaaggactgtactttagcttgtcgggcaaatacagcgctatgttggagtacaggaaatttctccacaggttggcatttagattgatttccatggcaaaattatttccaactcttatttcatttttatctgaaaattctgtagcatctttttcccatccattttcctgacctgttttaggttttaccttggtaagagtctctgtattaatcacacctcccagtggaaagcagtaatttggaagttcatcttcagttccatgattttcaataattctaacatctggatcatagctgtcaacagcctgattccacatagaaaagtacctggttctatcaccaatggaatcaagcaaaagctggtatgaaagctctgtgtttctgtcttgcaaatctacaacagcattcaactgcgatgcttggcccgccagaacacccatattacccgtgctgttgtaatacattagaccaataaaattgtccctaaaagcaatgtaattaggcctgttgggcatagattgttggcccattagttctcgtgagttaccttccttaatagtgggcatgtaagaaatatgagtgtctggggtttctatatctacatcttcactgtacaataccactttaggagtcaagttatcaccattgcctgcggctgcctcagtagttgagaaaaattgcatttccacttgactttctagctttccattttgttgctttacaagaatgccttgccctccattttcatttgtgggttttgcatatgaaccgtaacatggtttcattggggtagtcttttttaggactctcccagctgcatgattaatttctgtttcgtaccactgagattctcctatttgaggttcaggttgaaatgttttatcggcatatttaggtgtttgaccttcgacacctatttgaataccctcctttgtaatatttataccagaataaggcgcctgcccaaatacgtgagttttttgctgctcagcttgctcgtctacttcgtcttcgttgtcatcgtcctcttcttctaggtttatttcaagagcagtagcagcttcatcccattcgcaaggatttggggcacccttgggagccagggcgttgtaggcagtgccagagtagggcttaaaagtagggcccctgtccagcacgccgcggatgtcaaagtacgtggaagccatgtccagcacacggttatcacccacagctagggtgaaccgcgccttgtacgagtacgcagtatcctcacggtccacagggatgaaccgcagcgtcaaacgctgggaccggtctgtggtcacgtcgtgcgtaggcgccaccgtggggtttctaaacttgttattcaggctgaagtacgtctcggtggcgcgggcaaactgcaccagcccggggctcaggtactccgaggcgtcctggcccgagatgtgcatgtaagaccactgcggcatcatcgaaggggtagccatcttggaaagcgggcgcgcggcggctcagcagctcctctggcggcgacatggacgcatacatgacacacatacgacacgttagctatcagaagcatcgtcggcgcttcagggattgcacccccagacccacgatgctgttcagtgtgctttgccagttgccactggctacgggccgcaacgatcgcggaccgctggcggcgcggcgcagggacgcgcggctaggacgggttacaacaacggcggtcgggcctggcagcacaggtttctgctgggtgtcggcggggggaggcaggtccagcgttacgggtgtgtgctggcccagcactccggtagccatgggcgcgatgggacgggtggtgggcaggccttgctttagtgcctcctcgtacgagggaggctcgtctatttgcgtcaccagagtttcttccctgtcggggcgcggacgcttttcgccacgcccctctggagacactgtctccacggccggtggaggctcctctacgggagggcggggatcaagcttactgttaatcttattttgcactgcctggttggccaggtccaccaccccgctaatgccagaggccaggccatctaccaccttttgttggaaattttgctctttcaacttatccctcagcatctggcctgtgctgctgttccaggccttgctgccatagttcttaacggtggaaccgaaatttttaatgccgctccacagcgagccccagctgaaggcgccaccgctcatattgctggtgccgatatcttgccagtttcccatgaacgggcgcgagccgtgtcgcggggccagagacgcaaagttgatgtcttccattctacaaaatagttacaggaccaagcgagcgtgagagtccagactttttattttgatttttccacatgcaacttgtttttaatcagtgtctctgcgcctgcaaggccacggatgcaattccgggcacggcgccaatcgccgcggcgatcagtggaataaggaggggcaggataccgccgcgcatgcgacggtgcgacgcgcgccgccgccggtggtgcgcacgacgcatgccgcccgtcaggccgtggccggccatgcccctcctacggtgcattcttcctcggaatcccggcaccgggaaacggaggcggcaggtgagggccatatctgcaagaaccacaaagaccggcttttaaacgatgctggggtggtagcgcgctgttggcagcaccagggtcctgcctccttcgcgagccaccctgcgcacggaaatcggggccagcacgggctggcgacggcgacggcggcggcgggttccagtggtggttcggcgtcgggtagttgctcgtcttctggggcggtaggtgtagccacgatagccgggggtaggcgcaatggaaggatgtagggcatattcgggcagtagcgcgctggcggcgccgtacttcctcgaacggcgcgggcgccggggggctgaaacgcgaaacatccacgggtccgtttgcacctccgtagaggtcttggacgcggccgcagcgaccgcctgcaccgcggcatccgccaccgctgaggcaaccggggacgtttgtgtctccatgccctctgtggcggtggcaatactggtgctactggtagtgggtatctgaacgtccacggtctgcacgcccagtcccggcgccacctgcttgattggccgcacgcggacctcgggctccagcccaggttccacggtcattttttccaagacatcttccagtcgctggcgcttgggtaccatcagctgcacggtgggtgccaagtcaccagactcgcgctttaggccgcgcttttcttcggacggtgcaagcgcgggcagcacctgctgcagtgttacgggctttaggctaggtgttgggttgccctcgtccagcggcaacgccagcatgtccttatgccgctttccgtaggcaaactccccgaggcgctcgttggcctgctcaagcaggtcctcgtcgccgtacacctcatcatacacgcgcttgtaggtgcgggtggagcgctcaccgggcgtaaagactacggtggtgccgggtcgcaaaacacgttttacgcgtcgacctttccactgtacccgtcgcctgggcgcggtagcgtgcagcagttccacctcgtcgtcaagttcatcatcatcatctttctttttctttttgacccgctttagctttcggggcttgtaatcctgctcttccttcttcggggggccatagatctccggcgcgatgacctggagcatctcttctttgattttgcgcttggacatagcttcgttgcgcgccgccgccgctggatacatacaacagtacgagtctaagtagttttttcttgcaatctagttgcgcggggggcgggtgcgcacgggcacgcgcaggccgctaaccgagtcgcgcacccaatacacgttgcccctgcgaccctgagtcatagcactaatggccgcggctgctgcggcggccgctcgtcgcctggacctggggggcacagtgacaatacccgcggccagccttcgagcggcccgcatggccgcccgtcggccggtgcgacgtgcgcggttaagcagggccgccgccgcgcgttgggcggcagtgccgggtcggcggcggtggcgacgtgctacgcgcctccgccgtctcttcattttagcatagcgccgggctccgcgcaccacggtctgaatggccgcgtccactgtggacactggtggcggcgtgggcgtgtagttgcgcgcctcctccaccaccgcgtcgatggcgtcatcgacggtggtgcgcccagtgcggccgcgtttgtgcgcgccccagggcgcgcggtagtgcccgcgcacgcgcactgggtgttggtcggagcgcttcttggccccgccaaacatcttgcttgggaagcgcaggccccagcctgtgttattgctgggcgatataaggatggacatgcttgctcaaaaagtgcggctcgataggacgcgcggcgagactatgcccagggccttgtaaacgtaggggcaggtgcggcgtctggcgtcagtaatggtcactcgctggactcctccgatgctgttgcgcagcggtagcgtcccgtgatctgtgagagcaggaacgttttcactgacggtggtgatggtgggggctggcgggcgcgccaaaatctggttctcgggaaagcgattgaacacgtgggtcagagaggtaaactggcggatgagttgggagtagacggcctggtcgttgtagaagctcttggagtgcacgggcaacagctcggcgcccaccaccggaaagttgctgatctggcgcgtggagcggaaggtcacggggtcttgcatcatgtctggcaacgaccagtagacctgctccgagccgcaggttacgtcaggagtgcaaagcagggtccatgagcggattccggtctgagggtcgccgtagttgtatgcaaggtaccagctgcggtactgggtgaaggtgctgtcattgcttattaggttgtaactgcgtttcttgctgtcctctgtcaggggtttgatcaccggtttcttctgaggcttctcgacctcgggttgcgcagcgggggcggcagcttcggccgctgcttcggcctcagcgcgcttctcctcagcccgtgtggcaaaggtgtcgccgcgaatggcatgatcgttcatgtcctccaccggctgcattgccgcggctgccgcgttggagttctcttccgcgccgctgccactgctgttgctgccgcctgcgccacccccgccctgttcggtgtcatctttcaagctcgcctggtaggcgtccacatccaacagtgcgggaatgttaccaccctccagatcatcgtaggtgatcctaaagccctcctggaagggttgccgcttgcggatgcccaacaagttgctcaggcggctgtgggtgaagtccaccccgcatcctggcagcaaaatgatgtctggatggaaggcttcgtttgtatataccccaggcatgacaagaccagtgacggggtcaaaccccagtctgaagttgcgggtgtcaaactttaccccgatgtcgctttccagaaccccgttctgtctgcccactttcaagtagtgctccacgatcgcgttgttcataaggtctatggtcatggtctcggagtagttgccctcgggcagcgtgaactccacccactcgtatttcagctccacctgattgtccttagtaggcaagcgcgacaccatcacccgcgccttaaacttattggtaaacatgaactcgttcacatttggcatgttggtatgcaggatggttttcaggtcgccgccccagtgcgaccggtcgtcaagattgatggtctgtgtgcttgcctcccccgggctgtagtcattgttttgaatgaccgtggtcagaaagttgctgtggtcgttctggtagttcagggatgccacatccgttgacttgttgtccaccaggtacacacgggtggtgtcgaataggggtgccaactcagagtaacggatgctgtttctccccccggtaggccgcaggtaccgcggaggcacaaacggcgggtccaggggagcatcgaagggagaacccagcgccgccgccactggcgccgcgctcaccacactctcgtaggagggaggaggaccttcctcatacatcgccgcgcgccgcatactaaggggaatacaagaaaaccaacgctcggtgccatggccttggtgagttttttattttgcatcatgctttttttttttttaaaacattctccccagcctggggcgaaggtgcgcaaacgggttgccactccctcccaaatccaggacgctgctgtcgtctgccgagtcatcgtcctcccacaccagaccccgctgacggtcgtgcctttgacgacgggtgggcgggcgcgggcctggcacgtccctgtgctcctgcgcgtacgtcttccatctactcatcttgtccactaggctctctatcccgttgttgggaaatgccggaggcaggtttttttcgcgctgcggctgcagcagcgagttgtttaggtactcctcctcgcccagcaggcgcgggcgggtggtgcgagtgctggtaagagaccctatcaagcttggaaatgggctactagcatctgaccgcggggccgcagcgcctagatcggacaagctgcttggcctgcggaagctttcctttcgcagcgccgcctctgcctgctcgcgctgttgcaactctagcagggtctgcggttgcggggaaaacacgctgtcgtctatgtcgtcccagaggaatccatcgttaccctcgggcacctcgaatcccccggtgtagaaaccagggggcggtagccagtgcgggttcaagatggcattggtgaaatactcggggttcacggcggccgcgcgatgcaagtagtccattaggcggttgataaacggccggtttgaggcatacatgcccggttccatgttgcgcgcggtcatgtccagcgccacgctgggcgttaccccgtcgcgcatcaggttaaggctcacgctctgctgcacgtagcgcaaaatgcgctcctcctcgctgtttaaactgtgcaacgaggggatcttctgccgccggttggtcagcaggtagtttagggttgcctccaggctgcccgtgtcctcctgccccagcgcgcggctgacacttgtaatctcctggaaagtatgctcgtccacatgcgcctgacctatggcctcgcggtacagtgtcagcaagtgacctaggtatgtgtcccgggacacgctgccactgtccgtgaagggcgctattagcagcagcaacaggcgcgagttgggcgtcagcaagctagacacggtcgcgcggtcgcctgtgggagcccgcaccccccacagcccctgcaagtttttgaaagcctggctcaggtttacggtctgcaggccttgtctactggtctggaaaaaatagtctggcccagactggtacacctcactttgcggtgtctcagtcaccattagccgcagtgcgctcacaaagttggtgtagtcctcctgtccccgcggcacgttggcgggctgtgtactcaggaaggcgtttagtgcaaccatggagcccaggttgccctgctgctgcgcgcgctcacgctgcgccacggcctcgcgcacatcccccaccagccggtccaggttggtctgcacgttgccgctgttgtaacgagccacgcgctgaagcagcgcgtcgtagaccaggccggcctcgtcgggccggatggccctgttttcggccagcgcgtttacgatcgccagcaccttctcgtgcgtggggtttgcgcgcgccgggaccaccgcttccagaattgcggagagccggttggcctgcggctgctgccggaacgcgtcaggattgcgcgcagtcagcgacatgatgcggtccatgacctggcgccagtcgtccgtggagttaaggccggacggctggctctgcagcgccgcccgcaccgccgggtccgttgcgtcttgcatcatctgatcagaaacatcaccgcttagtactcgccgtcctctggctcgtactcatcgtcctcgtcatattcctccacgccgccgacgttgccagcgcgcgcgggtgccaccgccagcccaggtccggccccagctgcctccagggcgcgtcggcttggggcccagcgcaggtcagcgcccgcgtcaaagtaggactcggcctctctatcgccgctgcccgtgccagccagggccctttgcaggctgtgcatcagctcgcggtcgctgagctcgcgccgccggctcacgctcacggccttgtggatgcgctcgttgcgataaacgcccaggtcgtcgctcaaggtaagcaccttcagcgccatgcgcatgtagaacccctcgatctttacctccttgtctatgggaacgtaaggggtatggtatatcttgcgggcgtaaaacttgcccaggctaagcatggaatagttgatggcggccaccttgtcagccaggctcaagctgcgctcctgcaccactatgctctgcaggatgtttatcaaatcgagcagccagcggccctcgggctctactatgtttagcagcgcatccctgaatgcctcgttgtccctgctgtgctgcactataaggaacagctgcgccatgagcggcttgctatttgggttttgctccagcgcgcttacaaagtcccacagatgcatcagtcctatagccacctcctcgcgcgccacaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgggctcaaagtcctcctcgcgcagcaaccgctcgcgattcaggccatgccgcagctcgcgccctgcgtggaactttcgatcccgcatctcctcgggctcctctccctcgcggtcgcgaaacaggttctgccgcggcacgtacgcctcacgcgtatcacgcttcagctgcacccttgggtgccgctcaggagagggcgctcctagccgcgccaggccctcgccctcctccaagtccaggtagtgccgggcccggcgccgcgggggttcgtaatcaccatctgctgccgcgtcaaccgcggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttagatgcatcatccaaggacacccccgcggcccaccgcccgccgcgcggtaccgtagtcgcgccgcggggatgcggcctcttgcaagccatcgacgccgccaccaaccagcccctggaaattaggtatcacctggatctagcccgcgccctgacccgtctatgcgaggtaaacctgcaggagctcccgcctgacctgacgccgcgggagctccagaccatggacagctcccatctgcgcgatgttgtcatcaagctccgaccgccgcgcgcggacatctggactttgggctcgcgcggcgtggtggtccgatccaccgtaactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaatagccactcctccaattggtggcgggagatccagttcttcccgatcggctcgcatcctcgcaccgagcgtctctttgtcacctacgatgtagagacctatacttggatgggggcctttgggaagcagctcgtgcccttcatgctggtcatgaagttcggcggagatgagcctctagtgactgccgcgcgagacctagccgcgaaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctttcagagcacgggctcagctcccctgaagagctcacctacgaggaacttaaaaaattgccttccatcaagggcatcccgcgcttcttggaactttacattgtgggccacaacatcaacggctttgacgagatcgtgctcgccgcccaggtaattaacaaccgttccgaggtgccgggacccttccgcatcacacgcaactttatgcctcgcgcgggaaagatactcttcaacgatgtcaccttcgccctgccaaatccgcgttccaaaaagcgcacggactttttgctctgggagcagggcggatgcgacgacactgacttcaaataccagtacctcaaagtcatggtcagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagaccccctaccgccattctgctcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggatgcggccaccctcaaaggcatcaccgcgggccaggtgaatatcaaatcctcctcgtttttggaaactgacaatcttagcgcagaagtcatgcccgcttttcagagggagtactcaccccaacagctggccctcgcagacagcgatgcggaagagagtgaggacgaacgcgcccccacccccttttatagccccccttcaggaacacccggtcacgtggcctacacctacaaaccaatcaccttccttgatgccgaagagggcgacatgtgtcttcacaccctggagcgagtggaccccctagtggacaacgaccgctacccctcccacttagcctccttcgtgctggcctggacgcgagcctttgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgccctcaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagagcaggtgaccgagctgtgggaccgcctggaactgcttggtcaaacgctcaaaagcatgcctacggcggacggtctcaaaccgttgaaaaactttgcttccttgcaagaactgctatcgctgggcggcgagcgccttctggcggatttggtcagggaaaacatgcgagtcagggacatgcttaacgaagtggcccccctgctcagggatgacggcagctgcagctctcttaactaccagttgcagccggtaataggtgtgatttacgggcccaccggctgcggtaagtcgcagctgctcaggaacctgctttcttcccagctgatctcccctaccccggaaaccgttttcttcatcgccccgcaggtagacatgatccccccatctgaactcaaagcgtgggaaatgcaaatctgtgagggtaactacgcccctgggccggatggaaccattataccgcagtctggcaccctccgcccgcgctttgtaaaaatggcctatgacgatctcatcctggaacacaactatgacgttagtgatcccagaaatatcttcgcccaggccgccgcccgtgggcccattgccatcattatggacgaatgcatggaaaatcttggaggtcacaagggcgtctccaagttcttccacgcatttccttctaagctacatgacaaatttcccaagtgcaccggatacactgtgctggtggttctgcacaacatgaatccccggagggatatggctgggaacatagccaacctaaaaatacagtccaagatgcatctcatatccccacgtatgcacccatcccagcttaaccgctttgtaaacacttacaccaagggcctgcccctggcaatcagcttgctactgaaagacatttttaggcaccacgcccagcgctcctgctacgactggatcatctacaacaccaccccgcagcatgaagctctgcagtggtgctacctccaccccagagacgggcttatgcccatgtatctgaacatccagagtcacctttaccacgtcctggaaaaaatacacaggaccctcaacgaccgagaccgctggtcccgggcctaccgcgcgcgcaaaacccctaaataaagacagcaagacacttgcttgatccaaatccaaacagagtctggttttttatttatgttttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttgtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatgatcatcatcgtgatcttcctgatgtgcctgaagatcgtgctgaacaacatcatcatctggagcaccctgaaccagaccgtgttcctgaacaacatcttcaccatcaacgacacctacggcggcctgttctggaacacctactacgacaacaacagaagcaacttcacctactgcggcatcgccggcaactactgcagctgctgcggccacaacatcagcctgtacaacaccaccaacaactgcagcctgatcatcttccccaacaacaccgagatcttcaacagaacctacgagctggtgtacctggacaagaagatcaactacaccgtgaagctgctgaagagcgtggacagccccaccatcacctacaactgcaccaacagcctgatcacctgcaagaacaacaacggcaccaacgtgaacatctacctgatcatcaacaacaccatcgtgaacgacaccaacggcgacatcctgaactactactggaacggcaacaacaacttcaccgccacctgcatgatcaacaacaccatcagcagcctgaacgagaccgagaacatcaactgcaccaaccccatcctgaagtaccagaactacctgagcaccctgttctacatcatcatcttcatcgtgagcggcctgatcatcggcatcttcatcagcatcatcagcgtgctgagcatcagaagaaagagaaagaagcacgtggaggagatcgagagcccaccacccagcgagagcaacgaggaggacatcagccacgacgacaccaccagcatccacgagccaagccccagagaaccactgctgcctaagccctacagcagataccagtacaacacccccatctactacatgagacccagcacccagcccctgaaccccttccccctgcccaagccatgcccgccacctaaaccatgccctccacccaagccttgcccgcccccaaagccatgtccaccacccaaaccttgctctccacccaagccgtgtcgtccccccaaaccatgtcctccaccaaaaccatgtcctccgccgaagccatgcccacctcctaagccatgcccccccagcaagccctgccccagccccgagagctacagcccccccaagcccctgcccagcatccccctgctgcccaacatcccccccctgagcacccagaacatcagcctgatccacgtggacagaatcatctacccatacgacgtcccagactacgcttaagatatcggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaaccgcggccgcgccaccatgttcagcaacaagaagtacatcggcctgatcaacaagaaggagggcctgaagaagaagatcgacgactacagcatcctgatcatcggcatcctgatcggcacaaacatcctgtccctgatcatcaacatcatcggcgagatcaataagcctatctgctaccagaacgatgacaagatcttctactgtcctaaggactgggtgggctacaacaatgtgtgctactacttcggcaacgaggagaagaattacaacaatgccagcaactactgtaagcagctgaatagcaccctgaccaataataacacaatcctggtgaacctgacaaagacactgaacctgacaaaaacatacaaccacgagagcaattactgggtgaactacagcctgatcaagaacgagagcgtgctgctgagggattccggctactacaagaagcagaagcacgtgagcctgctgtacatctgtagcaagtgactcgagtctagagggcccgtttaaacccgctgatcacttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccggaaaaacacctgggcgagtctccacgtaaacggtcaaagtccccgcgggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgataagctatcaaacatgagaattcggcgcgccattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaacgcgcctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcacctgcacaccgcgccttaattaagaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcagcgaaccaatcagcagggtcatcgctagccagatcctctacgccggacgcatcgtggccaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgctgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcata

SEQ ID NO: 33 - DNA, 972 bp, Artificial Sequence
  Synthetic F317L
atggtggagacccagatggacaagctggggttcctgctgaaccacatcggcaagcaggtgaccaccaaggtgctgagcaacgcccacatcacccagaccatgaaggaaatcatcctggagaatcacagcgtggacggcggcgccgccaagaacgtgagcaagggcaagagcagccccaaagaaaaaaagcactggaccgagttcgagagctgggagcagctgagcaagagcaagcgcagcttcaaggagtactgggccgagcgcaacgagatcgtgaacaccctgctgctgaactgggacaacgtgcgcggcgccatcaagaagttcctggacgacgaccgcgagtggtgcggccggatcaacatgatcaacggcgtgcccgagatcgtggagatcatccccagcccctaccgcgccggcgagaacatctacttcggcagcgaggccatgatgcccgccgacatctacagccgcgtggccaacaagcccgccatgttcgtgttccacacccaccccaacctgggcagctgctgcggcggcatgcccagcatctgcgacatcagcaccaccctgcgctacctgctgatgggctggaccgccggccacctgatcatcagcagcaaccaggtgggcatgctgaccgtggacaagcgcatcatcgtggacctgtgggccaacgagaacccccgctggctgatggcccagaagatcctggacatcttcatgatgctgaccagccgccgcagcctggtgaacccctggaccctgcgcgacctgaagaagatcctgcaggactacggcatcgagtacatcatcttccccagcaacgacttcttcatctacgaggacgagcgcctgctgatgttcagcaagaagtggaccaacttcttcaccctgcacgagctgctggacgacctggagaccatcgagaccaaggccagcagcaccacccaccaccaccaccaccactga

SEQ ID NO: 34 - DNA, 495 bp, Artificial Sequence
  Synthetic A151R
atgaacaagaagatcatcgtgatgatggccctgctgcacaaggagaagctgatcgagtgcatctaccacgagctggaaaacggcggaaccatcctgctgctgaccaagaacatcgtggtctccgagatcagctacattggcaacacctacaaatacttcaccttcaacgacaaccacgacctcatcagcaaagaagacctgaagggcgccacctccaaaaacatcgccaaaatgatctacaactggatcatcaaaaacccccagaacaacaagatctggtccggcgaaccccgcacccagatctacttcgagaacgacctgtaccacaccaactacaaccacaaatgcatcaaggacttctggaacgtgagcacctccgtgggcccccacatctttaacgaccgctccatctggtgcaccaagtgcaccagcttttaccccttcaccaacatcatgtcccccaacatcttccagcaccatcaccaccaccactga

SEQ ID NO: 35 - DNA, 1101 bp, Artificial Sequence
  Synthetic P34
atggggaatcgcgggtcttctacctcctctcgccccctgccctcctccgaagccaatatctatgccaagctgcaggaccatatccagcgccagacccgccccttctccggaggaggatactttaacggaggcggcgacaaaaaccccgtgcagcacatcaaggactaccacatcgacagcgtgagctccaaagccaagctcagaatcatcgaaggaattatccgcgccatcgccaagatcggctttaaggtggacacaaaacagcccatcgaagacatcctcaaggacatcaagaaacagctgcccgacccccgcgccggctctacctttgtgaagaacgccgaaaaacaggaaaccgtctgcaagatgattgccgacgccatcaaccaggaattcatcgacctgggccaggacaagctgatcgacaccaccgaaggggccgcctccatctgccgccagatcgtcctctatatcaatagcctgacccacggactgcgggccgaatacctggacgtgcacggcagcatcgagaacaccctggaaaacatcaaactgctgaacgacgccatcaaacagctgcacgaacggatggtgaccgaagtgaccaaggccgcccccaacgaggaagtgattaacgctgtgacaatgatcgaagccgtgtaccgccgcctgctcaacgagcagaacctccagatcaacatcctcaccaacttcatcgacaacatcctgacccccacccagaaagaactggacaagctccagaccgacgaagtcgacatcatcaaactcctcaacgacaccaacagcgtcctcggcaccaaaaacttcggcaaagtgctgagctacaccctctgcaacctgggcatcgccgccagcgtcgccaacaagatcaacaaggccctccagaaagtgggactgaaggtggagcagtatctccagagcaagaactgggccgaattcgacaaagaactcgacctgaaacgcttctccggcctggtgagcgccgagaacatcgccgaattcgagaaggctgtgaacctgctgaggcagaccttcaacgaaaggcacaagatcctggagaacagctgcgccaaaaagggc

SEQ ID NO: 36 - DNA, 1587 bp, Artificial Sequence
  Synthetic pp62
atgcccagcaacatgaagcagttctgcaagatcagcgtgtggctgcagcagcacgaccccgacctcctggagatcatcaacaacctctgcatgctggggaacctgagcgccgccaagtacaagcacggcgtgacattcatctacccaaagcaggccaagatccgcgacgaaattaaaaagcacgcctacagcaacgaccccagccaggccatcaagaccctcgaatccctcatcctcccattctacatccccacccccgccgaattcaccggagagatcggcagctacaccggggtgaagctggaggtggaaaaaaccgaggccaacaaggtgatcctgaagaacggcgaggccgtgctggtgcccgccgccgacttcaagcccttccccgaccgccgcctggccgtgtggatcatggagagcggcagcatgcccctggagggccccccctacaagcgcaagaaggagggcggcggcaacgacccccccgtgcccaagcacatcagcccctacaccccccgcacccgcatcgccatcgaggtggagaaggccttcgacgactgcatgcgccagaactggtgcagcgtgaacaacccctacctggccaagagcgtgagcctgctgagcttcctgagcctgaaccaccccaccgagttcatcaaggtgctgcccctgatcgacttcgaccccctggtgaccttctacctgctgctggagccctacaagacccacggcgacgacttcctgatccccgagaccatcctgttcggccccaccggctggaacggcaccgacctgtaccagagcgccatgctggagttcaagaagttcttcacccagatcacccgccagaccttcatggacatcgccgacagcgccaccaaggaggtggacgtgcccatctgctacagcgaccccgagaccgtgcacagctacgccaaccacgtgcgcaccgagatcctgcaccacaacgccgtgaacaaggtgaccacccccaacctggtggtgcaggcctacaacgagctggagcagaccaacaccatccgccactacggccccatcttccccgagagcaccatcaacgccctgcgcttctggaagaagctgtggcaggacgagcagcgcttcgtgatccacggcctgcaccgcaccctgatggaccagcccacctacgagaccagcgagttcgccgagatcgtgcgcaacctgcgcttcagccgccccggcaacaactacatcaacgagctgaacatcaccagccccgccatgtacggcgacaagcacaccaccggcgacatcgcccccaacgaccgcttcgccatgctggtggccttcatcaacagcaccgacttcctgtacaccgccatccccgaggagaaggtgggcggcaacgagacccagaccagcagcctgaccgacctggtgcccacccgcctgcacagcttcctgaaccacaacctgagcaagctgaagatcctgaaccgcgcccagcagaccgtgcgcaacatcctgagcaacgactgcctgaaccagctgaagcactacgtgaagcacaccggcaagaacgagatcctgaagctgctgtaa

SEQ ID NO: 37 - DNA, 39784 bp, Artificial Sequence
  Synthetic pAd5LCL3-F317L-A151R-P34-pp62
agcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacacgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgatcgattaattaacgacccatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttttttaaaagaaaaggggggattggggggtacagtgcaggggaaagaatagtagacataatagcaacagacatacaaactaaagaattacaaaaacaaattacaaaattcaaaattttatcgtactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggatccgccaccatggggaatcgcgggtcttctacctcctctcgccccctgccctcctccgaagccaatatctatgccaagctgcaggaccatatccagcgccagacccgccccttctccggaggaggatactttaacggaggcggcgacaaaaaccccgtgcagcacatcaaggactaccacatcgacagcgtgagctccaaagccaagctcagaatcatcgaaggaattatccgcgccatcgccaagatcggctttaaggtggacacaaaacagcccatcgaagacatcctcaaggacatcaagaaacagctgcccgacccccgcgccggctctacctttgtgaagaacgccgaaaaacaggaaaccgtctgcaagatgattgccgacgccatcaaccaggaattcatcgacctgggccaggacaagctgatcgacaccaccgaaggggccgcctccatctgccgccagatcgtcctctatatcaatagcctgacccacggactgcgggccgaatacctggacgtgcacggcagcatcgagaacaccctggaaaacatcaaactgctgaacgacgccatcaaacagctgcacgaacggatggtgaccgaagtgaccaaggccgcccccaacgaggaagtgattaacgctgtgacaatgatcgaagccgtgtaccgccgcctgctcaacgagcagaacctccagatcaacatcctcaccaacttcatcgacaacatcctgacccccacccagaaagaactggacaagctccagaccgacgaagtcgacatcatcaaactcctcaacgacaccaacagcgtcctcggcaccaaaaacttcggcaaagtgctgagctacaccctctgcaacctgggcatcgccgccagcgtcgccaacaagatcaacaaggccctccagaaagtgggactgaaggtggagcagtatctccagagcaagaactgggccgaattcgacaaagaactcgacctgaaacgcttctccggcctggtgagcgccgagaacatcgccgaattcgagaaggctgtgaacctgctgaggcagaccttcaacgaaaggcacaagatcctggagaacagctgcgccaaaaagggcggaagcggagctactaacttcagcctgctgaagcaggctggagacgtggaggagaaccctggacctatgcccagcaacatgaagcagttctgcaagatcagcgtgtggctgcagcagcacgaccccgacctcctggagatcatcaacaacctctgcatgctggggaacctgagcgccgccaagtacaagcacggcgtgacattcatctacccaaagcaggccaagatccgcgacgaaattaaaaagcacgcctacagcaacgaccccagccaggccatcaagaccctcgaatccctcatcctcccattctacatccccacccccgccgaattcaccggagagatcggcagctacaccggggtgaagctggaggtggaaaaaaccgaggccaacaaggtgatcctgaagaacggcgaggccgtgctggtgcccgccgccgacttcaagcccttccccgaccgccgcctggccgtgtggatcatggagagcggcagcatgcccctggagggccccccctacaagcgcaagaaggagggcggcggcaacgacccccccgtgcccaagcacatcagcccctacaccccccgcacccgcatcgccatcgaggtggagaaggccttcgacgactgcatgcgccagaactggtgcagcgtgaacaacccctacctggccaagagcgtgagcctgctgagcttcctgagcctgaaccaccccaccgagttcatcaaggtgctgcccctgatcgacttcgaccccctggtgaccttctacctgctgctggagccctacaagacccacggcgacgacttcctgatccccgagaccatcctgttcggccccaccggctggaacggcaccgacctgtaccagagcgccatgctggagttcaagaagttcttcacccagatcacccgccagaccttcatggacatcgccgacagcgccaccaaggaggtggacgtgcccatctgctacagcgaccccgagaccgtgcacagctacgccaaccacgtgcgcaccgagatcctgcaccacaacgccgtgaacaaggtgaccacccccaacctggtggtgcaggcctacaacgagctggagcagaccaacaccatccgccactacggccccatcttccccgagagcaccatcaacgccctgcgcttctggaagaagctgtggcaggacgagcagcgcttcgtgatccacggcctgcaccgcaccctgatggaccagcccacctacgagaccagcgagttcgccgagatcgtgcgcaacctgcgcttcagccgccccggcaacaactacatcaacgagctgaacatcaccagccccgccatgtacggcgacaagcacaccaccggcgacatcgcccccaacgaccgcttcgccatgctggtggccttcatcaacagcaccgacttcctgtacaccgccatccccgaggagaaggtgggcggcaacgagacccagaccagcagcctgaccgacctggtgcccacccgcctgcacagcttcctgaaccacaacctgagcaagctgaagatcctgaaccgcgcccagcagaccgtgcgcaacatcctgagcaacgactgcctgaaccagctgaagcactacgtgaagcacaccggcaagaacgagatcctgaagctgctgtaactcgagtctagagggcccgtttaaacccgctgatcagcctcgataatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgagaagccttagagtctcctcttctgaggcggaaagaaccagctggggctctagcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatctttgagaccgcacagggtcttaataagggtgcagagatcctcaggtccttgacaaggtgagtgaatgcagccttcggtttctaccgagtgctgagttatggtaatgggcttttctcccaccatgaccaccaatttctgacgcttggttggcaacttgtagctaaggcggtgtccggtggtattactgtcgtaggtgactttggcctgctttaccagacaaaagataccccttttgcactggtgcaagttaaccatgtcttggagctcttgattcatgcgctgttgctcggccgctgccctgcgtctttctagcaggcgctgctctgtaataattccgtccatttctagctagagaaacctgaattagaatagcccgtagagttgcttgaattgttcataaaccccacagtagctgcgcctttggcctaataccctaagggttttctaagctcacctcctgttctggtaaacagagttattgaggtctgtccggaaaaagtctggtttacggtcaggcggtaggtgtggtgcagcggccggtgacgcactcgtacgttcccggcaggtaaggagggtggtgttttttctgatggagtagctgagctcggagaggttctctcgtagactcactccgtctgggttgaaactgttgtaaatcacagagggagagatgttaaaagtaccaggtaaggttcgccttggtttgcttgggcgggtgaagacggtggcgtttacaggatggcgataggagccccagtatattttaatttctgtatttattatactcagcacagagatggcaacaaagatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagactagggttctgtgagtttgattaaggtacggtgatctgtataagctatgtggtggtggggctatactactgaatgaaaaatgacttgaaattttctgcaattgaaaaataaacacgttgaaacataacacaaacgatacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggctggtgcacacggaccacgtcaaagacttcaaacaaaacataaagaagggtgggctcgtccatgggatccacctcaaaagtcatgtctagcgcgtgggcggagttggcgtagagaaggttttggcccaggtctgtgagtgcgcccatggacataaagttactggagaatgggatgcgccaaagggtgcgatcgcaaagaaactttttctgggtaatgctgtcaactgcggtcttgcctataagcggataggggaagttagcagggtaggcctgtccttcgcgcatggtgggggcaaggtagccaacaaatccagagttgttgtgttggtgtaggatgcccacctgttggtagtccttgtatttagtatcatccaccacctgacggctcatgggctggaagtttctaaagaaggagtacatgcggtccttgtagctctctgggatatagaagccctggtagccaatgttatagttagctagcatttgtaccaggaaccagtctttggtcatgttacactgggcaacgttgtaaccctccccgtcaactgagcgcttaatttcaaactcgttgggggtaagcaggcggtcattgccaggccagctgacagaagagtcaaaggtaatggccaccttcttaaaggtgtggttgaggtaaaaggttccatctaggtagggtatagagccagagtaggtgtaataagggtcgtagcccgagcccagtgatggggtttccttagtcttaaggcgcgtgaaggcccagccgcggaaagccgcccagttgcgggaggggatggatatgggcacgttggtagcgttggcgggtatagggtagagcatgttggcggcggagagatagtcgttaaaggactggtcgttggtgtcgtttctaagcatggcctcaagcgtggaggcggtgttgtgggccatggggaagaaggtggcgtaaaggcaaatgctatcaaacttaatgctggctccgtcaacccttaggtcatttcctagggagctctgcagaaccatgttaacatccttcctgaagttccactcgtaggtgtatgagcccggcaggagaaggaggtttttaatggcaaagaacttctgaggcacctggatgtggaagggcacatagcgaccattgcccagcaacattgagcggtagcgcaggccagcattgcggtggtggttaaatgggttgacgttgtccatatagtcaagggaccagcgtgctccaaggttaatgtagcagtccactagcccgggagccaccactcgcttgttcatgtagtcgtaggtgtttgggttatcagaaatttttacgttggaaggactgtactttagcttgtcgggcaaatacagcgctatgttggagtacaggaaatttctccacaggttggcatttagattgatttccatggcaaaattatttccaactcttatttcatttttatctgaaaattctgtagcatctttttcccatccattttcctgacctgttttaggttttaccttggtaagagtctctgtattaatcacacctcccagtggaaagcagtaatttggaagttcatcttcagttccatgattttcaataattctaacatctggatcatagctgtcaacagcctgattccacatagaaaagtacctggttctatcaccaatggaatcaagcaaaagctggtatgaaagctctgtgtttctgtcttgcaaatctacaacagcattcaactgcgatgcttggcccgccagaacacccatattacccgtgctgttgtaatacattagaccaataaaattgtccctaaaagcaatgtaattaggcctgttgggcatagattgttggcccattagttctcgtgagttaccttccttaatagtgggcatgtaagaaatatgagtgtctggggtttctatatctacatcttcactgtacaataccactttaggagtcaagttatcaccattgcctgcggctgcctcagtagttgagaaaaattgcatttccacttgactttctagctttccattttgttgctttacaagaatgccttgccctccattttcatttgtgggttttgcatatgaaccgtaacatggtttcattggggtagtcttttttaggactctcccagctgcatgattaatttctgtttcgtaccactgagattctcctatttgaggttcaggttgaaatgttttatcggcatatttaggtgtttgaccttcgacacctatttgaataccctcctttgtaatatttataccagaataaggcgcctgcccaaatacgtgagttttttgctgctcagcttgctcgtctacttcgtcttcgttgtcatcgtcctcttcttctaggtttatttcaagagcagtagcagcttcatcccattcgcaaggatttggggcacccttgggagccagggcgttgtaggcagtgccagagtagggcttaaaagtagggcccctgtccagcacgccgcggatgtcaaagtacgtggaagccatgtccagcacacggttatcacccacagctagggtgaaccgcgccttgtacgagtacgcagtatcctcacggtccacagggatgaaccgcagcgtcaaacgctgggaccggtctgtggtcacgtcgtgcgtaggcgccaccgtggggtttctaaacttgttattcaggctgaagtacgtctcggtggcgcgggcaaactgcaccagcccggggctcaggtactccgaggcgtcctggcccgagatgtgcatgtaagaccactgcggcatcatcgaaggggtagccatcttggaaagcgggcgcgcggcggctcagcagctcctctggcggcgacatggacgcatacatgacacacatacgacacgttagctatcagaagcatcgtcggcgcttcagggattgcacccccagacccacgatgctgttcagtgtgctttgccagttgccactggctacgggccgcaacgatcgcggaccgctggcggcgcggcgcagggacgcgcggctaggacgggttacaacaacggcggtcgggcctggcagcacaggtttctgctgggtgtcggcggggggaggcaggtccagcgttacgggtgtgtgctggcccagcactccggtagccatgggcgcgatgggacgggtggtgggcaggccttgctttagtgcctcctcgtacgagggaggctcgtctatttgcgtcaccagagtttcttccctgtcggggcgcggacgcttttcgccacgcccctctggagacactgtctccacggccggtggaggctcctctacgggagggcggggatcaagcttactgttaatcttattttgcactgcctggttggccaggtccaccaccccgctaatgccagaggccaggccatctaccaccttttgttggaaattttgctctttcaacttatccctcagcatctggcctgtgctgctgttccaggccttgctgccatagttcttaacggtggaaccgaaatttttaatgccgctccacagcgagccccagctgaaggcgccaccgctcatattgctggtgccgatatcttgccagtttcccatgaacgggcgcgagccgtgtcgcggggccagagacgcaaagttgatgtcttccattctacaaaatagttacaggaccaagcgagcgtgagagtccagactttttattttgatttttccacatgcaacttgtttttaatcagtgtctctgcgcctgcaaggccacggatgcaattccgggcacggcgccaatcgccgcggcgatcagtggaataaggaggggcaggataccgccgcgcatgcgacggtgcgacgcgcgccgccgccggtggtgcgcacgacgcatgccgcccgtcaggccgtggccggccatgcccctcctacggtgcattcttcctcggaatcccggcaccgggaaacggaggcggcaggtgagggccatatctgcaagaaccacaaagaccggcttttaaacgatgctggggtggtagcgcgctgttggcagcaccagggtcctgcctccttcgcgagccaccctgcgcacggaaatcggggccagcacgggctggcgacggcgacggcggcggcgggttccagtggtggttcggcgtcgggtagttgctcgtcttctggggcggtaggtgtagccacgatagccgggggtaggcgcaatggaaggatgtagggcatattcgggcagtagcgcgctggcggcgccgtacttcctcgaacggcgcgggcgccggggggctgaaacgcgaaacatccacgggtccgtttgcacctccgtagaggtcttggacgcggccgcagcgaccgcctgcaccgcggcatccgccaccgctgaggcaaccggggacgtttgtgtctccatgccctctgtggcggtggcaatactggtgctactggtagtgggtatctgaacgtccacggtctgcacgcccagtcccggcgccacctgcttgattggccgcacgcggacctcgggctccagcccaggttccacggtcattttttccaagacatcttccagtcgctggcgcttgggtaccatcagctgcacggtgggtgccaagtcaccagactcgcgctttaggccgcgcttttcttcggacggtgcaagcgcgggcagcacctgctgcagtgttacgggctttaggctaggtgttgggttgccctcgtccagcggcaacgccagcatgtccttatgccgctttccgtaggcaaactccccgaggcgctcgttggcctgctcaagcaggtcctcgtcgccgtacacctcatcatacacgcgcttgtaggtgcgggtggagcgctcaccgggcgtaaagactacggtggtgccgggtcgcaaaacacgttttacgcgtcgacctttccactgtacccgtcgcctgggcgcggtagcgtgcagcagttccacctcgtcgtcaagttcatcatcatcatctttctttttctttttgacccgctttagctttcggggcttgtaatcctgctcttccttcttcggggggccatagatctccggcgcgatgacctggagcatctcttctttgattttgcgcttggacatagcttcgttgcgcgccgccgccgctggatacatacaacagtacgagtctaagtagttttttcttgcaatctagttgcgcggggggcgggtgcgcacgggcacgcgcaggccgctaaccgagtcgcgcacccaatacacgttgcccctgcgaccctgagtcatagcactaatggccgcggctgctgcggcggccgctcgtcgcctggacctggggggcacagtgacaatacccgcggccagccttcgagcggcccgcatggccgcccgtcggccggtgcgacgtgcgcggttaagcagggccgccgccgcgcgttgggcggcagtgccgggtcggcggcggtggcgacgtgctacgcgcctccgccgtctcttcattttagcatagcgccgggctccgcgcaccacggtctgaatggccgcgtccactgtggacactggtggcggcgtgggcgtgtagttgcgcgcctcctccaccaccgcgtcgatggcgtcatcgacggtggtgcgcccagtgcggccgcgtttgtgcgcgccccagggcgcgcggtagtgcccgcgcacgcgcactgggtgttggtcggagcgcttcttggccccgccaaacatcttgcttgggaagcgcaggccccagcctgtgttattgctgggcgatataaggatggacatgcttgctcaaaaagtgcggctcgataggacgcgcggcgagactatgcccagggccttgtaaacgtaggggcaggtgcggcgtctggcgtcagtaatggtcactcgctggactcctccgatgctgttgcgcagcggtagcgtcccgtgatctgtgagagcaggaacgttttcactgacggtggtgatggtgggggctggcgggcgcgccaaaatctggttctcgggaaagcgattgaacacgtgggtcagagaggtaaactggcggatgagttgggagtagacggcctggtcgttgtagaagctcttggagtgcacgggcaacagctcggcgcccaccaccggaaagttgctgatctggcgcgtggagcggaaggtcacggggtcttgcatcatgtctggcaacgaccagtagacctgctccgagccgcaggttacgtcaggagtgcaaagcagggtccatgagcggattccggtctgagggtcgccgtagttgtatgcaaggtaccagctgcggtactgggtgaaggtgctgtcattgcttattaggttgtaactgcgtttcttgctgtcctctgtcaggggtttgatcaccggtttcttctgaggcttctcgacctcgggttgcgcagcgggggcggcagcttcggccgctgcttcggcctcagcgcgcttctcctcagcccgtgtggcaaaggtgtcgccgcgaatggcatgatcgttcatgtcctccaccggctgcattgccgcggctgccgcgttggagttctcttccgcgccgctgccactgctgttgctgccgcctgcgccacccccgccctgttcggtgtcatctttcaagctcgcctggtaggcgtccacatccaacagtgcgggaatgttaccaccctccagatcatcgtaggtgatcctaaagccctcctggaagggttgccgcttgcggatgcccaacaagttgctcaggcggctgtgggtgaagtccaccccgcatcctggcagcaaaatgatgtctggatggaaggcttcgtttgtatataccccaggcatgacaagaccagtgacggggtcaaaccccagtctgaagttgcgggtgtcaaactttaccccgatgtcgctttccagaaccccgttctgtctgcccactttcaagtagtgctccacgatcgcgttgttcataaggtctatggtcatggtctcggagtagttgccctcgggcagcgtgaactccacccactcgtatttcagctccacctgattgtccttagtaggcaagcgcgacaccatcacccgcgccttaaacttattggtaaacatgaactcgttcacatttggcatgttggtatgcaggatggttttcaggtcgccgccccagtgcgaccggtcgtcaagattgatggtctgtgtgcttgcctcccccgggctgtagtcattgttttgaatgaccgtggtcagaaagttgctgtggtcgttctggtagttcagggatgccacatccgttgacttgttgtccaccaggtacacacgggtggtgtcgaataggggtgccaactcagagtaacggatgctgtttctccccccggtaggccgcaggtaccgcggaggcacaaacggcgggtccaggggagcatcgaagggagaacccagcgccgccgccactggcgccgcgctcaccacactctcgtaggagggaggaggaccttcctcatacatcgccgcgcgccgcatactaaggggaatacaagaaaaccaacgctcggtgccatggccttggtgagttttttattttgcatcatgctttttttttttttaaaacattctccccagcctggggcgaaggtgcgcaaacgggttgccactccctcccaaatccaggacgctgctgtcgtctgccgagtcatcgtcctcccacaccagaccccgctgacggtcgtgcctttgacgacgggtgggcgggcgcgggcctggcacgtccctgtgctcctgcgcgtacgtcttccatctactcatcttgtccactaggctctctatcccgttgttgggaaatgccggaggcaggtttttttcgcgctgcggctgcagcagcgagttgtttaggtactcctcctcgcccagcaggcgcgggcgggtggtgcgagtgctggtaagagaccctatcaagcttggaaatgggctactagcatctgaccgcggggccgcagcgcctagatcggacaagctgcttggcctgcggaagctttcctttcgcagcgccgcctctgcctgctcgcgctgttgcaactctagcagggtctgcggttgcggggaaaacacgctgtcgtctatgtcgtcccagaggaatccatcgttaccctcgggcacctcgaatcccccggtgtagaaaccagggggcggtagccagtgcgggttcaagatggcattggtgaaatactcggggttcacggcggccgcgcgatgcaagtagtccattaggcggttgataaacggccggtttgaggcatacatgcccggttccatgttgcgcgcggtcatgtccagcgccacgctgggcgttaccccgtcgcgcatcaggttaaggctcacgctctgctgcacgtagcgcaaaatgcgctcctcctcgctgtttaaactgtgcaacgaggggatcttctgccgccggttggtcagcaggtagtttagggttgcctccaggctgcccgtgtcctcctgccccagcgcgcggctgacacttgtaatctcctggaaagtatgctcgtccacatgcgcctgacctatggcctcgcggtacagtgtcagcaagtgacctaggtatgtgtcccgggacacgctgccactgtccgtgaagggcgctattagcagcagcaacaggcgcgagttgggcgtcagcaagctagacacggtcgcgcggtcgcctgtgggagcccgcaccccccacagcccctgcaagtttttgaaagcctggctcaggtttacggtctgcaggccttgtctactggtctggaaaaaatagtctggcccagactggtacacctcactttgcggtgtctcagtcaccattagccgcagtgcgctcacaaagttggtgtagtcctcctgtccccgcggcacgttggcgggctgtgtactcaggaaggcgtttagtgcaaccatggagcccaggttgccctgctgctgcgcgcgctcacgctgcgccacggcctcgcgcacatcccccaccagccggtccaggttggtctgcacgttgccgctgttgtaacgagccacgcgctgaagcagcgcgtcgtagaccaggccggcctcgtcgggccggatggccctgttttcggccagcgcgtttacgatcgccagcaccttctcgtgcgtggggtttgcgcgcgccgggaccaccgcttccagaattgcggagagccggttggcctgcggctgctgccggaacgcgtcaggattgcgcgcagtcagcgacatgatgcggtccatgacctggcgccagtcgtccgtggagttaaggccggacggctggctctgcagcgccgcccgcaccgccgggtccgttgcgtcttgcatcatctgatcagaaacatcaccgcttagtactcgccgtcctctggctcgtactcatcgtcctcgtcatattcctccacgccgccgacgttgccagcgcgcgcgggtgccaccgccagcccaggtccggccccagctgcctccagggcgcgtcggcttggggcccagcgcaggtcagcgcccgcgtcaaagtaggactcggcctctctatcgccgctgcccgtgccagccagggccctttgcaggctgtgcatcagctcgcggtcgctgagctcgcgccgccggctcacgctcacggccttgtggatgcgctcgttgcgataaacgcccaggtcgtcgctcaaggtaagcaccttcagcgccatgcgcatgtagaacccctcgatctttacctccttgtctatgggaacgtaaggggtatggtatatcttgcgggcgtaaaacttgcccaggctaagcatggaatagttgatggcggccaccttgtcagccaggctcaagctgcgctcctgcaccactatgctctgcaggatgtttatcaaatcgagcagccagcggccctcgggctctactatgtttagcagcgcatccctgaatgcctcgttgtccctgctgtgctgcactataaggaacagctgcgccatgagcggcttgctatttgggttttgctccagcgcgcttacaaagtcccacagatgcatcagtcctatagccacctcctcgcgcgccacaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgggctcaaagtcctcctcgcgcagcaaccgctcgcgattcaggccatgccgcagctcgcgccctgcgtggaactttcgatcccgcatctcctcgggctcctctccctcgcggtcgcgaaacaggttctgccgcggcacgtacgcctcacgcgtatcacgcttcagctgcacccttgggtgccgctcaggagagggcgctcctagccgcgccaggccctcgccctcctccaagtccaggtagtgccgggcccggcgccgcgggggttcgtaatcaccatctgctgccgcgtcaaccgcggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttagatgcatcatccaaggacacccccgcggcccaccgcccgccgcgcggtaccgtagtcgcgccgcggggatgcggcctcttgcaagccatcgacgccgccaccaaccagcccctggaaattaggtatcacctggatctagcccgcgccctgacccgtctatgcgaggtaaacctgcaggagctcccgcctgacctgacgccgcgggagctccagaccatggacagctcccatctgcgcgatgttgtcatcaagctccgaccgccgcgcgcggacatctggactttgggctcgcgcggcgtggtggtccgatccaccgtaactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaatagccactcctccaattggtggcgggagatccagttcttcccgatcggctcgcatcctcgcaccgagcgtctctttgtcacctacgatgtagagacctatacttggatgggggcctttgggaagcagctcgtgcccttcatgctggtcatgaagttcggcggagatgagcctctagtgactgccgcgcgagacctagccgcgaaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctttcagagcacgggctcagctcccctgaagagctcacctacgaggaacttaaaaaattgccttccatcaagggcatcccgcgcttcttggaactttacattgtgggccacaacatcaacggctttgacgagatcgtgctcgccgcccaggtaattaacaaccgttccgaggtgccgggacccttccgcatcacacgcaactttatgcctcgcgcgggaaagatactcttcaacgatgtcaccttcgccctgccaaatccgcgttccaaaaagcgcacggactttttgctctgggagcagggcggatgcgacgacactgacttcaaataccagtacctcaaagtcatggtcagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagaccccctaccgccattctgctcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggatgcggccaccctcaaaggcatcaccgcgggccaggtgaatatcaaatcctcctcgtttttggaaactgacaatcttagcgcagaagtcatgcccgcttttcagagggagtactcaccccaacagctggccctcgcagacagcgatgcggaagagagtgaggacgaacgcgcccccacccccttttatagccccccttcaggaacacccggtcacgtggcctacacctacaaaccaatcaccttccttgatgccgaagagggcgacatgtgtcttcacaccctggagcgagtggaccccctagtggacaacgaccgctacccctcccacttagcctccttcgtgctggcctggacgcgagcctttgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgccctcaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagagcaggtgaccgagctgtgggaccgcctggaactgcttggtcaaacgctcaaaagcatgcctacggcggacggtctcaaaccgttgaaaaactttgcttccttgcaagaactgctatcgctgggcggcgagcgccttctggcggatttggtcagggaaaacatgcgagtcagggacatgcttaacgaagtggcccccctgctcagggatgacggcagctgcagctctcttaactaccagttgcagccggtaataggtgtgatttacgggcccaccggctgcggtaagtcgcagctgctcaggaacctgctttcttcccagctgatctcccctaccccggaaaccgttttcttcatcgccccgcaggtagacatgatccccccatctgaactcaaagcgtgggaaatgcaaatctgtgagggtaactacgcccctgggccggatggaaccattataccgcagtctggcaccctccgcccgcgctttgtaaaaatggcctatgacgatctcatcctggaacacaactatgacgttagtgatcccagaaatatcttcgcccaggccgccgcccgtgggcccattgccatcattatggacgaatgcatggaaaatcttggaggtcacaagggcgtctccaagttcttccacgcatttccttctaagctacatgacaaatttcccaagtgcaccggatacactgtgctggtggttctgcacaacatgaatccccggagggatatggctgggaacatagccaacctaaaaatacagtccaagatgcatctcatatccccacgtatgcacccatcccagcttaaccgctttgtaaacacttacaccaagggcctgcccctggcaatcagcttgctactgaaagacatttttaggcaccacgcccagcgctcctgctacgactggatcatctacaacaccaccccgcagcatgaagctctgcagtggtgctacctccaccccagagacgggcttatgcccatgtatctgaacatccagagtcacctttaccacgtcctggaaaaaatacacaggaccctcaacgaccgagaccgctggtcccgggcctaccgcgcgcgcaaaacccctaaataaagacagcaagacacttgcttgatccaaatccaaacagagtctggttttttatttatgttttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttgtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggtggagacccagatggacaagctggggttcctgctgaaccacatcggcaagcaggtgaccaccaaggtgctgagcaacgcccacatcacccagaccatgaaggaaatcatcctggagaatcacagcgtggacggcggcgccgccaagaacgtgagcaagggcaagagcagccccaaagaaaaaaagcactggaccgagttcgagagctgggagcagctgagcaagagcaagcgcagcttcaaggagtactgggccgagcgcaacgagatcgtgaacaccctgctgctgaactgggacaacgtgcgcggcgccatcaagaagttcctggacgacgaccgcgagtggtgcggccggatcaacatgatcaacggcgtgcccgagatcgtggagatcatccccagcccctaccgcgccggcgagaacatctacttcggcagcgaggccatgatgcccgccgacatctacagccgcgtggccaacaagcccgccatgttcgtgttccacacccaccccaacctgggcagctgctgcggcggcatgcccagcatctgcgacatcagcaccaccctgcgctacctgctgatgggctggaccgccggccacctgatcatcagcagcaaccaggtgggcatgctgaccgtggacaagcgcatcatcgtggacctgtgggccaacgagaacccccgctggctgatggcccagaagatcctggacatcttcatgatgctgaccagccgccgcagcctggtgaacccctggaccctgcgcgacctgaagaagatcctgcaggactacggcatcgagtacatcatcttccccagcaacgacttcttcatctacgaggacgagcgcctgctgatgttcagcaagaagtggaccaacttcttcaccctgcacgagctgctggacgacctggagaccatcgagaccaaggccagcagcaccacctacccctacgacgtgcccgactacgcctgagatatcggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaaccgcggccgcatgaacaagaagatcatcgtgatgatggccctgctgcacaaggagaagctgatcgagtgcatctaccacgagctggaaaacggcggaaccatcctgctgctgaccaagaacatcgtggtctccgagatcagctacattggcaacacctacaaatacttcaccttcaacgacaaccacgacctcatcagcaaagaagacctgaagggcgccacctccaaaaacatcgccaaaatgatctacaactggatcatcaaaaacccccagaacaacaagatctggtccggcgaaccccgcacccagatctacttcgagaacgacctgtaccacaccaactacaaccacaaatgcatcaaggacttctggaacgtgagcacctccgtgggcccccacatctttaacgaccgctccatctggtgcaccaagtgcaccagcttttaccccttcaccaacatcatgtcccccaacatcttccagcaccatcaccaccaccactgactcgagtctagagggcccgtttaaacccgctgatcacttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccggaaaaacacctgggcgagtctccacgtaaacggtcaaagtccccgcgggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgataagctatcaaacatgagaattcggcgcgccattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaacgcgcctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcacctgcacaccgcgccttaattaagaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcagcgaaccaatcagcagggtcatcgctagccagatcctctacgccggacgcatcgtggccaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgctgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcata

SEQ ID NO: 38 - RNA, 99 nt, Artificial Sequence
  Synthetic
ggcgggaaaacugaauaagguuuuagagcuagaaauagcaaguuaaaauaaggcuaguccguuaucaacuugaaaaaguggcaccgagucggugcuuuu

SEQ ID NO: 39 - RNA, 99 nt, Artificial Sequence
  Synthetic
gagaugauccagucguagcguuuuagagcuagaaauagcaaguuaaaauaaggcuaguccguuaucaacuugaaaaaguggcaccgagucggugcuuuu

SEQ ID NO: 40 - RNA, 99 nt, Artificial Sequence
  Synthetic
gcgggacauuucagaucggguuuuagagcuagaaauagcaaguuaaaauaaggcuaguccguuaucaacuugaaaaaguggcaccgagucggugcuuuu

SEQ ID NO: 41 - RNA, 99 nt, Artificial Sequence
  Synthetic
guaaggguacugcuaucggguuuuagagcuagaaauagcaaguuaaaauaaggcuaguccguuaucaacuugaaaaaguggcaccgagucggugcuuuu

SEQ ID NO: 42 - DNA, 116 bp, Artificial Sequence
  Synthetic
taatacgactcactatagtactaaacaattccttccgttttagagctagaaatagcaagttaaaataaggctagtccgttatcaacttgaaaaagtggcaccgagtcggtgctttt

SEQ ID NO: 43 - DNA, 116 bp, Artificial Sequence
  Synthetic
taatacgactcactataggttcgcgtgcggttttctgttttagagctagaaatagcaagttaaaataaggctagtccgttatcaacttgaaaaagtggcaccgagtcggtgctttt

SEQ ID NO: 44 - DNA, 33 bp, Artificial Sequence
  Synthetic
taatacgactcactatagtactaaacaattcct

SEQ ID NO: 45 - DNA, 33 bp, Artificial Sequence
  Synthetic
ttctagctctaaaacggaaggaattgtttagta

SEQ ID NO: 46 - DNA, 33 bp, Artificial Sequence
  Synthetic
taatacgactcactataggttcgcgtgcggttt

SEQ ID NO: 47 - DNA, 33 bp, Artificial Sequence
  Synthetic
ttctagctctaaaacagaaaaccgcacgcgaac

SEQ ID NO: 48 - RNA, 99 nt, Artificial Sequence
  Synthetic
guacuaaacaauuccuuccguuuuagagcuagaaauagcaaguuaaaauaaggcuaguccguuaucaacuugaaaaaguggcaccgagucggugcuuuu

SEQ ID NO: 49 - RNA, 99 nt, Artificial Sequence
  Synthetic
gguucgcgugcgguuuucuguuuuagagcuagaaauagcaaguuaaaauaaggcuaguccguuaucaacuugaaaaaguggcaccgagucggugcuuuu

SEQ ID NO: 50 - DNA, 41 bp, Artificial Sequence
  Synthetic
gagtgctactaaacaattccttcctggacccagaatattgg

SEQ ID NO: 51 - DNA, 48 bp, Artificial Sequence
  Synthetic
tggtgttattaccctgttatccctagcaattgaaaaataaacacgttg

SEQ ID NO: 52 - DNA, 422 bp, Artificial Sequence
  Synthetic
tggtgttattaccctgttatccctagcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactc

SEQ ID NO: 53 - DNA, 38 bp, Artificial Sequence
  Synthetic
tagggataacagggtaataacaccactcgacacggcac

SEQ ID NO: 54 - DNA, 44 bp, Artificial Sequence
  Synthetic
ggcgtaggttcgcgtgcggttttctgggtgttttttgtggactt

SEQ ID NO: 55 - DNA, 161 bp, Artificial Sequence
  Synthetic
ggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttattaccctgttatcccta

SEQ ID NO: 56 - DNA, 21 bp, Artificial Sequence
  Synthetic
agtgacgatttgaggaagttg

SEQ ID NO: 57 - DNA, 23 bp, Artificial Sequence
  Synthetic
tcaattgcagaaaatttcaagtc

SEQ ID NO: 58 - DNA, 116 bp, Artificial Sequence
  Synthetic
taatacgactcactatagaggtggcgttcgtaggcagttttagagctagaaatagcaagttaaaataaggctagtccgttatcaacttgaaaaagtggcaccgagtcggtgctttt

SEQ ID NO: 59 - DNA, 116 bp, Artificial Sequence
  Synthetic
taatacgactcactatagccccggtaataaggttcagttttagagctagaaatagcaagttaaaataaggctagtccgttatcaacttgaaaaagtggcaccgagtcggtgctttt

SEQ ID NO: 60 - DNA, 33 bp, Artificial Sequence
  Synthetic
taatacgactcactatagaggtggcgttcgtag

SEQ ID NO: 61 - DNA, 33 bp, Artificial Sequence
  Synthetic
ttctagctctaaaactgcctacgaacgccacct

SEQ ID NO: 62 - DNA, 33 bp, Artificial Sequence
  Synthetic
taatacgactcactatagccccggtaataaggt

SEQ ID NO: 63 - DNA, 33 bp, Artificial Sequence
  Synthetic
ttctagctctaaaactgaaccttattaccgggg

SEQ ID NO: 64 - RNA, 99 nt, Artificial Sequence
  Synthetic
gagguggcguucguaggcaguuuuagagcuagaaauagcaaguuaaaauaaggcuaguccguuaucaacuugaaaaaguggcaccgagucggugcuuuu

SEQ ID NO: 65 - RNA, 99 nt, Artificial Sequence
  Synthetic
gccccgguaauaagguucaguuuuagagcuagaaauagcaaguuaaaauaaggcuaguccguuaucaacuugaaaaaguggcaccgagucggugcuuuu

SEQ ID NO: 66 - DNA, 44 bp, Artificial Sequence
  Synthetic
tgagaataggtggcgttcgtaggcaaggctgacatccgctatgg

SEQ ID NO: 67 - DNA, 44 bp, Artificial Sequence
  Synthetic
tacaattcccaacacatacaagtttccttctcctataggcagaa

SEQ ID NO: 68 - DNA, 23 bp, Artificial Sequence
  Synthetic
acttgtatgtgttgggaattgta

SEQ ID NO: 69 - DNA, 23 bp, Artificial Sequence
  Synthetic
atcgtttgtgttatgtttcaacg

SEQ ID NO: 70 - DNA, 46 bp, Artificial Sequence
  Synthetic
cccacccttgccgtctgcgccgtatcgtttgtgttatgtttcaacg

SEQ ID NO: 71 - DNA, 43 bp, Artificial Sequence
  Synthetic
atggatcacaaccccaccatgaaccttattaccggggtaccca

SEQ ID NO: 72 - DNA, 19 bp, Artificial Sequence
  Synthetic
gttgggctcgcatgtgccg

SEQ ID NO: 73 - DNA, 20 bp, Artificial Sequence
  Synthetic
actcccatggatcacaaccc

SEQ ID NO: 74 - DNA, 42 bp, Artificial Sequence
  Synthetic
taatgcagctggcttatcgaaacgtggaatgcgagaccgtct

SEQ ID NO: 75 - DNA, 20 bp, Artificial Sequence
  Synthetic
gtatctgctccctgcttgtg

SEQ ID NO: 76 - DNA, 20 bp, Artificial Sequence
  Synthetic
cttcgcccaccccaacttgt

SEQ ID NO: 77 - DNA, 21 bp, Artificial Sequence
  Synthetic
agaggtcgacggtatacagac

SEQ ID NO: 78 - DNA, 47 bp, Artificial Sequence
  Synthetic
tgtctgtataccgtcgacctctccgaaaaacacctgggcgagtctcc

SEQ ID NO: 79 - DNA, 56 bp, Artificial Sequence
  Synthetic
acactatagaatacacggaattcttaattaaatcatcaataatataccttattttg

SEQ ID NO: 80 - DNA, 23 bp, Artificial Sequence
  Synthetic
gaattccgtgtattctatagtgt

SEQ ID NO: 81 - DNA, 22 bp, Artificial Sequence
  Synthetic
tttcgataagccagctgcatta

SEQ ID NO: 82 - DNA, 32 bp, Artificial Sequence
  Synthetic
ccggatatctgtcgtcatcatccttatagtcc

SEQ ID NO: 83 - DNA, 36 bp, Artificial Sequence
  Synthetic
aaatatgcggccgcggttgtggccattatcatcgtg

SEQ ID NO: 84 - DNA, 49 bp, Artificial Sequence
  Synthetic
cggatatctcagtggtggtggtgatggtgggtgctgtatctcagcacgg

SEQ ID NO: 85 - DNA, 35 bp, Artificial Sequence
  Synthetic
cgcggatccgccaccatggccagcggcggagcttt

SEQ ID NO: 86 - DNA, 38 bp, Artificial Sequence
  Synthetic
aaatatgcggccgcatggccgaattcaatattgatgaa

SEQ ID NO: 87 - DNA, 50 bp, Artificial Sequence
  Synthetic
cggctcgagtcagtggtggtggtgatggtgggcgtaatcgggcacgtcgt

SEQ ID NO: 88 - DNA, 55 bp, Artificial Sequence
  Synthetic
aggtgacactatagaatacacgttaattaaatcatcaataatataccttattttg

SEQ ID NO: 89 - DNA, 43 bp, Artificial Sequence
  Synthetic
caatccccccttttcttttaaaaaacaccactcgacacggcac

SEQ ID NO: 90 - DNA, 23 bp, Artificial Sequence
  Synthetic
ttttaaaagaaaaggggggattg

SEQ ID NO: 91 - DNA, 21 bp, Artificial Sequence
  Synthetic
tagagccccagctggttcttt

SEQ ID NO: 92 - DNA, 47 bp, Artificial Sequence
  Synthetic
ggaaagaaccagctggggctctagcaattgaaaaataaacacgttga

SEQ ID NO: 93 - DNA, 45 bp, Artificial Sequence
  Synthetic
taatacgactcactatagggagacccaaaatgtaaccactgtgag

SEQ ID NO: 94 - DNA, 22 bp, Artificial Sequence
  Synthetic
tctccctatagtgagtcgtatt

SEQ ID NO: 95 - DNA, 22 bp, Artificial Sequence
  Synthetic
cgtgtattctatagtgtcacct

SEQ ID NO: 96 - DNA, 46 bp, Artificial Sequence
  Synthetic
cgttgaaacataacacaaacgatacggcgcagacggcaagggtggg

SEQ ID NO: 97 - DNA, 37 bp, Artificial Sequence
  Synthetic
cgcggatccgccaccatggacttcatcctgaacatca

SEQ ID NO: 98 - DNA, 31 bp, Artificial Sequence
  Synthetic
ctccgcttccggcgtagtcgggcacgtcgta

SEQ ID NO: 99 - DNA, 41 bp, Artificial Sequence
  Synthetic
acgacgtgcccgactacgccggaagcggagctactaacttc

SEQ ID NO: 100 - DNA, 44 bp, Artificial Sequence
  Synthetic
ctggaagaactcgctgtccataggtccagggttctcctccacgt

SEQ ID NO: 101 - DNA, 31 bp, Artificial Sequence
  Synthetic
ccctggacctatggacagcgagttcttccag

SEQ ID NO: 102 - DNA, 30 bp, Artificial Sequence
  Synthetic
ccgctcgagttagagggagttttccaggtc

SEQ ID NO: 103 - DNA, 32 bp, Artificial Sequence; identical to SEQ ID NO: 82
  Synthetic

SEQ ID NO: 104 - DNA, 36 bp, Artificial Sequence; identical to SEQ ID NO: 83
  Synthetic

SEQ ID NO: 105 - DNA, 44 bp, Artificial Sequence
  Synthetic
aaggaaaaaagcggccgcgccaccatgctggtgatcttcctggg

SEQ ID NO: 106 - DNA, 30 bp, Artificial Sequence
  Synthetic
catgctcgagtcaggcgtagtcaggcacat

SEQ ID NO: 107 - DNA, 35 bp, Artificial Sequence
  Synthetic
cgcggatccgccaccatggagcaccccagcacaaa

SEQ ID NO: 108 - DNA, 44 bp, Artificial Sequence
  Synthetic
gggttttcacgaaaatctgcatggcgtaatcgggcacgtcgtaa

SEQ ID NO: 109 - DNA, 22 bp, Artificial Sequence
  Synthetic
atgcagattttcgtgaaaaccc

SEQ ID NO: 110 - DNA, 32 bp, Artificial Sequence
  Synthetic
ccggatatcttacttgtcttctggtttgttga

SEQ ID NO: 111 - DNA, 55 bp, Artificial Sequence; identical to SEQ ID NO: 88
  Synthetic

SEQ ID NO: 112 - DNA, 43 bp, Artificial Sequence; identical to SEQ ID NO: 89
  Synthetic

SEQ ID NO: 113 - DNA, 23 bp, Artificial Sequence
  Synthetic
ttttaaaagaaaaggggggattg

SEQ ID NO: 114 - DNA, 21 bp, Artificial Sequence
  Synthetic
tagagccccagctggttcttt

SEQ ID NO: 115 - DNA, 47 bp, Artificial Sequence; identical to SEQ ID NO: 92
  Synthetic

SEQ ID NO: 116 - DNA, 45 bp, Artificial Sequence; identical to SEQ ID NO: 93
  Synthetic

SEQ ID NO: 117 - DNA, 22 bp, Artificial Sequence
  Synthetic
tctccctatagtgagtcgtatt

SEQ ID NO: 118 - DNA, 22 bp, Artificial Sequence
  Synthetic
cgtgtattctatagtgtcacct

SEQ ID NO: 119 - DNA, 46 bp, Artificial Sequence; identical to SEQ ID NO: 96
  Synthetic

SEQ ID NO: 120 - DNA, 48 bp, Artificial Sequence
  Synthetic
ccaagctgtgaccggcgcctacggatccgccaccatgacaacccacat

SEQ ID NO: 121 - DNA, 42 bp, Artificial Sequence
  Synthetic
gaagttagtagctccgcttccggcgtaatcaggcacgtcgta

SEQ ID NO: 122 - DNA, 42 bp, Artificial Sequence
  Synthetic
tacgacgtgcctgattacgccggaagcggagctactaacttc

SEQ ID NO: 123 - DNA, 41 bp, Artificial Sequence
  Synthetic
gcccagaaacaccaccagcataggtccagggttctcctcca

SEQ ID NO: 124 - DNA, 20 bp, Artificial Sequence
  Synthetic
atgctggtggtgtttctggg

SEQ ID NO: 125 - DNA, 43 bp, Artificial Sequence
  Synthetic
cgggtttaaacgggccctctagactcgagtcacaggtccttct

SEQ ID NO: 126 - DNA, 20 bp, Artificial Sequence
  Synthetic
tggtgcctcctgaactgcgt

SEQ ID NO: 127 - DNA, 20 bp, Artificial Sequence
  Synthetic
taagggtcaatgtccatgcc

SEQ ID NO: 128 - DNA, 45 bp, Artificial Sequence
  Synthetic
aaggaaaaaagcggccgcgccaccatggtgagcaggtttctgatc

SEQ ID NO: 129 - DNA, 30 bp, Artificial Sequence
  Synthetic
catgctcgagtcaggcgtaatcgggcacat

SEQ ID NO: 130 - DNA, 35 bp, Artificial Sequence
  Synthetic
cgcgatccgccaccatgggcaacagactgatcaag

SEQ ID NO: 131 - DNA, 44 bp, Artificial Sequence
  Synthetic
aagggttttcacgaaaatctgcatggcgtagtcgggcacgtcgt

SEQ ID NO: 132 - DNA, 22 bp, Artificial Sequence
  Synthetic
atgcagattttcgtgaaaaccc

SEQ ID NO: 133 - DNA, 32 bp, Artificial Sequence; identical to SEQ ID NO: 110
  Synthetic

SEQ ID NO: 134 - DNA, 49 bp, Artificial Sequence
  Synthetic
ccaagctgtgaccggcgcctacggatccgccaccatggagacacagaag

SEQ ID NO: 135 - DNA, 43 bp, Artificial Sequence
  Synthetic
agccgctggtggtgttctccatggcgtagtcaggcacatcgta

SEQ ID NO: 136 - DNA, 21 bp, Artificial Sequence
  Synthetic
atggagaacaccaccagcggc

SEQ ID NO: 137 - DNA, 43 bp, Artificial Sequence
  Synthetic
tgaagttagtagctccgcttccgatgtacacccagaggcagaa

SEQ ID NO: 138 - DNA, 21 bp, Artificial Sequence
  Synthetic
ggaagcggagctactaacttc

SEQ ID NO: 139 - DNA, 43 bp, Artificial Sequence
  Synthetic
acaaagtctgttgttccgcccataggtccagggttctcctcca

SEQ ID NO: 140 - DNA, 21 bp, Artificial Sequence
  Synthetic
atgggcggaacaacagacttt

SEQ ID NO: 141 - DNA, 45 bp, Artificial Sequence
  Synthetic
cgggtttaaacgggccctctagactcgagttagatgattctctgc

SEQ ID NO: 142 - DNA, 37 bp, Artificial Sequence
  Synthetic
cgcggatccgccaccatgatcatcatcgtgatcttcc

SEQ ID NO: 143 - DNA, 32 bp, Artificial Sequence
  Synthetic
ccggatatcttaagcgtagtctgggacgtcgt

SEQ ID NO: 144 - DNA, 45 bp, Artificial Sequence
  Synthetic
ataagaatgcggccgcgccaccatgttcagcaacaagaagtacat

SEQ ID NO: 145 - DNA, 30 bp, Artificial Sequence
  Synthetic
aaactcgagtcacttgctacagatgtacag

SEQ ID NO: 146 - DNA, 48 bp, Artificial Sequence
  Synthetic
ccaagctgtgaccggcgcctacggatccgccaccatgtggaaggtgaa

SEQ ID NO: 147 - DNA, 19 bp, Artificial Sequence
  Synthetic
ggcgtaatcgggcacgtcg

SEQ ID NO: 148 - DNA, 43 bp, Artificial Sequence
  Synthetic
ctacgacgtgcccgattacgccggaagcggagctactaacttc

SEQ ID NO: 149 - DNA, 43 bp, Artificial Sequence
  Synthetic
aactgctctctgggctccaccataggtccagggttctcctcca

SEQ ID NO: 150 - DNA, 20 bp, Artificial Sequence
  Synthetic
atggtggagcccagagagca

SEQ ID NO: 151 - DNA, 42 bp, Artificial Sequence
  Synthetic
gggttttcacgaaaatctgcatggcgtaatcgggcacatcgt

SEQ ID NO: 152 - DNA, 22 bp, Artificial Sequence
  Synthetic
atgcagattttcgtgaaaaccc

SEQ ID NO: 153 - DNA, 51 bp, Artificial Sequence
  Synthetic
gggtttaaacgggccctctagactcgagttacttgtcttctggtttgttga

SEQ ID NO: 154 - DNA, 37 bp, Artificial Sequence
  Synthetic
cgcggatccgccaccatggtggagacccagatggaca

SEQ ID NO: 155 - DNA, 30 bp, Artificial Sequence
  Synthetic
ccggatatctcagtggtggtggtggtggtg

SEQ ID NO: 156 - DNA, 38 bp, Artificial Sequence
  Synthetic
aaatatgcggccgcatgaacaagaagatcatcgtgatg

SEQ ID NO: 157 - DNA, 53 bp, Artificial Sequence
  Synthetic
cggctcgagtcagtggtggtggtgatggtgctggaagatgttgggggacatga

SEQ ID NO: 158 - DNA, 56 bp, Artificial Sequence
  Synthetic
tccaagctgtgaccggcgcctacggatccgccaccatggggaatcgcgggtcttct

SEQ ID NO: 159 - DNA, 21 bp, Artificial Sequence
  Synthetic
gccctttttggcgcagctgtt

SEQ ID NO: 160 - DNA, 44 bp, Artificial Sequence
  Synthetic
agaacagctgcgccaaaaagggcggaagcggagctactaacttc

SEQ ID NO: 161 - DNA, 43 bp, Artificial Sequence
  Synthetic
aactgcttcatgttgctgggcataggtccagggttctcctcca

SEQ ID NO: 162 - DNA, 21 bp, Artificial Sequence
  Synthetic
atgcccagcaacatgaagcag

SEQ ID NO: 163 - DNA, 52 bp, Artificial Sequence
  Synthetic
gggtttaaacgggccctctagactcgagttacagcagcttcaggatctcgtt

SEQ ID NO: 164 - DNA, 20 bp, Artificial Sequence
  Synthetic
gctactaaacaattccttcc

SEQ ID NO: 165 - DNA, 20 bp, Artificial Sequence
  Synthetic
aggttcgcgtgcggttttct

SEQ ID NO: 166 - DNA, 20 bp, Artificial Sequence
  Synthetic
ataggtggcgttcgtaggca

SEQ ID NO: 167 - DNA, 20 bp, Artificial Sequence
  Synthetic
taccccggtaataaggttca

SEQ ID NO: 168 - DNA, 46 bp, Artificial Sequence
  Synthetic
acacacaagcagggagcagatacaagggtgggaaagaatatataag

SEQ ID NO: 169 - DNA, 44 bp, Artificial Sequence
  Synthetic
taaacaagttggggtgggcgaagtgatcagcgggtttaaacggg